(19) United States
(12) Patent Application Publication
(10) Pub. No.: US 2026/0200917 A1
(43) Pub. Date: Jul. 16, 2026

(54) TRIAZOLOPYRIDINYL COMPOUNDS AS KINASE INHIBITORS

(71) Applicant: BRISTOL-MYERS SQUIBB COMPANY, Princeton, NJ (US)

(72) Inventors: Jianliang Shi, Furlong, PA (US); Kenneth M. Boy, Southborough, MA (US); Min Cao, Medford, MA (US); Murali T. G. Dhar, Newtown, PA (US); David B. Frennesson, Naugatuck, CT (US); Peiying Liu, Marlborough, MA (US); Varnavas Mouchlis, Waltham, MA (US); Millie May Ness, Somerville, MA (US); Brian Lee Venables, Durham, CT (US); Karin Irmgard Worm, Jackson, NJ (US); Yong-Jin Wu, Madison, CT (US); Zhongxing Zhang, Malden, MA (US)

(21) Appl. No.: 19/447,033

(22) Filed: Jan. 13, 2026

Related U.S. Application Data
(60) Provisional application No. 63/744,890, filed on Jan. 14, 2025.

(51) Int. Cl. C07D 471/04; A61K 31/437; A61K 31/444; A61K 31/497; A61K 31/506; C07B 59/00

(52) U.S. Cl. CPC C07D 471/04; A61K 31/437; A61K 31/444; A61K 31/497; A61K 31/506; C07B 59/002; C07B 2200/05

(57) ABSTRACT

Compounds having formula (I), and enantiomers, and diastereomers, stereoisomers, pharmaceutically acceptable salts thereof,

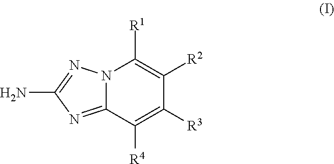

are useful as kinase modulators, including RIPK1 modulation. All the variables are as defined herein.

### CROSS REFERENCE TO RELATED APPLICATION

This application claims the benefit of U.S. Provisional Application No. 63/744,890 filed Jan. 14, 2025, the content of which is hereby incorporated herein by reference in its entirety.

### FIELD OF THE INVENTION

The present invention relates to novel compounds that inhibit receptor interacting protein kinases and methods of making and using the same. Specifically, the present invention relates to triazolopyridinyl compounds as receptor interacting protein kinase 1 (RIPK1) inhibitors.

### BACKGROUND OF THE INVENTION

Apoptosis and necrosis represent two different mechanisms of cell death. Apoptosis is a highly regulated process involving the caspase family of cysteine proteases, and characterized by cellular shrinkage, chromatin condensation, and DNA degradation. In contrast, necrosis is associated with cellular and organelle swelling and plasma membrane rupture with ensuing release of intracellular contents and secondary inflammation (Kroemer et al., (2009) Cell Death Differ 16:3-11). Necrosis has been considered a passive, unregulated form of cell death; however, recent evidence indicates that some necrosis can be induced by regulated signal transduction pathways such as those mediated by receptor interacting protein kinases (RIPKs) especially in conditions where caspases are inhibited or cannot be activated efficiently (Golstein P & Kroemer G (2007) Trends Biochem. Sci. 32:37-43; Festjens et al. (2006) Biochim. Biophys. Acta 1757:1371-1387). Stimulation of the Fas and TNFR family of death domain receptors (DRs) is known to mediate apoptosis in most cell types through the activation of the extrinsic caspase pathway. In addition, in certain cells deficient for caspase-8 or treated with pan-caspase inhibitor Z-VAD, stimulation of death domain receptors (DR) causes a receptor interacting protein kinase 1 (RIPK1) dependent programmed necrotic cell death instead of apoptosis (Holler et al. (2000) Nat. Immunol. 1:489-495; Degterev et al. (2008) Nat. Chem. Biol. 4:313-321). This novel mechanism of cell death is termed “programmed necrosis” or “necroptosis” (Degterev et al., (2005) Nat Chem Biol 1:112-119).

Necroptosis can be triggered by a number of mechanisms including of TNF receptor activation, Toll-like receptor engagement, genotoxic stress and viral infection.

Downstream of the various stimuli, the signaling pathway that results in necroptosis is dependent on RIPK1 and RIPK3 kinase activity. (He et al., (2009) Cell 137:1100-1111; Cho et. al., (2009) Cell 137:1112-1123; Zhang et al., (2009) Science 325:332-336).

Dysregulation of the necroptosis signaling pathway has been linked to neuroinflammation and neurodegeneration such as multiple sclerosis (MS), amyotrophic lateral sclerosis (ALS), Parkinson's and Alzheimer's disease, spinal cord injury, and traumatic brain injury (TBI), detachment of the retina, ischemia, Gaucher's disease, and AAV (ANCA-associated vasculitis) and other inflammatory diseases such as macrophage necrosis in atherosclerosis development, virus induced inflammation, systemic inflammatory response syndrome and ethanol induced liver injury—(Yan et al., (2019) Nature Rev. Neuro. 20, 19-33; Trichonas et al., (2010) Proc. Natl. Acad. Sci. 107, 21695-21700; Lin et al., (2013) Cell Rep. 3, 200-210; Cho et al., (2009) Cell, 137, 1112-1123; Duprez et al., (2011) Immunity 35, 908-918; Roychowdhury et al., Hepatology 57, 1773-1783; Vandenabeele et al., (2010) Nature 10, 700-714; Vandenabeele et al., (2010) Sci. Signaling 3, 1-8; Zhang et al., (2010) Cellular & Mol. Immunology 7, 243-249; Moriwaki et al., (2013) Genes Dev. 27, 1640-1649; Ito et al., (2016) Science 353, 603-608; Vitner et al., (2014) Nature Med. 20, 204-208) (Schreiber et al., (2017) Proc. Natl. Acad. Sci. 114, E9618-E9625). To be useful in the treatment of neurodegenerative diseases, compounds should preferably be brain penetrant. Brain penetrance can be predicted by a brain transporter assay and measured through direct determination of brain concentration.

A potent, selective, small molecule inhibitor of RIPK1 activity would block RIPK1-dependent pro-inflammatory signaling and thereby provide a therapeutic benefit in inflammatory diseases characterized by increased and/or dysregulated RIPK1 kinase activity.

### SUMMARY OF THE INVENTION

The present invention provides novel triazolopyridinyl compounds including stereoisomers, tautomers, isotopes, prodrugs, pharmaceutically acceptable salts, salts, or solvates thereof, which are useful as inhibitors of RIPK1.

The present invention also provides processes and intermediates for making the compounds of the present invention.

The present invention also provides pharmaceutical compositions comprising a pharmaceutically acceptable carrier and at least one of the compounds of the present invention or stereoisomers, tautomers, isotopes, prodrugs, pharmaceutically acceptable salts, salts, or solvates thereof.

The compounds of the invention may be used in the treatment and/or prophylaxis of conditions associated with aberrant RIPK1 activity.

The compounds of the present invention may be used in therapy.

The compounds of the present invention may be used for the manufacture of a medicament for the treatment and/or prophylaxis of a condition associated with aberrant RIPK1 activity.

In another aspect, the present invention is directed to a method of treating diseases mediated at least partially by RIPK1 including inflammatory diseases and neurological inflammatory diseases, which method comprises administering to a patient in need of such treatment a compound of the present invention as described above.

In another aspect, the present invention is directed to a method of treating diseases mediated at least partially by RIPK1 including inflammatory diseases and neurological inflammatory diseases, such as multiple sclerosis (MS), amyotrophic lateral sclerosis (ALS), Parkinson's and Alzheimer's disease, frontotemporal dementia (FTD) spinal cord injury, and traumatic brain injury (TBI), detachment of the retina, ischemia, Gaucher's disease, and AAV (ANCA-associated vasculitis) and other inflammatory diseases such as macrophage necrosis in atherosclerosis development, virus induced inflammation, systemic inflammatory response syndrome and ethanol induced liver injury, which method comprises administering to a patient in need of such treatment a compound of the present invention as described above.

The compounds of the invention can be used alone, in combination with other compounds of the present invention, or in combination with one or more, or with one to two other agent(s).

These and other features of the invention will be set forth in expanded form as the disclosure continues.

### DETAILED DESCRIPTION OF EMBODIMENTS OF THE INVENTION

In one aspect, the present invention provides, inter alia, compounds of Formula (I) or stereoisomers, tautomers, isotopes, salts, pharmaceutically acceptable salts, solvates, or prodrugs thereof, wherein

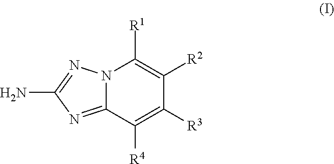

wherein, independently for each occurrence:
R¹ is —H, -T, —F, —OH, C₁₋₃ alkyl, or C₁₋₃ alkoxy;
one of R² or R³ is —H, -T, —F, —OCH₃, or C₁₋₃ alkyl; and the other is

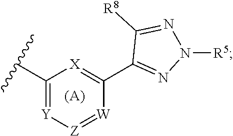

W and Y are —N═ or —C(R¹⁰)═;
Z is —N═ or —C(R¹¹)═;
X is —N═ or —C(R⁹)═;
wherein ring A contains 0-2 nitrogen atoms;
R⁴ is —H, —F, —OH, C₁₋₃ alkyl, or C₁₋₃ alkoxy;
R⁵ is, —(CR^{a}R^{b})—(CR^{c}H)ₙ—R^{5a}
n, at each occurrence, is independently 0 or 1;
R^{a} is —H, -D, —F, C₁₋₄ alkyl, C₃₋₆ cycloalkyl, C₃₋₆ cycloalkyl substituted with 0-2 —F or C₁₋₃ alkyl, C₁₋₃ deuteroalkyl, C₁₋₄ haloalkyl, C₁₋₄ hydroxyalkyl, C₂₋₄ alkoxyalkyl, or oxetanyl;
R^{b} is —H, -D, or C₁₋₃ alkyl;
alternatively, R^{a} and R^{b}, along with the carbon to which they are attached, join to form a C₃₋₆ cycloalkyl substituted with 0-2 —F;
R^{c} is —H, C₁₋₃ alkyl, or —OH;
R^{5a} is C₆₋₁₀ carbocycle or a 5-6 membered heterocycle, the carbocycle or heterocycle being substituted with 0-3 R⁶;
R⁶ is H, -T, —F, —¹⁸F, —Cl, —I, —CN, C₁₋₃ alkyl, C₁₋₃ alkoxy, nitro, C₁₋₃ haloalkyl, or C₁₋₃ haloalkoxy;
R⁸ is —H, -T, -halo, —CN, C₁₋₃ alkyl, C₁₋₃ alkoxy, or C₁₋₃ haloalkyl;
R⁹ is —H, -halo, C₁₋₃ alkyl, C₁₋₃ alkoxy, or C₁₋₃ haloalkyl;
R¹⁰ is —H, -T, -halo, C₁₋₃ alkyl, C₁₋₃ alkoxy, or C₁₋₃ haloalkyl; and
R¹¹ is —H, —CN, —C(O)NH₂, —C(O)OH, -halo, C₁₋₃ alkyl, C₁₋₃ alkoxy, or C₁₋₃ haloalkyl . . . .

Another embodiment provides a compound of Formula (I), or any other aspect or embodiment, or stereoisomers, tautomers, isotopes, salts, pharmaceutically acceptable salts, solvates, or prodrugs thereof, wherein
X and Z are N;
Y is C(R¹⁰); and
W is C(R¹⁰).

Another embodiment provides a compound of Formula (I), or any other aspect or embodiment, or stereoisomers, tautomers, isotopes, salts, pharmaceutically acceptable salts, solvates, or prodrugs thereof, wherein
X is N;
Y is C(R¹⁰);
Z is C(R¹¹); and
W is C(R¹⁰);

Another embodiment provides a compound of Formula (I), or any other aspect or embodiment, or stereoisomers, tautomers, isotopes, salts, pharmaceutically acceptable salts, solvates, or prodrugs thereof, wherein
X is C(R⁹)
Y is C(R¹⁰);
Z is C(R¹¹); and
W is C(R¹⁰);

Another embodiment provides a compound of Formula (I), or any other aspect or embodiment, or stereoisomers, tautomers, isotopes, salts, pharmaceutically acceptable salts, solvates, or prodrugs thereof, wherein
R² is H; and
R³ is

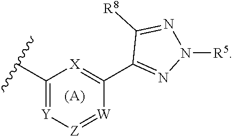

Another embodiment provides a compound of Formula (I), or any other aspect or embodiment, or stereoisomers, tautomers, isotopes, salts, pharmaceutically acceptable salts, solvates, or prodrugs thereof, wherein
R^{5a} is phenyl, pyridyl, oxanyl, tetrahydronaphthalene, or dihydroindene, any of which are substituted with 0-2 R⁶; and
R⁶ is H, —F, —Cl, —CN, C₁₋₃ alkyl, C₁₋₃ alkoxy, or C₁₋₃ haloalkyl.

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is —(CR^{a}R^{b})—R^{5a}; and
R^{a} is —H, -D, C₁₋₄ alkyl, C₁₋₃ alkoxy, or C₁₋₂ haloalkyl.

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is —(CR^{a} H)—R^{5a}.

Another embodiment provides a compound of Formula (I), or any other aspect or embodiment, or stereoisomers, tautomers, isotopes, salts, pharmaceutically acceptable salts, solvates, or prodrugs thereof, wherein
R⁸ is H or CH₃;
R⁹ is H or F;
R¹⁰ is H, F, Cl, CH₃, OCH₃, or CF₃; and
R¹¹ is H, F, Cl, or CF₃.

Another embodiment provides a compound of Formula (I), or any other aspect or embodiment, or stereoisomers, tautomers, isotopes, salts, pharmaceutically acceptable salts, solvates, or prodrugs thereof, wherein
R⁴ is H, —F, or CH₃;
R⁸ is H or CH₃;
R⁹ is H or F;
R¹⁰ is H, CH₃, or CF₃; and
R¹¹ is H.

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is —(CR^{a}R^{b})—R^{5a};
R^{5a} is phenyl or pyridyl, any of which are substituted with 0-2 R⁶;
R⁶ is H, —F, —Cl, CN, CH₃, or OCH₃.

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is

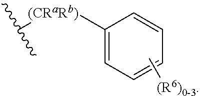

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is

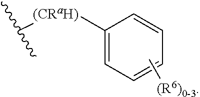

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is

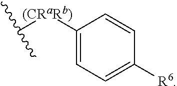

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is

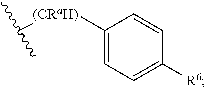

R^{a} is H, CH₃, CH₂CH₃, (CF₂)CH₃, or CH₂CHF₂.

Another embodiment provides a compound of Formula (I), or any other aspect or embodiment, or stereoisomers, tautomers, isotopes, salts, pharmaceutically acceptable salts, solvates, or prodrugs thereof, wherein
R³ is

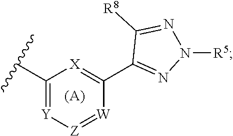

W and Y are —C(R¹⁰)
Z is —C(R¹¹)═.

Another embodiment provides a compound of Formula (I), or any other aspect or embodiment, or stereoisomers, tautomers, isotopes, salts, pharmaceutically acceptable salts, solvates, or prodrugs thereof, wherein
R³ is

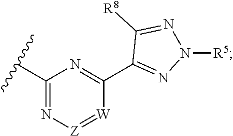

W is —C(R¹⁰)═;
Z is —N═ or —C(R^{U})═.

Another embodiment provides a compound of Formula (I), or any other aspect or embodiment, or stereoisomers, tautomers, isotopes, salts, pharmaceutically acceptable salts, solvates, or prodrugs thereof, wherein
R³ is

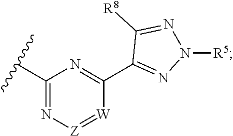

W is —C(R¹⁰)═; and
Z is —C(R¹¹)═.

Another embodiment provides a compound of Formula (I), or any other aspect or embodiment, or stereoisomers, tautomers, isotopes, salts, pharmaceutically acceptable salts, solvates, or prodrugs thereof, wherein
R³ is

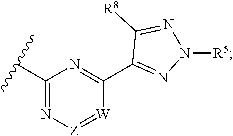

W and Y are —C(R¹⁰)═.

Another embodiment provides a compound of Formula (I), or any other aspect or embodiment, or stereoisomers, tautomers, isotopes, salts, pharmaceutically acceptable salts, solvates, or prodrugs thereof, wherein
R³ is

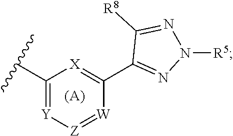

W and Y are —N═ or —C(R¹⁰)═;
Z is —N═ or —C(R¹¹)═;
X is —N═ or —C(R⁹)═.

Another embodiment provides a compound of Formula (I), or any other aspect or embodiment, or stereoisomers, tautomers, isotopes, salts, pharmaceutically acceptable salts, solvates, or prodrugs thereof, wherein
R³ is

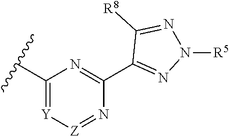

Y is —C(R¹⁰)═; and
Z is —C(R¹¹)═.

Another embodiment provides a compound of Formula (I), or any other aspect or embodiment, or stereoisomers, tautomers, isotopes, salts, pharmaceutically acceptable salts, solvates, or prodrugs thereof, wherein
R¹ is —H or C₁₋₃ alkyl; and
R⁴ is —H, —F, C₁₋₃ alkyl, or C₁₋₃ alkoxy.

Another embodiment provides a compound of Formula (I), or any other aspect or embodiment, or stereoisomers, tautomers, isotopes, salts, pharmaceutically acceptable salts, solvates, or prodrugs thereof, wherein one of R² or R³ is —H, —F, —OCH₃, or —CH₃; and the other is

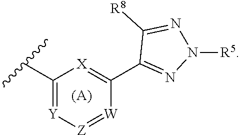

Another embodiment provides a compound of Formula (I), or any other aspect or embodiment, or stereoisomers, tautomers, isotopes, salts, pharmaceutically acceptable salts, solvates, or prodrugs thereof, wherein
W and Y are —N═ or —C(R¹⁰)
Z is —N═ or —C(R¹¹)═; and
X is —N═ or —C(F)═.

Another embodiment provides a compound of Formula (I), or any other aspect or embodiment, or stereoisomers, tautomers, isotopes, salts, pharmaceutically acceptable salts, solvates, or prodrugs thereof, wherein
R¹⁰ is —H, —F, or CH₃;
R^{II} is —H, —F, or CH₃; and
X is —N═ or —C(F)═.

Another embodiment provides a compound of Formula (I), or any other aspect or embodiment, or stereoisomers, tautomers, isotopes, salts, pharmaceutically acceptable salts, solvates, or prodrugs thereof, wherein
R⁸ is —H, or CH₃.

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is —(CR^{a}R^{b})—R^{5a};
R^{a} is —H, C₁₋₂ alkyl, or C₁₋₂ fluoroalkyl;
R is —H;
R^{5a} is phenyl substituted with 0-2 R⁶; and
R⁶ is H, —F, Cl, CN, CH₃, or OCH₃.

Another embodiment provides a compound of Formula (I), or any other aspect or embodiment, or stereoisomers, tautomers, isotopes, salts, pharmaceutically acceptable salts, solvates, or prodrugs thereof, wherein the compound of Formula (I) is

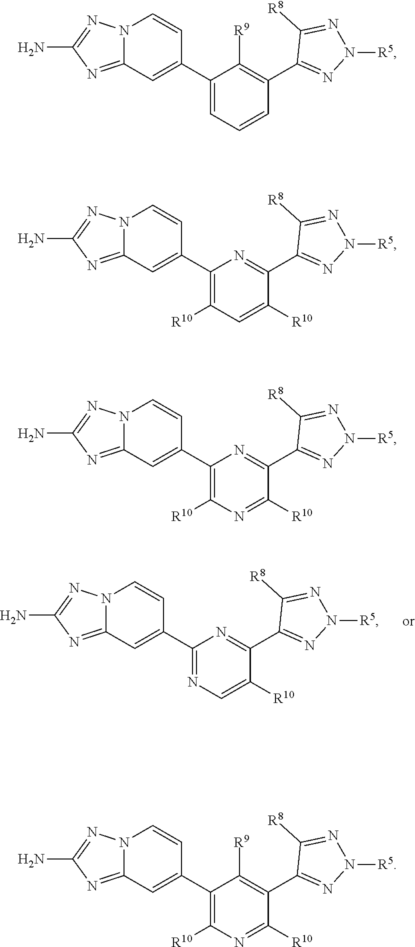

Another embodiment provides a compound of Formula (I), or any other aspect or embodiment, or stereoisomers, tautomers, isotopes, salts, pharmaceutically acceptable salts, solvates, or prodrugs thereof,
wherein R⁵ is

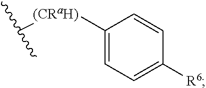

and
R^{a} is C₁₋₃ alkyl, or C₁₋₃ fluoroalkyl;R⁶ is H, —F, —Cl, —CN, C₁₋₃ alkyl, or C₁₋₃ alkoxy.

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is

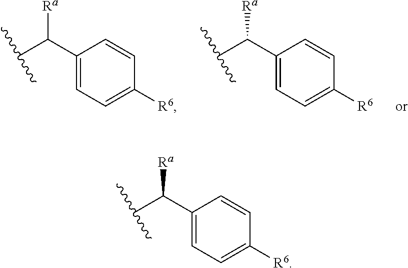

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is

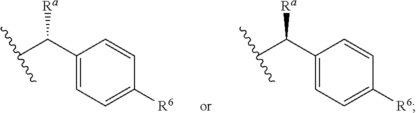

or R⁵ is

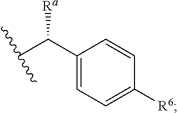

or R⁵ is

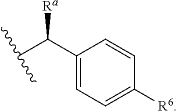

Another embodiment provides a compound of Formula (I), or stereoisomers, tautomers, isotopes, salts, pharmaceutically acceptable salts, solvates, or prodrugs thereof, wherein the compound is selected from the examples.

The present invention is also directed to pharmaceutical compositions useful in treating diseases associated with kinase modulation, including the modulation of receptor interacting protein kinases such as RIPK1, comprising compounds of formula (I), or pharmaceutically acceptable salts thereof, and pharmaceutically acceptable carriers or diluents.

The invention further relates to methods of treating diseases associated with kinase modulation, including the modulation of receptor interacting protein kinases such as RIPK1, comprising administering to a patient in need of such treatment a therapeutically effective amount of a compound according to formula (I), or pharmaceutically acceptable salt thereof.

The present invention also provides processes and intermediates for making the compounds of the present invention or stereoisomers, tautomers, pharmaceutically acceptable salts, solvates, or prodrugs thereof.

The present invention also provides a method for treating proliferative diseases, allergic diseases, autoimmune diseases and inflammatory diseases and fibrotic diseases, comprising administering to a host in need of such treatment a therapeutically effective amount of at least one of the compounds of the present invention or stereoisomers, tautomers, pharmaceutically acceptable salts, solvates, or prodrugs thereof.

The present invention also provides a method for treating a disease, comprising administering to a patient in need of such treatment a therapeutically effective amount of a compound of formula (I), or pharmaceutically acceptable salt thereof, wherein the disease is inflammatory bowel disease, Crohn's disease or ulcerative colitis, psoriasis, systemic lupus erythematosus (SLE), rheumatoid arthritis, multiple sclerosis (MS), Alzheimer's disease (AD), amyotrophic lateral sclerosis (ALS), transplant rejection, nonalcoholic steatohepatitis (NASH), or ischemia reperfusion.

The present invention also provides a method of treating a condition comprising administering to a patient in need of such treatment a therapeutically effective amount of a compound of formula (I), or pharmaceutically acceptable salt thereof, wherein the condition is selected from systemic lupus erythematosus (SLE), multiple sclerosis (MS), Alzheimer's disease (AD), amyotrophic lateral sclerosis (ALS), transplant rejection, acute myelogenous leukemia, chronic myelogenous leukemia, metastatic melanoma, Kaposi's sarcoma, multiple myeloma, solid tumors, ocular neovascularization, and infantile haemangiomas, B cell lymphoma, systemic lupus erythematosus (SLE), psoriatic arthritis, multiple vasculitides, idiopathic thrombocytopenic purpura (ITP), myasthenia gravis, allergic rhinitis, multiple sclerosis (MS), transplant rejection, Type I diabetes, membranous nephritis, autoimmune hemolytic anemia, autoimmune thyroiditis, cold and warm agglutinin diseases, Evan's syndrome, hemolytic uremic syndrome/thrombotic thrombocytopenic purpura (HUS/TTP), sarcoidosis, Sjogren's syndrome, peripheral neuropathies, pemphigus vulgaris and asthma, nonalcoholic steatohepatitis (NASH), or ischemia reperfusion.

The present invention also provides a method of treating a condition comprising administering to a patient in need of such treatment a therapeutically effective amount of a compound of formula (I), or pharmaceutically acceptable salt thereof, wherein the condition is selected from macrophage necrosis in atherosclerosis development, virus induced inflammation, systemic inflammatory response syndrome and ethanol induced liver injury, neurodegeneration such as detachment of the retina, retinal degeneration, wet and dry age-related macular degeneration (AMD), ischemia, amyotrophic lateral sclerosis (ALS), and Gaucher's disease.

The present invention also provides a method for treating a disease, comprising administering to a patient in need of such treatment a therapeutically effective amount of a compound of formula (I), or pharmaceutically acceptable salt thereof, wherein the disease is multiple sclerosis (MS), amyotrophic lateral sclerosis (ALS), Parkinson's and Alzheimer's disease, spinal cord injury, and traumatic brain injury (TBI), detachment of the retina, ischemia, Gaucher's disease, and AAV (ANCA-associated vasculitis) and other inflammatory diseases such as macrophage necrosis in atherosclerosis development, virus induced inflammation, systemic inflammatory response syndrome and ethanol induced liver injury.

The present invention also provides a method of treating a condition comprising administering to a patient in need of such treatment a therapeutically effective amount of a compound of formula (I), or pharmaceutically acceptable salt thereof, wherein the condition is selected from multiple sclerosis (MS), Alzheimer's disease (AD), and amyotrophic lateral sclerosis (ALS), Parkinson's and Alzheimer's disease.

The present invention also provides a method of treating a condition comprising administering to a patient in need of such treatment a therapeutically effective amount of a compound of formula (I), or pharmaceutically acceptable salt thereof, wherein the condition is selected from inflammatory bowel disease, Crohn's disease, ulcerative colitis, and psoriasis. In another embodiment, the condition is selected from inflammatory bowel disease, Crohn's disease, and ulcerative colitis.

The present invention also provides a method of treating a condition comprising administering to a patient in need of such treatment a therapeutically effective amount of a compound of formula (I), or pharmaceutically acceptable salt thereof, wherein the condition is selected from nonalcoholic steatohepatitis (NASH), and ischemia reperfusion.

The present invention also provides a method for treating multiple sclerosis (MS), comprising administering to a patient in need of such treatment a therapeutically effective amount of a compound of formula (I) or pharmaceutically acceptable salt thereof.

The present invention also provides a method for treating Alzheimer's disease (AD), comprising administering to a patient in need of such treatment a therapeutically effective amount of a compound of formula (I) or pharmaceutically acceptable salt thereof.

The present invention also provides a method for treating amyotrophic lateral sclerosis (ALS), comprising administering to a patient in need of such treatment a therapeutically effective amount of a compound of formula (I) or pharmaceutically acceptable salt thereof.

The present invention also provides a method of treating diseases, comprising administering to a patient in need of such treatment a therapeutically effective amount of a compound of formula (I), or pharmaceutically acceptable salt thereof, in combination with other therapeutic agents.

The present invention also provides the compounds of formula (I), embodiments, compounds of the present invention or stereoisomers, tautomers, isotopes, salts, pharmaceutically acceptable salts, solvates, or prodrugs thereof, for use in therapy.

In another embodiment, compounds of formula (I), or pharmaceutically acceptable salt thereof, are selected from exemplified examples or combinations of exemplified examples or other embodiments herein.

The present invention also provides the use of the compounds of the present invention or stereoisomers, tautomers, isotopes, salts, pharmaceutically acceptable salts, solvates, or prodrugs thereof, for the manufacture of a medicament for the treatment of cancers, an allergic disease, an autoimmune disease or an inflammatory disease.

The present invention may be embodied in other specific forms without departing from the spirit or essential attributes thereof. This invention encompasses all combinations of preferred aspects and/or embodiments of the invention noted herein. It is understood that any and all embodiments of the present invention may be taken in conjunction with any other embodiment or embodiments to describe additional embodiments. It is also to be understood that each individual element of the embodiments is its own independent embodiment. Furthermore, any element of an embodiment is meant to be combined with any and all other elements from any embodiment to describe an additional embodiment.

The following are definitions of terms used in this specification and appended claims. The initial definition provided for a group or term herein applies to that group or term throughout the specification and claims, individually or as part of another group, unless otherwise indicated.

Unless specifically stated otherwise herein, references made in the singular may also include the plural. For example, “a” and “an” may refer to either one, or one or more.

When any variable (e.g., R³) occurs more than one time in any constituent or formula for a compound, its definition at each occurrence is independent of its definition at every other occurrence. Thus, for example, if a group is shown to be substituted with 0-2 R³, then said group may optionally be substituted with up to two R³ groups and R³ at each occurrence is selected independently from the definition of R³. Also, combinations of substituents and/or variables are permissible only if such combinations result in stable compounds.

Unless otherwise indicated, any carbon or heteroatom with unsatisfied valences is assumed to have hydrogen atoms sufficient to satisfy the valences.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

In cases wherein there are nitrogen atoms (e.g., amines) on compounds of the present invention, these can be converted to N-oxides by treatment with an oxidizing agent (e.g., MCPBA and/or hydrogen peroxides) to afford other compounds of this invention. Thus, all shown and claimed nitrogen atoms are considered to cover both the shown nitrogen and its N-oxide (N→O) derivative.

In accordance with a convention used in the art,

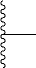

is used in structural formulas herein to depict the bond that is the point of attachment of the moiety or substituent to the core or backbone structure.

A dash “-” that is not between two letters or symbols is used to indicate a point of attachment for a substituent. For example, —CONH₂ is attached through the carbon atom.

The term “optionally substituted” in reference to a particular moiety of the compound of Formula (I), (e.g., an optionally substituted heteroaryl group) refers to a moiety having 0, 1, 2, or more substituents. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “alkyl” or “alkylene” is intended to include both branched and straight-chain saturated aliphatic hydrocarbon groups having the specified number of carbon atoms. For example, “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁-C₆ alkyl” denotes alkyl having 1 to 6 carbon atoms. Alkyl groups can be unsubstituted or substituted so that one or more of its hydrogens are replaced by another chemical group. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

When the term “alkyl” is used together with another group, such as in “arylalkyl”, this conjunction defines with more specificity at least one of the substituents that the substituted alkyl will contain. For example, “arylalkyl” refers to a substituted alkyl group as defined above where at least one of the substituents is an aryl, such as benzyl. Thus, the term aryl(C₀₋₄)alkyl includes a substituted lower alkyl having at least one aryl substituent and also includes an aryl directly bonded to another group, i.e., aryl(C₀)alkyl.

The term “heteroarylalkyl” refers to a substituted alkyl group as defined above where at least one of the substituents is a heteroaryl.

“Alkenyl” or “alkenylene” is intended to include hydrocarbon chains of either straight or branched configuration and having one or more double carbon-carbon bonds that may occur in any stable point along the chain. For example, “C₂₋₆ alkenyl” (or alkenylene), is intended to include C₂, C₃, C₄, C₅, and C₆ alkenyl groups. Examples of alkenyl include, but are not limited to, ethenyl, 1-propenyl, 2-propenyl, 2-butenyl, 3-butenyl, 2-pentenyl, 3, pentenyl, 4-pentenyl, 2-hexenyl, 3-hexenyl, 4-hexenyl, 5-hexenyl, 2-methyl-2-propenyl, 4-methyl-3-pentenyl, and the like.

“Alkynyl” or “alkynylene” is intended to include hydrocarbon chains of either straight or branched configuration and having one or more triple carbon-carbon bonds that may occur in any stable point along the chain. For example, “C₂₋₆ alkynyl” (or alkynylene), is intended to include C₂, C₃, C₄, C₅, and C₆ alkynyl groups; such as ethynyl, propynyl, butynyl, pentynyl, hexynyl and the like.

When reference is made to a substituted alkenyl, alkynyl, alkylene, alkenylene, or alkynylene group, these groups are substituted with one to three substituents as defined above for substituted alkyl groups.

The term “alkoxy” refers to an oxygen atom substituted by alkyl or substituted alkyl, as defined herein. For example, the term “alkoxy” includes the group —O—C₁₋₆alkyl such as methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. “Lower alkoxy” refers to alkoxy groups having one to four carbons.

It should be understood that the selections for all groups, including for example, alkoxy, thioalkyl, and aminoalkyl, will be made by one skilled in the field to provide stable compounds.

The term “substituted”, as used herein, means that any one or more hydrogens on the designated atom or group is replaced with a selection from the indicated group, provided that the designated atom's normal valence is not exceeded. When a substituent is oxo, or keto, (i.e., ═O) then 2 hydrogens on the atom are replaced. Unless otherwise specified, substituents are named into the core structure. For example, it is to be understood that when (cycloalkyl)alkyl is listed as a possible substituent, the point of attachment of this substituent to the core structure is in the alkyl portion. Ring double bonds, as used herein, are double bonds that are formed between two adjacent ring atoms (e.g., C═C, C═N, or N═N).

Combinations of substituents and/or variables are permissible only if such combinations result in stable compounds or useful synthetic intermediates. A stable compound or stable structure is meant to imply a compound that is sufficiently robust to survive isolation from a reaction mixture to a useful degree of purity, and subsequent formulation into an efficacious therapeutic agent. It is preferred that the presently recited compounds do not contain a N-halo, S(O)₂H, or S(O)H group.

The term “carbocyclyl” or “carbocyclic” refers to a saturated or unsaturated, or partially unsaturated, monocyclic or bicyclic ring in which all atoms of all rings are carbon. Thus, the term includes cycloalkyl and aryl rings, or bicyclic rings containing both saturated or unsaturated rings, or rings being partially unsaturated. Monocyclic carbocycles have 3 to 6 ring atoms, still more typically 5 or 6 ring atoms. Bicyclic carbocycles have 7 to 12 ring atoms, e.g., arranged as a bicyclo[4,5], [5,5], [5,6] or [6,6] system, or 9 or 10 ring atoms arranged as a bicyclo[5,6] or [6,6] system. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocycle (e.g., [2.2.2]bicyclooctane). Carbocycles can include cyclopropyl, cyclobutyl, cyclopentyl, cyclohexyl, and phenyl. When the term “carbocycle” is used, it is intended to include “aryl”. A bridged ring occurs when one or more carbon atoms link two nonadjacent carbon atoms. Preferred bridges are one or two carbon atoms. It is noted that a bridge always converts a monocyclic ring into a bicyclic ring. When a ring is bridged, the substituents recited for the ring may also be present on the bridge.

The term “aryl” refers to monocyclic or bicyclic aromatic hydrocarbon groups having 6 to 12 carbon atoms in the ring portion, such as phenyl, and naphthyl groups, each of which may be substituted. A preferred aryl group is optionally substituted phenyl.

The term “cycloalkyl” refers to cyclized alkyl groups, including monocyclic, bicyclic or polycyclic ring systems. C₃₋₇ cycloalkyl is intended to include C₃, C₄, C₅, C₆, and C₇ cycloalkyl groups. Example cycloalkyl groups include, but are not limited to, cyclopropyl, cyclobutyl, cyclopentyl, cyclohexyl, norbornyl, and the like, which optionally may be substituted at any available atoms of the ring(s).

The terms “heterocycloalkyl”, “heterocyclo”, “heterocycle”, “heterocyclic”, or “heterocyclyl” may be used interchangeably and refer to substituted and unsubstituted aromatic or nonaromatic, or partially unsaturated, 3-to 7-membered monocyclic groups, 7-to 11-membered bicyclic groups, and 10-to 15-membered tricyclic groups, in which at least one of the rings has at least one heteroatom (0, S or N), said heteroatom containing ring preferably having 1, 2, or 3 heteroatoms selected from O, S, and N. Each ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The fused rings completing the bicyclic and tricyclic groups may contain only carbon atoms and may be saturated, partially saturated, or unsaturated. The heterocyclo group may be attached at any available nitrogen or carbon atom. The term “heterocycle” includes “heteroaryl” groups. As valence allows, if said further ring is cycloalkyl or heterocyclo it is additionally optionally substituted with ═O (oxo).

Exemplary monocyclic heterocyclyl groups include azetidinyl, pyrrolidinyl, oxetanyl, imidazolinyl, oxazolidinyl, isoxazolinyl, thiazolidinyl, isothiazolidinyl, tetrahydrofuranyl, piperidyl, piperazinyl, 2-oxopiperazinyl, 2-oxopiperidyl, 2-oxopyrrolodinyl, 2-oxoazepinyl, azepinyl, 1-pyridonyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like, including the exemplary groups listed under “heteroaryl”. Exemplary bicyclic heterocyclo groups include quinuclidinyl.

The term “heteroaryl” refers to substituted and unsubstituted aromatic 5-or 6-membered monocyclic groups, 9-or 10-membered bicyclic groups, and 11-to 14-membered tricyclic groups which have at least one heteroatom (0, S or N) in at least one of the rings, said heteroatom-containing ring preferably having 1, 2, or 3 heteroatoms selected from O, S, and N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated.

The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or nonaromatic. The heteroaryl group may be attached at any available nitrogen or carbon atom of any ring. As valence allows, if said further ring is cycloalkyl or heterocyclo it is additionally optionally substituted with ═O (oxo).

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridyl, dihydroisoindolyl, tetrahydroquinolinyl, and the like.

Exemplary tricyclic heteroaryl groups include carbazolyl, benzindolyl, phenanthrollinyl, acridinyl, phenanthridinyl, xanthenyl and the like.

Unless otherwise indicated, when reference is made to a specifically named aryl (e.g., phenyl), cycloalkyl (e.g., cyclohexyl), heterocyclo (e.g., pyrrolidinyl, piperidinyl, and morpholinyl) or heteroaryl (e.g., tetrazolyl, imidazolyl, pyrazolyl, triazolyl, thiazolyl, and furyl) the reference is intended to include rings having 0 to 3, preferably 0-2, substituents, as appropriate.

The term “halo” or “halogen” refers to chloro, bromo, fluoro and iodo.

The term “haloalkyl” means a substituted alkyl having one or more halo substituents. For example, “haloalkyl” includes mono, bi, and trifluoromethyl.

The term “haloalkoxy” means an alkoxy group having one or more halo substituents. For example, “haloalkoxy” includes OCF₃.

The term “deuteroalkyl” means a substituted alkyl having one or more deuterium atom. For example, the term “deuteroalkyl” includes mono, bi, and trideuteromethyl.

The term “heteroatoms” shall include oxygen, sulfur and nitrogen.

When the term “unsaturated” is used herein to refer to a ring or group, the ring or group may be fully unsaturated or partially unsaturated.

One skilled in the field will understand that, when the designation “CO₂” is used herein, this is intended to refer to the group

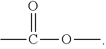

Throughout the specification, groups and substituents thereof may be chosen by one skilled in the field to provide stable moieties and compounds and compounds useful as pharmaceutically acceptable compounds and/or intermediate compounds useful in making pharmaceutically acceptable compounds.

The compounds of formula (I) may exist in a free form (with no ionization) or can form salts which are also within the scope of this invention. Unless otherwise indicated, reference to an inventive compound is understood to include reference to the free form and to salts thereof. The term “salt(s)” denotes acidic and/or basic salts formed with inorganic and/or organic acids and bases. In addition, the term “salt(s) may include zwitterions (inner salts), e.g., when a compound of formula (I), contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt.

However, other salts may be useful, e.g., in isolation or purification steps which may be employed during preparation, and thus, are contemplated within the scope of the invention. Salts of the compounds of the formula (I) may be formed, for example, by reacting a compound of the formula (I) with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. In one embodiment, salts include monohydrochloride, hydrogensulfate, methanesulfonate, phosphate or nitrate salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, PA, 1990, the disclosure of which is hereby incorporated by reference.

The present invention is intended to include all isotopes of atoms occurring in the present compounds. Isotopes include those atoms having the same atomic number but different mass numbers. By way of general example and without limitation, isotopes of hydrogen include deuterium and tritium. As an example, an alkyl substituent is intended to cover alkyl groups have either hydrogen, deuterium, and/or some combination thereof.

Isotopes of carbon include ¹³C and ¹⁴C. Isotopically labeled compounds of the invention can generally be prepared by conventional techniques known to those skilled in the art or by processes analogous to those described herein, using an appropriate isotopically labeled reagent in place of the non-labeled reagent otherwise employed.

Prodrugs and solvates of the inventive compounds are also contemplated. The term “prodrug” denotes a compound which, upon administration to a subject, undergoes chemical conversion by metabolic or chemical processes to yield a compound of the formula (I), and/or a salt and/or solvate thereof. Any compound that will be converted in vivo to provide the bioactive agent (i.e., the compound for formula (I)) is a prodrug within the scope and spirit of the invention. For example, compounds containing a carboxy group can form physiologically hydrolyzable esters which serve as prodrugs by being hydrolyzed in the body to yield formula (I) compounds per se. Such prodrugs are preferably administered orally since hydrolysis in many instances occurs principally under the influence of the digestive enzymes. Parenteral administration may be used where the ester per se is active, or in those instances where hydrolysis occurs in the blood. Examples of physiologically hydrolyzable esters of compounds of formula (I) include C₁₋₆alkylbenzyl, 4-methoxybenzyl, indanyl, phthalyl, methoxymethyl, C₁₋₆alkanoyloxy-C₁₋₆alkyl, e.g. acetoxymethyl, pivaloyloxymethyl or propionyloxymethyl, C₁₋₆alkoxycarbonyloxy-C₁₋₆alkyl, e.g. methoxycarbonyl-oxymethyl or ethoxycarbonyloxymethyl, glycyloxymethyl, phenylglycyloxymethyl, (5-methyl-2-oxo-1,3-dioxolen-4-yl)-methyl and other well-known physiologically hydrolyzable esters used, for example, in the penicillin and cephalosporin arts. Such esters may be prepared by conventional techniques known in the art.

Various forms of prodrugs are well known in the art. For examples of such prodrug derivatives, see:
a) Design of Prodrugs, edited by H. Bundgaard, (Elsevier, 1985) and Methods in Enzymology, Vol. 112, pp. 309-396, edited by K. Widder, et al. (Academic Press, 1985);
b) A Textbook of Drug Design and Development, edited by Krosgaard-Larsen and H. Bundgaard, Chapter 5, “Design and Application of Prodrugs,” by H. Bundgaard, pp. 113-191 (1991); and
c) H. Bundgaard, Advanced Drug Delivery Reviews, Vol. 8, pp. 1-38 (1992), each of which is incorporated herein by reference.

Compounds of the formula (I) and salts thereof may exist in their tautomeric form, in which hydrogen atoms are transposed to other parts of the molecules and the chemical bonds between the atoms of the molecules are consequently rearranged. It should be understood that all tautomeric forms, insofar as they may exist, are included within the invention.

Compounds of this invention may have one or more asymmetric centers. Unless otherwise indicated, all chiral (enantiomeric and diastereomeric) and racemic forms of compounds of the present invention are included in the present invention. Many geometric isomers of olefins, C═N double bonds, and the like can also be present in the compounds, and all such stable isomers are contemplated in the present invention. Cis and trans geometric isomers of the compounds of the present invention are described and may be isolated as a mixture of isomers or as separated isomeric forms. The present compounds can be isolated in optically active or racemic forms. The racemic forms can be resolved by physical methods, such as, for example, fractional crystallization, separation, or crystallization of diastereomeric derivatives or separation by chiral column chromatography. All chiral, (enantiomeric and diastereomeric) and racemic forms and all geometric isomeric forms of a structure are intended, unless the specific stereochemistry or isomer form is specifically indicated. All geometric isomers, tautomers, atropisomers, hydrates, solvates, polymorphs, and isotopically labeled forms of the compounds referred to herein, and mixtures thereof, are considered within the scope of the present invention. Methods of solvation are generally known in the art.

For some examples of the present invention, the absolute stereochemistry of the enantiomers and/or diastereomers has not been specifically identified. However, the racemic mixtures and all enantiomers and diastereomers are included in the present invention. Even where the specific enantiomers and/or diastereomers are isolated, but the absolute stereochemistry was not specifically determined and drawn, one of skill in the art can easily identify and draw the structures of the individual stereoisomers or diastereomers. For example, Example 3 is represented by the structure:

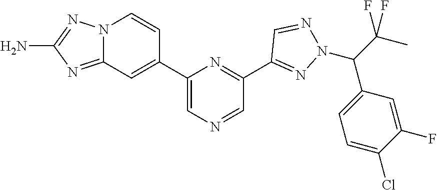 [SMILES: CC(F)(F)C(c1ccc(Cl)c(F)c1)n1ncc(-c2cncc(-c3ccn4nc(N)nc4c3)n2)n1]

The enantiomers are:

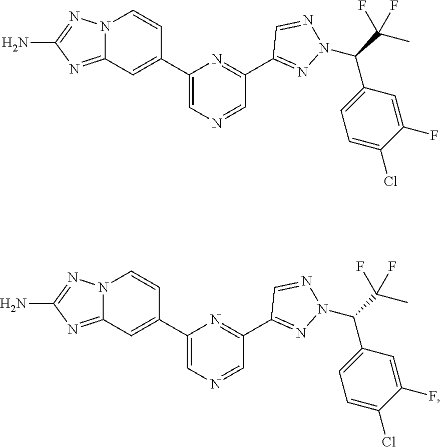 [SMILES: CC(F)(F)[C@@H](c1ccc(Cl)c(F)c1)n1ncc(-c2cncc(-c3ccn4nc(N)nc4c3)n2)n1 ; CC(F)(F)[C@H](c1ccc(Cl)c(F)c1)n1ncc(-c2cncc(-c3ccn4nc(N)nc4c3)n2)n1]

which one of skill in the art is able to identify, even if the structure of each of the examples is not specifically described.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

### Utility

The compounds of the invention modulate kinase activity, including the modulation of RIPK1. Accordingly, compounds of formula (I) have utility in treating conditions associated with the modulation of kinase activity, and particularly the selective inhibition of RIPK1 activity. Compounds of the present invention are brain penetrant and are therefore useful in the treatment of neuroinflammation and neurodegeneration associated diseases.

As used herein, the terms “treating” or “treatment” encompass the treatment of a disease state in a mammal, particularly in a human, and include: (a) preventing or delaying the occurrence of the disease state in a mammal, in particular, when such mammal is predisposed to the disease state but has not yet been diagnosed as having it; (b) inhibiting the disease state, i.e., arresting its development; and/or (c) achieving a full or partial reduction of the symptoms or disease state, and/or alleviating, ameliorating, lessening, or curing the disease or disorder and/or its symptoms.

In view of their activity as inhibitors of RIPK1, compounds of Formula (I) are useful in treating RIPK1-associated conditions including, but not limited to, inflammatory diseases such as Crohn's disease and ulcerative colitis, inflammatory bowel disease, asthma, graft versus host disease, chronic obstructive pulmonary disease; autoimmune diseases such as Graves' disease, rheumatoid arthritis, systemic lupus erythematosis, psoriasis; destructive bone disorders such as bone resorption disease, osteoarthritis, osteoporosis, multiple myeloma related bone disorder; proliferative disorders such as acute myelogenous leukemia, chronic myelogenous leukemia; angiogenic disorders such as angiogenic disorders including solid tumors, ocular neovascularization, and infantile haemangiomas; infectious diseases such as sepsis, septic shock, and Shigellosis; neurodegenerative diseases such as Alzheimer's disease, Parkinson's disease, cerebral ischemias or neurodegenerative disease caused by traumatic injury, multiple sclerosis (MS), amyotrophic lateral sclerosis (ALS), spinal cord injury, and traumatic brain injury (TBI), detachment of the retina, ischemia, Gaucher's disease, and AAV (ANCA-associated vasculitis) and other inflammatory diseases such as macrophage necrosis in atherosclerosis development, virus induced inflammation, systemic inflammatory response syndrome and ethanol induced liver injury, oncologic and viral diseases such as metastatic melanoma, Kaposi's sarcoma, multiple myeloma, and HIV infection and CMV retinitis, AIDS; fibrotic conditions such as, nonalcoholic steatohepatitis (NASH); and cardiac conditions such as, ischemia reperfusion; respectively.

More particularly, the specific conditions or diseases that may be treated with the inventive compounds include, without limitation, pancreatitis (acute or chronic), asthma, allergies, adult respiratory distress syndrome, chronic obstructive pulmonary disease, glomerulonephritis, rheumatoid arthritis, systemic lupus erythematosis, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, ALS, multiple sclerosis, inflammatory bowel disease, ulcerative colitis, Crohn's disease, psoriasis, graft vs. host disease, inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic β-cell disease; diseases characterized by massive neutrophil infiltration; rheumatoid spondylitis, gouty arthritis and other arthritic conditions, cerebral malaria, chronic pulmonary inflammatory disease, silicosis, pulmonary sarcoidosis, bone resorption disease, allograft rejections, fever and myalgias due to infection, cachexia secondary to infection, myeloid formation, scar tissue formation, ulcerative colitis, pyresis, influenza, osteoporosis, osteoarthritis, acute myelogenous leukemia, chronic myelogenous leukemia, metastatic melanoma, Kaposi's sarcoma, multiple myeloma, sepsis, septic shock, and Shigellosis; Alzheimer's disease, Parkinson's disease, cerebral ischemias or neurodegenerative disease caused by traumatic injury; angiogenic disorders including solid tumors, ocular neovascularization, and infantile haemangiomas; viral diseases including acute hepatitis infection (including hepatitis A, hepatitis B and hepatitis C), HIV infection and CMV retinitis, AIDS, ARC or malignancy, and herpes; stroke, myocardial ischemia, ischemia in stroke heart attacks, organ hypoxia, vascular hyperplasia, cardiac and renal reperfusion injury, thrombosis, cardiac hypertrophy, thrombin induced platelet aggregation, endotoxemia and/or toxic shock syndrome, conditions associated with prostaglandin endoperoxidase syndase-2, and pemphigus vulgaris. In another aspect, methods of treatment are those wherein the condition is selected from inflammatory bowel disease, Crohn's disease and ulcerative colitis, allograft rejection, rheumatoid arthritis, psoriasis, ankylosing spondylitis, psoriatic arthritis, and pemphigus vulgaris, and nonalcoholic steatohepatitis (NASH), and ischemia reperfusion. In a further aspect, methods of treatment are those wherein the condition is selected from multiple sclerosis, amyotrophic lateral sclerosis, and Alzheimer's.

Alternatively preferred methods of treatment are those wherein the condition is selected from ischemia reperfusion injury, including cerebral ischemia reperfusions injury arising from stroke and cardiac ischemia reperfusion injury arising from myocardial infarction.

Additionally, due to their brain penetration, compounds of the present invention are useful in the treatment of neurological diseases which are “RIPK1-associated conditions” or “RIPK1-associated disease or disorders”, wherein the condition is selected from multiple sclerosis (MS), Alzheimer's disease (AD), and amyotrophic lateral sclerosis (ALS), Parkinson's, and frontotemporal dementia (FTD) disease.

When the terms “RIPK1-associated condition” or “RIPK1-associated disease or disorder” are used herein, each is intended to encompass all of the conditions identified above as if repeated at length, as well as any other condition that is affected by RIPK1 kinase activity.

The present invention thus provides methods for treating such conditions, comprising administering to a subject in need thereof a therapeutically effective amount of at least one compound of Formula (I) or a salt thereof. “Therapeutically effective amount” is intended to include an amount of a compound of the present invention that is effective when administered alone or in combination to inhibit RIPK1.

The methods of treating RIPK1 kinase associated conditions may comprise administering compounds of Formula (I) alone or in combination with each other and/or other suitable therapeutic agents useful in treating such conditions. Accordingly, “therapeutically effective amount” is also intended to include an amount of the combination of compounds claimed that is effective to inhibit RIPK1 and/or treat diseases associated with RIPK1.

Exemplary of such other therapeutic agents include corticosteroids, rolipram, calphostin, cytokine suppressive anti-inflammatory drugs (CSAIDs), Interleukin-10, glucocorticoids, salicylates, nitric oxide, and other immunosuppressants; nuclear translocation inhibitors, such as deoxyspergualin (DSG); nonsteroidal anti-inflammatory drugs (NSAIDs) such as ibuprofen, celecoxib and rofecoxib; steroids such as prednisone or dexamethasone; anti-inflammatory antibodies such as vedolizumab and ustekinumab, anti-inflammatory kinase inhibitors such as TYK2 inhibitors, antiviral agents such as abacavir; antiproliferative agents such as methotrexate, leflunomide, FK506 (tacrolimus, Prograf); cytotoxic drugs such as azathiprine and cyclophosphamide; TNF-α inhibitors such as tenidap, anti-TNF antibodies or soluble TNF receptor, rapamycin (sirolimus or Rapamune) or derivatives thereof, and agonists of FGF21.

Combination strategies include therapies intended to target the accumulation and/or downstream effects of amyloid beta and/or tau, including but not limited to antibodies that bind to these toxic species, ligand-directed degradation or other degraders such as CELMoD agents. In addition to small molecule and/or antibody-based therapeutic combinations, other options include siRNA and/or ASO approaches as well as the use of delivery technologies designed to augment central exposure to peripherally administered therapeutic agents in addition to ICV or intrathecal administration.

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds. The present invention also provides pharmaceutical compositions capable of treating RIPK1 kinase associated conditions, including IL-1, IL-6, IL-8, IFNγ and TNF-α-mediated conditions, as described above.

The inventive compositions may contain other therapeutic agents as described above and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

Accordingly, the present invention further includes compositions comprising one or more compounds of Formula (I) and a pharmaceutically acceptable carrier.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include without limitation the type and nature of the active agent being formulated; the subject to which the agent containing composition is to be administered; the intended route of administration of the composition; and, the therapeutic indication being targeted. Pharmaceutically acceptable carriers include both aqueous and nonaqueous liquid media, as well as a variety of solid and semisolid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th ed., 1985, which is incorporated herein by reference in its entirety.

The compounds of Formula (I) may be administered by any means suitable for the condition to be treated, which may depend on the need for site-specific treatment or quantity of drug to be delivered. Topical administration is generally preferred for skin related diseases, and systematic treatment preferred for cancerous or precancerous conditions, although other modes of delivery are contemplated. For example, the compounds may be delivered orally, such as in the form of tablets, capsules, granules, powders, or liquid formulations including syrups; topically, such as in the form of solutions, suspensions, gels or ointments; sublingually; bucally; parenterally, such as by subcutaneous, intravenous, intramuscular or intrasternal injection or infusion techniques (e.g., as sterile injectable aqueous or nonaqueous solutions or suspensions); nasally such as by inhalation spray; topically, such as in the form of a cream or ointment; rectally such as in the form of suppositories; or liposomally. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions or, particularly in the case of extended release, with devices such as subcutaneous implants or osmotic pumps.

Exemplary compositions for topical administration include a topical carrier such as PLASTIBASE® (mineral oil gelled with polyethylene).

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may also be orally delivered by sublingual and/or buccal administration, e.g., with molded, compressed, or freeze-dried tablets. Exemplary compositions may include fast dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934©). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

The therapeutically effective amount of a compound of the present invention may be determined by one of ordinary skill in the art, and includes exemplary dosage amounts for a mammal of from about 0.05 to 1000 mg/kg; 1-1000 mg/kg; 1-50 mg/kg; 5-250 mg/kg; 250-1000 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like. Thus, when the term “patient” is used herein, this term is intended to include all subjects, most preferably mammalian species, that are affected by mediation of RIPK1 enzyme levels.

### Biological Assays

Necroptosis is a regulated pathway of cell death that is induced by both inflammatory factors (TNFalpha) as well as viral triggers such as TLR agonists. The process of necroptosis induction occurs following activation and phosphorylation of RIPK1 to form a complex with RIPK3 (referred to as necrosome). Mixed Lineage Kinase domain-like protein (MLKL) is recruited to RIPK3 and is a downstream target of RIPK3 kinase, leading to MLKL phosphorylation at Thr357 and Ser358. Phosphorylated MLKL (pMLKL) leads to MLKL oligomerization, translocation to the plasma membrane, and subsequent pore formation leading to membrane integrity defects (Moriwaki, K., and F. K. Chan. 2013. RIP3: a molecular switch for necrosis and inflammation. Genes Dev. 27: 1640-1649). Thus, understanding the potency of RIPK1 compounds based on RIPK1 direct binding as well as a functional readout of necroptosis activity (pMLKL) is important for evaluating RIPK1 inhibitor activity and potency.

### MLKL Phosphorylation High-Content Assay

HT29-L23 human colorectal adenocarcinoma cells were maintained in RPMI 1640 medium containing 10% heat-inactivated FBS, 1% Penicillin-Streptomycin and 10 mM HEPES. Cells were seeded at 2,000 cells/well in 384 well tissue culture-treated microplates (Greiner #781090-3B) and incubated at 37° C. (5% CO₂/95% O₂) for 2 d. On the day of the assay, the cells were treated with test compounds at final concentrations of 6.25 to 0.106 μM for 30 min at 37° C. (5% CO₂/95% O₂). Necroptosis was induced using a mixture of human TNFα (35 ng/mL) (Peprotech #300-01A), SMAC mimetic (from US 2015/0322111 A1) (700 nM) and Z-VAD (140 nM) (BD pharmingen #51-6936). Following 6 h incubation at 37° C. (5% CO₂/95% O₂), the cells were fixed with 4% formaldehyde (ACROS 11969-0010) for 15 min at rt, then permeabilized with phosphate buffered saline (PBS) containing 0.2% Triton-X-100 for 10 min. MLKL phosphorylation was detected using anti-MLKL (phospho S358) antibody (Abcam #ab187091) (1:1000 dilution in Blocking Buffer [PBS supplemented with 0.1% BSA]) with ON incubation at 4° C. After washing three times in PBS, goat anti-rabbit Alexa-488 (1:1000 dilution) (Life Technologies, A11008) and Hoechst 33342 (Life Technologies, H3570) (1:2000 dilution) in Blocking Buffer were added for 1 h at rt. Following another three cycles of washes in PBS, the microplates were sealed, and cellular images were acquired in the Cellomics ArrayScan VTI high-content imager equipped with an X1 camera. Fluorescent images were taken using a 10× objective and the 386-23 BGRFRN_BGRFRN and 485-20 BGRFRN_BGRFRN filter sets, for nuclei and MLKL phosphorylation, respectively. The image sets were analyzed using the Compartmental Analysis Bioapplication software (Cellomics). The level of MLKL phosphorylation was quantified as MEAN_CircRingAvglntenRatio. The maximal inhibitory response was defined by the activity induced by NecIs (CAS #: 852391-15-2, 6.25 μM). The IC50 value was defined as the concentration of compound that produces 5000 of the maximal inhibition. The data were fitted using the 4-parameter logistic equation to calculate the IC₅₀ and Yₘₐₓ, values.

Compounds were tested in the above identified assays with the following results:

| | LE pMLKL HC |
|---|---|
| Example # | IC50 (nM) |
| (Ent 1)-1 | 0.7 |
| 2 | 4.2 |
| (Ent 1)-3 | 1.5 |
| (Ent 2)-3 | 9.4 |
| (Ent 1)-4 | 0.3 |
| (Ent 2)-4 | 11 |
| (Ent 1)-5 | 7.9 |
| 6 | 3.4 |
| 7 | 12 |
| (Ent 1)-8 | 0.3 |
| (Ent 2)-8 | 40 |
| (Ent-1)-9 | 0.2 |
| (Ent 2)-9 | 48 |
| (Ent 1)-10 | 5.3 |
| (Ent 1)-11 | 2.4 |
| 12 | 1.1 |
| 13 | 1.7 |
| 14 | 9.8 |
| (Ent 1)-15 | 1.5 |
| (Ent 2)-15 | 54 |
| (Ent 2)-16 | 2.7 |
| (Ent 2)-17 | 1.2 |
| (Ent 2)-18 | 1.6 |
| (Ent 1)-19 | 22 |
| 20 | 4.1 |
| (Ent 2)-21 | 8.1 |
| (Ent 2)-22 | 2.3 |
| 23 | 20 |
| (Ent 2)-24 | 7.3 |
| (Ent 1)-25 | 1.3 |
| (Ent 2)-25 | 1.3 |
| (R)-26 | 5.4 |
| (R)-27 | 0.4 |
| (R)-28 | 15 |
| (R)-29 | 0.5 |
| (Ent 1)-30 | 0.3 |
| (Ent 1)-31 | 3.7 |
| (Ent 1)-32 | 1.2 |
| (Ent 2)-32 | 20 |
| (Ent 1)-33 | 1.1 |
| (Ent 2)-33 | 30 |
| (Ent-1)-34 | 8.6 |
| (Ent 2)-34 | 48 |
| (ENT-1)-35 | 810 |
| (ENT-2)-35 | 0.3 |
| (Ent 1)-36 | 4.5 |
| (Ent-2)-36 | 65 |
| (Ent 1)-37 | 90 |
| (Ent 1)-38 | 1.3 |
| (Ent 1)-39 | 11 |
| (Ent 1)-40 | 100 |
| (Ent 2)-41 | 0.9 |
| (Ent 2)-42 | 1.3 |
| (Ent 1)-43 | 1.6 |
| (Ent 2)-43 | 31 |
| (Ent 1)-44 | 7.4 |
| (Ent 1)-45 | 0.2 |
| (Ent 2)-45 | 72 |
| (Ent 1)-46 | 2.5 |
| (Ent 1)-47 | 3.1 |
| (Ent 1)-48 | 0.3 |
| (Ent 2)-48 | 62 |
| (Ent 2)-49 | 2.2 |
| (Ent 1)-50 | 0.8 |
| (Ent 2)-50 | 38 |
| (Ent 1)-51 | 36 |
| (Ent 2)-52 | 3.5 |
| (Ent 1)-53 | 1.1 |
| (Ent 2)-53 | 23 |
| (Ent 1)-54 | 0.9 |
| (Ent 2)-54 | 10 |
| (Ent 1)-55 | 32 |
| (Ent 1)-56 | 23 |
| (Ent 2)-57 | 16 |
| (Ent 2)-58 | 3.1 |
| (Ent 1)-59 | 6.8 |
| (Ent 1)-60 | 0.6 |
| (Ent 1)-61 | 6.5 |
| (Ent 1)-62 | 6.9 |
| (Ent 2)-63 | 66 |
| (R)-64 | 30 |
| (R)-65 | 0.9 |
| (Ent 2)-66 | 88 |
| (R)-67 | 1.2 |
| (R)-68 | 32 |
| 69 | 27 |
| (±)-70 | 2.5 |
| (Ent1)-71 | 2.5 |
| (Ent1)-72 | 33 |
| (Ent 1)-73 | 12 |

### Transporter Assays

Compounds may be assessed as substrates of MDR1 P-glycoprotein (P-gp) and mouse Breast Cancer Resistance Protein (mBcrp) at 0.1 μM, for 2 hours, in bi-directional transport assays using recombinant MIDCK cell lines as described previously (Feng et al. Drug Metabolism and Disposition 2008, Vol. 36:268-27S). MDCK-MDR1 and MDCK-mBcrp cells were acquired from the National Institutes of Health (Bethesda, MD) and the Netherlands Cancer Institute respectively, and used under license agreement. Data are expressed as efflux ratios, where transport across the cell monolayer in the basolateral to apical direction is divided by transport in the apical to basolateral direction. Efflux ratios >2 indicate the test compound is a substrate of the transporter tested.

### Determining Actual Brain Concentrations

Aliquots of brain homogenate can be frozen at −80° C. in polypropylene tubes. The total concentration of Compound A in brain tissue can be measured using liquid chromatography tandem mass spectrometry (LC/MS) analysis. The samples for the LC/MS analysis can be prepared using a protein precipitation procedure described below.

Acetonitrile (100 μL), containing an internal standard [1 mM], along with the sample (30 μL), can be added to a 96-well Filter Plate (Multiscreen Solvinert 0.45 mm Low Binding Hydrophilic PTFE, Millipore) fitted on top of the final autosampler plate (96-well 1 ml Collection Plate, Waters) including an insert (Ultra Amp 96-well 0.2 ml Ultraplate, Sorenson Bioscience). The assembly can be vortex mixed for 5 min at room temperature, centrifuged at 3,700 rpm at 4° C. for 10 min, then capped. The supernatant (3 μL) can be injected to an Ultra Performance LC System (Waters Acquity iClass) interfaced with a Quadrapole MS/MS (Thermo Scientific TSQ Quantiva) tandem mass spectrometer. The analyte can be separated on a C₁₈ column (Waters Acquity HSS T3, 2.1×50 mm, 1.8 μm) at 40° C., with a gradient flow rate of 0.7 ml/min, consisting of two buffer solutions (A: water, 5 mM Ammonium Formate, 0.1% formic acid; B: acetonitrile, 0.1% formic acid). The detection is possible by using multiple reaction monitoring (MRM) in the positive electrospray ionization mode, representing the precursor (M+H)+species.

The assays described above (transporter and brain assay) may be used to illustrate that the compounds of the present invention are brain penetrant, and therefore are useful for the treatment of neurodegenerative diseases as described above.

### Methods of Preparation

Compounds of Formula (I), and intermediates used in the preparation of compounds of Formula (I), can be prepared using procedures shown in the following examples and related procedures. The methods and conditions used in these examples, and the actual compounds prepared in these examples, are not meant to be limiting, but are meant to demonstrate how the compounds of Formula (I) can be prepared. Starting materials and reagents used in these examples, when not prepared by a procedure described herein, are generally either commercially available, or are reported in the chemical literature, or may be prepared by using procedures described in the chemical literature.

Abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “L” for microliter or microliters, “N” for normal, “M” for molar, “mmol” for millimole or millimoles, “ρmol” for micromole or micromoles, “min” for minute or minutes, “h” for hour or hours, “RT” for room temperature, “ON” for overnight, “atm” for atmosphere, “psi” for pounds per square inch, “conc.” for concentrate, “approx.” for approximate or approximately, “sat” or “saturated” for saturated, “CV” or “CVs” for column volume or column volumes, “MW” for molecular weight, “mp” for melting point, “ee” for enantiomeric excess, “MS” or “Mass Spec” for mass spectrometry, “m/z” for mass per unit charge, “ESI” for electrospray ionization mass spectroscopy, “HR” for high resolution, “HRMS” for high resolution mass spectrometry, “APCI” for atmospheric pressure chemical ionization, “LCMS” or “LC/MS” for liquid chromatography mass spectrometry, “HPLC” for high pressure liquid chromatography, “RP HPLC” for reverse phase HPLC, “prep” for preparative, “SFC” for supercritical fluid chromatography, “TLC” or “tlc” for thin layer chromatography, “Rf” for retention factor, “UV” for ultraviolet, “NMR” for nuclear magnetic resonance spectroscopy, “nOe” for nuclear Overhauser effect spectroscopy, “¹H” for proton, “6” for delta, “s” for singlet, “d” for doublet, “t” for triplet, “q” for quartet, “m” for multiplet, “br” for broad, “MHz” for megahertz, and “a”, “p”, “R”, “S”, “E”, and “Z” are stereochemical designations familiar to one skilled in the art.
Me methyl
Et ethyl
Pr propyl
i-Pr isopropyl
Bu butyl
n-BuLi n-butyllithium
i-Bu isobutyl
t-Bu tert-butyl
Ph phenyl
Bn benzyl
BISPIN bis(pinacolato)diboron
Boc tert-butyloxycarbonyl
AcOH or HOAc acetic acid
BOP benzotriazol-1-yloxytris(dimethylamino)phosphonium hexafluorophosphate
CBz carbobenzyloxy
DAST Diethylaminosulfur trifluoride
DCE 1,2-dichloroethane
DCM dichloromethane
DEA diethylamine
DIAD Diisopropyl azodicarboxylate
DIEA/DIPEA/Hünig's Base diisopropylethylamine
DMAP 4-dimethylaminopyridine
DME 1,2-dimethoxyethane
DMF dimethyl formamide
DMSO dimethyl sulfoxide
EDC/EDCI N-(3-dimthylaminopropyl)-N′-ethylcarbodiimide
Et₃N or TEA triethylamine
EtOAc ethyl acetate
Et₂O diethyl ether
EtOH ethanol
HCl hydrochloric acid
HATU 0-(7-azabenzotriazol-1-yl)-N,N,N′,N′-tetramethyluronium hexafluorophosphate
Hex hexane
HOBt or HOBT 1-hydroxybenzotriazole
i-PrOH or IPA isopropanol
KOAc potassium acetate
LAH lithium aluminum hydride
LDA lithium diisopropylamide
LG leaving group
MeCN or ACN acetonitrile
MeOH methanol
Mel iodomethane
MgSO₄ magnesium sulfate
NBS N-bromosuccinimide
NCS N-chlorosuccinimide
NH₄OAc ammonium acetate
NIS N-iodosuccinimide
NH₄OH ammonium hydroxide
OTf triflate or trifluoromethanesulfonate
Pd₂(dba)₃ tris(dibenzylideneacetone)dipalladium(0)
Pd(OAc)₂ palladium(II) acetate
Pd/C palladium on carbon
PdCl₂(dtbpf) [1,1′-Bis(di-tert-butylphosphino)ferrocene]dichloropalladium(II)
Pd(dppf)Cl₂ [1,1′-bis(diphenylphosphino)-ferrocene]dichloropalladium(II)
PG protecting group
PTFE polytetrafluoroethylene or Teflon
SiO₂ silica oxide or silica gel
S-Phos or SPhos dicyclohexyl(2′,6′-dimethoxy-[1,1′-biphenyl]-2-yl)phosphine
TBAF tetra-N-butylammonium fluoride
TBAI tetra-N-butylammonium iodide
TFA trifluoroacetic acid
THF tetrahydrofuran
X-Phos or XPhos 2-dicyclohexylphosphino-2′,4′,6′-triisopropylbiphenyl
XPhos Pd G2 2nd generation XPhos precatalyst, chloro(2-dicyclohexylphosphino-2′,4′,6′-triisopropyl-1,1′-biphenyl)[2-(2′-amino-1,1′-biphenyl)]palladium(II)

The compounds of the present invention may be synthesized by many methods available to those skilled in the art of organic chemistry (Maffrand, J. P. et al., Heterocycles, 16(1):35-7 (1981)). General synthetic schemes for preparing compounds of the present invention are described below. These schemes are illustrative and are not meant to limit the possible techniques one skilled in the art may use to prepare the compounds disclosed herein. The numbering of R groups within the scheme are for illustrative purposes and are not intended to limit the claims. Different methods to prepare the compounds of the present invention will be evident to those skilled in the art. Additionally, the various steps in the synthesis may be performed in an alternate sequence in order to give the desired compound or compounds.

Examples of compounds of the present invention prepared by methods described in the general schemes are given in the intermediates and examples section set out hereinafter. Example compounds are typically prepared as racemic mixtures. Preparation of homochiral examples may be carried out by techniques known to one skilled in the art. For example, homochiral compounds may be prepared by separation of racemic products by chiral phase preparative HPLC or SFC. Alternatively, the example compounds may be prepared by methods known to give enantiomerically enriched products. These include, but are not limited to, the incorporation of chiral auxiliary functionalities into racemic intermediates which serve to control the diastereoselectivity of transformations, providing enantio-enriched products upon cleavage of the chiral auxiliary.

Scheme 1 illustrates two possible approaches to the synthesis of intermediates exemplified by 3. In Method 1, a Suzuki coupling reaction between 1-alkyl-4-halotriazoles 1 and haloarylboronic acids 2 (Miyaura, N. and Suzuki, A. Chemical Reviews, 95:2457-2483, 1995) can be performed to provide intermediates 3. Alternatively, Method 2 can provide intermediates 3 via Suzuki coupling reaction between 1-alkyltriazole-4-boronates 4 and dihaloaryl compounds 5.

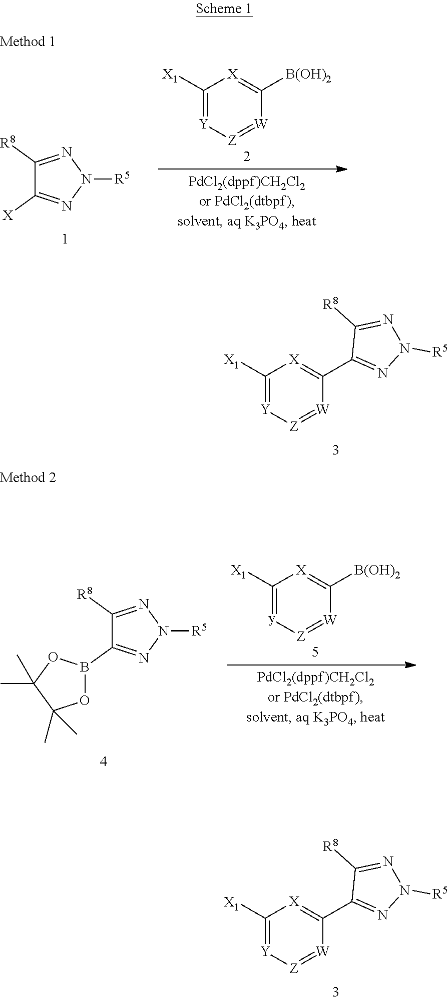

Scheme 2 summarizes one approach to compounds 7. Suzuki coupling of intermediates 3 with boronate esters 6 can provide products 7 in a single step.

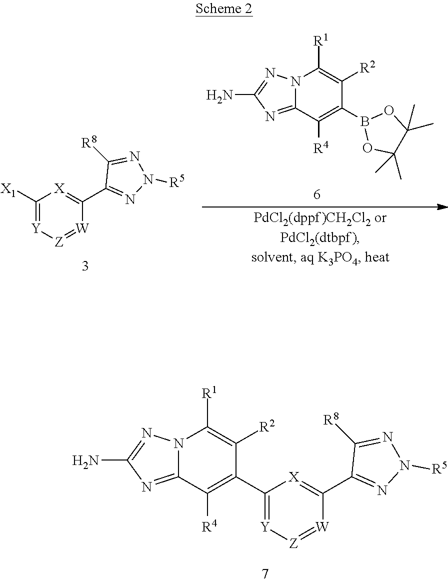

Scheme 3 illustrates an alternative approach to compounds 7. Suzuki coupling of intermediates 3 with N-protected boronate esters 8, where N-protection of 8 can be achieved either via incorporation of two tert-butoxycarbonyl groups on the nitrogen atom, or via incorporation of the nitrogen atom into a 2,5-dimethylpyrrole ring to serve as protection, or via some other N-protecting group or groups known to those skilled in the art, can provide products such as 9. Compounds such as 9 can be converted to products 7 in a subsequent step through use of various N-deprotection conditions known to those skilled in the art.

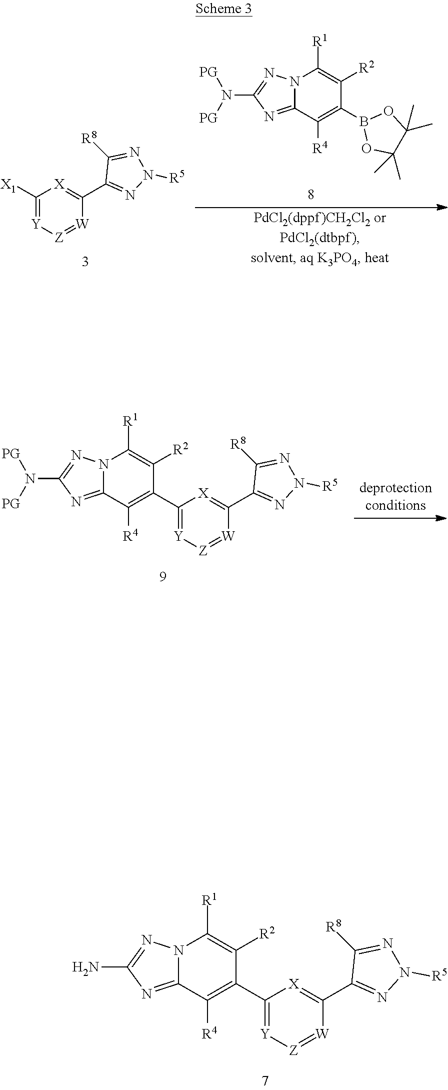

Scheme 4 exemplifies yet another approach to compounds 7. Intermediates 3 can be converted to boronate esters 10 via standard conditions. Compounds such as 10 can then act as reactants in a Suzuki coupling step with halides 11 to provide products 7.

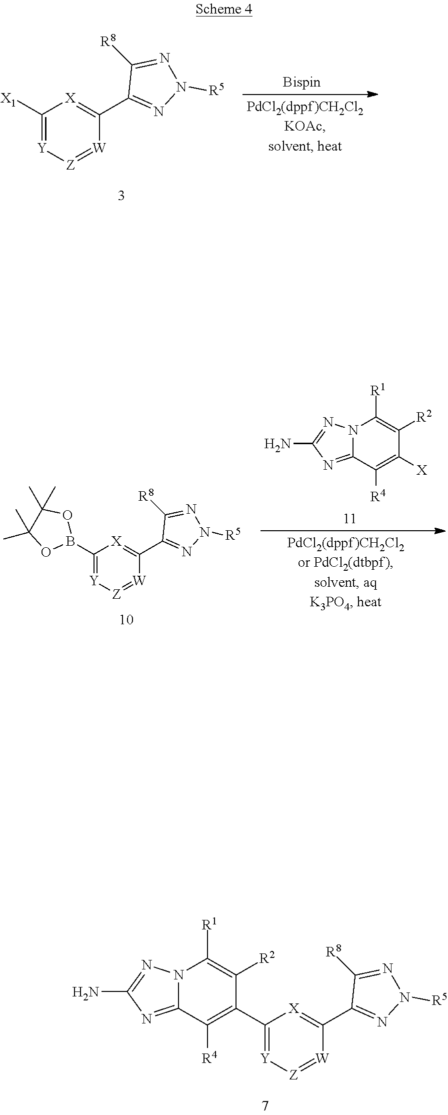

Scheme 5 illustrates yet another approach to compounds 7. Compounds 10 are made to react with N-protected halides 12 via a Suzuki reaction to give materials such as 9. N-protection of 12 can be achieved either via incorporation of two tert-butoxycarbonyl groups on the nitrogen atom, or via incorporation of the nitrogen atom into a 2,5-dimethylpyrrole ring to serve as protection, or via some other N-protecting group or groups known to those skilled in the art. Subsequent N-deprotection of 9 to give products 7 can be achieved through use of various N-deprotection conditions known to those skilled in the art.

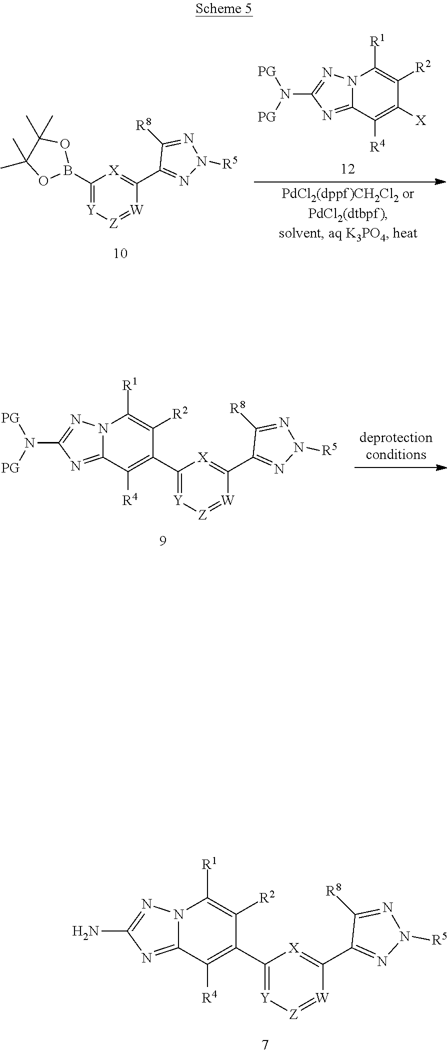

Scheme 6 provides yet another approach to compounds 7. Here, a Suzuki coupling between N-protected boronate 8 and dihaloaryl 5 provides intermediates such as 13. Careful selection of the two halogen substituents of starting materials 5 by one skilled in the art can provide for regiochemical control of products 13 in terms of various substitutions on the six-membered ring of 13. Further modification of halides 13 by another Suzuki coupling with boronate esters such as 4 gives rise to compounds 9, which, upon removal of N-protection, provide products 7.

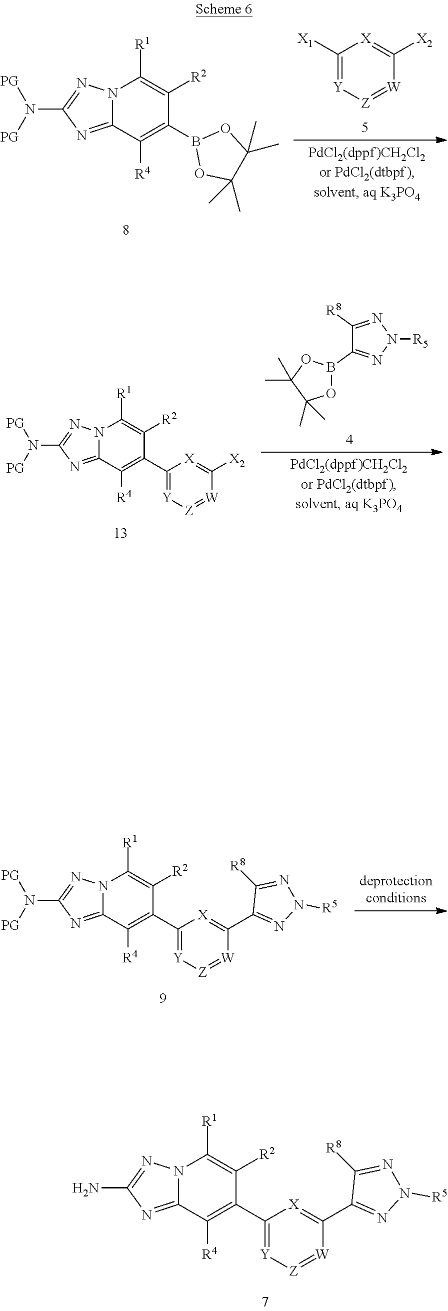

Scheme 7 represents yet another method for the procurement of compounds such as 7. These preparations involve the transformation of intermediates 13 into boronate esters 14. Esters such as 14 can then participate in a Suzuki coupling with substituted halotriazoles 1 to furnish compounds such as 9. Finally, an N-deprotection step provides products 7.

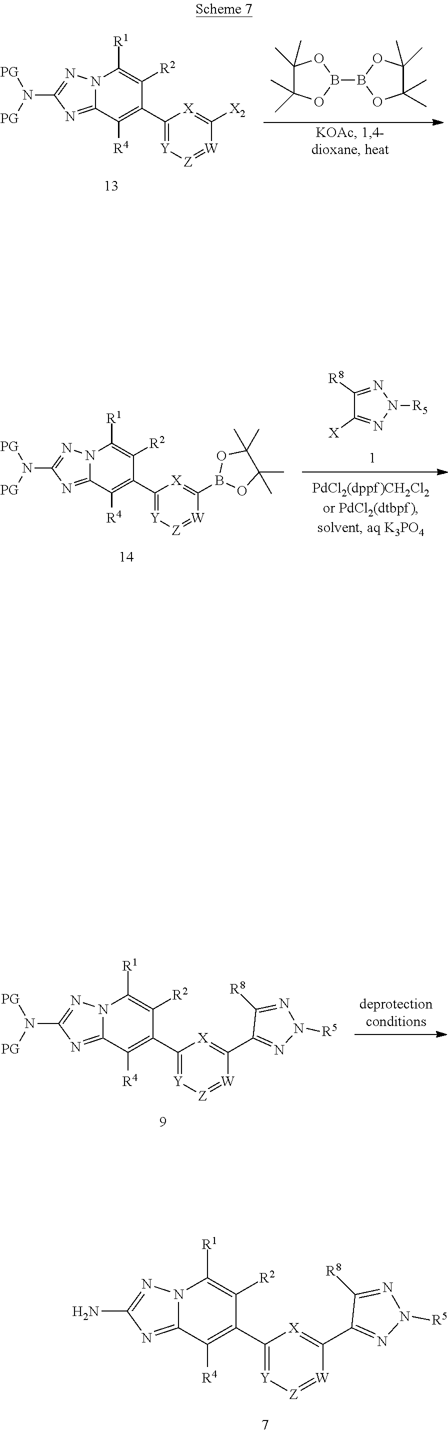

Scheme 8 summarizes an alternate method for the preparation of compounds 7. The dimethylpyrrole protected ATP halide 15 is first converted to pinacol boronate ester 16 via typical conditions. Ester 16 then serves as a Suzuki coupling partner with various dihaloaryl species 5 to provide compounds such as 17, which further becomes a coupling partner with 1-H-triazole-4-boronic acid 18 in a second Suzuki coupling reaction to provide products 19. Functionalization of 19 via various synthetic methods including, but not limited to, alkylation or Mitsunobu reaction, gives rise to compounds such as 20. Deprotection of the dimethylpyrrole protecting group of 20 using one of a variety of conditions known to those skilled in the art gives rise to products 7.

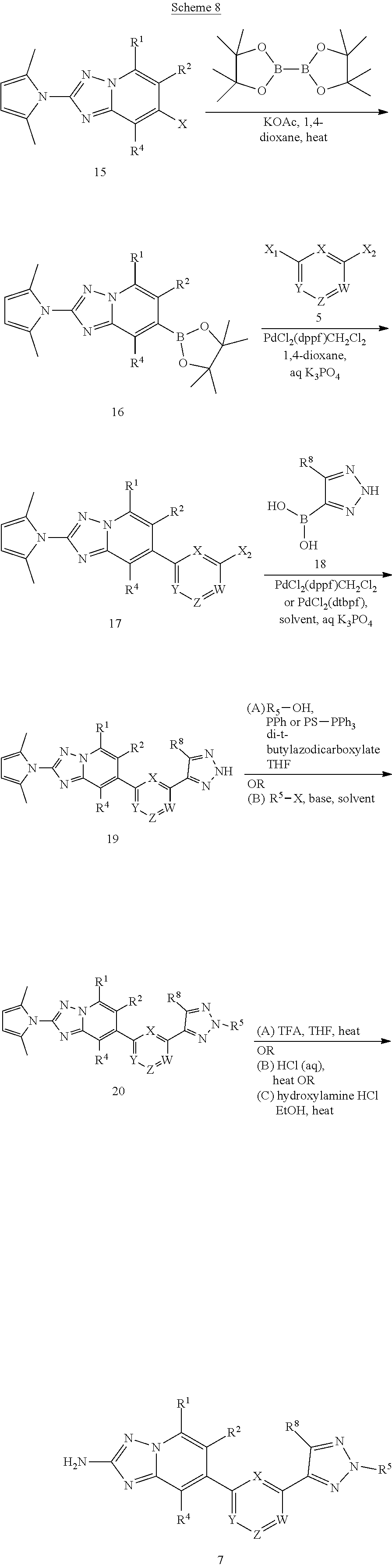

### EXAMPLES

### Intermediate 1: 4-bromo-2-(1-(3,5-difluorophenyl)-2,2-difluoropropyl)-2H-1,2,3-triazole

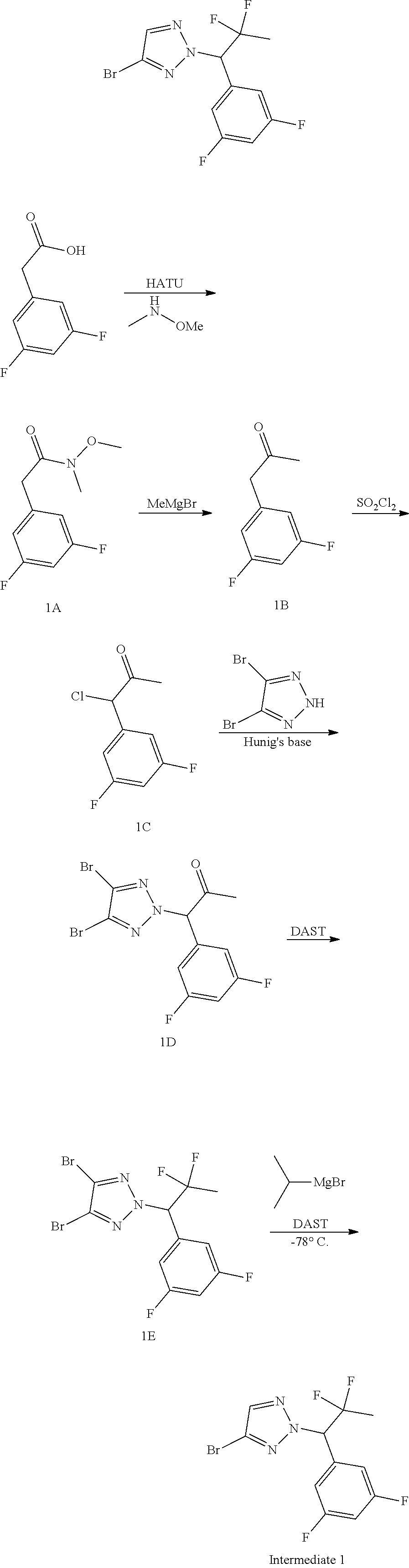

### Intermediate 1A: 2-(3,5-difluorophenyl)-N-methoxy-N-methylacetamide

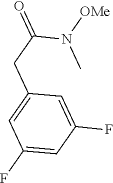

To a solution of 2-(3,5-difluorophenyl)acetic acid (5.2 g, 30.2 mmol) in DMF (60 mL) were added N,O-dimethylhydroxylamine, HCl (3.54 g, 36.3 mmol) and HATU (17.23 g, 45.3 mmol). Then Hunig's base (15.83 mL, 91 mmol) was added dropwise. The resulting yellow solution was stirred at rt overnight. It was diluted with ethyl acetate and water. The layers were separated. The aqueous layer was extracted with ethyl acetate. The combined organic layers were washed with water and brine, and dried over sodium sulfate. The crude product was purified by silica gel chromatography (80 g RediSep® column, eluting with a gradient from 5-45% ethyl acetate in hexanes, 13 cv). Fractions containing the product were combined and evaporated to afford 2-(3,5-difluorophenyl)-N-methoxy-N-methylacetamide (5.6 g, 26.0 mmol, 86% yield) as a colorless oil. ¹H NMR (400 MHz, CHLOROFORM-d) δ 6.90-6.81 (m, 2H), 6.78-6.61 (m, 1H), 3.77 (s, 2H), 3.69 (s, 3H), 3.22 (s, 3H).

### Intermediate 1B: 1-(3,5-difluorophenyl)propan-2-one

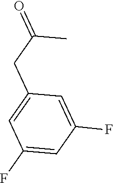

To a solution of 2-(3,5-difluorophenyl)-N-methoxy-N-methylacetamide (2.40 g, 11.15 mmol) in THF (30 mL) at 0° C. was added methylmagnesium bromide (3 M solution solution in diethyl ether) (7.81 mL, 23.42 mmol) dropwise. The resulting yellow solution was stirred at 0° C. for 1 h. It was quenched with saturated ammonium chloride and diluted with ethyl acetate. The layers were separated. The aqueous layer was extracted with ethyl acetate. The combined organic layers were washed with water and brine, and dried over sodium sulfate. The crude product was purified by silica gel chromatography (80 g RediSep® column, eluting with a gradient from 5-45% ethyl acetate in hexanes, 13 cv). Fractions containing the product were combined and evaporated to afford 1-(3,5-difluorophenyl)propan-2-one (1.52 g, 8.93 mmol, 80% yield) as a colorless oil. ¹H NMR (400 MHz, CHLOROFORM-d) δ 6.77-6.72 (m, 3H), 3.71 (s, 2H), 2.21 (s, 3H).

### Intermediate 1C: (1) 1-chloro-1-(3,5-difluorophenyl)propan-2-one

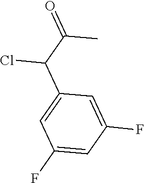

To a solution of 1-(3,5-difluorophenyl)propan-2-one (1.52 g, 8.93 mmol) in DCM (15 mL) at 0° C. was added sulfuryl dichloride (1.089 mL, 13.40 mmol) dropwise. The reaction mixture was stirred at rt for 4 h. It was quenched with water, and extracted with DCM. The organic layers were combined and washed twice with saturated sodium bicarbonate, water, and finally brine. The solution was dried over sodium sulfate, filtered, and concentration to give 1-chloro-1-(3,5-difluorophenyl)propan-2-one (1.85 g, 9.04 mmol, 101% yield), which was used for the next step without further purification. LCMS (ESI, m/z) 202.7/204.7 [M+H]⁺

### Intermediate 1D: (±) 1-(4,5-dibromo-2H-1,2,3-triazol-2-yl)-1-(3,5-difluorophenyl)propan-2-one

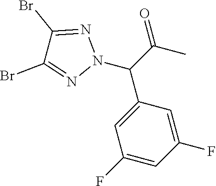 [SMILES: CC(=O)C(c1cc(F)cc(F)c1)n1nc(Br)c(Br)n1]

To a solution of (+) 1-chloro-1-(3,5-difluorophenyl)propan-2-one (1.827 g, 8.93 mmol) in DCM (15 mL) was added 4,5-dibromo-2H-1,2,3-triazole (2.026 g, 8.93 mmol). Hunig's base (3.12 mL, 17.86 mmol) was then added dropwise. The resulting dark solution was stirred at rt overnight. It was quenched with water, extracted with ethyl acetate, washed with brine, and dried over sodium sulfate. The crude product was purified by silica gel chromatography (80 g RediSep® column, eluting with a gradient from 5-35% ethyl acetate in hexanes, 13 cv). Fractions containing the product were combined and evaporated to afford 1-(4,5-dibromo-2H-1,2,3-triazol-2-yl)-1-(3,5-difluorophenyl)propan-2-one (1.85 g, 4.68 mmol, 52.40% yield). ¹H NMR (400 MHz, CHLOROFORM-d) δ 7.07-7.00 (m, 2H), 6.97-6.88 (m, 1H), 6.23-6.19 (m, 1H), 2.23 (s, 3H).

### Intermediate 1E: (1) 4,5-dibromo-2-(1-(3,5-difluorophenyl)-2,2-difluoropropyl)-2H-1,2,3-triazole

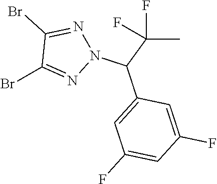 [SMILES: CC(F)(F)C(c1cc(F)cc(F)c1)n1nc(Br)c(Br)n1]

To a solution of (±) 1-(4,5-dibromo-2H-1,2,3-triazol-2-yl)-1-(3,5-difluorophenyl)propan-2-one (1.03 g, 2.61 mmol) in DCM (10 mL) at 0° C. under nitrogen was added DAST (0.861 mL, 6.52 mmol) dropwise. The reaction mixture was stirred at rt overnight. It was quenched with ice-water, extracted with DCM, washed with brine, and dried over sodium sulfate. The crude product was purified by silica gel chromatography (40 g RediSep® column, eluting with a gradient from 0-10% ether in hexanes, 12 cv). Fractions containing the product were combined and evaporated to afford 4,5-dibromo-2-(1-(3,5-difluorophenyl)-2,2-difluoropropyl)-2H-1,2,3-triazole (0.82 g, 75.9%, 85% pure) which was further purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 min; Flow Rate: 42.5 mL/min; Column Temperature: 25° C. Fraction collection was triggered by UV (214 nm). Fractions containing the desired product were combined and dried via centrifugal evaporation to provide (±) 4,5-dibromo-2-(1-(3,5-difluorophenyl)-2,2-difluoropropyl)-2H-1,2,3-triazole. ¹H NMR (400 MHz, CHLOROFORM-d) δ 7.24-7.10 (m, 2H), 6.98-6.85 (m, 1H), 5.93-5.77 (m, 1H), 1.73 (t, J=18.7 Hz, 3H).

### Intermediate 1: (1) 4-bromo-2-(1-(3,5-difluorophenyl)-2,2-difluoropropyl)-2H-1,2,3-triazole

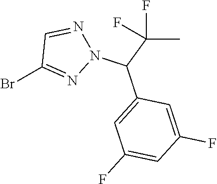 [SMILES: CC(F)(F)C(c1cc(F)cc(F)c1)n1ncc(Br)n1]

To a solution of (I) 4,5-dibromo-2-(1-(3,5-difluorophenyl)-2,2-difluoropropyl)-2H-1,2,3-triazole (170 mg, 0.408 mmol) in THF (5 mL) at −78° C. under nitrogen was added isopropylmagnesium bromide (0.75 M THF solution) (2.066 mL, 1.549 mmol) dropwise. The reaction mixture was stirred at −78° C. for 2 h. It was quenched with sat'd ammonium chloride, extracted with ethyl acetate, washed with brine, and dried over sodium sulfate. The crude product was purified by silica gel chromatography (24 g RediSep® column, eluting with a gradient from 0-10% ether in hexanes, 12 cv). Fractions containing the product were combined and evaporated to afford (±) 4-bromo-2-(1-(3,5-difluorophenyl)-2,2-difluoropropyl)-2H-1,2,3-triazole (138 mg, 80%). ¹H NMR (400 MHz, CHLOROFORM-d) δ 7.70 (s, 1H), 7.25-7.10 (m, 2H), 6.94-6.82 (m, 1H), 6.01-5.84 (m, 1H), 1.81-1.63 (m, 3H).

### Intermediate 2: (1) 4-bromo-2-(1-(4-chloro-3-fluorophenyl)-2,2-difluoropropyl)-2H-1,2,3-triazole

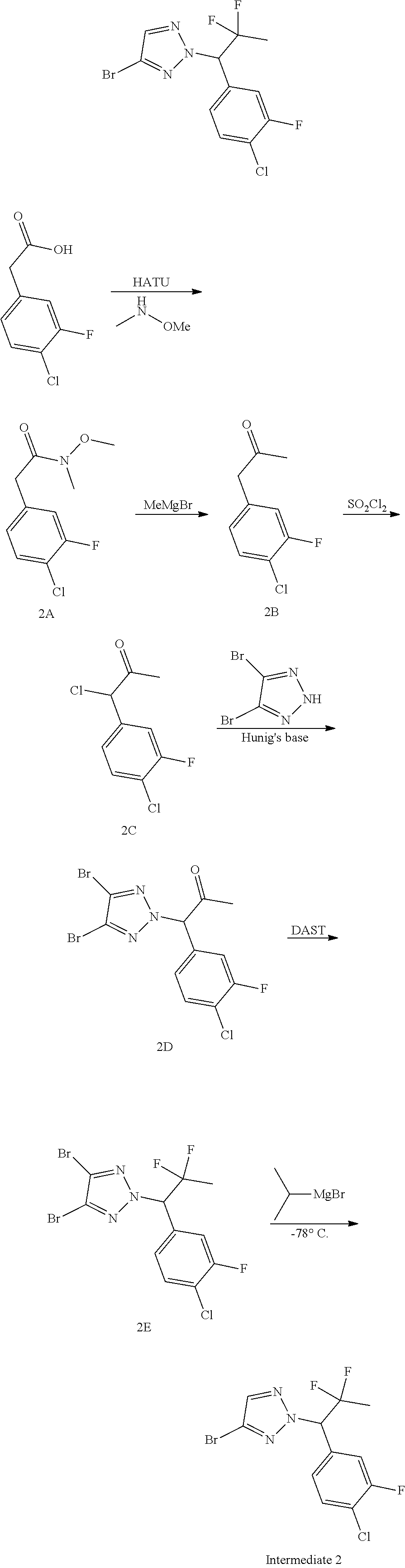

### Intermediate 2A: 2-(4-chloro-3-fluorophenyl)-N-methoxy-N-methylacetamide

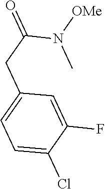

To a solution of 2-(4-chloro-3-fluorophenyl)acetic acid (4.6 g, 24.39 mmol) in DMF (50 mL) were added N,O-dimethylhydroxylamine, HCl (2.379 g, 24.39 mmol), HATU (13.91 g, 36.6 mmol), and Hunig's base (12.78 mL, 73.2 mmol). The reaction mixture was stirred at rt overnight. It was diluted with ethyl acetate and water. The layers were separated. The aqueous layer was extracted with ethyl acetate. The combined organic layers were washed with water and brine, dried over sodium sulfate. The crude product was purified by silica gel chromatography (80 g RediSep® column, eluting with a gradient from 5-45% ethyl acetate in hexanes, 13 cv). Fractions containing the product were combined and evaporated to afford 2-(4-chloro-3-fluorophenyl)-N-methoxy-N-methylacetamide (4.86 g, 20.98 mmol, 86% yield) as a tan solid. ¹H NMR (400 MHz, CHLOROFORM-d) δ 7.39-7.30 (m, 1H), 7.17-7.09 (m, 1H), 7.07-6.96 (m, 1H), 3.76 (s, 2H), 3.69 (s, 3H), 3.22 (s, 3H).

### Intermediate 2B: 1-(4-chloro-3-fluorophenyl)propan-2-one

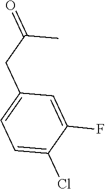

To a solution of 2-(4-chloro-3-fluorophenyl)-N-methoxy-N-methylacetamide (2.87 g, 12.39 mmol) in THF (60 ml) at 0° C. was added methylmagnesium bromide (3 M solution solution in diethyl ether) (4.54 ml, 13.63 mmol) dropwise. The resulting solution was stirred at 0° C. for 1 h. It was quenched with saturated ammonium chloride and diluted with ethyl acetate. The layers were separated. The aqueous layer was extracted with ethyl acetate. The combined organic layers were washed with water and brine, then dried over sodium sulfate. The crude product was purified by silica gel chromatography (80 g RediSep® column, eluting with a gradient from 5-45% ethyl acetate in hexanes, 13 cv).

Fractions containing the product were combined and evaporated to afford 1-(4-chloro-3-fluorophenyl)propan-2-one (1.38 g, 60%). ¹H NMR (400 MHz, CHLOROFORM-d) δ 7.43-7.33 (m, 1H), 7.05-6.99 (m, 1H), 6.96-6.91 (m, 1H), 3.71 (s, 2H), 2.22 (s, 3H).

### Intermediate 2C: (1) 1-chloro-1-(4-chloro-3-fluorophenyl)propan-2-one

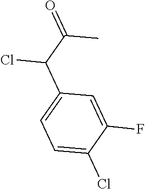

To a solution of 1-(4-chloro-3-fluorophenyl)propan-2-one (2.2 g, 11.79 mmol) in DCM (15 mL) at 0° C. was added sulfuryl dichloride (1.054 mL, 12.97 mmol) dropwise. The reaction mixture was stirred at rt overnight. It was quenched with water and extracted with DCM. The organic phase was washed with sat'd sodium bicarbonate and water to pH 7, dried over sodium sulfate, then concentrated to give 1-chloro-1-(4-chloro-3-fluorophenyl)propan-2-one (2.66 g, 12.03 mmol, 102% yield). The crude product was used for the next step without further purification.

LCMS (ESI, m/z) 218.7 [M−H]⁺.

### Intermediate 2D: (1) 1-(4-chloro-3-fluorophenyl)-1-(4,5-dibromo-2H-1,2,3-triazol-2-yl)propan-2-one

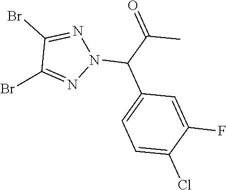 [SMILES: CC(=O)C(c1ccc(Cl)c(F)c1)n1nc(Br)c(Br)n1]

To a solution of 1-chloro-1-(4-chloro-3-fluorophenyl)propan-2-one (1.753 g, 7.93 mmol) in DCM (15 mL) were added 4,5-dibromo-2H-1,2,3-triazole (1.799 g, 7.93 mmol) and Hunig's base (2.77 mL, 15.86 mmol). The resulting dark solution was stirred at rt overnight. It was quenched with water, extracted with DCM, washed with brine, and dried over sodium sulfate. The crude product was purified by silica gel chromatography (80 g RediSep® column, eluting with a gradient from 5-35% ethyl acetate in hexanes, 13 cv). Fractions containing the product were combined and evaporated to afford 1-(4-chloro-3-fluorophenyl)-1-(4,5-dibromo-2H-1,2,3-triazol-2-yl)propan-2-one (1.6 g, 3.89 mmol, 49.0% yield). LCMS (ESI, m/z) 409.4 [M+H]⁺.

### Intermediate 2E: (1) 4,5-dibromo-2-(1-(4-chloro-3-fluorophenyl)-2,2-difluoropropyl)-2H-1,2,3-triazole

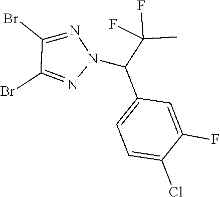 [SMILES: CC(F)(F)C(c1ccc(Cl)c(F)c1)n1nc(Br)c(Br)n1]

To a solution of (1) 1-(4-chloro-3-fluorophenyl)-1-(4,5-dibromo-2H-1,2,3-triazol-2-yl)propan-2-one (1.17 g, 2.84 mmol) in DCM (15 mL) at 0° C. was added DAST (1.127 mL, 8.53 mmol) dropwise. The reaction mixture was stirred at rt overnight. It was quenched with ice-water, extracted with DCM, washed with brine, and dried over sodium sulfate. The crude product was purified by silica gel chromatography (40 g RediSep® column, eluting with a gradient from 0-10% ethyl acetate in hexanes, 12 cv) to give 4,5-dibromo-2-(1-(4-chloro-3-fluorophenyl)-2,2-difluoropropyl)-2H-1,2,3-triazole (460 mg, 37.3%) in ~70% purity. It was further purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 min; Flow Rate: 42.5 mL/min; Column Temperature: 25° C. Fraction collection was triggered by UV (214 nm). Fractions containing the desired product were combined and dried via centrifugal evaporation to provide the product. ¹H NMR (400 MHz, CHLOROFORM-d) δ 7.55-7.42 (m, 2H), 7.38-7.31 (m, 1H), 5.91-5.80 (m, 1H), 1.71 (t, J=18.7 Hz, 3H).

### Intermediate 2: (1) 4-bromo-2-(1-(4-chloro-3-fluorophenyl)-2,2-difluoropropyl)-2H-1,2,3-triazole

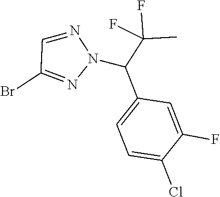 [SMILES: CC(F)(F)C(c1ccc(Cl)c(F)c1)n1ncc(Br)n1]

To a solution of (1) 4,5-dibromo-2-(1-(3-chloro-4-fluorophenyl)-2,2-difluoropropyl)-2H-1,2,3-triazole (459 mg, 1.059 mmol) in THF (10 mL) at −78° C. under nitrogen was added isopropylmagnesium bromide (5.4 mL, 4.02 mmol) (0.75 M solutiuon solution in THF). The reaction mixture was stirred at −78° C. for 2 h. It was quenched with sat's ammonium chloride and extracted with ethyl acetate. The combined organic layers were washed with brine, dried over anhydrous sodium sulfate, and filtered. The filtrate was evaporated in vacuo to give the crude residue. The crude product was purified by silica gel chromatography (40 g RediSep® column, eluting with a gradient from 0-10% EtOAc in hexanes, 12 cv). Fractions containing the product were combined and evaporated to afford (±) 4-bromo-2-(1-(3-chloro-4-fluorophenyl)-2,2-difluoropropyl)-2H-1,2,3-triazole (320 mg, 0.903 mmol, 85% yield). ¹H NMR (400 MHz, CHLOROFORM-d) δ 7.70 (s, 1H), 7.57-7.49 (m, 1H), 7.48-7.41 (m, 1H), 7.36-7.30 (m, 1H), 6.01-5.87 (m, 1H), 1.70 (t, J=18.7 Hz, 3H).

### Intermediate 3: (±) 2-(2,2-difluoro-1-(4-methoxyphenyl)propyl)-4-(4,4,5,5-tetramethyl-1,3,2-dioxaborolan-2-yl)-2H-1,2,3-triazole

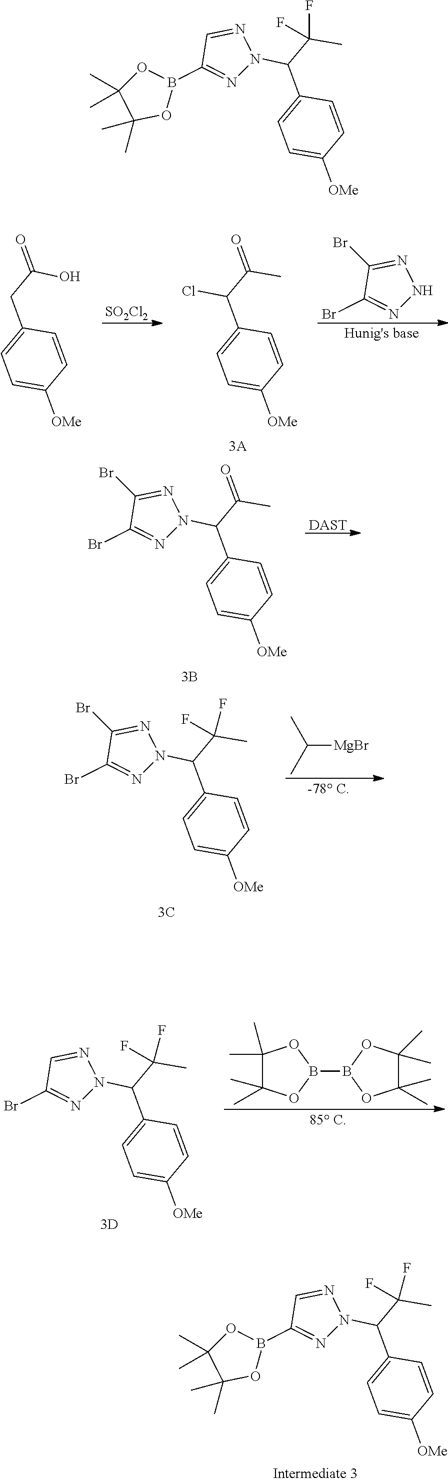

### Intermediate 3A: (1) 1-chloro-1-(4-methoxyphenyl)propan-2-one

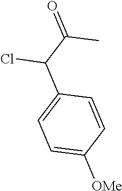

To a solution of 1-(4-methoxyphenyl)propan-2-one (4 g, 24.36 mmol) in DCM (45 mL) at 0° C. was added sulfuryl dichloride (2.97 mL, 36.5 mmol) dropwise. The reaction mixture was stirred at rt overnight. It was quenched with sodium bicarbonate, extracted with DCM, washed with water and brine, dried over sodium sulfate. The crude product was used for next step. ¹H NMR (400 MHz, CHLOROFORM-d) δ 7.38-7.31 (m, 2H), 6.96-6.90 (m, 2H), 5.35 (s, 1H), 3.83 (s, 3H), 2.23 (s, 3H).

### Intermediate 3B: (±) 1-(4,5-dibromo-2H-1,2,3-triazol-2-yl)-1-(4-methoxyphenyl)propan-2-one

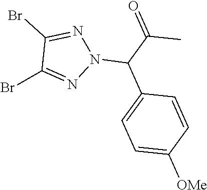 [SMILES: COc1ccc(C(C(C)=O)n2nc(Br)c(Br)n2)cc1]

To a solution of 1-chloro-1-(4-methoxyphenyl)propan-2-one (4 g, 20.14 mmol) in DCM (25 mL) were added 4,5-dibromo-2H-1,2,3-triazole (5.48 g, 24.16 mmol) and Hunig's base (7.03 mL, 40.3 mmol). The reaction mixture was stirred at rt overnight. It was quenched with water, extracted with ethyl acetate, washed with brine, and dried over sodium sulfate. The crude product was purified by silica gel chromatography (80 g RediSep® column, eluting with a gradient from 5-25% ethyl acetate in hexanes, 13 cv). Fractions containing the product were combined and evaporated to afford (±) 1-(4,5-dibromo-2H-1,2,3-triazol-2-yl)-1-(4-methoxyphenyl)propan-2-one (6.05 g, 15.55 mmol, 77% yield) as a tan solid. ¹H NMR (400 MHz, CHLOROFORM-d) δ 7.50-7.37 (m, 2H), 7.07-6.90 (m, 2H), 6.25 (s, 1H), 3.86 (s, 3H), 2.19 (s, 3H). LCMS (ESI, m/z) 247.0 [M+H]⁺.

### Intermediate 3C: (1) 4,5-dibromo-2-(2,2-difluoro-1-(4-methoxyphenyl)propyl)-2H-1,2,3-triazole

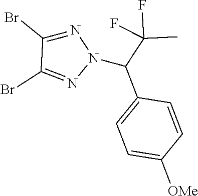 [SMILES: COc1ccc(C(n2nc(Br)c(Br)n2)C(C)(F)F)cc1]

To a solution of (I) 1-(4,5-dibromo-2H-1,2,3-triazol-2-yl)-1-(4-methoxyphenyl)propan-2-one (1.6 g, 4.11 mmol) in DCM (20 mL) at 0° C. under nitrogen was added DAST (1.141 mL, 8.64 mmol) dropwise. The reaction mixture was stirred at rt overnight. It was quenched with ice-water, extracted with DCM, washed with brine, and dried over sodium sulfate. The crude product was purified by silica gel chromatography (80 g RediSep® column, eluting with a gradient from 0-15% EtOAc in hexanes, 12 cv) to give (±) 4,5-dibromo-2-(2,2-difluoro-1-(4-methoxyphenyl)propyl)-2H-1,2,3-triazole (0.8 g, 45%), which was further purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 min; Flow Rate: 42.5 mL/min; Column Temperature: 25° C. Fraction collection was triggered by UV (214 nm). Fractions containing the desired product were combined and dried via centrifugal evaporation to provide (±) 4,5-dibromo-2-(2,2-difluoro-1-(4-methoxyphenyl)propyl)-2H-1,2,3-triazole. ¹H NMR (400 MHz, CHLOROFORM-d) δ 7.63-7.50 (m, 2H), 6.99-6.90 (m, 2H), 5.88-5.77 (m, 1H), 3.84 (s, 3H), 1.78-1.63 (m, 3H).

### Intermediate 3E: (I) 4-bromo-2-(2,2-difluoro-1-(4-methoxyphenyl)propyl)-2H-1,2,3-triazole

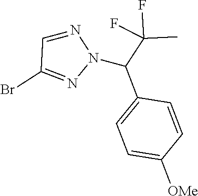 [SMILES: COc1ccc(C(n2ncc(Br)n2)C(C)(F)F)cc1]

To a solution of (±) 4,5-dibromo-2-(2,2-difluoro-1-(4-methoxyphenyl)propyl)-2H-1,2,3-triazole (800 mg, 1.946 mmol) in THF (10 mL) at −78° C. under nitrogen was added isopropylmagnesium bromide (0.75 M solution in THF) (9.86 mL, 7.40 mmol) dropwise. The reaction mixture was stirred at −78° C. for 2 h. It was quenched with sat'd ammonium chloride, extracted with ethyl acetate, washed with brine, and dried over sodium sulfate. It was purified by silica gel chromatography (40 g RediSep® column, eluting with a gradient from 5-45% ethyl acetate in hexanes, 13 cv). Fractions containing the product were combined and evaporated to afford (±) 4-bromo-2-(2,2-difluoro-1-(4-methoxyphenyl)propyl)-2H-1,2,3-triazole (160 mg, 0.482 mmol, 24.75% yield). ¹H NMR (400 MHz, CHLOROFORM-d) δ 7.66 (s, 1H), 7.58 (d, J=8.6 Hz, 2H), 6.98-6.89 (m, 2H), 6.00-5.82 (m, 1H), 3.83 (s, 3H), 1.77-1.63 (m, 3H).

### Intermediate 3: (±) 2-(2,2-difluoro-1-(4-methoxyphenyl)propyl)-4-(4,4,5,5-tetramethyl-1,3,2-dioxaborolan-2-yl)-2H-1,2,3-triazole

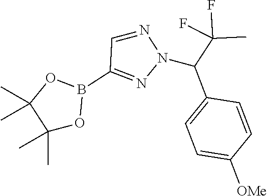 [SMILES: COc1ccc(C(n2ncc(B3OC(C)(C)C(C)(C)O3)n2)C(C)(F)F)cc1]

To a pressure vial were added (I) 4-bromo-2-(2,2-difluoro-1-(4-methoxyphenyl)propyl)-2H-1,2,3-triazole (150 mg, 0.452 mmol), 4,4,4′,4′,5,5,5′,5′-octamethyl-2,2′-bi(1,3,2-dioxaborolane) (161 mg, 0.632 mmol), potassium acetate (111 mg, 1.129 mmol), and dioxane (6 mL). It was purged with nitorgen and heated at 85° C. overnight. It was cooled to rt. The crude material was used without workup. LCMS (ESI, m/z) 297.9 [M+H]⁺. Intermediate 4: (1) 2-chloro-6-(2-(1-(4-chloro-3-fluorophenyl)-2,2-difluoropropyl)-2H-1,2,3-triazol-4-yl)pyridine

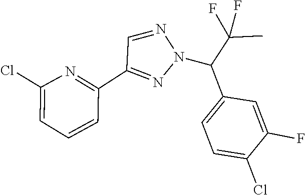 [SMILES: CC(F)(F)C(c1ccc(Cl)c(F)c1)n1ncc(-c2cccc(Cl)n2)n1]

To a pressure vial were added 2-chloro-6-(4,4,5,5-tetramethyl-1,3,2-dioxaborolan-2-yl)pyridine (215 mg, 0.898 mmol), (±) 4-bromo-2-(1-(4-chloro-3-fluorophenyl)-2,2-difluoropropyl)-2H-1,2,3-triazole (245 mg, 0.691 mmol), potassium phosphate (2 M water solution) (1.04 mL, 2.073 mmol), PdCl₂(dppf)-CH₂Cl₂ adduct (56 mg, 0.069 mmol), and dioxane (4 mL). It was purged with nitrogen and heated at 85° C. for 1 h. The reaction mixture was diluted water and ethyl acetate. The layers were separated. The aqueous layer was extracted with ethyl acetate. The combined organic layers were washed with water, brine, dried over anhydrous sodium sulfate, filtered and evaporated to dryness. It was purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 min; Flow Rate: 42.5 mL/min; Column Temperature: 25° C. Fraction collection was triggered by UV (214 nm). Fractions containing the desired product were combined and dried via centrifugal evaporation to provide (±) 2-chloro-6-(2-(1-(4-chloro-3-fluorophenyl)-2,2-difluoropropyl)-2H-1,2,3-triazol-4-yl)pyridine (76 mg, 28.4%). LCMS (ESI, m/z) 388.9 [M+H]⁺.

### Intermediate 5: (1) 3-bromo-5-(2-(2,2-difluoro-1-(4-methoxyphenyl)propyl)-2H-1,2,3-triazol-4-yl)pyridine

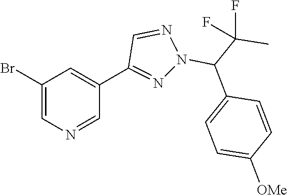 [SMILES: COc1ccc(C(n2ncc(-c3cncc(Br)c3)n2)C(C)(F)F)cc1]

To a pressure vial were added 3,5-dibromopyridine (0.213 g, 0.900 mmol), 2-(2,2-difluoro-1-(4-methoxyphenyl)propyl)-4-(4,4,5,5-tetramethyl-1,3,2-dioxaborolan-2-yl)-2H-1,2,3-triazole (0.228 g, 0.6 mmol), dioxane (6 mL), potassium phosphate (2 M water solution) (0.90 mL, 1.80 mmol), and PdCl₂(dppf)-CH₂Cl₂ adduct (0.049 g, 0.060 mmol). It was purged with nitrogen and heated at 65° C. for 3 h. It was diluted with ethyl acetate and water, extracted with ethyl acetate. The combined organic layers were washed with brine, dried over anhydrous sodium sulfate and filtered, and the filtrate was evaporated in vacuo to give the crude residue. The crude product was purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 min; Flow Rate: 42.5 mL/min; Column Temperature: 25° C. Fraction collection was triggered by UV (214 nm). Fractions containing the desired product were combined and dried via centrifugal evaporation to afford (±) 3-bromo-5-(2-(2,2-difluoro-1-(4-methoxyphenyl)propyl)-2H-1,2,3-triazol-4-yl)pyridine (95 mg, 38.6%).

LCMS (ESI, m/z) 410.8 [M+H]⁺.

### Intermediate 6: (±) 2-chloro-4-(2-(2,2-difluoro-1-(4-methoxyphenyl)propyl)-2H-1,2,3-triazol-4-yl)pyrimidine

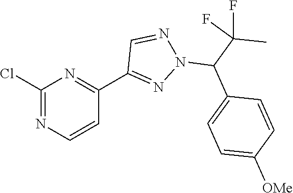 [SMILES: COc1ccc(C(n2ncc(-c3ccnc(Cl)n3)n2)C(C)(F)F)cc1]

The titled compound (I) 2-chloro-4-(2-(2,2-difluoro-1-(4-methoxyphenyl)propyl)-2H-1,2,3-triazol-4-yl)pyrimidine was obtained in a similar fashion as described for the preparation of Intermediate 5, where 4-bromo-2-chloropyrimidine was used in place of 2,6-dibromopyridine. The crude was purified via preparative HPLC with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 minutes; Flow Rate: 42.5 mL/min; Column Temperature: 25° C. Fraction collection was triggered by UV signals. Fractions containing the desired product were combined and dried via centrifugal evaporation to give (I) 2-chloro-4-(2-(2,2-difluoro-1-(4-methoxyphenyl)propyl)-2H-1,2,3-triazol-4-yl)pyrimidine (54 mg, 30.7%). LCMS (ESI, m/z) 366.0 [M+H]⁺.

### Intermediate 7: (±) 2-chloro-4-(2-(2,2-difluoro-1-(4-methoxyphenyl)propyl)-2H-1,2,3-triazol-4-yl)-5-methylpyrimidine

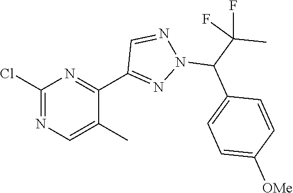 [SMILES: COc1ccc(C(n2ncc(-c3nc(Cl)ncc3C)n2)C(C)(F)F)cc1]

The titled compound (I) 2-chloro-4-(2-(2,2-difluoro-1-(4-methoxyphenyl)propyl)-2H-1,2,3-triazol-4-yl)-5-methylpyrimidine was obtained in a similar fashion as described for the preparation of Intermediate 5, where 4-bromo-2-chloro-5-methylpyrimidine was used in place of 2,6-dibromopyridine. The crude was purified via preparative HPLC with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 minutes; Flow Rate: 42.5 mL/min; Column Temperature: 25° C. Fraction collection was triggered by UV signals. Fractions containing the desired product were combined and dried via centrifugal evaporation to give (±) 2-chloro-4-(2-(2,2-difluoro-1-(4-methoxyphenyl)propyl)-2H-1,2,3-triazol-4-yl)-5-methylpyrimidine (57 mg, 33.1%).

LCMS (ESI, m/z) 380.0 [M+H]⁺.

### Intermediate 8: (±)-2-chloro-4-(2-(2,2-difluoro-1-(4-fluorophenyl)propyl)-2H-1,2,3-triazol-4-yl)-5-methylpyrimidine

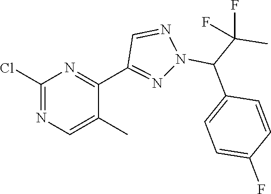 [SMILES: Cc1cnc(Cl)nc1-c1cnn(C(c2ccc(F)cc2)C(C)(F)F)n1]

The titled compound (±)-2-chloro-4-(2-(2,2-difluoro-1-(4-methoxyphenyl)propyl)-2H-1,2,3-triazol-4-yl)-5-methylpyrimidine was obtained in a similar fashion as described for the preparation of Intermediate 5, where 4-bromo-2-chloro-5-methylpyrimidine and 2-(2,2-difluoro-1-(4-fluorophenyl)propyl)-4-(4,4,5,5-tetramethyl-1,3,2-dioxaborolan-2-yl)-2H-1,2,3-triazole were used in place of 3,5-dibromopyridine and 2-(2,2-difluoro-1-(4-methoxyphenyl)propyl)-4-(4,4,5,5-tetramethyl-1,3,2-dioxaborolan-2-yl)-2H-1,2,3-triazole. The crude was purified via preparative HPLC with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 minutes; Flow Rate: 42.5 mL/min; Column Temperature: 25° C. Fraction collection was triggered by UV signals. Fractions containing the desired product were combined and dried via centrifugal evaporation to give (±) 2-chloro-4-(2-(2,2-difluoro-1-(4-methoxyphenyl)propyl)-2H-1,2,3-triazol-4-yl)-5-methylpyrimidine (56 mg, 41.4%). LCMS (ESI, m/z) 380.0 [M+H]⁺.

### Intermediate 9: tert-butyl (tert-butoxycarbonyl)(7-(6-chloro-3-methylpyridin-2-yl)-[1,2,4]triazolo[1,5-a]pyridin-2-yl)carbamate

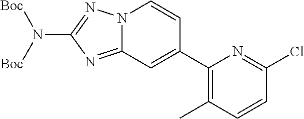 [SMILES: Cc1ccc(Cl)nc1-c1ccn2nc(N(C(=O)OC(C)(C)C)C(=O)OC(C)(C)C)nc2c1]

To a pressure vial were added tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (650 mg, 1.412 mmol) (Intermediate 15), 2-bromo-6-chloro-3-methylpyridine (330 mg, 1.598 mmol), dioxane (6 mL), potassium phosphate (2 M water solution) (2.118 mL, 4.24 mmol), and PdCl₂(dppf)-CH₂Cl₂ adduct (153 mg, 0.141 mmol). It was purged with nitrogen and heated at 65° C. for 1 h. The reaction was diluted with ethyl acetate and water. The layers were separated and the aqueous layer was extracted with ethyl acetate. The combined organic layers were washed with water, brine, dried over anhydrous sodium sulfate, filtered and evaporated to dryness. The crude material was purified purified by silica gel chromatography (40 g RediSep© column, eluting with a gradient from 5-45% ethyl acetate in hexanes, 14 cv). Fractions containing the product were combined and concentrated to afford tert-butyl (tert-butoxycarbonyl)(7-(6-chloro-3-methylpyridin-2-yl)-[1,2,4]triazolo[1,5-a]pyridin-2-yl)carbamate (440 mg, 0.957 mmol, 67.8% yield). LCMS (ESI, m/z): 460.4 [M+H]⁺.

### Intermediate 10: tert-butyl (tert-butoxycarbonyl)(7-(3-methyl-6-(4,4,5,5-tetramethyl-1,3,2-dioxaborolan-2-yl)pyridin-2-yl)-[1,2,4]triazolo[1,5-a]pyridin-2-yl)carbamate

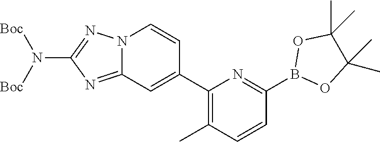 [SMILES: Cc1ccc(B2OC(C)(C)C(C)(C)O2)nc1-c1ccn2nc(N(C(=O)OC(C)(C)C)C(=O)OC(C)(C)C)nc2c1]

To a solution of tert-butyl (tert-butoxycarbonyl)(7-(6-chloro-3-methylpyridin-2-yl)-[1,2,4]triazolo[1,5-a]pyridin-2-yl)carbamate (120 mg, 0.261 mmol) (Intermediate 9), 4,4,4′,4′,5,5,5′,5′-octamethyl-2,2′-bi(1,3,2-dioxaborolane) (80 mg, 0.313 mmol), and potassium acetate (64.0 mg, 0.652 mmol) in dioxane (4 mL) was added PdCl₂(dppf)-CH₂Cl₂ adduct (21.31 mg, 0.026 mmol). It was purged with nitrogen and heated at 90° C. overnight. The reaction mixture was cooled to rt and the crude product was used for next step without workup. LCMS (ESI, m/z): 467.2 [M−H]⁺. Intermediates 11 and 12: tert-butyl (tert-butoxycarbonyl)(7-(6-chloro-3-methylpyrazin-2-yl)-[1,2,4]triazolo[1,5-a]pyridin-2-yl)carbamate and tert-butyl (tert-butoxycarbonyl)(7-(6-chloro-5-methylpyrazin-2-yl)-[1,2,4]triazolo[1,5-a]pyridin-2-yl)carbamate

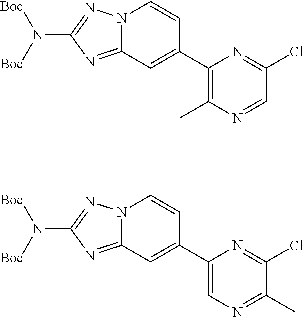 [SMILES: Cc1ncc(Cl)nc1-c1ccn2nc(N(C(=O)OC(C)(C)C)C(=O)OC(C)(C)C)nc2c1 ; Cc1ncc(-c2ccn3nc(N(C(=O)OC(C)(C)C)C(=O)OC(C)(C)C)nc3c2)nc1Cl]

To a pressure vial were added tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (339 mg, 0.736 mmol), 3,5-dichloro-2-methylpyrazine (120 mg, 0.736 mmol), dioxane (8 mL), potassium phosphate (2 M water solution) (1.1 mL, 2.21 mmol), and PdCl₂(dppf)-CH₂Cl₂ adduct (60.1 mg, 0.074 mmol). It was purged with nitrogen and stirred at rt overnight. The reaction was diluted water and ethyl acetate. The layers were separated. The aqueous layer was extracted with ethyl acetate. The combined organic layers were washed with water, brine, dried over anhydrous sodium sulfate, filtered and evaporated to dryness. It was purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 min; Flow Rate: 42.5 mL/min; Column Temperature: 25° C. Fraction collection was triggered by UV (214 nm). Fractions containing the desired product were combined and dried via centrifugal evaporation to provide tert-butyl (tert-butoxycarbonyl)(7-(6-chloro-3-methylpyrazin-2-yl)-[1,2,4]triazolo[1,5-a]pyridin-2-yl)carbamate (119 mg, 34%) as the first-eluting isomer and tert-butyl (tert-butoxycarbonyl)(7-(6-chloro-5-methylpyrazin-2-yl)-[1,2,4]triazolo[1,5-a]pyridin-2-yl)carbamate (95 mg, 28%) as the second-eluting isomer.

Intermediate 11: ¹H NMR (400 MHz, CHLOROFORM-d) δ 8.69-8.66 (m, 1H), 8.61-8.58 (m, 1H), 7.99-7.96 (m, 1H), 7.43-7.39 (m, 1H), 2.75 (s, 3H), 1.51 (s, 18H). LCMS (ESI, m/z): 461.3 [M+H]⁺.

Intermediate 12: ¹H NMR (400 MHz, CHLOROFORM-d) δ 9.95 (s, 1H), 8.72-8.60 (m, 1H), 8.33 (s, 1H), 7.84-7.75 (m, 1H), 2.77 (s, 3H), 1.50 (s, 18H). LCMS (ESI, m/z): 461.3 [M+H]⁺.

Intermediates 13 and 14: 7-(6-chloro-3-methylpyrazin-2-yl)-2-(2,5-dimethyl-1H-pyrrol-1-yl)-8-methyl-[1,2,4]triazolo[1,5-a]pyridine and 7-(6-chloro-5-methylpyrazin-2-yl)-2-(2,5-dimethyl-1H-pyrrol-1-yl)-8-methyl-[1,2,4]triazolo[1,5-a]pyridine

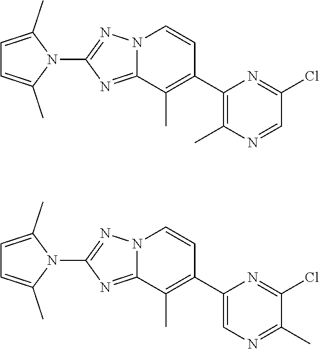 [SMILES: Cc1ncc(Cl)nc1-c1ccn2nc(-n3c(C)ccc3C)nc2c1C ; Cc1ncc(-c2ccn3nc(-n4c(C)ccc4C)nc3c2C)nc1Cl]

Intermediates 13 and 14 were obtained in a similar fashion as described for the preparation of Intermediates 11 and 12 where 2-(2,5-dimethyl-1H-pyrrol-1-yl)-8-methyl-7-(4,4,5,5-tetramethyl-1,3,2-dioxaborolan-2-yl)-[1,2,4]triazolo[1,5-a]pyridine (Intermediate 16) was used in place of tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate. The crude material was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 minutes; Flow Rate: 42.5 mL/min; Column Temperature: 25° C. Fraction collection was triggered by UV signals. Fractions containing the desired product were combined and dried via centrifugal evaporation to give 7-(6-chloro-3-methylpyrazin-2-yl)-2-(2,5-dimethyl-1H-pyrrol-1-yl)-8-methyl-[1,2,4]triazolo[1,5-a]pyridine (85 mg, 26.1%) as the first-eluting isomer and 7-(6-chloro-5-methylpyrazin-2-yl)-2-(2,5-dimethyl-1H-pyrrol-1-yl)-8-methyl-[1,2,4]triazolo[1,5-a]pyridine (75 mg, 23%) as the second-eluting isomer.

Intermediate 13: ¹H NMR (400 MHz, CHLOROFORM-d) δ 8.63 (s, 1H), 8.58-8.49 (m, 1H), 7.02-6.91 (m, 1H), 6.01-5.88 (m, 2H), 2.52 (s, 3H), 2.48 (s, 3H), 2.39 (s, 6H). LCMS (ESI, m/z): 353.1 [M+H]⁺.

Intermediate 14: ¹H NMR (400 MHz, CHLOROFORM-d) δ 8.72-8.63 (m, 1H), 8.58-8.50 (m, 1H), 7.35-7.21 (m, 2H), 6.01-5.88 (m, 2H), 2.79 (s, 3H), 2.78 (s, 3H), 2.39 (s, 6H). LCMS (ESI, m/z): 353.1 [M+H]⁺.

### Intermediate 15: tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate

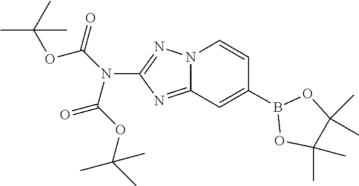 [SMILES: CC(C)(C)OC(=O)N(C(=O)OC(C)(C)C)c1nc2cc(B3OC(C)(C)C(C)(C)O3)ccn2n1]

Step 1: To a suspension of 7-bromo-[1,2,4]triazolo[1,5-a]pyridin-2-amine (3.0 g, 14.1 mmol) in DCM (25 mL) was added a solution of Boc₂O (8.17 mL, 35.2 mmol) and DMAP (688 mg, 5.63 mmol) in acetonitrile (25 mL). The mixture was stirred at rt for 4 h, at which time the mixture was a solution. The volume was reduced to ~⅓ volume, and water was added. Solvents were further removed to yield a suspension which was collected by filtration. After drying in air, the desired material was obtained as a light yellow solid (5.70 g, 98% yield). ¹H NMR (400 MHz, CHLOROFORM-d) δ 8.41 (d, J=7.3 Hz, 1H), 7.92 (d, J=2.0 Hz, 1H), 7.18 (dd, J=7.3, 2.0 Hz, 1H), 1.48 (s, 18H).

Step 2: In a 150 ml pressure vessel, a mixture of the product from step 1 (2.0 g, 4.84 mmol), Bis(pinacolato)diboron (1.475 g, 5.81 mmol), potassium acetate (1.425 g, 14.52 mmol), and PdCl₂(dppf)-CH₂Cl₂ adduct (0.198 g, 0.242 mmol) in dioxane (40 mL) was cooled to −78° C. and solidified, vacuuming/purging with N₂ for 3 cycles. The mixture was stirred at 100° C. for 1.5 h. The reaction mixture was diluted with EtOAc (100 mL), filtered through a pad of Celite, and the filtrate was concentrated under vacuum. The residue was triturated with DCM/hexane (~1:3). The solid was collected and dried under vacuum to give tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate as grey solid (1.9 g, 85% yield). ¹H NMR (400 MHz, CHLOROFORM-d) δ 8.52 (dd, J=6.8, 0.8 Hz, 1H), 8.16 (s, 1H), 7.37 (dd, J=6.8, 1.0 Hz, 1H), 1.45 (s, 18H), 1.39 (s, 12H).

### Intermediate 16: 2-(2,5-dimethyl-1H-pyrrol-1-yl)-8-methyl-7-(4,4,5,5-tetramethyl-1,3,2-dioxaborolan-2-yl)-[1,2,4]triazolo[1,5-a]pyridine

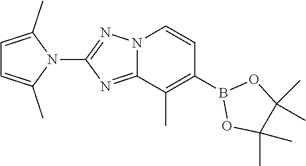 [SMILES: Cc1c(B2OC(C)(C)C(C)(C)O2)ccn2nc(-n3c(C)ccc3C)nc12]

Step 1: To a stirred suspension of 7-bromo-8-methyl-[1,2,4]triazolo[1,5-a]pyridin-2-amine (1.00 g, 4.40 mmol) in toluene (30 mL) was added hexane-2,5-dione (3.02 g, 26.4 mmol). The reaction mixture was heated to 120° C. for 16 h using a Dean-Stark apparatus. Solvent was removed to yield crude material which was purified by column chromatography using silica gel (80 g) eluting with 0-50% of EtOAc/petroleum ether to afford 7-bromo-2-(2,5-dimethyl-1H-pyrrol-1-yl)-8-methyl-[1,2,4]triazolo[1,5-a]pyridine (570 mg, 1.87 mmol, 42.4% yield) as an off white solid.

LCMS (ESI, m/z) 304.9 [M+H]⁺

Step 2: In a Chemglass reaction vial, to a stirred solution of 7-bromo-2-(2,5-dimethyl-1H-pyrrol-1-yl)-8-methyl-[1,2,4]triazolo[1,5-a]pyridine (560 mg, 1.84 mmol) in dioxane (10 mL) was added bis(pinacolato)diboron (559 mg, 2.20 mmol) and potassium acetate (540 mg, 5.50 mmol). The reaction mixture was purged with nitrogen gas for 2 min and 1,1′-bis(diphenylphosphino)-ferrocenedichloro palladium(II) dichloromethane complex (150 mg, 0.183 mmol) was added. The vial was sealed and the mixture was then stirred at 100° C. for 1.5 h. Solvent was evaporated and the crude residue was purified by column chromatography using silica gel, eluting with 0-20% of EtOAc/petroleum ether to yield 2-(2,5-dimethyl-1H-pyrrol-1-yl)-8-methyl-7-(4,4,5,5-tetramethyl-1,3,2-dioxaborolan-2-yl)-[1,2,4]triazolo[1,5-a]pyridine (550 mg, 1.423 mmol, 78% yield) as a white solid. LCMS (ESI, m/z) 353.2 [M+H]⁺.

### Intermediate 17: tert-butyl (tert-butoxycarbonyl)(7-(6-(4,4,5,5-tetramethyl-1,3,2-dioxaborolan-2-yl)pyrazin-2-yl)-[1,2,4]triazolo[1,5-a]pyridin-2-yl)carbamate

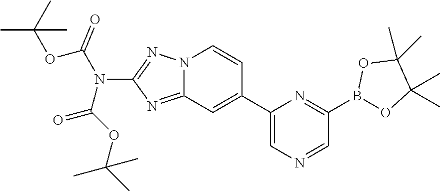 [SMILES: CC(C)(C)OC(=O)N(C(=O)OC(C)(C)C)c1nc2cc(-c3cncc(B4OC(C)(C)C(C)(C)O4)n3)ccn2n1]

Intermediate 17A: In a 150 mL Chemglass pressure vessel were combined tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (3.50 g, 7.60 mmol) (Intermediate 15) and 2-bromo-6-chloropyrazine (1.47 g, 7.60 mmol) in 1,4-dioxane (70 mL). To the mixture was added potassium phosphate tribasic, 2M aqueous solution (11.4 mL, 22.8 mmol). The mixture was sparged with nitrogen by bubbling with rapid stirring for 5 min. Then, dichloro[1,1′-bis(di-tert-butylphosphino)ferrocene]palladium(II) (0.297 g, 0.456 mmol) was added, and the mixture was sparged with a stream of nitrogen with stirring for 5 additional min. The vessel was sealed and the mixture was heated to 65° C. for 5.5 h. The crude mixture was concentrated in vacuo to a residue, and the residue was reconstituted in EtOAc (20 mL) and the resulting black suspension was filtered through a pad of Celite and rinsed through with 1:1 EtOAc:DCM. Concentration in vacuo gave a red oil which solidified upon standing. The residue was dissolved in minimum DCM (~20 mL) and loaded atop a hexanes pre-equilibrated 220 g silica cartridge. Elution gradient 100% hexanes to 100% EtOAc over 10 column volumes, hold 100% EtOAc for 4 CVs. Product eluted as a sharp peak around 55-60% EtOAc in hexanes. Product fractions were combined and concentrated in vacuo to a nearly white powder which was placed under high vacuum for several hours to provide tert-butyl (tert-butoxycarbonyl)(7-(6-chloropyrazin-2-yl)-[1,2,4]triazolo[1,5-a]pyridin-2-yl)carbamate (2.36 g, 5.29 mmol, 69.6% yield) (Intermediate 17A) as an off-white powder. ¹H NMR (500 MHz, CHLOROFORM-d) δ 9.07 (s, 1H), 8.70-8.67 (m, 2H), 8.37 (s, 1H), 7.83 (dd, J=7.3, 1.9 Hz, 1H), 1.51 (s, 18H). MS ESI m/z 446.9 (M+H)⁺

Step 2: To a solution of tert-butyl (tert-butoxycarbonyl)(7-(6-chloropyrazin-2-yl)-[1,2,4]triazolo[1,5-a]pyridin-2-yl)carbamate (180 mg, 0.403 mmol) (Intermediate 17A) in dioxane (4 mL) was added 4,4,4′,4′,5,5,5′,5′-octamethyl-2,2′-bi(1,3,2-dioxaborolane) (123 mg, 0.483 mmol), potassium acetate (99 mg, 1.007 mmol), and PdCl₂(dppf)-CH₂Cl₂ adduct (32.9 mg, 0.040 mmol). The mixture was purged with nitrogen and heated at 85° C. for 1 h and cooled to rt. The crude material was used without workup. LCMS (ESI, m/z) 457.1 [M+H]⁺.

### Intermediate 18: tert-butyl (tert-butoxycarbonyl)(7-(6-(4,4,5,5-tetramethyl-1,3,2-dioxaborolan-2-yl)pyrazin-2-yl)-[1,2,4]triazolo[1,5-a]pyridin-2-yl)carbamate

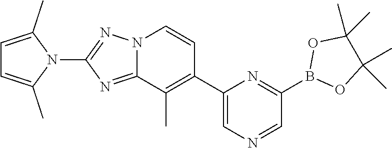 [SMILES: Cc1c(-c2cncc(B3OC(C)(C)C(C)(C)O3)n2)ccn2nc(-n3c(C)ccc3C)nc12]

### Step 1: 7-(6-chloropyrazin-2-yl)-2-(2,5-dimethyl-1H-pyrrol-1-yl)-8-methyl-[1,2,4]triazolo[1,5-a]pyridine

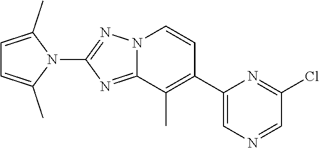 [SMILES: Cc1c(-c2cncc(Cl)n2)ccn2nc(-n3c(C)ccc3C)nc12]

To a solution of 2-(2,5-dimethyl-1H-pyrrol-1-yl)-8-methyl-7-(4,4,5,5-tetramethyl-1,3,2-dioxaborolan-2-yl)-[1,2,4]triazolo[1,5-a]pyridine (340 mg, 0.965 mmol) (Intermediate 16) and 2-bromo-6-chloropyrazine (224 mg, 1.158 mmol) in dioxane (8 mL) was added 2 M potassium phosphate tribasic (1.45 mL, 2.90 mmol), and the resulting mixture was sparged with N₂ for 5 min. PdCl₂(dppf)-CH₂Cl₂ adduct (79 mg, 0.097 mmol) was added and the mixture was heated in a sealed vial at 70° C. for 15 min. The mixture was cooled to rt, and EtOAc was added. The top layer was passed through a pad of Celite and washed with EtOAc. The filtrate was concentrated, and the resulting residue was purified on 40 g silica gel, eluting from 5% to 35% EtOAc/hexane over 13 CV. Fractions containing the product were combined and evaporated to afford 7-(6-chloropyrazin-2-yl)-2-(2,5-dimethyl-1H-pyrrol-1-yl)-8-methyl-[1,2,4]triazolo[1,5-a]pyridine (245 mg, 75% yield). ¹H NMR (400 MHz, CHLOROFORM-d) δ 8.78 (s, 1H), 8.69 (s, 1H), 8.56 (d, J=7.1 Hz, 1H), 7.30 (d, J=7.3 Hz, 1H), 5.95 (s, 2H), 2.80 (s, 3H), 2.42-2.38 (m, 6H). LCMS (ESI, m/z) 339.0/340.8 [M+H]⁺.

Step 2: 2-(2,5-dimethyl-1H-pyrrol-1-yl)-8-methyl-7-(6-(4,4,5,5-tetramethyl-1,3,2-dioxaborolan-2-yl)pyrazin-2-yl)-[1,2,4]triazolo[1,5-a]pyridine

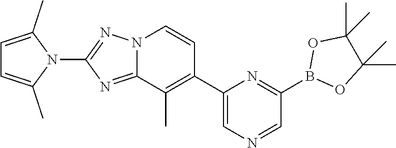 [SMILES: Cc1c(-c2cncc(B3OC(C)(C)C(C)(C)O3)n2)ccn2nc(-n3c(C)ccc3C)nc12]

To a pressure vial were added 4,4,4′,4′,5,5,5′,5′-octamethyl-2,2′-bi(1,3,2-dioxaborolane) (234 mg, 0.921 mmol), 7-(6-chloropyrazin-2-yl)-2-(2,5-dimethyl-1H-pyrrol-1-yl)-8-methyl-[1,2,4]triazolo[1,5-a]pyridine (240 mg, 0.708 mmol), potassium acetate (174 mg, 1.771 mmol), dioxane (6 mL), and PdCl₂(dppf)-CH₂Cl₂ adduct (57.8 mg, 0.071 mmol). The reaction mixture was purged with nitrogen and heated at 85° C. for 90 min. It was cooled to rt and the crude material was used for next step without workup.

LCMS (ESI, m/z) 348.8 [M+H]⁺.

### Intermediate 19: (±)-4-bromo-2-(1-(4-fluorophenyl)-2,2-difluoropropyl)-2H-1,2,3-triazole

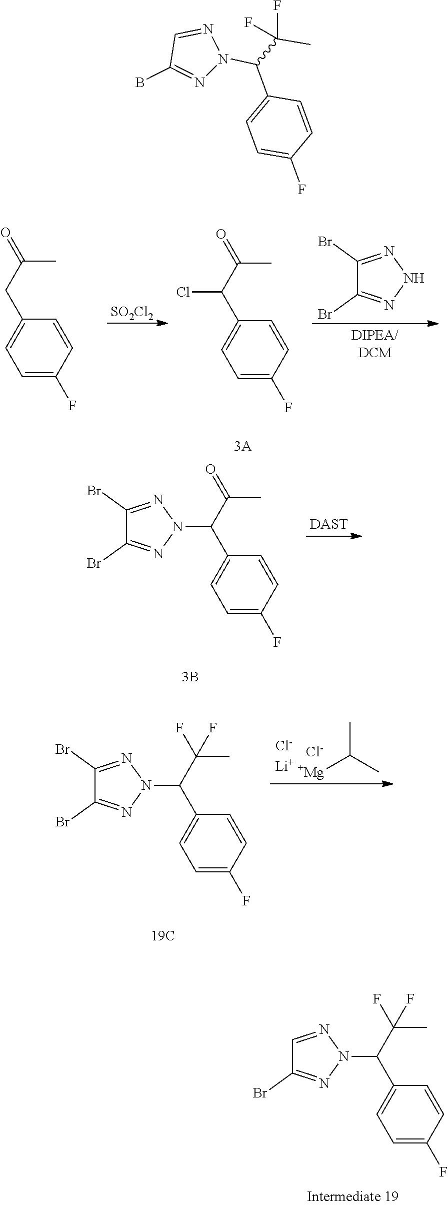

### Intermediate 19A: (±) 1-chloro-1-(4-fluorophenyl)propan-2-one

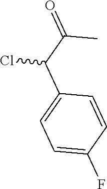

To a solution of 1-(4-fluorophenyl)propan-2-one (5 g, 32.9 mmol) in DCM (50 mL) was slowly added sulfuryl chloride/DCM (39.4 mL, 39.4 mmol) in a wet ice bath under N₂. The mixture was stirred at rt for 2 h. Another 1 eq. of sulfuryl chloride/DCM was added at rt. The mixture was stirred at rt for 20 h. Cold water was added slowly to quench the reaction. The mixture was washed with 3× water, dried over Na₂SO₄, then concentrated to give a fairly clean 1-chloro-1-(4-fluorophenyl)propan-2-one (6.1 g, 32.7 mmol, 99% yield). ¹H NMR (500 MHz, CHLOROFORM-d) δ 7.45-7.40 (m, 2H), 7.14-7.09 (m, 2H), 5.34 (s, 1H), 2.27 (s, 3H).

### Intermediate 19B: (1)-1-(4,5-dibromo-2H-1,2,3-triazol-2-yl)-1-(4-fluorophenyl)propan-2-one

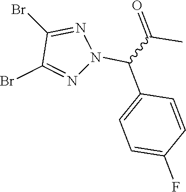 [SMILES: CC(=O)C(c1ccc(F)cc1)n1nc(Br)c(Br)n1]

To a solution of 1-chloro-1-(4-fluorophenyl)propan-2-one (9.0 g, 48.2 mmol) in CH₂Cl₂ (100 mL) was added 4,5-dibromo-2H-1,2,3-triazole (16.41 g, 72.3 mmol) followed by DIPEA (16.85 mL, 96 mmol). The mixture was stirred at rt for 20 h, then refluxed for 4 h. The mixture was concentrated and purified via silica gel chromatography (220 g, hexanes-20% EtOAc) to give clean 1-(4,5-dibromo-2H-1,2,3-triazol-2-yl)-1-(4-fluorophenyl)propan-2-one (8.9 g, 23.61 mmol, 48.9% yield). ¹H NMR (400 MHz, CHLOROFORM-d) δ 7.52-7.46 (m, 2H), 7.21-7.12 (m, 2H), 6.27 (s, 1H), 2.20 (s, 3H). ESI m/z 377.8 (M+H)+.

### Intermediate 19C: (±)-4,5-dibromo-2-(2,2-difluoro-1-(4-fluorophenyl)propyl)-2H-1,2,3-triazole

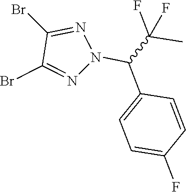 [SMILES: CC(F)(F)C(c1ccc(F)cc1)n1nc(Br)c(Br)n1]

To a solution of (1)-1-(4,5-dibromo-2H-1,2,3-triazol-2-yl)-1-(4-fluorophenyl)propan-2-one (7.8 g, 20.69 mmol) in DCM (50 mL) was slowly added DAST (7.11 mL, 53.8 mmol) at 0° C. under N₂. The mixture was stirred at rt for 20 h, then at 35° C. for 5 h. The reaction was quenched with dropwise NaHCO₃/water carefully at 0° C., and extracted with 2× DCM. The organic layer was concentrated, then purified via silica gel chromatography (220 g, hexanes-30% EtOAc) to give semi-pure (±)-4,5-dibromo-2-(2,2-difluoro-1-(4-fluorophenyl)propyl)-2H-1,2,3-triazole (7.6 g, 19.05 mmol, 92% yield). ¹H NMR (500 MHz, CHLOROFORM-d) δ 7.64 (dd, J=8.6, 5.3 Hz, 2H), 7.15-7.10 (m, 2H), 5.87 (t, J=11.3 Hz, 1H), 1.70 (t, J=18.7 Hz, 3H). ESI m/z 399.7 (M+H)+.

### Intermediate 19: (±)-4-bromo-2-(2,2-difluoro-1-(4-fluorophenyl)propyl)-2H-1,2,3-triazole

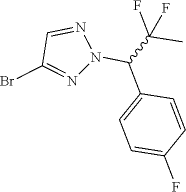 [SMILES: CC(F)(F)C(c1ccc(F)cc1)n1ncc(Br)n1]

To a solution of semi-pure (±)-4,5-dibromo-2-(2,2-difluoro-1-(4-fluorophenyl)propyl)-2H-1,2,3-triazole (7.5 g, 18.80 mmol) (Intermediate 19C) in THF (50 mL) was added Isopropylmagnesium chloride lithium chloride complex solution in THF (18.80 mL, 24.44 mmol) at −70° C. under N₂. The mixture was stirred at −70° C. to −60° C. for 1.5 h. The reaction was quenched with NH₄Cl/water and extracted with 2× EtOAc. The organic layer was concentrated and purified via silica gel chromatography (160 g, hexanes-20% EtOAc) to give a semi-pure (±)-4-bromo-2-(2,2-difluoro-1-(4-fluorophenyl)propyl)-2H-1,2,3-triazole (2.66 g, 8.31 mmol, 44.2% yield). ¹H NMR (400 MHz, CHLOROFORM-d) δ 7.68 (s, 1H), 7.67-7.63 (m, 2H), 7.14-7.09 (m, 2H), 5.98-5.91 (m, 1H), 1.70 (t, J=18.7 Hz, 3H). ESI m/z 321.6 (M+H)+.

### Intermediate 20: (R)-4-bromo-2-(1-(4-fluorophenyl)-2,2-difluoropropyl)-2H-1,2,3-triazole

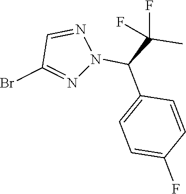 [SMILES: CC(F)(F)[C@@H](c1ccc(F)cc1)n1ncc(Br)n1]

Step 1: Racemic Intermediate 19C was separated using Thar Preparative SFC-350 (S.N. 3617161) with the following preparative SFC conditions: Chiralcel OJ-H (5×25 cm, 5 μm, s/n repacked OJ404); BPR pressure: 100 bars; Temperature: 40° C.; Flow rate: 250 mL/min; Mobile Phase: CO₂/isopropanol with 0.1% NH₄OH (95/5); Detector Wavelength: 220 nm. The first eluting peak was identified as desired single enantiomer (R)-4,5-dibromo-2-(2,2-difluoro-1-(4-fluorophenyl)propyl)-2H-1,2,3-triazole.

Step 2: The homochiral title compound was prepared from the product of step 1 using similar conditions to those described in the final step of the preparation of Intermediate 19.

### Intermediate 21: (±)-2-chloro-6-(2-(2,2-difluoro-1-(4-methoxyphenyl)propyl)-2H-1,2,3-triazol-4-yl)pyridine

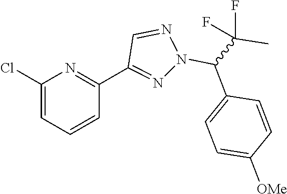 [SMILES: COc1ccc(C(n2ncc(-c3cccc(Cl)n3)n2)C(C)(F)F)cc1]

To a pressure vial were added 2-chloro-6-(4,4,5,5-tetramethyl-1,3,2-dioxaborolan-2-yl)pyridine (104 mg, 0.434 mmol), (±)-4-bromo-2-(2,2-difluoro-1-(4-methoxyphenyl)propyl)-2H-1,2,3-triazole (120 mg, 0.361 mmol) (Intermediate 3D), dioxane (6 mL), potassium phosphate (2 M water solution) (0.54 mL, 1.08 mmol), and PdCl₂(dppf)-CH₂Cl₂ adduct (29.5 mg, 0.036 mmol). It was purged with nitrogen and heated at 85° C. for 1 h. It was diluted with ethyl acetate and water, extracted with ethyl acetate. The combined organic layers were washed with brine, dried over anhydrous sodium sulfate and filtered, and the filtrate was evaporated in vacuo to give the crude residue. The crude product was purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 min; Flow Rate: 42.5 mL/min; Column Temperature: 25° C. Fraction collection was triggered by UV (214 nm). Fractions containing the desired product were combined and dried via centrifugal evaporation to provide afford (±)-2-chloro-6-(2-(2,2-difluoro-1-(4-methoxyphenyl)propyl)-2H-1,2,3-triazol-4-yl)pyridine (71 mg, 53.9%). LCMS (ESI, m/z) 365.2 [M+H]⁺.

### Intermediate 22: tert-butyl (tert-butoxycarbonyl)(7-(5-chloro-6-methylpyridin-3-yl)-[1,2,4]triazolo[1,5-a]pyridin-2-yl)carbamate

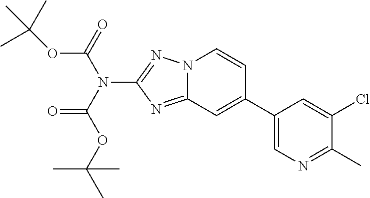 [SMILES: Cc1ncc(-c2ccn3nc(N(C(=O)OC(C)(C)C)C(=O)OC(C)(C)C)nc3c2)cc1Cl]

To a solution of tert-butyl (tert-butoxycarbonyl)(7-(4,4,5,5-tetramethyl-1,3,2-dioxaborolan-2-yl)-[1,2,4]triazolo[1,5-a]pyridin-2-yl) carbamate (1.0957 g, 2.380 mmol) and 5-bromo-3-chloro-2-methylpyridine (0.491 g, 2.380 mmol) in Dioxane (21.64 ml) was added sparged 2 M K3O4P, (3.57 ml, 7.14 mmol). 1,1′-Bis(di-tert-butylphosphino)ferrocene palladium dichloride (0.093 g, 0.143 mmol) was added, and the reaction vessel was flushed with nitrogen. The vessel was sealed and heated to 65° C. overnight. LC/MS showed the formation of the desired product. Solvents were removed, and resuspended in DCM and water. The mixture was extracted 3 times with DCM, and the combined organic extracts were dried with MgSO₄, filtered, and concentrated in vacuo. The residue was purified by flash chromatography (0 to 100% EtOAc/hexane over 10 CV) to afford tert-butyl (tert-butoxycarbonyl)(7-(5-chloro-6-methylpyridin-3-yl)-[1,2,4]triazolo[1,5-a]pyridin-2-yl)carbamate (945.62 mg, 2.056 mmol, 86% yield). MS ESI m/z (M+H)+460.5/462.1.

### Intermediate 23: tert-butyl (tert-butoxycarbonyl)(7,8-dimethyl-[1,2,4]triazolo[1,5-a]pyridin-2-yl)carbamate

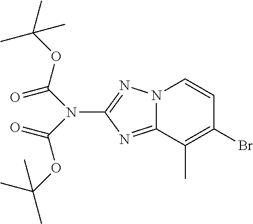 [SMILES: Cc1c(Br)ccn2nc(N(C(=O)OC(C)(C)C)C(=O)OC(C)(C)C)nc12]

To a solution of 7-bromo-8-methyl-[1,2,4]triazolo[1,5-a]pyridin-2-amine (2.5 g, 11.01 mmol) in Acetonitrile (25.02 ml) was added Boc-anhydride (7.67 ml, 33.0 mmol) followed by catalytic DMAP (0.135 g, 1.101 mmol). The mixture was stirred at 70° C. overnight. Solvents were removed and purified by SG chromatography (0 to 60% EtOAc/hexane over 10 CV) to abtain tert-butyl (7-bromo-8-methyl-[1,2,4]triazolo[1,5-a]pyridin-2-yl)(tert-butoxycarbonyl)carbamate (3.05957 g, 7.16 mmol, 65.0% yield). The combined fractions were suspended in 5% EtOAc/hexane, and the resulting white solid was collected by vacuum filtration. The filtrate was concentrated and similarly treated to provide a second off-white crop 660.96 mg (14%). MS ESI m/z (M+H)+426.8/428.7.

### Intermediate 24: tert-butyl (tert-butoxycarbonyl)(8-methyl-7-(4,4,5,5-tetramethyl-1,3,2-dioxaborolan-2-yl)-[1,2,4]triazolo[1,5-a]pyridin-2-yl)carbamate

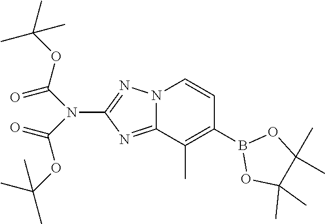 [SMILES: Cc1c(B2OC(C)(C)C(C)(C)O2)ccn2nc(N(C(=O)OC(C)(C)C)C(=O)OC(C)(C)C)nc12]

Dioxane (25.9 ml) was added to tert-butyl (7-bromo-8-methyl-[1,2,4]triazolo[1,5-a]pyridin-2-yl)(tert-butoxycarbonyl)carbamate (Intermediate 23) (2.77173 g, 6.49 mmol), then bis(pinacolato)diboron (2.059 g, 8.11 mmol) was added. Finally, potassium acetate (1.910 g, 19.46 mmol) was added, and the mixture briefly sparged with N₂. [1,1′-bis(diphenylphosphino)ferrocene]dichloropalladium(II), complex with dichloromethane (0.397 g, 0.486 mmol) was added, and the vial was flushed with N₂, sealed, and heated at 90° C. LC/MS showed the desired material had formed (confirmed by 1H NMR) and a peak corresponding to the boronic acid (likely LC/MS artifact). Solvents were removed and the residue was suspended in EtOAc/hexane to afford solid tert-butyl (tert-butoxycarbonyl)(8-methyl-7-(4,4,5,5-tetramethyl-1,3,2-dioxaborolan-2-yl)-[1,2,4]triazolo[1,5-a]pyridin-2-yl)carbamate (2.61583 g, 5.51 mmol, 85% yield). MS ESI m/z (M+H)+475.3. ¹H NMR (400 MHz, CHLOROFORM-d) δ 8.35 (d, J=6.8 Hz, 1H), 7.35 (d, J=6.9 Hz, 1H), 2.87 (s, 3H), 1.47 (s, 18H), 1.40 (s, 12H).

### Intermediate 25: (R)-2-(2,2-difluoro-1-(4-fluorophenyl)propyl)-4-(4,4,5,5-tetramethyl-1,3,2-dioxaborolan-2-yl)-2H-1,2,3-triazole

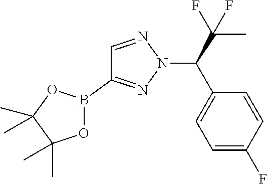 [SMILES: CC(F)(F)[C@@H](c1ccc(F)cc1)n1ncc(B2OC(C)(C)C(C)(C)O2)n1]

To a solution of (R)-4-bromo-2-(2,2-difluoro-1-(4-fluorophenyl)propyl)-2H-1,2,3-triazole (131.0 mg, 0.409 mmol) (Intermediate 20) and bis(pinacolato)diboron (130 mg, 0.512 mmol) in dioxane (3274 μl) was added potassium acetate (100 mg, 1.023 mmol) followed by [1,1′-bis(diphenylphosphino)ferrocene]dichloropalladium(II), complex with dichloromethane (33.4 mg, 0.041 mmol). The vessel was flushed with nitrogen, and the contents heated overnight at 85° C. LC/MS showed the desired material had formed, and the whole reaction mixture was taken forward into subsequent chemistry.

### Intermediate 26: (R)-3-chloro-5-(2-(2,2-difluoro-1-(4-fluorophenyl)propyl)-2H-1,2,3-triazol-4-yl)-2-methylpyridine

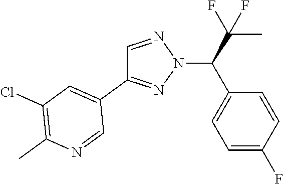 [SMILES: Cc1ncc(-c2cnn([C@H](c3ccc(F)cc3)C(C)(F)F)n2)cc1Cl]

To a solution of (R)-2-(2,2-difluoro-1-(4-fluorophenyl)propyl)-4-(4,4,5,5-tetramethyl-1,3,2-dioxaborolan-2-yl)-2H-1,2,3-triazole (150 mg, 0.409 mmol) (Intermediate 25) and 5-bromo-3-chloro-2-methylpyridine (84 mg, 0.409 mmol) in dioxane (3718 μl) was added sparged 2M 97%, K3O4P, (614 μl, 1.227 mmol), then 1,1′-Bis(di-tert-butylphosphino)ferrocene palladium dichloride (21.33 mg, 0.033 mmol) was added and the reaction vessel was flushed with nitrogen. The vessel was sealed and heated to 65° C. overnight. LC/MS shows the desired product had formed. EtOAc and water were added, and the phases were separated. The aqueous layer was extracted twice more with EtOAc, then the combined organic layers were dried over MgSO₄, filtered, and concentrated in vacuo. Chromatography (0 to 40% EtOAc/hexane over 10 CV) afforded (R)-3-chloro-5-(2-(2,2-difluoro-1-(4-fluorophenyl)propyl)-2H-1,2,3-triazol-4-yl)-2-methylpyridine (97.8 mg, 0.267 mmol, 65.2% yield). MS ESI m/z (M+H)+367.1/368.8. ¹H NMR (400 MHz, CHLOROFORM-d) δ 8.80 (d, J=2.0 Hz, 1H), 8.09 (d, J=2.0 Hz, 1H), 8.00 (s, 1H), 7.70 (dd, J=8.6, 5.3 Hz, 2H), 7.13 (t, J=8.2 Hz, 2H), 6.04 (t, J=11.4 Hz, 1H), 2.69 (s, 3H), 1.74 (m, =18.7 Hz, 3H).

### Intermediate 27: tert-butyl (tert-butoxycarbonyl)(7-(5-chloro-2-(trifluoromethyl)pyridin-3-yl)-[1,2,4]triazolo[1,5-a]pyridin-2-yl)carbamate

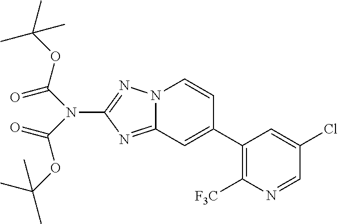 [SMILES: CC(C)(C)OC(=O)N(C(=O)OC(C)(C)C)c1nc2cc(-c3cc(Cl)cnc3C(F)(F)F)ccn2n1]

To a solution of tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (0.535 g, 1.162 mmol) and 3-bromo-5-chloro-2-(trifluoromethyl)pyridine (0.303 g, 1.162 mmol) in dioxane (10.57 ml) was added sparged 2M 97%, K3O4P, (1.743 ml, 3.49 mmol) and 1,1′-bis(di-tert-butylphosphino)ferrocene palladium dichloride (0.045 g, 0.070 mmol) The reaction vessel was flushed with nitrogen, sealed, and heated to 65° C. overnight. LC/MS showed a mix of the desired product and mono-deprotected product. Dioxane was partially removed, and the residue was diluted with EtOAc. The phases were split, and the material was extracted 3 times into EtOAc. The combined organic layers were dried over MgSO₄, filtered, and concentrated in vacuo. The residue was purified by flash chromatography (0 to 70% EtOAc/hexane over 10 CV, 2 CV hold at 70%) to afford tert-butyl (tert-butoxycarbonyl)(7-(5-chloro-2-(trifluoromethyl)pyridin-3-yl)-[1,2,4]triazolo[1,5-a]pyridin-2-yl)carbamate (191.6 mg, 0.373 mmol, 32.1% yield) and tert-butyl (7-(5-chloro-2-(trifluoromethyl)pyridin-3-yl)-[1,2,4]triazolo[1,5-a]pyridin-2-yl)carbamate (160.7 mg, 0.388 mmol, 33.4% yield). MS ESI m/z (M+H)+514.4. ¹H NMR (400 MHz, CHLOROFORM-d) δ 8.78 (d, J=2.2 Hz, 1H), 8.64 (dd, J=7.0, 0.8 Hz, 1H), 7.81 (d, J=2.1 Hz, 1H), 7.71 (s, 1H), 7.05 (dd, J=7.1, 1.7 Hz, 1H), 1.53 (s, 18H).

### Intermediate 28: tert-butyl (tert-butoxycarbonyl)(7-(5-chloro-2-(trifluoromethyl)pyridin-3-yl)-8-methyl-[1,2,4]triazolo[1,5-a]pyridin-2-yl)carbamate

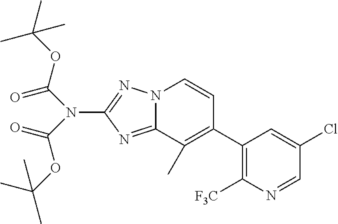 [SMILES: Cc1c(-c2cc(Cl)cnc2C(F)(F)F)ccn2nc(N(C(=O)OC(C)(C)C)C(=O)OC(C)(C)C)nc12]

To a solution of tert-butyl (tert-butoxycarbonyl)(8-methyl-7-(4,4,5,5-tetramethyl-1,3,2-dioxaborolan-2-yl)-[1,2,4]triazolo[1,5-a]pyridin-2-yl)carbamate (0.3 g, 0.632 mmol) and 3-bromo-5-chloro-2-(trifluoromethyl)pyridine (0.165 g, 0.632 mmol) in dioxane (5.75 ml) was added sparged 2M 97%, K3O4P, (0.949 ml, 1.897 mmol). 1,1′-bis(di-tert-butylphosphino)ferrocene palladium dichloride (0.025 g, 0.038 mmol) was added, and the reaction vessel was flushed with nitrogen. The vessel was sealed and heated to 65° C. overnight. LC/MS showed the desired product had formed. Solvents were removed and the remaining material was extracted 3 times into EtOAc (some water was added to aid the extraction). The combined organic extracts were dried over MgSO₄, filtered, and concentrated. The resulting residue was purified by SG chromatography (0 to 50% EtOAc/hexane over 15 CV followed by 10 CV at 50% EtOAc/hexane) to afford tert-butyl (tert-butoxycarbonyl)(7-(5-chloro-2-(trifluoromethyl)pyridin-3-yl)-8-methyl-[1,2,4]triazolo[1,5-a]pyridin-2-yl)carbamate (77.87 mg, 0.148 mmol, 23.32% yield). MS ESI m/z (M+H)+528.3/530.0. ¹H NMR (400 MHz, CHLOROFORM-d) δ 8.79 (d, J=2.2 Hz, 1H), 8.47 (d, J=7.0 Hz, 1H), 7.69 (d, J=2.2 Hz, 1H), 6.86 (d, J=7.0 Hz, 1H), 2.40 (s, 3H), 1.55 (s, 18H).

### Intermediate 29: (±)-6-chloro-2-(2-(2,2-difluoro-1-(4-fluorophenyl)propyl)-2H-1,2,3-triazol-4-yl)-3-methylpyridine

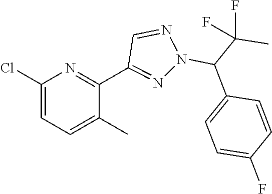 [SMILES: Cc1ccc(Cl)nc1-c1cnn(C(c2ccc(F)cc2)C(C)(F)F)n1]

To a pressure vial were added 2-bromo-6-chloro-3-methylpyridine (97 mg, 0.468 mmol), (±)-2-(2,2-difluoro-1-(4-fluorophenyl)propyl)-4-(4,4,5,5-tetramethyl-1,3,2-dioxaborolan-2-yl)-2H-1,2,3-triazole (172 mg, 0.468 mmol) (Racemic Intermediate 25), dioxane (4 mL), potassium phosphate (2 M water solution) (0.702 mL, 1.404 mmol), and PdCl₂(dppf)-CH₂Cl₂ adduct (38.2 mg, 0.047 mmol). The reaction mixture was purged with nitrogen and heated at 85° C. for 90 min. It was cooled to rt, diluted with EtOAc, washed with water and brine, dried over anhydrous sodium sulfate and filtered, and the filtrate was evaporated in vacuo to give the crude residue. The crude product was purified purified by silica gel chromatography (25 g RediSep® column, eluting with a gradient from 0-25% EtOAc in. hexanes, 14 cv). Fractions containing the product were combined and concentrated to afford (±)-6-chloro-2-(2-(2,2-difluoro-1-(4-fluorophenyl)propyl)-2H-1,2,3-triazol-4-yl)-3-methylpyridine (145 mg, 0.395 mmol, 84% yield). ¹H NMR (400 MHz, CHLOROFORM-d) δ 8.32 (s, 1H), 7.72-7.66 (m, 2H), 7.62-7.54 (m, 1H), 7.23 (d, J=8.0 Hz, 1H), 7.16-7.07 (m, 2H), 6.05 (br d, J=12.8 Hz, 1H), 2.66 (s, 3H), 1.73 (t, J=18.7 Hz, 3H). LCMS (ESI, m/z) 358.2 [M+H]⁺.

### Intermediate 30: (2-amino-[1,2,4]triazolo[1,5-a]pyridin-7-yl)boronic acid

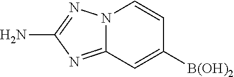 [SMILES: Nc1nc2cc(B(O)O)ccn2n1]

A mixture of 7-bromo-[1,2,4]triazolo[1,5-a]pyridin-2-amine (10 g, 46.9 mmol), bis(pinacolato)diboron (14.30 g, 56.3 mmol), potassium acetate (13.82 g, 141 mmol), and [1,1′-BIS(DIPHENYLPHOSPHINO)FERROCENE]DICHLOROPALLADIUM(II) (1.717 g, 2.347 mmol) in 1,4-dioxane (100 mL) was purged with N₂ and stirred at 100° C. for 2 h. LCMS showed that the reaction was complete. The resulting reaction solution was cooled to rt and aliquotted for use in subsequent reactions.

### Intermediate 31: 4-bromo-2-(1-(4-fluorophenyl)propyl)-2H-1,2,3-triazole

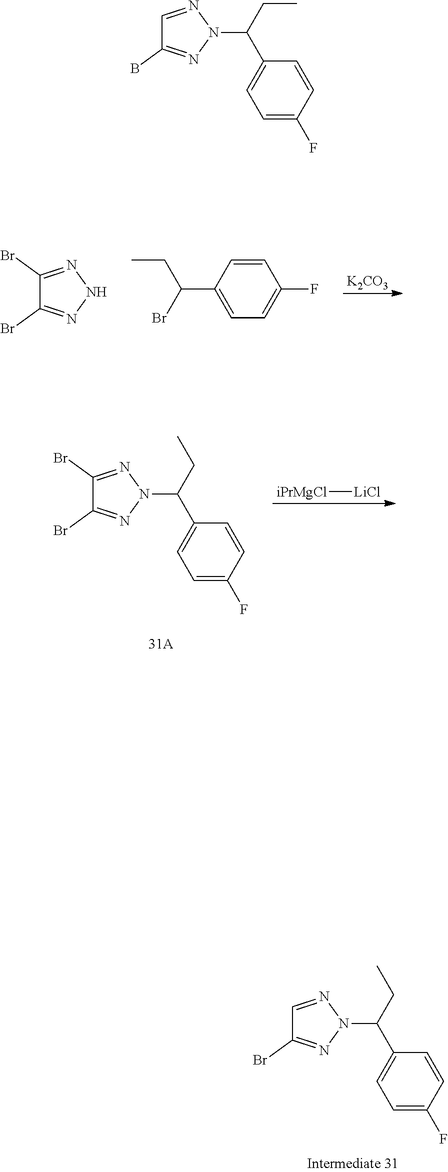

Step 1: To a solution of 4,5-dibromo-2H-1,2,3-triazole (1000 mg, 4.42 mmol) in DMF (10 mL) was added K2CO3 (764 mg, 5.53 mmol) followed by 1-(1-bromopropyl)-4-fluorobenzene (800 mg, 3.69 mmol). The mixture was stirred at rt for 4 h. EtOAc was added, washed with water, then concentrated. The residue was purified via silica gel chromatography (40 g, hexanes-100% EtOAc) to give only one major spot 4,5-dibromo-2-(1-(4-fluorophenyl)propyl)-2H-1,2,3-triazole (920 mg, 2.53 mmol, 68.8% yield). ¹H NMR (499 MHz, CHLOROFORM-d) δ 7.42-7.37 (m, 2H), 7.08-7.03 (m, 2H), 5.44 (dd, J=9.1, 6.6 Hz, 1H), 2.53 (ddq, J=14.2, 9.1, 7.2 Hz, 1H), 2.28-2.18 (m, 1H), 0.93 (t, J=7.4 Hz, 3H). MS ESI m/z 361.8 (M+H)+.

Step 2: To a solution of 4,5-dibromo-2-(1-(4-fluorophenyl)propyl)-2H-1,2,3-triazole (510 mg, 1.405 mmol) (Intermediate 31A) in THF (6 mL) was added Isopropylmagnesium chloride lithium chloride complex solution in THF (1.189 mL, 1.545 mmol) at −20° C. under N₂. The mixture was allowed to warm up to 0° C. over 1 h. Another 0.5 eq. of Isopropylmagnesium chloride lithium chloride complex solution in THF was added at −10° C. The mixture was stirred at 0° C. for 45 min. EtOAc was added, washed with water then concentrated. The residue was purified via silica gel chromatography (24 g, hexanes-30% EtOAc) to give 4-bromo-2-(1-(4-fluorophenyl)propyl)-2H-1,2,3-triazole (340 mg, 1.197 mmol, 85% yield). ¹H NMR (500 MHz, CHLOROFORM-d) δ 7.57 (s, 1H), 7.41-7.37 (m, 2H), 7.07-7.02 (m, 2H), 5.49 (dd, J=9.2, 6.5 Hz, 1H), 2.54 (ddq, J=14.2, 9.2, 7.2 Hz, 1H), 2.28-2.19 (m, 1H), 0.92 (t, J=7.4 Hz, 3H). MS ESI m/z 285.8 (M+H)+.

### Intermediate 32: (2-(1-(4-fluorophenyl)propyl)-2H-1,2,3-triazol-4-yl)boronic acid

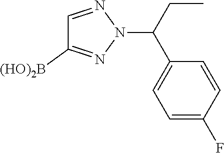 [SMILES: CCC(c1ccc(F)cc1)n1ncc(B(O)O)n1]

To a solution of 4-bromo-2-(1-(4-fluorophenyl)propyl)-2H-1,2,3-triazole (685 mg, 2.411 mmol) (Intermediate 31) in THF (10 mL) was added Isopropylmagnesium chloride lithium chloride complex solution in THF (2.78 mL, 3.62 mmol) at 0° C. under N₂. The mixture was stirred at rt for 1 h. Another 0.5 eq. of Isopropylmagnesium chloride lithium chloride complex solution in THF was added. The mixture was stirred at rt for 1 h. Trimethyl borate (0.970 mL, 8.68 mmol) was added at −20° C. The mixture was allowed to warm up to 0° C. over 1 h. 1 N HCl was added and extracted with 2× EtOAc. The organic layer was dried over Na₂SO₄, then concentrated to give a crude (2-(1-(4-fluorophenyl)propyl)-2H-1,2,3-triazol-4-yl)boronic acid (600 mg, 2.411 mmol, 100% yield). The unpurified material was used for subsequent chemistry. ESI m/z 249.8 (M+H)+.

### Intermediate 33: (+)-(2-(2,2-difluoro-1-(4-fluorophenyl)propyl)-2H-1,2,3-triazol-4-yl)boronic acid

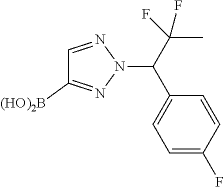 [SMILES: CC(F)(F)C(c1ccc(F)cc1)n1ncc(B(O)O)n1]

Step 1: To a solution of (±)-4,5-dibromo-2-(2,2-difluoro-1-(4-fluorophenyl)propyl)-2H-1,2,3-triazole (226 mg, 0.566 mmol) (Intermediate 19C) in THF (2 mL) was added Isopropylmagnesium chloride lithium chloride complex solution in THF (0.654 mL, 0.850 mmol) at −70° C. under N₂. The mixture was stirred at −70° C. to −60° C. for 2 h. Trimethyl borate (0.190 mL, 1.699 mmol) was added. The mixture was allowed to warm up to rt and stirred at rt for 1 h. Hexane was added and extracted with 2×1N NaOH. The basic aqueous layer was neutralized with 6N HCl to neutral, then extracted with 2× EtOAc. The organic layer was dried over Na₂SO₄ then concentrated to give a crude (5-bromo-2-(2,2-difluoro-1-(4-fluorophenyl)propyl)-2H-1,2,3-triazol-4-yl)boronic acid (130 mg, 0.357 mmol, 63.1% yield). MS ESI m/z 365.5 (M+H)⁺.

Step 2: To a solution of the crude (5-bromo-2-(2,2-difluoro-1-(4-fluorophenyl)propyl)-2H-1,2,3-triazol-4-yl)boronic acid (130 mg, 0.357 mmol) in EtOH (3 mL) was added 10% wt Pd—C(76 mg, 0.071 mmol) under N₂. Then ammonium formate (225 mg, 3.57 mmol) was added. The mixture was stirred at 90° C. for 1 h. Another portion of ammonium formate (225 mg, 3.57 mmol) was added. The mixture was stirred at 90° C. for 2 h. The Pd/C was filtered off. The filtrate was concentrated, dissolved in EtOAc, washed twice with water, dried over Na₂SO₄ then concentrated again. A crude (2-(2,2-difluoro-1-(4-fluorophenyl)propyl)-2H-1,2,3-triazol-4-yl)boronic acid (90 mg, 0.316 mmol, 88% yield) was obtained. MS ESI m/z 285.6 (M+H)⁺.

### Intermediate 34: 4-(3-chloro-2-fluorophenyl)-2-(1-(4-fluorophenyl)propyl)-2H-1,2,3-triazole

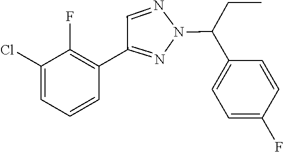 [SMILES: CCC(c1ccc(F)cc1)n1ncc(-c2cccc(Cl)c2F)n1]

To a solution of (3-chloro-2-fluorophenyl)boronic acid (104 mg, 0.597 mmol), 4-bromo-2-(1-(4-fluorophenyl)propyl)-2H-1,2,3-triazole (106 mg, 0.373 mmol) (Intermediate 31), and PdCl₂(dppf)-CH₂Cl₂ adduct (30.5 mg, 0.037 mmol) in dioxane (2 mL) was added tripotassium phosphate (0.560 mL, 1.119 mmol). The mixture was stirred at 95° C. for 40 min. Water was added, then extracted with EtOAc. The organic layer was concentrated. The residue was purified via silica gel chromatography (4 g, hexanes-60% EtOAc) to give 4-(3-chloro-2-fluorophenyl)-2-(1-(4-fluorophenyl)propyl)-2H-1,2,3-triazole (115 mg, 0.345 mmol, 92% yield).

¹H NMR (400 MHz, CHLOROFORM-d) δ 8.02 (d, J=4.3 Hz, 1H), 7.94 (ddd, J=8.0, 6.4, 1.7 Hz, 1H), 7.47-7.37 (m, 3H), 7.17 (td, J=7.9, 1.0 Hz, 1H), 7.08-7.02 (m, 2H), 5.59 (dd, J=9.2, 6.4 Hz, 1H), 2.62 (ddq, J=14.2, 9.2, 7.2 Hz, 1H), 2.36-2.23 (m, 1H), 0.95 (t, J=7.3 Hz, 3H). MS ESI m/z 333.9 (M+H)⁺.

### Intermediate 35: 2-chloro-6-(2-(1-(4-fluorophenyl)propyl)-2H-1,2,3-triazol-4-yl)pyridine

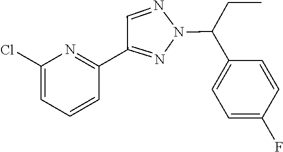 [SMILES: CCC(c1ccc(F)cc1)n1ncc(-c2cccc(Cl)n2)n1]

To a solution of 2-chloro-6-(4,4,5,5-tetramethyl-1,3,2-dioxaborolan-2-yl)pyridine (115 mg, 0.480 mmol), 4-bromo-2-(1-(4-fluorophenyl)propyl)-2H-1,2,3-triazole (150 mg, 0.528 mmol) (Intermediate 31), and PdCl₂(dppf)-CH₂Cl₂ adduct (27.4 mg, 0.034 mmol) in Dioxane (2 mL) was added tripotassium phosphate (0.720 mL, 1.440 mmol). The mixture was stirred at 95° C. for 40 min. Water was added, then extracted twice with EtOAc. The organic layers were concentrated. The residue was purified via silica gel chromatography (12 g, hexanes-20% EtOAc) to give 2-chloro-6-(2-(1-(4-fluorophenyl)propyl)-2H-1,2,3-triazol-4-yl)pyridine (93 mg, 0.294 mmol, 61.1% yield). MS ESI m/z 317.0 (M+H)⁺.

### Intermediate 36: 2-chloro-6-(2-(1-(4-fluorophenyl)propyl)-2H-1,2,3-triazol-4-yl)pyrazine

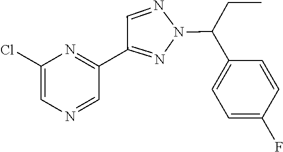 [SMILES: CCC(c1ccc(F)cc1)n1ncc(-c2cncc(Cl)n2)n1]

Prepared in a similar fashion to Intermediate 35 using 2-chloro-6-(4,4,5,5-tetramethyl-1,3,2-dioxaborolan-2-yl)pyrazine and 4-bromo-2-(1-(4-fluorophenyl)propyl)-2H-1,2,3-triazole (Intermediate 31) as starting materials. ¹H NMR (400 MHz, CHLOROFORM-d) δ 9.13-9.10 (m, 1H), 8.58-8.49 (m, 1H), 8.30-8.21 (m, 1H), 7.48-7.42 (m, 2H), 7.09-7.02 (m, 2H), 5.61 (dd, J=9.2, 6.4 Hz, 1H), 2.62 (ddq, J=14.2, 9.2, 7.2 Hz, 1H), 2.37-2.25 (m, 1H), 0.95 (t, J=7.4 Hz, 3H). ESI m/z 317.8 (M+H)⁺.

### Intermediate 37: 2-chloro-4-(2-(1-(4-fluorophenyl)propyl)-2H-1,2,3-triazol-4-yl)pyrimidine

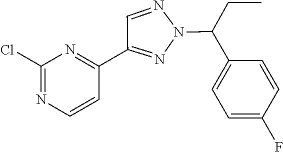 [SMILES: CCC(c1ccc(F)cc1)n1ncc(-c2ccnc(Cl)n2)n1]

To a solution of crude (2-(1-(4-fluorophenyl)propyl)-2H-1,2,3-triazol-4-yl)boronic acid (140 mg, 0.562 mmol) (Intermediate 32), 4-bromo-2-chloropyrimidine (141 mg, 0.731 mmol) and PdCl₂(dppf)-CH₂Cl₂ adduct (22.95 mg, 0.028 mmol) in Dioxane (2 mL) was added 2.0 M Na2CO3/H2O (0.843 mL, 1.686 mmol). The mixture was stirred at 70° C. for 30 min. EtOAc was added, washed with water then concentrated. The residue was purified via silica gel chromatography (12 g, hexanes-40% EtOAc) to give 2-chloro-4-(2-(1-(4-fluorophenyl)propyl)-2H-1,2,3-triazol-4-yl)pyrimidine (120 mg, 0.378 mmol, 67.2% yield). ¹H NMR (500 MHz, CHLOROFORM-d) δ 8.65 (d, J=5.1 Hz, 1H), 8.32 (s, 1H), 7.85 (d, J=5.1 Hz, 1H), 7.45-7.40 (m, 2H), 7.08-7.02 (m, 2H), 5.60 (dd, J=9.2, 6.5 Hz, 1H), 2.60 (ddq, J=14.2, 9.2, 7.2 Hz, 1H), 2.34-2.25 (m, 1H), 0.93 (t, J=7.4 Hz, 3H). ESI m/z 317.8 (M+H)⁺.

### Intermediate 38: (±)-2-chloro-6-(2-(2,2-difluoro-1-(4-fluorophenyl)propyl)-2H-1,2,3-triazol-4-yl)pyridine

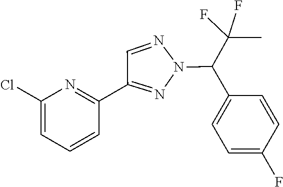 [SMILES: CC(F)(F)C(c1ccc(F)cc1)n1ncc(-c2cccc(Cl)n2)n1]

To a solution of 2-chloro-6-(4,4,5,5-tetramethyl-1,3,2-dioxaborolan-2-yl)pyridine (145 mg, 0.605 mmol), 4-bromo-2-(2,2-difluoro-1-(4-fluorophenyl)propyl)-2H-1,2,3-triazole (388 mg, 1.211 mmol) (Intermediate 19), and PdCl₂(dppf)-CH₂Cl₂ adduct (34.6 mg, 0.042 mmol) in Dioxane (3 mL) was added tripotassium phosphate (0.908 mL, 1.816 mmol). The mixture was stirred at 95° C. for 40 min. Water was added, then extracted twice with EtOAc. The organic layers were concentrated and purified via silica gel chromatography (12 g, hexanes-50% EtOAc) to give 2-chloro-6-(2-(2,2-difluoro-1-(4-fluorophenyl)propyl)-2H-1,2,3-triazol-4-yl)pyridine (54 mg, 0.153 mmol, 25.3% yield). ¹H NMR (400 MHz, CHLOROFORM-d) δ 8.32 (s, 1H), 7.92 (dd, J=7.7, 0.9 Hz, 1H), 7.74 (t, J=7.8 Hz, 1H), 7.69 (dd, J=8.6, 5.2 Hz, 2H), 7.32 (dd, J=7.9, 0.8 Hz, 1H), 7.15-7.08 (m, 2H), 6.06 (dd, J=12.5, 10.3 Hz, 1H), 1.74 (t, J=18.7 Hz, 3H). ESI m/z 352.8 (M+H)⁺.

### Intermediate 39: 2-chloro-6-(2-(2,2-difluoro-1-(4-fluorophenyl)propyl)-2H-1,2,3-triazol-4-yl)pyrazine

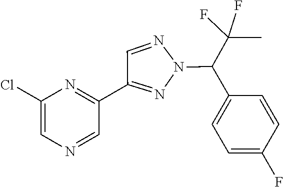 [SMILES: CC(F)(F)C(c1ccc(F)cc1)n1ncc(-c2cncc(Cl)n2)n1]

Step 1: To a solution of 2,6-dichloropyrazine (2.69 g, 18.06 mmol) in Dioxane (40 mL) was added bis(pinacolato)diboron (5.04 g, 19.86 mmol), tricyclohexylphosphine (0.405 g, 1.445 mmol), PdOAc2 (0.162 g, 0.722 mmol) and potassium acetate (4.43 g, 45.1 mmol). The mixture was stirred at 112° C. for 3 h. Hexanes was added, and the mixture was filtered through a short column of silica gel and rinsed with EtOAc. The filtrate was concentrated to give a crude 2-chloro-6-(4,4,5,5-tetramethyl-1,3,2-dioxaborolan-2-yl)pyrazine. The crude material was used directly for subsequent chemistry.

Step 2: To a solution of the crude 2-chloro-6-(4,4,5,5-tetramethyl-1,3,2-dioxaborolan-2-yl)pyrazine (301 mg, 1.250 mmol), 4-bromo-2-(2,2-difluoro-1-(4-fluorophenyl)propyl)-2H-1,2,3-triazole (500 mg, 1.562 mmol) (Intermediate 19), and PdCl₂(dppf)-CH₂Cl₂ adduct (63.8 mg, 0.078 mmol) in Dioxane (3 mL) was added tripotassium phosphate (2.343 mL, 4.69 mmol). The mixture was stirred at 95° C. for 30 min. Another 3×300 mg of the crude 2-chloro-6-(4,4,5,5-tetramethyl-1,3,2-dioxaborolan-2-yl)pyrazine was added every 30 min while the reaction was stirred at 95° C. Water was added, then the mixture was extracted twice with EtOAc. The organic layers were concentrated. The residue was purified via silica gel chromatography (24 g, hexanes-50% EtOAc). The desired fractions were identified via LCMS. A slightly impure 2-chloro-6-(2-(2,2-difluoro-1-(4-fluorophenyl)propyl)-2H-1,2,3-triazol-4-yl)pyrazine (44 mg, 0.124 mmol, 7.96% yield) was obtained. ¹H NMR (400 MHz, CHLOROFORM-d) δ 9.16 (s, 1H), 8.58 (s, 1H), 8.34 (s, 1H), 7.75-7.68 (m, 2H), 7.17-7.10 (m, 2H), 6.08 (dd, J=12.2, 10.5 Hz, 1H), 1.75 (t, J=18.7 Hz, 3H). ESI m/z 353.7 (M+H)⁺.

### Intermediate 40: 4-bromo-2-(3,3-difluoro-1-(4-fluorophenyl)propyl)-2H-1,2,3-triazole

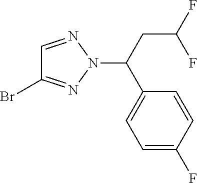

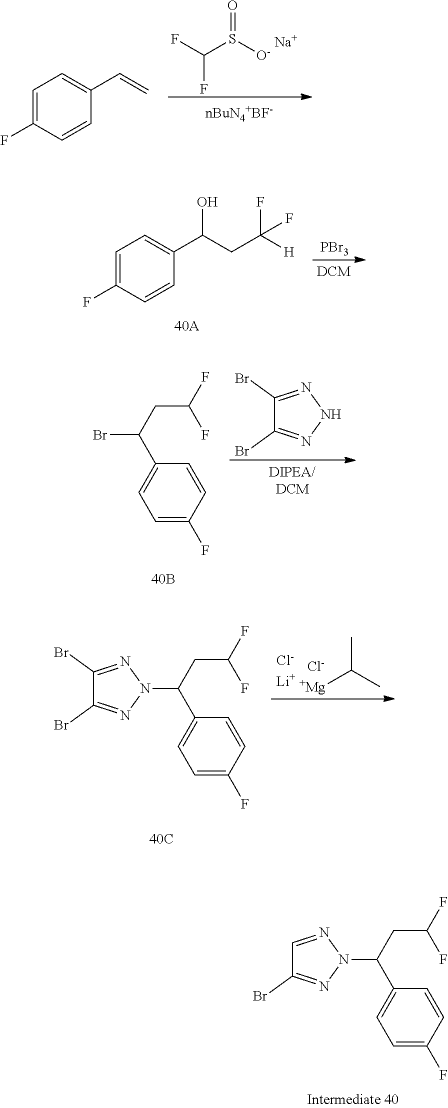

Step 1: 1-fluoro-4-vinylbenzene (98 mg, 0.8 mmol), difluoromethanesulfinic acid sodium (166 mg, 1.200 mmol), tetrabutylammonium tetrafluoroborate (65.9 mg, 0.200 mmol), and acetone (7.2 mL)/water (0.8 mL) were added to a 10 mL IKA vial equipped with a graphite anode (IKA Graphite SK-50, 8 mm×52 mm×2 mm, immersion length is 25 mm) and a Stainless-Steel cathode (8 mm×52 mm×2 mm, immersion length is 25 mm). The mixture was purged with N₂ before applying electricity. The electrochemical reactor was operated under 5 mA, 3.5 F/mol (2.5 mA/cm for 7.5 h) constant current mode at room temperature under magnetic stirring (800 rpm). The mixture was concentrated and purified via silica gel chromatography (4 g, hexanes-100% EtOAc) to give 3,3-difluoro-1-(4-fluorophenyl)propan-1-ol (115 mg, 0.605 mmol, 76% yield) (Intermediate 40A). ¹H NMR (400 MHz, CHLOROFORM-d) δ 7.37 (dd, J=8.5, 5.3 Hz, 2H), 7.12-7.05 (m, 2H), 6.17-5.86 (m, 1H), 4.97 (dd, J=9.7, 3.8 Hz, 1H), 2.42-2.26 (m, 1H), 2.23-2.09 (m, 1H), 2.04-1.91 (br, 1H).

Step 2: To a solution of 3,3-difluoro-1-(4-fluorophenyl)propan-1-ol (710 mg, 3.73 mmol) (Intermediate 40A) in DCM (15 mL) was added phosphorus tribromide in DCM (4.48 mL, 4.48 mmol). The mixture was stirred at rt for 18 h. DCM was added, washed twice with NaHCO₃/water and water, dried over Na₂SO₄, then concentrated to give 1-(1-bromo-3,3-difluoropropyl)-4-fluorobenzene (740 mg, 2.92 mmol, 78% yield) (Intermediate 40B).

¹H NMR (400 MHz, CHLOROFORM-d) δ 7.45-7.39 (m, 2H), 7.12-7.06 (m, 2H), 6.12-5.81 (m, 1H), 5.07 (dd, J=9.8, 5.4 Hz, 1H), 2.92-2.75 (m, 1H), 2.62 (tdt, J=14.8, 11.7, 5.9 Hz, 1H).

Step 3: To a solution of 4,5-dibromo-2H-1,2,3-triazole (163 mg, 0.717 mmol) and 1-(1-bromo-3,3-difluoropropyl)-4-fluorobenzene (165 mg, 0.652 mmol) (Intermediate 40B) in DCM (3 mL) was added DIPEA (0.171 mL, 0.978 mmol). The mixture was stirred at 45° C. for 2 h. Another 2 eq. of 4,5-dibromo-2H-1,2,3-triazole and 3 eq. of DIPEA were added. The mixture was stirred at 45° C. for 20 h. The mixture was concentrated and purified via silica gel chromatography (24 g, hexanes-50% EtOAc) to give 4,5-dibromo-2-(3,3-difluoro-1-(4-fluorophenyl)propyl)-2H-1,2,3-triazole (175 mg, 0.439 mmol, 67.3% yield). ¹H NMR (400 MHz, CHLOROFORM-d) δ 7.43-7.36 (m, 2H), 7.13-7.05 (m, 2H), 5.89-5.56 (m, 2H), 3.26-3.10 (m, 1H), 2.80-2.65 (m, 1H).

Step 4: To a solution of 4,5-dibromo-2-(3,3-difluoro-1-(4-fluorophenyl)propyl)-2H-1,2,3-triazole (300 mg, 0.752 mmol) (Intermediate 40C) in THF (5 mL) was added Isopropylmagnesium chloride lithium chloride complex solution in THF (0.868 mL, 1.128 mmol) at 0° C. under N₂. The mixture was allowed to warm up to rt and stirred at rt for 1 h. Another 0.5 eq. of Isopropylmagnesium chloride lithium chloride complex solution in THF was added. After stirring at rt for 20 min, Hexanes/EtOAc was added, quenched with NH₄Cl/water, washed with water, then concentrated. The residue was purified via silica gel chromatography (12 g, hexanes-30% EtOAc) to give 4-bromo-2-(3,3-difluoro-1-(4-fluorophenyl)propyl)-2H-1,2,3-triazole (120 mg, 0.375 mmol, 49.9% yield) (Intermediate 40). ¹H NMR (400 MHz, CHLOROFORM-d) δ 7.61 (s, 1H), 7.42-7.35 (m, 2H), 7.12-7.04 (m, 2H), 5.90-5.55 (m, 2H), 3.29-3.12 (m, 1H), 2.80-2.64 (m, 1H). ESI m/z=319.7 (M+H)⁺.

### Intermediate 41: tert-butyl (tert-butoxycarbonyl)(7-(6-chloropyridin-2-yl)-[1,2,4]triazolo[1,5-a]pyrazin-2-yl)carbamate

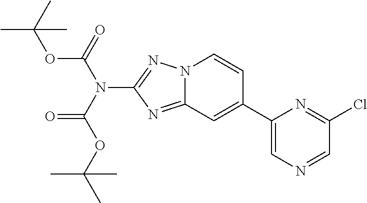 [SMILES: CC(C)(C)OC(=O)N(C(=O)OC(C)(C)C)c1nc2cc(-c3cncc(Cl)n3)ccn2n1]

To a solution of 2-bromo-6-chloropyrazine (1.994 g, 10.31 mmol), (2-(bis(tert-butoxycarbonyl)amino)-[1,2,4]triazolo[1,5-a]pyridin-7-yl)boronic acid (4.52 g, 11.95 mmol) (Intermediate 52), and 1,1′-Bis(di-tert-butylphosphino)ferrocene palladium dichloride (0.576 g, 0.884 mmol) in dioxane (30 mL) was added tripotassium phosphate (17.93 mL, 35.9 mmol). The mixture was stirred at 70° C. for 1 h. Water was added and extracted twice with EtOAc. The organic layers were concentrated and purified via silica gel chromatography (80 g, hexanes-100% EtOAc) to give tert-butyl (tert-butoxycarbonyl)(7-(6-chloropyrazin-2-yl)-[1,2,4]triazolo[1,5-a]pyridin-2-yl)carbamate (3.6 g, 8.06 mmol, 67.4% yield). ¹H NMR (400 MHz, CHLOROFORM-d) δ 9.07 (s, 1H), 8.70-8.65 (m, 2H), 8.37 (dd, J=1.9, 0.9 Hz, 1H), 7.83 (dd, J=7.2, 2.0 Hz, 1H), 1.51 (s, 18H). MS ESI m/z 447.1 (M+H)⁺.

### Intermediate 42: tert-butyl (tert-butoxycarbonyl)(7-(6-(4,4,5,5-tetramethyl-1,3,2-dioxaborolan-2-yl)pyrazin-2-yl)-[1,2,4]triazolo[1,5-a]pyridin-2-yl)carbamate

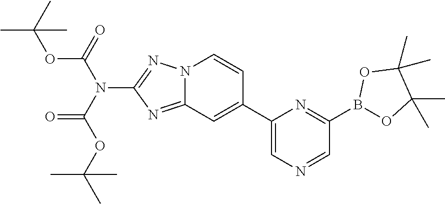 [SMILES: CC(C)(C)OC(=O)N(C(=O)OC(C)(C)C)c1nc2cc(-c3cncc(B4OC(C)(C)C(C)(C)O4)n3)ccn2n1]

To a solution of tert-butyl (tert-butoxycarbonyl)(7-(6-chloropyrazin-2-yl)-[1,2,4]triazolo[1,5-a]pyridin-2-yl)carbamate (0.197 g, 0.44 mmol) (Intermediate 41), bis(pinacolato)diboron (0.134 g, 0.528 mmol), and PdCl₂(dppf) (0.016 g, 0.022 mmol) in Dioxane (5 mL) was added potassium acetate (0.108 g, 1.100 mmol). The mixture was vacuumed, and nitrogen filled three times, then stirred at 85° C. for 1 h. The reaction mixture was used as the crude tert-butyl (tert-butoxycarbonyl)(7-(6-(4,4,5,5-tetramethyl-1,3,2-dioxaborolan-2-yl)pyrazin-2-yl)-[1,2,4]triazolo[1,5-a]pyridin-2-yl)carbamate for subsequent chemistry.

### Intermediate 43: 2-chloro-6-(2-(3,3-difluoro-1-(4-fluorophenyl)propyl)-2H-1,2,3-triazol-4-yl)pyridine

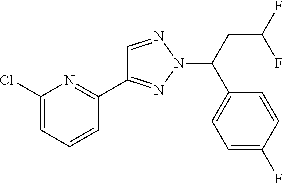 [SMILES: Fc1ccc(C(CC(F)F)n2ncc(-c3cccc(Cl)n3)n2)cc1]

The title compound was prepared in a similar fashion to Intermediate 38 using Intermediate 40. MS ESI m/z 353.0 (M+H)⁺.

### Intermediate 44: 7-(6-chloropyridin-2-yl)-8-fluoro-[1,2,4]triazolo[1,5-a]pyridin-2-amine

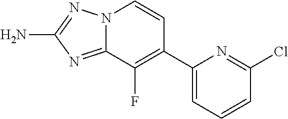 [SMILES: Nc1nc2c(F)c(-c3cccc(Cl)n3)ccn2n1]

To a solution of 8-fluoro-7-iodo-[1,2,4]triazolo[1,5-a]pyridin-2-amine (278 mg, 1.0 mmol), 2-chloro-6-(4,4,5,5-tetramethyl-1,3,2-dioxaborolan-2-yl)pyridine (287 mg, 1.200 mmol) and PdCl₂(dppf)-CH₂Cl₂ adduct (40.8 mg, 0.050 mmol) in dioxane (4.0 mL) was added tripotassium phosphate (1.0 mL, 3.00 mmol). The mixture was stirred at 95° C. for 1 h. Another 0.2 eq. of boronate and 0.02 eq. of PdCl₂(dppf) were added. After stirring at 100° C. for 30 min, water was added and extracted five times with EtOAc. The organic layer was dried over Na₂SO₄ then concentrated to give a crude 7-(6-chloropyridin-2-yl)-8-fluoro-[1,2,4]triazolo[1,5-a]pyridin-2-amine (250 mg, 0.948 mmol, 95% yield), which was used for subsequent chemistry without additional purification. MS ESI m/z 263.7 (M+H)⁺.

### Intermediate 45: 7-(6-chloropyridin-2-yl)-8-methyl-[1,2,4]triazolo[1,5-a]pyridin-2-amine

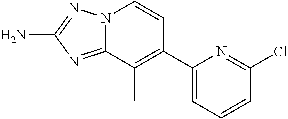 [SMILES: Cc1c(-c2cccc(Cl)n2)ccn2nc(N)nc12]

The titled intermediate was prepared in a similar fashion to Intermediate 44 using 7-bromo-8-methyl-[1,2,4]triazolo[1,5-a]pyridin-2-amine. MS ESI m/z 259.8 (M+H)⁺.

### Intermediate 46: 7-(6-chloropyridin-2-yl)-6-fluoro-[1,2,4]triazolo[1,5-a]pyridin-2-amine

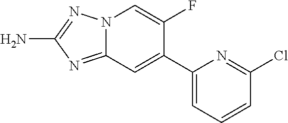 [SMILES: Nc1nc2cc(-c3cccc(Cl)n3)c(F)cn2n1]

The titled intermediate was prepared in a similar fashion to Intermediate 44 using 6-fluoro-7-iodo-[1,2,4]triazolo[1,5-a]pyridin-2-amine. MS ESI m/z 263.7 (M+H)⁺.

### Intermediate 47: 2-chloro-4-(2-(2,2-difluoro-1-(4-fluorophenyl)propyl)-2H-1,2,3-triazol-4-yl)pyrimidine

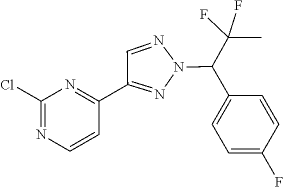 [SMILES: CC(F)(F)C(c1ccc(F)cc1)n1ncc(-c2ccnc(Cl)n2)n1]

To a solution of crude (2-(2,2-difluoro-1-(4-fluorophenyl)propyl)-2H-1,2,3-triazol-4-yl)boronic acid (90 mg, 0.316 mmol) (Intermediate 33), 4-bromo-2-chloropyrimidine (110 mg, 0.568 mmol) and PdCl₂(dppf)-CH₂Cl₂ adduct (12.89 mg, 0.016 mmol) in dioxane (2 mL) was added tripotassium phosphate (0.474 mL, 0.947 mmol). The mixture was stirred at 70° C. for 30 min. EtOAc was added, washed with water then concentrated. The residue was purified via silica gel chromatography (4 g, hexanes-100% EtOAc) to give 2-chloro-4-(2-(2,2-difluoro-1-(4-fluorophenyl)propyl)-2H-1,2,3-triazol-4-yl)pyrimidine (70 mg, 0.198 mmol, 62.7% yield). ¹H NMR (400 MHz, CHLOROFORM-d) δ 8.70 (d, J=5.1 Hz, 1H), 8.42 (s, 1H), 7.89 (d, J=5.1 Hz, 1H), 7.69 (dd, J=8.6, 5.2 Hz, 2H), 7.16-7.10 (m, 2H), 6.07 (dd, J=12.2, 10.6 Hz, 1H), 1.74 (t, J=18.7 Hz, 3H). MS ESI m/z 353.8 (M+H)⁺.

### Intermediate 48: 2-chloro-6-(2-(2,2-difluoro-1-(4-fluorophenyl)propyl)-2H-1,2,3-triazol-4-yl)pyrazine

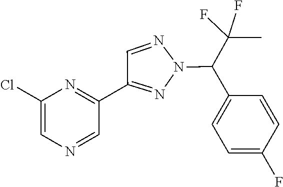 [SMILES: CC(F)(F)C(c1ccc(F)cc1)n1ncc(-c2cncc(Cl)n2)n1]

To a solution of the crude (2-(2,2-difluoro-1-(4-fluorophenyl)propyl)-2H-1,2,3-triazol-4-yl)boronic acid (103 mg, 0.36 mmol) (Intermediate 33), 2-bromo-6-chloropyrazine (125 mg, 0.648 mmol), and 1,1′-Bis(di-tert-butylphosphino)ferrocene palladium dichloride (11.73 mg, 0.018 mmol) in dioxane (2 mL) was added tripotassium phosphate (0.540 mL, 1.080 mmol). The mixture was stirred at 40° C. for 1 h. Water was added and extracted twice with EtOAc. The organic layer was concentrated and purified via silica gel chromatography (12 g, hexanes-25% EtOAc) to give 2-chloro-6-(2-(2,2-difluoro-1-(4-fluorophenyl)propyl)-2H-1,2,3-triazol-4-yl)pyrazine (90 mg, 0.254 mmol, 70.7% yield). ¹H NMR (400 MHz, CHLOROFORM-d) δ 9.16 (s, 1H), 8.58 (s, 1H), 8.35 (s, 1H), 7.74-7.68 (m, 2H), 7.17-7.10 (m, 2H), 6.08 (dd, J=12.2, 10.5 Hz, 1H), 1.75 (t, J=18.7 Hz, 3H). MS ESI m/z 353.8 (M+H)⁺.

### Intermediate 49: tert-butyl (tert-butoxycarbonyl)(7-(2-chloropyrimidin-4-yl)-[1,2,4]triazolo[1,5-a]pyridin-2-yl)carbamate

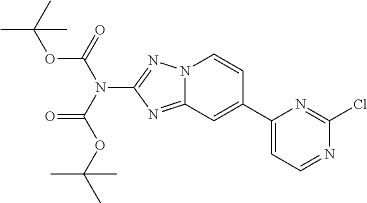 [SMILES: CC(C)(C)OC(=O)N(C(=O)OC(C)(C)C)c1nc2cc(-c3ccnc(Cl)n3)ccn2n1]

To a solution of (2-(bis(tert-butoxycarbonyl)amino)-[1,2,4]triazolo[1,5-a]pyridin-7-yl)boronic acid (450 mg, 1.190 mmol) (Intermediate 52), 4-bromo-2-chloropyrimidine (345 mg, 1.785 mmol) and PdCl₂(dppf)-CH₂Cl₂ adduct (48.6 mg, 0.059 mmol) in dioxane (5 mL) was added tripotassium phosphate (1.785 mL, 3.57 mmol). The mixture was stirred at 70° C. for 30 min. Water was added and extracted twice with EtOAc. The organic layer was concentrated and purified via silica gel chromatography (12 g, hexanes-100% EtOAc) to give tert-butyl (tert-butoxycarbonyl)(7-(2-chloropyrimidin-4-yl)-[1,2,4]triazolo[1,5-a]pyridin-2-yl)carbamate.

¹H NMR (400 MHz, CHLOROFORM-d) δ 8.82 (d, J=5.2 Hz, 1H), 8.69 (dd, J=7.2, 0.9 Hz, 1H), 8.42 (dd, J=1.9, 0.9 Hz, 1H), 7.90 (dd, J=7.2, 1.9 Hz, 1H), 7.78 (d, J=5.1 Hz, 1H), 1.51 (s, 18H). MS ESI m/z 447.2 (M+H)⁺.

### Intermediate 50: 7-(6-chloropyridin-2-yl)-6-fluoro-[1,2,4]triazolo[1,5-a]pyrazin-2-amine

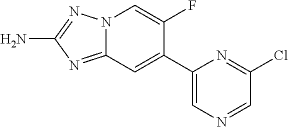 [SMILES: Nc1nc2cc(-c3cncc(Cl)n3)c(F)cn2n1]

A mixture of 6-fluoro-7-iodo-[1,2,4]triazolo[1,5-a]pyridin-2-amine (83 mg, 0.299 mmol), 2-chloro-6-(4,4,5,5-tetramethyl-1,3,2-dioxaborolan-2-yl)pyrazine (431 mg, 1.791 mmol), and PdCl₂(dppf)-CH₂Cl₂ adduct (12.19 mg, 0.015 mmol) in dioxane (2 mL) and tripotassium phosphate (0.746 mL, 1.493 mmol) was stirred at 100° C. for 1 h. Water was added and extracted four times with EtOAc. The organic layers were concentrated and purified via silica gel chromatography (4 g, hexanes-100% EtOAc-10% MeOH) to give 7-(6-chloropyrazin-2-yl)-6-fluoro-[1,2,4]triazolo[1,5-a]pyridin-2-amine (32 mg, 0.121 mmol, 40.5% yield). MS ESI m/z 264.7 (M+H)⁺.

### Intermediate 51: 7-(6-chloropyridin-2-yl)-8-fluoro-[1,2,4]triazolo[1,5-a]pyrazin-2-amine

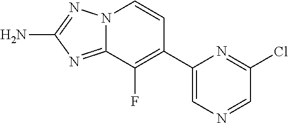 [SMILES: Nc1nc2c(F)c(-c3cncc(Cl)n3)ccn2n1]

The titled intermediate was prepared in a similar fashion to Intermediate 50 using 8-fluoro-7-iodo-[1,2,4]triazolo[1,5-a]pyridin-2-amine as starting material.

MS ESI m/z 264.7 (M+H)⁺.

### Intermediate 52: (2-(bis(tert-butoxycarbonyl)amino)-[1,2,4]triazolo[1,5-a]pyridin-7-yl)boronic acid

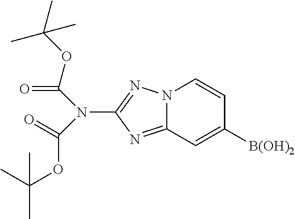 [SMILES: CC(C)(C)OC(=O)N(C(=O)OC(C)(C)C)c1nc2cc(B(O)O)ccn2n1]

To a stirred solution of 7-bromo-2-((bis-tert-butoxycarbonyl)amino)-[1,2,4]triazolo[1,5-a]pyridine (1.000 g, 2.420 mmol) in 1,4-dioxane (15 mL) was added bis(pinacolato)diboron (0.922 g, 3.63 mmol), potassium acetate (0.712 g, 7.26 mmol) and PdCl₂(dppf)-CH₂Cl₂ adduct (0.198 g, 0.242 mmol). The reaction mixture was sparged with N₂ for 5 min. The reaction mixture was heated at 100° C. for 2 h. The reaction 20 mixture was partitioned between water and ethyl acetate. The organic layer was washed with brine solution, dried over anhydrous sodium sulphate. filtered and concentrated under vacuum to give crude product (1.168 g, 2.378 mmol. 98% crude yield) as a dark brown semi-solid. The material was carried forward without further purification.

MS ESI m/z 379.4 (M+H).

### Intermediate 53: tert-butyl (tert-butoxycarbonyl)(8-methyl-7-(4,4,5,5-tetramethyl-1,3,2-dioxaborolan-2-yl)-[1,2,4]triazolo[1,5-a]pyridin-2-yl)carbamate

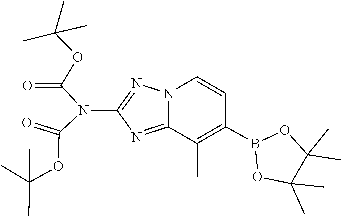 [SMILES: Cc1c(B2OC(C)(C)C(C)(C)O2)ccn2nc(N(C(=O)OC(C)(C)C)C(=O)OC(C)(C)C)nc12]

Step 1: To a solution of 7-bromo-8-methyl-[1,2,4]triazolo[1,5-a]pyridin-2-amine (210 mg, 0.925 mmol) and DMAP (22.60 mg, 0.185 mmol) in MeCN (5 mL) was added Boc-anhydride (1.074 mL, 4.62 mmol). The mixture was stirred at rt for 20 h. The reaction was concentrated then purified via silica gel chromatography (24 g, hexanes-80% EtOAc) to give tert-butyl (7-bromo-8-methyl-[1,2,4]triazolo[1,5-a]pyridin-2-yl)(tert-butoxycarbonyl)carbamate (330 mg, 0.772 mmol, 84% yield). ¹H NMR (500 MHz, CHLOROFORM-d) δ 8.26 (dd, J=7.2, 0.7 Hz, 1H), 7.21-7.17 (m, 1H), 2.69 (s, 3H), 1.48 (s, 18H). MS ESI m/z 428.6 (M+H)⁺.

Step 2: To a solution of tert-butyl (7-bromo-8-methyl-[1,2,4]triazolo[1,5-a]pyridin-2-yl)(tert-butoxycarbonyl)carbamate (78 mg, 0.183 mmol), bispin (69.5 mg, 0.274 mmol), and PdCl₂(dppf)-CH₂Cl₂ adduct (14.91 mg, 0.018 mmol) in dioxane (1.0 mL) was added potassium acetate (44.8 mg, 0.456 mmol). The mixture was stirred at 105° C. for 2 h. Another 180 mg of bispin was added. The mixture was stirred at 105° C. for 1 h. Water was added and extracted twice with EtOAc. The organic layer was concentrated then purified via silica gel chromatography (12 g, hexanes-50% EtOAc) to give tert-butyl (tert-butoxycarbonyl)(8-methyl-7-(4,4,5,5-tetramethyl-1,3,2-dioxaborolan-2-yl)-[1,2,4]triazolo[1,5-a]pyridin-2-yl)carbamate (70 mg, 0.148 mmol, 81% yield). ¹H NMR (400 MHz, CHLOROFORM-d) δ 8.34 (dd, J=6.9, 0.7 Hz, 1H), 7.33 (d, J=6.9 Hz, 1H), 2.86 (s, 3H), 1.45 (s, 18H), 1.39 (s, 12H). MS ESI m/z 475.0 (M+H)⁺.

### Intermediate 54: (±)-2-(1-(4-fluorophenyl)ethyl)-4-(4,4,5,5-tetramethyl-1,3,2-dioxaborolan-2-yl)-2H-1,2,3-triazole

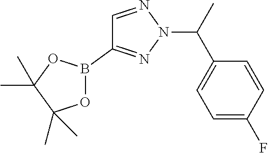 [SMILES: CC(c1ccc(F)cc1)n1ncc(B2OC(C)(C)C(C)(C)O2)n1]

Step 1: Under the nitrogen atmosphere to a mixture of 1-(1-bromoethyl)-4-fluorobenzene (20 g, 98 mmol) and DMF (200 mL) was added 4,5-dibromo-2H-1,2,3-triazole (26.8 g, 118 mmol) and K₂CO₃ (27.2 g, 197 mmol). The mixture was stirred at rt for 2 h. Water (150 ml) was added and extracted three times with EtOAc (250 mL). The combined organic layers were dried over Na₂SO₄ then concentrated. The residue was purified by column chromatography (80 g snap Biotage, SiO₂, eluted with 10% EtOAc) to give 4,5-dibromo-2-(1-(4-fluorophenyl)ethyl)-2H-1,2,3-triazole (26 g, 72.4 mmol, 73.5% yield).

Step 2: Under the nitrogen atmosphere to a mixture of 4,5-dibromo-2-(1-(4-fluorophenyl)ethyl)-2H-1,2,3-triazole (13.0 g, 37.2 mmol) and THF (130 mL) was added isopropylmagnesium chloride (37.2 mL, 74.5 mmol) in cooling condition. The mixture was stirred at rt for 2 h. Water (100 mL) was added and extracted three times with EtOAc (100 mL). The combined organic layers were dried over Na₂SO₄ then concentrated in vacuum. The residue was purified by column chromatography (120 g snap Biotage, SiO₂, eluted with 10% EtOAc) to give 4-bromo-2-(1-(4-fluorophenyl)ethyl)-2H-1,2,3-triazole (7.5 g, 26.9 mmol, 72.3% yield) as a colourless liquid.

Step 3: To a solution of 4-bromo-2-(1-(4-fluorophenyl)ethyl)-2H-1,2,3-triazole (14.8 g, 54.8 mmol) in THF (150 mL) was added isopropylmagnesium chloride (54.8 mL, 110 mmol) at 0° C. under N₂. The mixture was stirred at rt for 1 h. Another 0.5 eq. of Isopropylmagnesium chloride solution in THF was added. The mixture was stirred at rt for 1 h. Trimethyl borate (20.5 g, 197 mmol) was added at −20° C. The mixture was allowed to warm up to 0° C. over 1 h. 1.5 N HCl (50 mL) solution was added and extracted three times with EtOAc (150 mL). The combined organic layers were dried over Na₂SO₄ then concentrated. The residue was purified by column chromatography (120 g snap Biotage, SiO₂, eluted with 70% EtOAc) to give (2-(1-(4-fluorophenyl)ethyl)-2H-1,2,3-triazol-4-yl)boronic acid (5.6 g, 23.37 mmol, 42.6% yield) as an off-white-solid. 1H-NMR (400 MHz, DMSO-d₆): δ 8.3 (Bs, 2H), δ 7.9(dd, 1H), 7.29-7.31 (dd, 2H), 7.08-7.31 (dd, 2H), 5.93-5.94 (m, 1H), 1.8 (q, 3H).

### Intermediate 55: (±)-4-bromo-2-(2,2-difluoro-1-(4-fluorophenyl)propyl)-5-methyl-2H-1,2,3-triazole

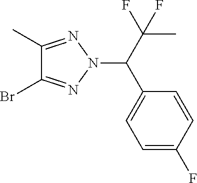

To a solution of 4,5-dibromo-2-(2,2-difluoro-1-(4-fluorophenyl)propyl)-2H-1,2,3-triazole (0.230 g, 0.576 mmol) (Intermediate 19C), Trimethylboroxine (0.072 g, 0.288 mmol), and PdCl₂(dppf) (0.021 g, 0.029 mmol) in Dioxane (2 mL) was added Cs2CO3 (0.338 g, 1.038 mmol) and Water (0.2 mL). The mixture was stirred at 110° C. for 6 h. Water was added and extracted twice with EtOAc. The organic layers were concentrated and purified via silica gel chromatography (12 g, hexanes-30% EtOAc). Identified via LCMS, the product was eluted out first at around 9% EtOAc/Hexanes to give a crude 4-bromo-2-(2,2-difluoro-1-(4-fluorophenyl)propyl)-5-methyl-2H-1,2,3-triazole (115 mg, 0.344 mmol, 59.7% yield) contaminated with SM and double coupled side product. MS ESI m/z 333.8 (M+H)⁺.

### Intermediate 56: (±)-4-bromo-2-(1-(4-chlorophenyl)-2,2-difluoropropyl)-2H-1,2,3-triazole

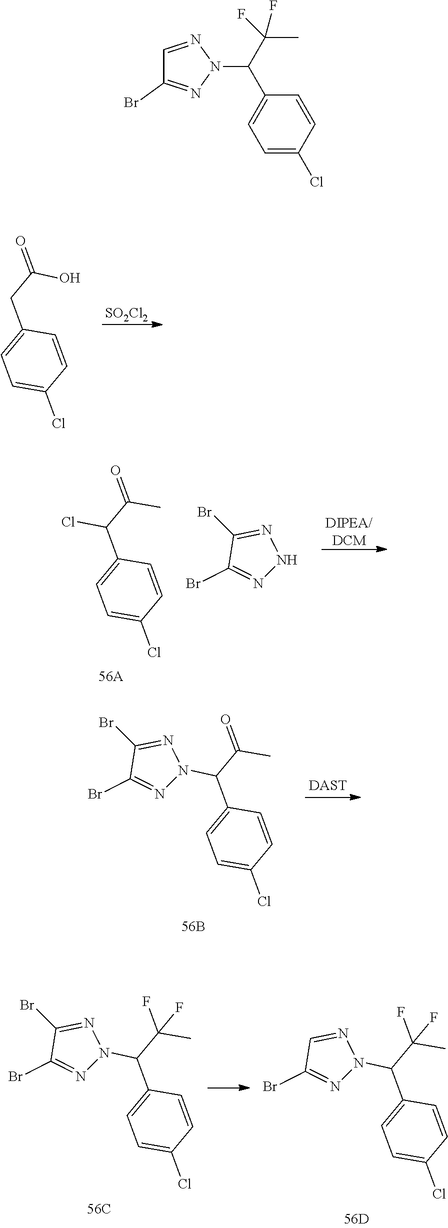

Intermediate 56A: Prepared in a similar fashion to Intermediate 1C.

¹H NMR (500 MHz, CHLOROFORM-d) δ 7.42-7.37 (m, 4H), 5.32 (s, 1H), 2.27 (s, 3H).

Intermediate 56B: Prepared in a similar fashion to Intermediate 1D.

¹H NMR (400 MHz, CHLOROFORM-d) δ 7.45 (m, 4H), 6.25 (s, 1H), 2.20 (s, 3H).

Intermediate 56C: Prepared in a similar fashion to Intermediate 1E.

¹H NMR (400 MHz, CHLOROFORM-d) δ 7.61-7.56 (m, 2H), 7.43-7.40 (m, 2H), 5.86 (dd, J=11.9, 10.5 Hz, 1H), 1.71 (t, J=18.6 Hz, 3H). MS ESI m/z 417.5 (M+H)⁺.

Intermediate 56: Prepared in a similar fashion to Intermediate 1.

¹H NMR (500 MHz, CHLOROFORM-d) δ 7.68 (s, 1H), 7.61-7.57 (m, 2H), 7.42-7.38 (m, 2H), 5.96-5.91 (m, 1H), 1.70 (t, J=18.7 Hz, 3H). MS ESI m/z 337.7 (M+H)⁺.

### Intermediate 57: 2-chloro-4-(2-(1-(4-chlorophenyl)-2,2-difluoropropyl)-2H-1,2,3-triazol-4-yl)pyrimidine

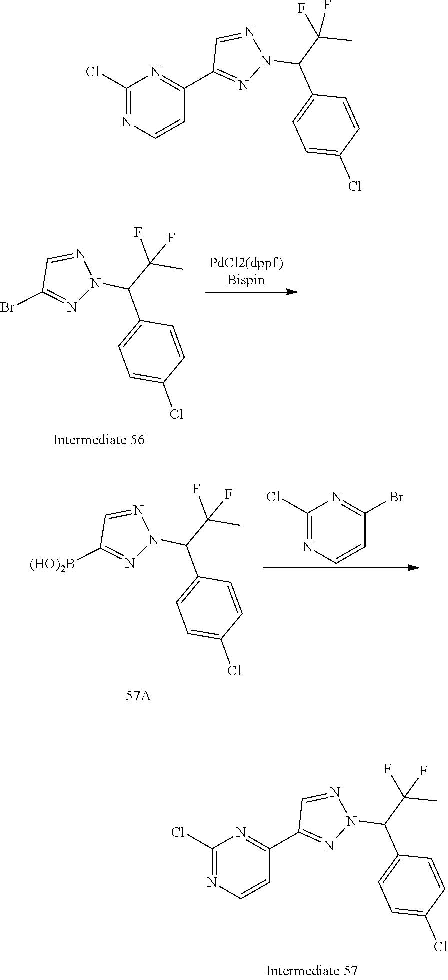 [SMILES: CC(F)(F)C(c1ccc(Cl)cc1)n1ncc(-c2ccnc(Cl)n2)n1 ; CC(F)(F)C(c1ccc(Cl)cc1)n1ncc(Br)n1 ; "PdCl2(dppf)Bispin" ; CC(F)(F)C(c1ccc(Cl)cc1)n1ncc(B(O)O)n1 ; Clc1nccc(Br)n1 ; CC(F)(F)C(c1ccc(Cl)cc1)n1ncc(-c2ccnc(Cl)n2)n1]

Step 1: To a solution of semi-pure 4-bromo-2-(1-(4-chlorophenyl)-2,2-difluoropropyl)-2H-1,2,3-triazole (300 mg, 0.891 mmol) in dioxane (5 mL) was added bispin (340 mg, 1.337 mmol), potassium acetate (262 mg, 2.67 mmol), and PdCl₂(dppf)-CH₂Cl₂ adduct (36.4 mg, 0.045 mmol). The mixture was stirred at 90-100° C. for 2 h. Another 1 eq. of bispin and 0.05 eq. of PdCl₂(dppf) were added. The reaction was stirred at 105° C. for 2 h, at 88° C. for 16 h, then at 120° C. for 4 h. The reaction mixture was used directly for the next step.

Step 2: To the crude reaction mixture of (2-(1-(4-chlorophenyl)-2,2-difluoropropyl)-2H-1,2,3-triazol-4-yl)boronic acid (268 mg, 0.89 mmol) was added tripotassium phosphate (1.335 mL, 2.67 mmol), 4-bromo-2-chloropyrimidine (207 mg, 1.068 mmol) and PdCl₂(dppf)-CH₂Cl₂ adduct (21.80 mg, 0.027 mmol) at rt. The mixture was stirred at 50° C. for 30 min. Water was added, and washed twice with EtOAc. The organic layer was concentrated then purified via silica gel chromatography (12 g, hexanes-50% EtOAc) to give semi-pure 2-chloro-4-(2-(1-(4-chlorophenyl)-2,2-difluoropropyl)-2H-1,2,3-triazol-4-yl)pyrimidine (76 mg, 0.205 mmol, 23.07% yield). MS ESI m/z 369.8 (M+H)⁺.

### Intermediate 58: tert-butyl (tert-butoxycarbonyl)(6-(4,4,5,5-tetramethyl-1,3,2-dioxaborolan-2-yl)-[1,2,4]triazolo[1,5-a]pyridin-2-yl)carbamate

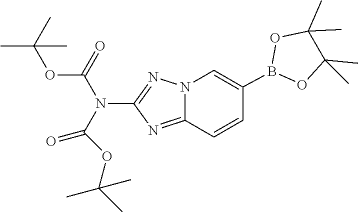 [SMILES: CC(C)(C)OC(=O)N(C(=O)OC(C)(C)C)c1nc2ccc(B3OC(C)(C)C(C)(C)O3)cn2n1]

Step 1: To a mixture of 6-bromo-[1,2,4]triazolo[1,5-a]pyridin-2-amine (3.3 g, 15.49 mmol) and DMAP (0.568 g, 4.65 mmol) in AcCN (100 mL) was added Boc-anhydride (8.99 mL, 38.7 mmol) slowly at rt. The reaction was stirred 45° C. for 5 h. The mixture was concentrated. The residue was purified via silica gel chromatography (120 g, hexanes-100% EtOAc) to give tert-butyl (6-bromo-[1,2,4]triazolo[1,5-a]pyridin-2-yl)(tert-butoxycarbonyl)carbamate (6.0 g, 14.52 mmol, 94% yield). MS ESI m/z 414.8 (M+H)⁺.

Step 2: To a solution of tert-butyl (6-bromo-[1,2,4]triazolo[1,5-a]pyridin-2-yl)(tert-butoxycarbonyl)carbamate (5.6 g, 13.55 mmol) (Intermediate 58A), bispin (5.51 g, 21.68 mmol), and PdCl₂(dppf)-CH₂Cl₂ adduct (0.664 g, 0.813 mmol) in dioxane (50 mL) was added potassium acetate (3.99 g, 40.7 mmol). The reaction was stirred 100° C. for 2 h. EtOAc was added, then filtered though celite. The filtrate was concentrated. The residue was suspended in hexane. The resulting solid was filtered to give a crude tert-butyl (tert-butoxycarbonyl)(6-(4,4,5,5-tetramethyl-1,3,2-dioxaborolan-2-yl)-[1,2,4]triazolo[1,5-a]pyridin-2-yl)carbamate (6.1 g, 13.25 mmol, 98% yield). MS ESI m/z 378.8 (M+H)⁺.

### Intermediate 59: tert-butyl (tert-butoxycarbonyl)(7-(5-chloro-6-methylpyridin-3-yl)-8-methyl-[1,2,4]triazolo[1,5-a]pyridin-2-yl)carbamate

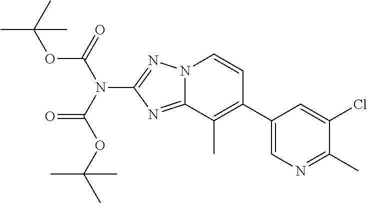 [SMILES: Cc1ncc(-c2ccn3nc(N(C(=O)OC(C)(C)C)C(=O)OC(C)(C)C)nc3c2C)cc1Cl]

The titled compound was prepared in a similar manner as Intermediate 22 utilizing tert-butyl (tert-butoxycarbonyl)(8-methyl-7-(4,4,5,5-tetramethyl-1,3,2-dioxaborolan-2-yl)-[1,2,4]triazolo[1,5-a]pyridin-2-yl)carbamate (Intermediate 53). ¹H NMR (400 MHz, CHLOROFORM-d) δ 8.48 (d, J=7.0 Hz, 1H), 8.45 (d, J=2.1 Hz, 1H), 7.70 (d, J=2.0 Hz, 1H), 6.96 (d, J=7.0 Hz, 1H), 2.74 (s, 3H), 2.61 (s, 3H), 1.51 (s, 18H). MS ESI m/z 474.4/476.1 (M+H)⁺.

### Intermediate 60: (R)-5-chloro-3-(2-(2,2-difluoro-1-(4-fluorophenyl)propyl)-2H-1,2,3-triazol-4-yl)-2-(trifluoromethyl)pyridine

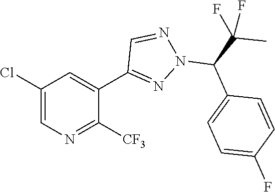 [SMILES: CC(F)(F)[C@@H](c1ccc(F)cc1)n1ncc(-c2cc(Cl)cnc2C(F)(F)F)n1]

The titled compound was prepared in a similar manner as Intermediate 26 utilizing 3-bromo-5-chloro-2-(trifluoromethyl)pyridine. ¹H NMR (400 MHz, CHLOROFORM-d) δ 8.70 (d, J=2.2 Hz, 1H), 8.15 (d, J=2.3 Hz, 1H), 7.97 (s, 1H), 7.71 (dd, J=8.7, 5.2 Hz, 2H), 7.14 (t, J=8.1 Hz, 2H), 6.06 (t, J=11.2 Hz, 1H), 1.73 (t, J=18.6 Hz, 3H). MS ESI m/z 420.9/422.7 (M+H)⁺.

### Intermediate 61: methyl 2-(4-bromo-2H-1,2,3-triazol-2-yl)-2-(4-fluorophenyl)acetate

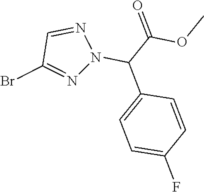 [SMILES: COC(=O)C(c1ccc(F)cc1)n1ncc(Br)n1]

Step 1: To a soln of methyl 2-bromo-2-(4-fluorophenyl)acetate (2 g, 8.10 mmol) in DCM (14.80 ml) was added 4,5-dibromo-2H-1,2,3-triazole (1.836 g, 8.10 mmol) followed by dropwise DIPEA (1.979 ml, 11.33 mmol) at rt. The mixture was stirred at 35° C. for 18 h. Two regioisomers were generated based on LCMS. The desired isomer was purified by silica gel chromatography.

Step 2: Methyl 2-(4,5-dibromo-2H-1,2,3-triazol-2-yl)-2-(4-fluorophenyl)acetate (2.5 g, 6.36 mmol) (Intermediae 61A) was dissolved in THF and cooled to −78° C. Isopropylmagnesium chloride lithium chloride complex solution in THF (9.79 ml, 12.72 mmol) was added slowly and stirred for 10 mins. The reaction was complete and quenched with NH₄Cl/water then extracted with EtOAc. The organic layer was dried over Na₂SO₄ and concentrated to a colorless oil. The reaction was loaded onto a normal phase silica gel column (0-100% Hex:EtOAc) and the desired fractions were collected and concentrated to afford methyl 2-(4-bromo-2H-1,2,3-triazol-2-yl)-2-(4-fluorophenyl)acetate (500 mg, 25% yield). MS ESI m/z 313.7/315.7 (M+H)⁺.

### Intermediate 62: (±)-4-bromo-2-(1-(4-fluorophenyl)-2,2-dimethylpropyl)-2H-1,2,3-triazole

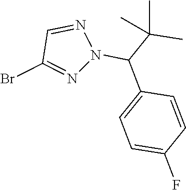 [SMILES: CC(C)(C)C(c1ccc(F)cc1)n1ncc(Br)n1]

Step 1: Trimethylacetaldehyde (4 g, 46.4 mmol) and THF (116 ml) were added to a vial. The vial was brought to 0° C. in an ice bath and (4-fluorophenyl)magnesium bromide (1 eq, 1 M) was added slowly. The reaction was stirred for 2 hours at room temp. The reaction was dissolved in EtOAc and a workup was performed with water. The organic layer was extracted and concentrated. ¹H NMR (500 MHz, CHLOROFORM-d) δ 7.30 (dd, J=8.4, 5.5 Hz, 2H), 7.02 (t, J=8.2 Hz, 2H), 4.41 (s, 1H), 0.93 (s, 9H).

Step 2: To a vial was added triphenylphosphine (1595 mg, 6.08 mmol), 4-bromo-2H-1,2,3-triazole (300 mg, 2.028 mmol), 1-(4-fluorophenyl)-2,2-dimethylpropan-1-ol (369 mg, 2.028 mmol) (Intermediate 62A), and THF (6 mL). The reaction was brought to 0° C. and diisopropyl diazene-1,2-dicarboxylate (1.194 mL, 6.08 mmol) was added slowly. The reaction was stirred at room temp for 3 hours. LCMS showed the product. The reaction was filtered and concentrated. The reaction was loaded onto a normal phase silica gel colum and run (0-40% Hex:EtOAc). The desired fractions were collected and concentrated. The semi-pure (±)-4-bromo-2-(1-(4-fluorophenyl)-2,2-dimethylpropyl)-2H-1,2,3-triazole (300 mg, 47% yield) was used without further purification. MS ESI m/z 311.9/313.8 (M+H)⁺.

### Intermediate 63: (±)-4-(1-(4-bromo-2H-1,2,3-triazol-2-yl)ethyl)benzonitrile

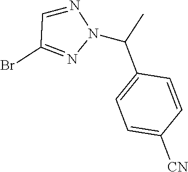 [SMILES: CC(c1ccc(C#N)cc1)n1ncc(Br)n1]

Step 1: To a soln of 4-(1-bromoethyl)benzonitrile (1000 mg, 4.76 mmol) in DCM (10 ml) was added 4,5-dibromo-2H-1,2,3-triazole (1080 mg, 4.76 mmol) followed by dropwise DIPEA (1.164 ml, 6.66 mmol) at rt. The mixture was stirred at 35° C. for 18 h. The reaction was concentrated down and loaded onto a normal phase column (0-100% Hex:EtOAc). The desired fractions were concentrated to afford (±)-4-(1-(4,5-dibromo-2H-1,2,3-triazol-2-yl)ethyl)benzonitrile (1.1 g, 65% yield). MS ESI m/z 354.7/356.6/358.6 (M+H)⁺.

Step 2: At 22° C. under N₂ atmosphere, a solution of ammonium chloride (0.826 g, 15.45 mmol) in 2 mL water was added dropwise over 2 min to a clear solution of 4-(1-(4,5-dibromo-2H-1,2,3-triazol-2-yl)ethyl)benzonitrile (1.1 g, 3.09 mmol) in MeOH (12 mL), THF (12 mL) above zinc (0.505 g, 7.72 mmol). After addition, the reaction mixture was stirred vigorously for 7 h at 25° C. under N₂ atmosphere. After 7 h, LCMS showed product formation. The reaction mixture was filtered through celite and concentrated under reduced pressure. The resulting crude material was extracted twice with 250 mL of EtOAc and washed with 100 mL of 1 N HCl and 200 mL brine. The solution was dried over sodium sulphate, filtered, and concentrated to get a crude gummy product. MS ESI m/z 276.8/278.8 (M+H)⁺.

### Example 1: (Ent 1)-7-(6-(2-(1-(3,5-difluorophenyl)-2,2-difluoropropyl)-2H-1,2,3-triazol-4-yl)pyrazin-2-yl)-[1,2,4]triazolo[1,5-α]pyridin-2-amine

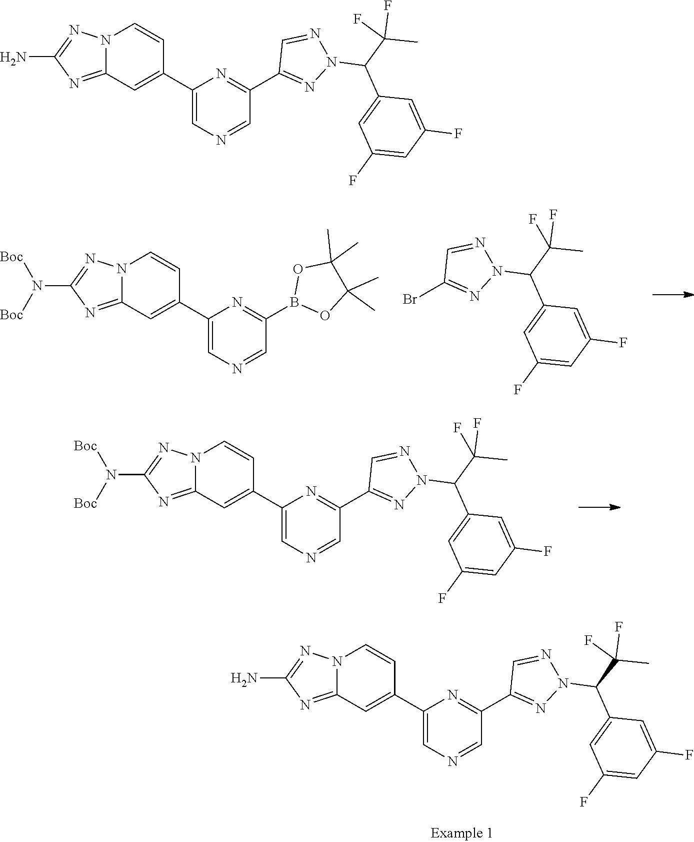

Step 1: To a pressure vial were added tert-butyl (tert-butoxycarbonyl)(7-(6-(4,4,5,5-tetramethyl-1,3,2-dioxaborolan-2-yl)pyrazin-2-yl)-[1,2,4]triazolo[1,5-a]pyridin-2-yl)carbamate (Intermediate 17) (175 mg, 0.325 mmol), (±) 4-bromo-2-(1-(3,5-difluorophenyl)-2,2-difluoropropyl)-2H-1,2,3-triazole (Intermediate 1) (110 mg, 0.325 mmol), potassium phosphate (2 M water solution) (0.604 mL, 1.208 mmol), and PdCl₂(dppf)-CH₂Cl₂ adduct (32.9 mg, 0.040 mmol). It was heated at 85° C. for 1 h and cooled to rt. The reaction was diluted water and ethyl acetate. The layers were separated. The aqueous layer was extracted with ethyl acetate. The combined organic layers were washed with water, brine, dried over anhydrous sodium sulfate, filtered and evaporated to dryness. It was purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 min; Flow Rate: 42.5 mL/min; Column Temperature: 25° C. Fraction collection was triggered by UV (214 nm). Fractions containing the desired product were combined and dried via centrifugal evaporation to provide (±) tert-butyl (tert-butoxycarbonyl)(7-(6-(2-(1-(3,5-difluorophenyl)-2,2-difluoropropyl)-2H-1,2,3-triazol-4-yl)pyrazin-2-yl)-[1,2,4]triazolo[1,5-a]pyridin-2-yl)carbamate (75 mg, 28%). LCMS (ESI, m/z): 670.2 [M+H]⁺.

Step 2: To a solution of (±) tert-butyl (tert-butoxycarbonyl)(7-(6-(2-(1-(3,5-difluorophenyl)-2,2-difluoropropyl)-2H-1,2,3-triazol-4-yl)pyrazin-2-yl)-[1,2,4]triazolo[1,5-a]pyridin-2-yl)carbamate (75 mg, 0.112 mmol) in DCM (1 mL) was added TFA (1.0 mL) and the reaction mixture was stirred for 2 h. It was concentrated via nitrogen stream to a residue. The residue was dissolved in DMF and filtered to give a 2 mL sample which was purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 22-52% B over 20 min; Flow Rate: 20 mL/min; Column Temperature: 25° C. Fraction collection was triggered by UV (220 nm) and MS (ESI+). Fractions containing the desired product were combined and dried via centrifugal evaporation to provide (±) 7-(6-(2-(1-(3,5-difluorophenyl)-2,2-difluoropropyl)-2H-1,2,3-triazol-4-yl)pyrazin-2-yl)-[1,2,4]triazolo[1,5-a]pyridin-2-amine (25.4 mg, 0.054 mmol, 48.3% yield). This racemic material was then further purified via preparative SFC with the following conditions: Column: Chiralpak 1A, 21 mm×250 mm, 5 m particles; Mobile Phase A: supercritical CO₂; Mobile Phase B: i-PrOH with 0.1% NH₄O H; Elution gradient: isocratic 50% B over 18 minutes; Flow Rate: 75 mL/min; Column Temperature: 40° C. Fraction collection was triggered by UV (235 nm). Fractions containing the first isomer eluting from the chiral column were combined and dried via centrifugal evaporation to provide (Ent 1)-7-(6-(2-(1-(3,5-difluorophenyl)-2,2-difluoropropyl)-2H-1,2,3-triazol-4-yl)pyrazin-2-yl)-[1,2,4]triazolo[1,5-a]pyridin-2-amine (9.3 mg, 16.9%). ¹H NMR (500 MHz, DMSO-d₆) δ 9.51-9.41 (m, 1H), 9.25-9.18 (m, 1H), 8.87-8.78 (m, 1H), 8.78-8.68 (m, 1H), 8.36-8.24 (m, 1H), 7.86-7.74 (m, 1H), 7.63-7.49 (m, 2H), 7.48-7.34 (m, 1H), 6.97-6.79 (m, 1H), 6.17 (s, 2H), 1.72 (br t, J=19.1 Hz, 3H). LCMS (ESI, m/z): 470.2 [M+H]⁺.

### Example 2: (±)-7-(6-(2-(1-(3,5-difluorophenyl)-2,2-difluoropropyl)-2H-1,2,3-triazol-4-yl)pyrazin-2-yl)-8-methyl-[1,2,4]triazolo[1,5-a]pyridin-2-amine

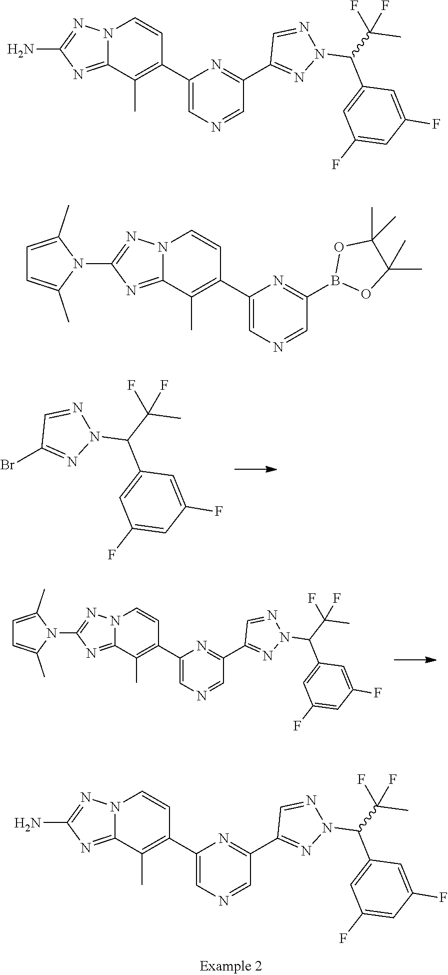 [SMILES: Cc1c(-c2cncc(-c3cnn(C(c4cc(F)cc(F)c4)C(C)(F)F)n3)n2)ccn2nc(N)nc12 ; Cc1c(-c2cncc(B3OC(C)(C)C(C)(C)O3)n2)ccn2nc(-n3c(C)ccc3C)nc12 ; CC(F)(F)C(c1cc(F)cc(F)c1)n1ncc(Br)n1 ; Cc1c(-c2cncc(-c3cnn(C(c4cc(F)cc(F)c4)C(C)(F)F)n3)n2)ccn2nc(-n3c(C)ccc3C)nc12 ; Cc1c(-c2cncc(-c3cnn(C(c4cc(F)cc(F)c4)C(C)(F)F)n3)n2)ccn2nc(N)nc12]

Step 1: (±)-7-(6-(2-(1-(3,5-difluorophenyl)-2,2-difluoropropyl)-2H-1,2,3-triazol-4-yl)pyrazin-2-yl)-2-(2,5-dimethyl-1H-pyrrol-1-yl)-8-methyl-[1,2,4]triazolo[1,5-a]pyridine was obtained in a similar fashion as described for the preparation of Example 1, step 1, where 2-(2,5-dimethyl-1H-pyrrol-1-yl)-8-methyl-7-(6-(4,4,5,5-tetramethyl-1,3,2-dioxaborolan-2-yl)pyrazin-2-yl)-[1,2,4]triazolo[1,5-a]pyridine (Intermediate 18) was used in place of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-a]pyridin-2-yl)carbamate. LCMS (ESI, m/z): 470.2 [M+H]+.

Step 2: To a pressure vial were added (±)-7-(6-(2-(1-(3,5-difluorophenyl)-2,2-difluoropropyl)-2H-1,2,3-triazol-4-yl)pyrazin-2-yl)-2-(2,5-dimethyl-1H-pyrrol-1-yl)-8-methyl-[1,2,4]triazolo[1,5-a]pyridine (28 mg, 0.05 mmol), THF (1 mL), water (0.5 mL), and TFA (0.5 mL). The reaction mixture was heated at 70° C. for 2 h. The mixture was concentrated via nitrogen stream to a residue. The residue was dissolved in DMF and filtered to give a 2 mL sample which was purified via preparative reverse phase chromatography with the following conditions: Column: Waters XBridge C18, 19 mm×200 mm, 5 m particles; Mobile Phase A: 5:95 acetonitrile: water with 10-mM ammonium acetate; Mobile Phase B: 95:5 acetonitrile: water with 10-mM ammonium acetate; Gradient: 24-54% B over 20 min; Flow Rate: 20 mL/min; Column Temperature: 25° C. Fraction collection was triggered by UV (220 nm) and MS (ESI+). Fractions containing the desired product were combined and dried via centrifugal evaporation to provide (±)-7-(6-(2-(1-(3,5-difluorophenyl)-2,2-difluoropropyl)-2H-1,2,3-triazol-4-yl)pyrazin-2-yl)-8-methyl-[1,2,4]triazolo[1,5-a]pyridin-2-amine (4.3 mg, 6.4% yield). ¹H NMR (500 MHz, DMSO-d₆) δ 9.29-9.20 (m, 1H), 9.02-8.92 (m, 1H), 8.70-8.62 (m, 1H), 8.61-8.50 (m, 1H), 7.64-7.53 (m, 2H), 7.48-7.33 (m, 1H), 7.21-7.10 (m, 1H), 6.96-6.81 (m, 1H), 6.13 (s, 2H), 2.57 (s, 3H), 1.80-1.63 (m, 3H). LCMS (ESI, m/z): 484.1 [M+H]+.

### Example 3: (Ent 1 and Ent 2)-7-(6-(2-(1-(4-chloro-3-fluorophenyl)-2,2-difluoropropyl)-2H-1,2,3-triazol-4-yl)pyrazin-2-yl)-[1,2,4]triazolo[1,5-α]pyridin-2-amine

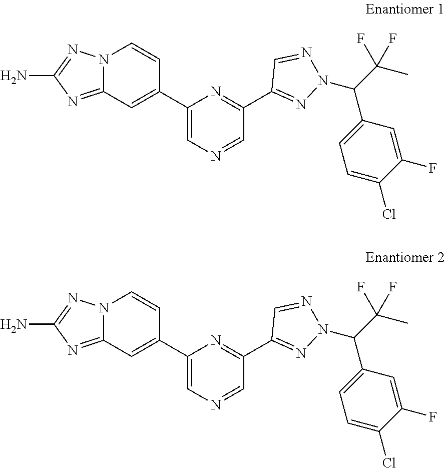 [SMILES: CC(F)(F)C(c1ccc(Cl)c(F)c1)n1ncc(-c2cncc(-c3ccn4nc(N)nc4c3)n2)n1 ; CC(F)(F)C(c1ccc(Cl)c(F)c1)n1ncc(-c2cncc(-c3ccn4nc(N)nc4c3)n2)n1]

(The two unassigned enantiomers are

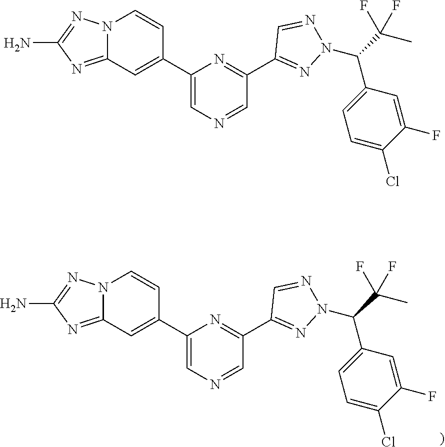 [SMILES: CC(F)(F)[C@H](c1ccc(Cl)c(F)c1)n1ncc(-c2cncc(-c3ccn4nc(N)nc4c3)n2)n1 ; CC(F)(F)[C@@H](c1ccc(Cl)c(F)c1)n1ncc(-c2cncc(-c3ccn4nc(N)nc4c3)n2)n1]

The title compounds (Ent 1 and Ent 2)-7-(6-(2-(1-(4-chloro-3-fluorophenyl)-2,2-difluoropropyl)-2H-1,2,3-triazol-4-yl)pyrazin-2-yl)-[1,2,4]triazolo[1,5-α]pyridin-2-amine were obtained in a similar fashion as described for the preparation of Example 1, where (±)-4-bromo-2-(1-(4-chloro-3-fluorophenyl)-2,2-difluoropropyl)-2H-1,2,3-triazole (Intermediate 2) was used in place of (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-57% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(1-(4-chloro-3-fluorophenyl)-2,2-difluoropropyl)-2H-1,2,3-triazol-4-yl)pyrazin-2-yl)-[1,2,4]triazolo[1,5-α]pyridin-2-amine (29 mg, 68%). This racemic material was then further purified via preparative SFC with the following conditions: Column: Chiralpak AD-H, 30 mm×250 mm, 5 m particles; Flow Rate:75.00 mL/min; Mobile Phase A: supercritical CO₂; Mobile Phase B: i-PrOH with 0.1% NH₄O H; Elution gradient: isocratic 10% B over 28 minutes; Column Temperature: 40° C. Fraction collection was triggered by UV (275 nm). Fractions containing the first isomer to elute from the chiral column were combined and dried via centrifugal evaporation to provide (Ent 1)-7-(6-(2-(1-(4-chloro-3-fluorophenyl)-2,2-difluoropropyl)-2H-1,2,3-triazol-4-yl)pyrazin-2-yl)-[1,2,4]triazolo[1,5-α]pyridin-2-amine (8.4 mg, 19.8%) and the second enantiomer was likewise obtained. (Ent-1): ¹H NMR (500 MHz, DMSO-d₆) δ 9.53-9.39 (m, 1H), 9.25-9.11 (m, 1H), 8.83-8.76 (m, 1H), 8.75-8.67 (m, 1H), 8.36-8.27 (m, 1H), 7.89-7.82 (m, 1H), 7.82-7.76 (m, 1H), 7.76-7.67 (m, 2H), 6.95-6.83 (m, 1H), 6.24-6.12 (m, 2H), 1.83-1.62 (m, 3H). LCMS (ESI, m/z): 486.2 [M+H]+.

### Example 4: (Ent 1 and Ent 2)-7-(6-(2-(1-(4-chloro-3-fluorophenyl)-2,2-difluoropropyl)-2H-1,2,3-triazol-4-yl)pyrazin-2-yl)-8-methyl-[1,2,4]triazolo[1,5-α]pyridin-2-amine

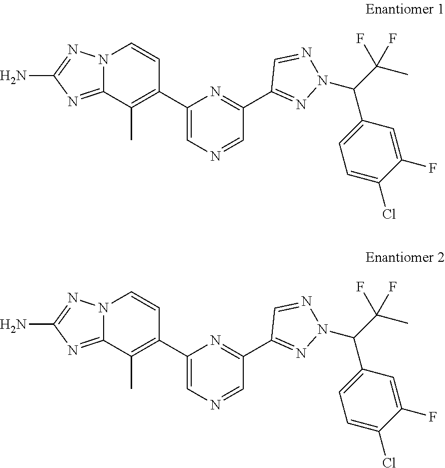 [SMILES: Cc1c(-c2cncc(-c3cnn(C(c4ccc(Cl)c(F)c4)C(C)(F)F)n3)n2)ccn2nc(N)nc12 ; Cc1c(-c2cncc(-c3cnn(C(c4ccc(Cl)c(F)c4)C(C)(F)F)n3)n2)ccn2nc(N)nc12]

(The two unassigned enantiomers are

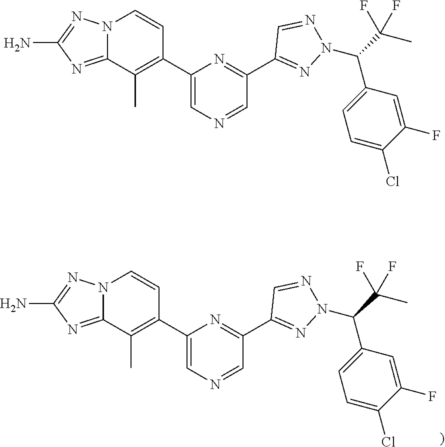 [SMILES: Cc1c(-c2cncc(-c3cnn([C@@H](c4ccc(Cl)c(F)c4)C(C)(F)F)n3)n2)ccn2nc(N)nc12 ; Cc1c(-c2cncc(-c3cnn([C@H](c4ccc(Cl)c(F)c4)C(C)(F)F)n3)n2)ccn2nc(N)nc12]

The title compounds (Ent 1 and Ent 2)-7-(6-(2-(1-(4-chloro-3-fluorophenyl)-2,2-difluoropropyl)-2H-1,2,3-triazol-4-yl)pyrazin-2-yl)-8-methyl-[1,2,4]triazolo[1,5-α]pyridin-2-amine was obtained in a similar fashion as described for the preparation of Example 2, where (±)-4-bromo-2-(1-(4-chloro-3-fluorophenyl)-2,2-difluoropropyl)-2H-1,2,3-triazole (Intermediate 2) was used in place of (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 28-60% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(1-(4-chloro-3-fluorophenyl)-2,2-difluoropropyl)-2H-1,2,3-triazol-4-yl)pyrazin-2-yl)-8-methyl-[1,2,4]triazolo[1,5-α]pyridin-2-amine (13.2 mg, 43.5%). This racemic material was then further purified via preparative SFC with the following conditions: Column: Chiralpak IG 21 mm×250 mm, 5 m particles; Flow Rate:75.00 mL/min; Column Temperature: 40° C. Fraction collection was triggered by UV (230 nm). Mobile Phase A: supercritical CO₂; Mobile Phase B: i-PrOH with 0.1% NH₄OH; Elution gradient: isocratic 30% B over 30 minutes; Fraction collection was triggered by UV (225 nm). Fractions containing the first isomer eluting from the chiral column were combined and dried via centrifugal evaporation to provide (Ent 1)-7-(6-(2-(1-(4-chloro-3-fluorophenyl)-2,2-difluoropropyl)-2H-1,2,3-triazol-4-yl)pyrazin-2-yl)-8-methyl-[1,2,4]triazolo[1,5-α]pyridin-2-amine (3.8 mg, 120.3%) and the second enantiomer was likewise obtained. (Ent 1): ¹H NMR (500 MHz, DMSO-d₆) δ 9.24 (s, 1H), 8.99 (s, 1H), 8.65 (s, 1H), 8.57 (d, J=6.9 Hz, 1H), 7.87 (br d, J=10.1 Hz, 1H), 7.80-7.68 (m, 2H), 7.15 (d, J=6.9 Hz, 1H), 6.94-6.80 (m, 1H), 6.18-6.07 (m, 2H), 2.56 (s, 3H), 1.72 (br t, J=19.1 Hz, 3H). LCMS (ESI, m/z): 500.2 [M+H]⁺.

### Example 5: (Ent 1)-7-(6-(2-(1-(4-chloro-3-fluorophenyl)-2,2-difluoropropyl)-2H-1,2,3-triazol-4-yl)pyridin-2-yl)-[1,2,4]triazolo[1,5-α]pyridin-2-amine

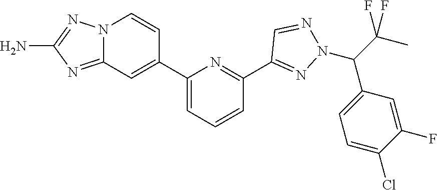 [SMILES: CC(F)(F)C(c1ccc(Cl)c(F)c1)n1ncc(-c2cccc(-c3ccn4nc(N)nc4c3)n2)n1]

The title compound (Ent 1)-7-(6-(2-(1-(4-chloro-3-fluorophenyl)-2,2-difluoropropyl)-2H-1,2,3-triazol-4-yl)pyridin-2-yl)-[1,2,4]triazolo[1,5-α]pyridin-2-amine was obtained as the first eluting enantiomer from the preparative SFC column in a similar fashion as described for the preparation of Example 1, where tert-butyl (tert-butoxycarbonyl)(7-(4,4,5,5-tetramethyl-1,3,2-dioxaborolan-2-yl)-[1,2,4]triazolo[1,5-α]pyridin-2-yl)carbamate (Intermediate 15) and (±)-2-chloro-6-(2-(1-(4-chloro-3-fluorophenyl)-2,2-difluoropropyl)-2H-1,2,3-triazol-4-yl)pyridine (Intermediate 4) were used in place of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate and (±)-4-bromo-2-(1-(3,5-difluorophenyl)-2,2-difluoropropyl)-2H-1,2,3-triazole, respectively, in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 32-62% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(1-(4-chloro-3-fluorophenyl)-2,2-difluoropropyl)-2H-1,2,3-triazol-4-yl)pyridin-2-yl)-[1,2,4]triazolo[1,5-α]pyridin-2-amine (25.6 mg, 58.7%). This racemic material was then further purified via preparative SFC with the following conditions: Column: Whelk-01 (R,R), 21 mm×250 mm, 5 m particles; Flow Rate:75.00 mL/min; Column Temperature: 40° C. Fraction collection was triggered by UV (230 nm). Mobile Phase A: supercritical CO₂; Mobile Phase B: i-PrOH with 0.1% NH₄O H; Elution gradient: isocratic 10% B over 50 minutes; Flow Rate: 75 mL/min; Column Temperature: 40° C. Fraction collection was triggered by UV (240 nm). Fractions containing the first isomer eluting from the chiral column were combined and dried via centrifugal evaporation to provide (Ent 1)-7-(6-(2-(1-(4-chloro-3-fluorophenyl)-2,2-difluoropropyl)-2H-1,2,3-triazol-4-yl)pyridin-2-yl)-[1,2,4]triazolo[1,5-α]pyridin-2-amine (7.9 mg, 17.5%). ¹H NMR (500 MHz, DMSO-d₆) δ 8.71-8.60 (m, 2H), 8.25-8.16 (m, 2H), 8.12-8.04 (m, 1H), 8.04-7.96 (m, 1H), 7.89-7.82 (m, 1H), 7.80-7.63 (m, 3H), 6.89-6.73 (m, 1H), 6.10 (s, 2H), 1.81-1.55 (m, 3H). LCMS (ESI, m/z): 485.4 [M+H]⁺.

### Example 6: (±)-7-(6-(2-(1-(4-chloro-3-fluorophenyl)-2,2-difluoropropyl)-2H-1,2,3-triazol-4-yl)pyridin-2-yl)-8-methyl-[1,2,4]triazolo[1,5-α]pyridin-2-amine

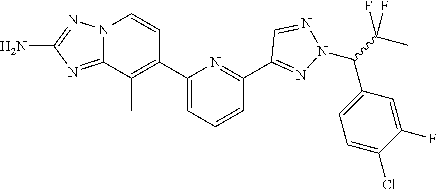 [SMILES: Cc1c(-c2cccc(-c3cnn(C(c4ccc(Cl)c(F)c4)C(C)(F)F)n3)n2)ccn2nc(N)nc12]

The title compound (±)-7-(6-(2-(1-(4-chloro-3-fluorophenyl)-2,2-difluoropropyl)-2H-1,2,3-triazol-4-yl)pyridin-2-yl)-8-methyl-[1,2,4]triazolo[1,5-α]pyridin-2-amine was obtainedin a similar fashion as described for the preparation of Example 2, where 2-chloro-6-(2-(1-(4-chloro-3-fluorophenyl)-2,2-difluoropropyl)-2H-1,2,3-triazol-4-yl)pyridine (Intermediate 4) and 2-(2,5-dimethyl-1H-pyrrol-1-yl)-8-methyl-7-(4,4,5,5-tetramethyl-1,3,2-dioxaborolan-2-yl)-[1,2,4]triazolo[1,5-α]pyridine (Intermediate 16) were used in place of 2-(2,5-dimethyl-1H-pyrrol-1-yl)-7-(6-(4,4,5,5-tetramethyl-1,3,2-dioxaborolan-2-yl)pyrazin-2-yl)-8-methyl-[1,2,4]triazolo[1,5-α]pyridine and (±)-4-bromo-2-(1-(3,5-difluorophenyl)-2,2-difluoropropyl)-2H-1,2,3-triazole, respectively, in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(1-(4-chloro-3-fluorophenyl)-2,2-difluoropropyl)-2H-1,2,3-triazol-4-yl)pyridin-2-yl)-8-methyl-[1,2,4]triazolo[1,5-α]pyridin-2-amine (7.9 mg, 25.9%). ¹H NMR (500 MHz, DMSO-d₆) δ 8.51-8.43 (m, 2H), 8.11-8.03 (m, 1H), 8.02-7.97 (m, 1H), 7.86-7.76 (m, 1H), 7.76-7.60 (m, 3H), 7.10-6.97 (m, 1H), 6.82-6.69 (m, 1H), 6.01 (s, 2H), 2.50 (s, 3H), 1.78-1.54 (m, 3H). LCMS (ESI, m/z): 499.2 [M+H]⁺.

### Example 7: (±)-7-(6-(2-(1-(4-chloro-3-fluorophenyl)-2,2-difluoropropyl)-2H-1,2,3-triazol-4-yl)-3-methylpyridin-2-yl)-[1,2,4]triazolo[1,5-α]pyridin-2-amine

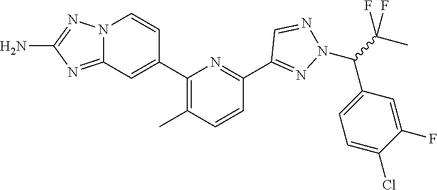 [SMILES: Cc1ccc(-c2cnn(C(c3ccc(Cl)c(F)c3)C(C)(F)F)n2)nc1-c1ccn2nc(N)nc2c1]

The title compound (±)-7-(6-(2-(1-(4-chloro-3-fluorophenyl)-2,2-difluoropropyl)-2H-1,2,3-triazol-4-yl)-3-methylpyridin-2-yl)-[1,2,4]triazolo[1,5-α]pyridin-2-amine was obtained in a similar fashion as described for the preparation of Example 1, where tert-butyl (tert-butoxycarbonyl)(7-(3-methyl-6-(4,4,5,5-tetramethyl-1,3,2-dioxaborolan-2-yl)pyridin-2-yl)-[1,2,4]triazolo[1,5-α]pyridin-2-yl)carbamate (Intermediate 10) and (±)-4-bromo-2-(1-(4-chloro-3-fluorophenyl)-2,2-difluoropropyl)-2H-1,2,3-triazole (Intermediate 2) were used in place of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate and (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 26-100% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(1-(4-chloro-3-fluorophenyl)-2,2-difluoropropyl)-2H-1,2,3-triazol-4-yl)-3-methylpyridin-2-yl)-[1,2,4]triazolo[1,5-α]pyridin-2-amine (4.9 mg, 20.2%). ¹HNMR (500 MHz, DMSO-d₆) δ 8.69-8.58 (m, 1H), 8.49-8.40 (m, 1H), 7.98-7.89 (m, 2H), 7.87-7.80 (m, 1H), 7.78-7.64 (m, 2H), 7.64-7.53 (m, 1H), 7.19-7.08 (m, 1H), 6.85-6.72 (m, 1H), 6.08 (s, 2H), 2.57-2.50 (m, 3H), 2.45 (s, 3H). LCMS (ESI, m/z): 498.9 [M+H]⁺.

### Example 8: (Ent 1 and Ent 2)-7-(6-(2-(2,2-difluoro-1-(4-methoxyphenyl)propyl)-2H-1,2,3-triazol-4-yl)pyrazin-2-yl)-[1,2,4]triazolo[1,5-α]pyridin-2-amine

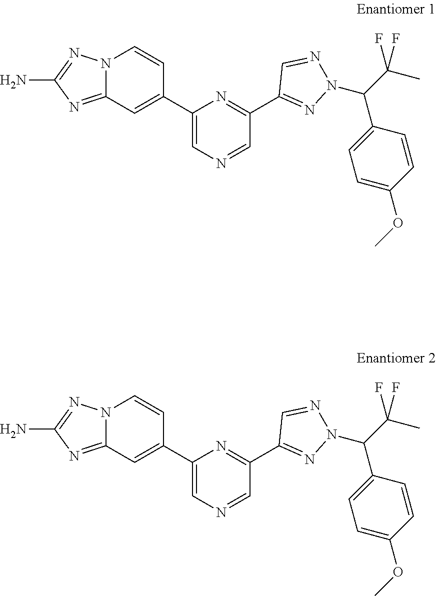 [SMILES: COc1ccc(C(n2ncc(-c3cncc(-c4ccn5nc(N)nc5c4)n3)n2)C(C)(F)F)cc1 ; COc1ccc(C(n2ncc(-c3cncc(-c4ccn5nc(N)nc5c4)n3)n2)C(C)(F)F)cc1]

(The two unassigned enantiomers are

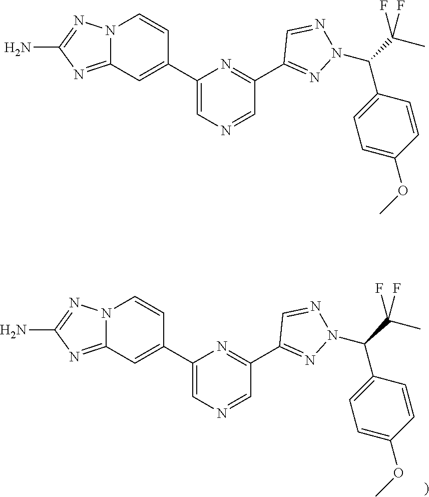 [SMILES: COc1ccc([C@H](n2ncc(-c3cncc(-c4ccn5nc(N)nc5c4)n3)n2)C(C)(F)F)cc1 ; COc1ccc([C@@H](n2ncc(-c3cncc(-c4ccn5nc(N)nc5c4)n3)n2)C(C)(F)F)cc1]

The title compounds (Ent 1 and Ent 2)-7-(6-(2-(2,2-difluoro-1-(4-methoxyphenyl)propyl)-2H-1,2,3-triazol-4-yl)pyrazin-2-yl)-[1,2,4]triazolo[1,5-α]pyridin-2-amine were obtained in a similar fashion as described for the preparation of Example 1, where (±)-4-bromo-2-(2,2-difluoro-1-(4-methoxyphenyl)propyl)-2H-1,2,3-triazole (Intermediate 3D) was used in place of (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-55% B over 22 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-methoxyphenyl)propyl)-2H-1,2,3-triazol-4-yl)pyrazin-2-yl)-[1,2,4]triazolo[1,5-α]pyridin-2-amine (19.8 mg, 54.5%). This racemic material was then further purified via preparative SFC with the following conditions: Column: Whelk-01 (R,R), 21 mm×250 mm, 5 m particles; Flow Rate:75.00 mL/min; Column Temperature: 40° C. Fraction collection was triggered by UV (230 nm). Mobile Phase A: supercritical CO₂; Mobile Phase B: i-PrOH with 0.1% NH₄OH; Elution gradient: isocratic 15% B over 45 minutes; Fraction collection was triggered by UV (275 nm). Fractions containing the first isomer eluting from the chiral column were combined and dried via centrifugal evaporation to provide (Ent 1)-7-(6-(2-(2,2-difluoro-1-(4-methoxyphenyl)propyl)-2H-1,2,3-triazol-4-yl)pyrazin-2-yl)-[1,2,4]triazolo[1,5-α]pyridin-2-amine (6.9 mg, 18.6%) and the second enantiomer was likewise obtained. (Ent 1): ¹H NMR (500 MHz, DMSO-d₆) δ 9.49-9.37 (m, 1H), 9.23-9.11 (m, 1H), 8.77-8.65 (m, 2H), 8.36-8.25 (m, 1H), 7.83-7.69 (m, 3H), 7.10-6.95 (m, 2H), 6.71-6.54 (m, 1H), 6.16 (s, 2H), 3.85-3.69 (m, 3H), 1.78-1.59 (m, 3H). LCMS (ESI, m/z): 464.2 [M+H]⁺.

### Example 9: (Ent 1 and Ent 2)-7-(6-(2-(2,2-difluoro-1-(4-methoxyphenyl)propyl)-2H-1,2,3-triazol-4-yl)pyrazin-2-yl)-8-methyl-[1,2,4]triazolo[1,5-α]pyridin-2-amine

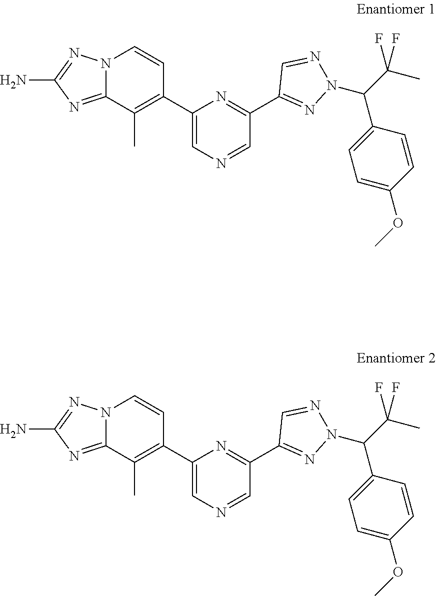 [SMILES: COc1ccc(C(n2ncc(-c3cncc(-c4ccn5nc(N)nc5c4C)n3)n2)C(C)(F)F)cc1 ; COc1ccc(C(n2ncc(-c3cncc(-c4ccn5nc(N)nc5c4C)n3)n2)C(C)(F)F)cc1]

(The two unassigned enantiomers are

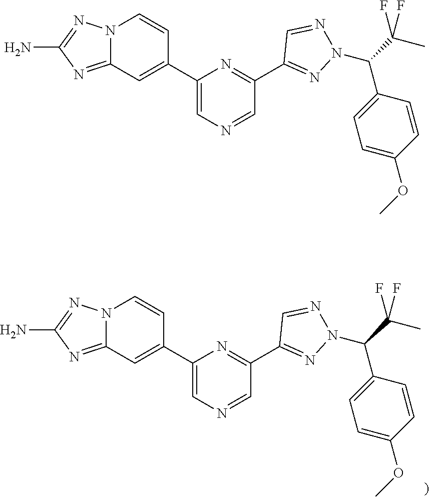 [SMILES: COc1ccc([C@H](n2ncc(-c3cncc(-c4ccn5nc(N)nc5c4)n3)n2)C(C)(F)F)cc1 ; COc1ccc([C@@H](n2ncc(-c3cncc(-c4ccn5nc(N)nc5c4)n3)n2)C(C)(F)F)cc1]

The title compounds (Ent 1 and Ent 2)-7-(6-(2-(2,2-difluoro-1-(4-methoxyphenyl)propyl)-2H-1,2,3-triazol-4-yl)pyrazin-2-yl)-8-methyl-[1,2,4]triazolo[1,5-α]pyridin-2-amine were obtained in a similar fashion as described for the preparation of Example 2, where (±)-4-bromo-2-(2,2-difluoro-1-(4-methoxyphenyl)propyl)-2H-1,2,3-triazole (Intermediate 3D) was used in place of (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 23-63% B over 22 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-methoxyphenyl)propyl)-2H-1,2,3-triazol-4-yl)pyrazin-2-yl)-8-methyl-[1,2,4]triazolo[1,5-α]pyridin-2-amine (17.7 mg, 54.2%). This racemic material was then further purified via preparative SFC with the following conditions: Column: Whelk-01 (R,R), 21 mm×250 mm, 5 m particles; Flow Rate:75.00 mL/min; Column Temperature: 40° C. Fraction collection was triggered by UV (230 nm). Mobile Phase A: supercritical CO₂; Mobile Phase B: i-PrOH with 0.1% NH₄OH; Elution gradient: isocratic 15% B over 38 minutes. Fractions containing the first isomer eluting from the chiral column were combined and dried via centrifugal evaporation to provide (Ent 1)-7-(6-(2-(2,2-difluoro-1-(4-methoxyphenyl)propyl)-2H-1,2,3-triazol-4-yl)pyrazin-2-yl)-8-methyl-[1,2,4]triazolo[1,5-α]pyridin-2-amine (6.2 mg, 18.46%) and the second enantiomer was likewise obtained. (Ent 1): ¹H NMR (500 MHz, DMSO-d₆) δ 9.26-9.13 (m, 1H), 8.99-8.88 (m, 1H), 8.61-8.50 (m, 2H), 7.80-7.66 (m, 2H), 7.17-7.09 (m, 1H), 7.06-6.96 (m, 2H), 6.67-6.52 (m, 1H), 6.10 (s, 2H), 3.77 (s, 3H), 2.54 (s, 3H), 1.75-1.62 (m, 3H). LCMS (ESI, m/z): 478.2 [M+H]⁺.

### Example 10: (Ent 1)-7-(6-(2-(2,2-difluoro-1-(4-methoxyphenyl)propyl)-2H-1,2,3-triazol-4-yl)pyridin-2-yl)-[1,2,4]triazolo[1,5-α]pyridin-2-amine

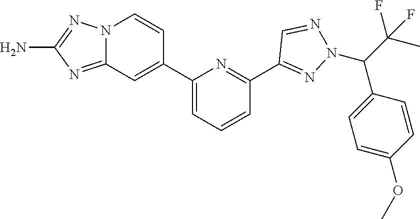 [SMILES: COc1ccc(C(n2ncc(-c3cccc(-c4ccn5nc(N)nc5c4)n3)n2)C(C)(F)F)cc1]

The title compound (Ent 1)-7-(6-(2-(2,2-difluoro-1-(4-methoxyphenyl)propyl)-2H-1,2,3-triazol-4-yl)pyridin-2-yl)-[1,2,4]triazolo[1,5-α]pyridin-2-amine enantiomer was obtained as the first eluting enantiomer from the preparative SFC column in a similar fashion as described for the preparation of Example 1, where 2-chloro-6-(2-(2,2-difluoro-1-(4-methoxyphenyl)propyl)-2H-1,2,3-triazol-4-yl)pyridine (Intermediate 21) and tert-butyl (tert-butoxycarbonyl)(7-(4,4,5,5-tetramethyl-1,3,2-dioxaborolan-2-yl)-[1,2,4]triazolo[1,5-α]pyridin-2-yl)carbamate (Intermediate 15) were used in place of (±)-4-bromo-2-(1-(3,5-difluorophenyl)-2,2-difluoropropyl)-2H-1,2,3-triazole and tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 22 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-methoxyphenyl)propyl)-2H-1,2,3-triazol-4-yl)pyridin-2-yl)-[1,2,4]triazolo[1,5-α]pyridin-2-amine (18.9 mg, 71%). This racemic material was then further purified via preparative SFC with the following conditions: Column: Whelk-01 (R,R), 21 mm×250 mm, 5 m particles; Flow Rate:75.00 mL/min; Column Temperature: 40° C. Fraction collection was triggered by UV (230 nm). Mobile Phase A: supercritical CO₂; Mobile Phase B: i-PrOH with 0.1% NH₄OH; Elution gradient: isocratic 20% B over 20 minutes. Fractions containing the first isomer eluting from the chiral column were combined and dried via centrifugal evaporation to provide (Ent 1)-7-(6-(2-(2,2-difluoro-1-(4-methoxyphenyl)propyl)-2H-1,2,3-triazol-4-yl)pyridin-2-yl)-[1,2,4]triazolo[1,5-α]pyridin-2-amine (5.4 mg, 20.3%). ¹H NMR (500 MHz, DMSO-d₆) δ 8.68-8.65 (m, 1H), 8.62 (s, 1H), 8.26-8.16 (m, 2H), 8.13-8.03 (m, 1H), 8.03-7.93 (m, 1H), 7.82-7.68 (m, 3H), 7.07-6.98 (m, 2H), 6.66-6.44 (m, 1H), 6.15-6.04 (m, 2H), 3.78 (s, 3H), 1.79-1.61 (m, 3H). LCMS (ESI, m/z): 463.2 [M+H]⁺.

### Example 11: (Ent 1)-7-(4-(2-(2,2-difluoro-1-(4-methoxyphenyl)propyl)-2H-1,2,3-triazol-4-yl)pyrimidin-2-yl)-[1,2,4]triazolo[1,5-α]pyridin-2-amine

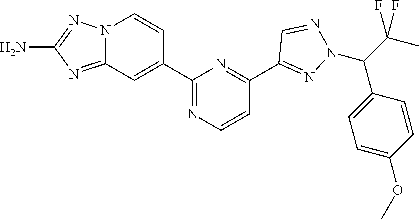 [SMILES: COc1ccc(C(n2ncc(-c3ccnc(-c4ccn5nc(N)nc5c4)n3)n2)C(C)(F)F)cc1]

The title compound (Ent 1)-7-(4-(2-(2,2-difluoro-1-(4-methoxyphenyl)propyl)-2H-1,2,3-triazol-4-yl)pyrimidin-2-yl)-[1,2,4]triazolo[1,5-α]pyridin-2-amine was obtained as the first eluting enantiomer from the preparative SFC column in a similar fashion as described for the preparation of Example 1, where 2-chloro-4-(2-(2,2-difluoro-1-(4-methoxyphenyl)propyl)-2H-1,2,3-triazol-4-yl)pyrimidine (Intermediate 6) and tert-butyl (tert-butoxycarbonyl)(7-(4,4,5,5-tetramethyl-1,3,2-dioxaborolan-2-yl)-[1,2,4]triazolo[1,5-α]pyridin-2-yl)carbamate (Intermediate 15) were used in place of (±)-4-bromo-2-(1-(3,5-difluorophenyl)-2,2-difluoropropyl)-2H-1,2,3-triazole and tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate, respectively, in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 19-55% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(4-(2-(2,2-difluoro-1-(4-methoxyphenyl)propyl)-2H-1,2,3-triazol-4-yl)pyrimidin-2-yl)-[1,2,4]triazolo[1,5-α]pyridin-2-amine (27.2 mg, 87%). This racemic material was then further purified via preparative SFC with the following conditions: Column: Chiralpak AD-H 30 mm×250 mm, 5 m particles; Flow Rate:75.00 mL/min; Column Temperature: 40° C. Fraction collection was triggered by UV (230 nm). Mobile Phase A: supercritical CO₂; Mobile Phase B: i-PrOH with 0.1% NH₄OH; Elution gradient: isocratic 20% B over 20 minutes. Fractions containing the first isomer eluting from the chiral column were combined and dried via centrifugal evaporation to provide (Ent 1)-7-(4-(2-(2,2-difluoro-1-(4-methoxyphenyl)propyl)-2H-1,2,3-triazol-4-yl)pyrimidin-2-yl)-[1,2,4]triazolo[1,5-α]pyridin-2-amine (7.7 mg, 24.5%). ¹H NMR (500 MHz, DMSO-d₆) δ 9.12-9.00 (m, 1H), 8.91-8.79 (m, 1H), 8.76-8.63 (m, 1H), 8.49-8.38 (m, 1H), 8.01-7.90 (m, 2H), 7.81-7.70 (m, 2H), 7.11-6.99 (m, 2H), 6.75-6.54 (m, 1H), 6.19 (s, 2H), 3.78 (s, 3H), 1.78-1.62 (m, 3H). LCMS (ESI, m/z): 464.2 [M+H]⁺.

### Example 12: (±)-7-(4-(2-(2,2-difluoro-1-(4-methoxyphenyl)propyl)-2H-1,2,3-triazol-4-yl)pyrimidin-2-yl)-8-methyl-[1,2,4]triazolo[1,5-α]pyridin-2-amine racemate

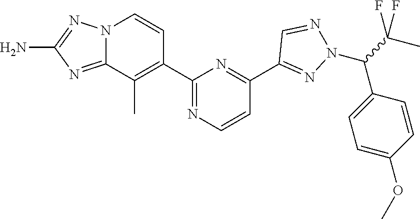 [SMILES: COc1ccc(C(n2ncc(-c3ccnc(-c4ccn5nc(N)nc5c4C)n3)n2)C(C)(F)F)cc1]

The title compound (±)-7-(4-(2-(2,2-difluoro-1-(4-methoxyphenyl)propyl)-2H-1,2,3-triazol-4-yl)pyrimidin-2-yl)-8-methyl-[1,2,4]triazolo[1,5-α]pyridin-2-amine was obtained in a similar fashion as described for the preparation of Example 2, where 2-chloro-4-(2-(2,2-difluoro-1-(4-methoxyphenyl)propyl)-2H-1,2,3-triazol-4-yl)pyrimidine (Intermediate 6) and 2-(2,5-dimethyl-1H-pyrrol-1-yl)-7-(4,4,5,5-tetramethyl-1,3,2-dioxaborolan-2-yl)-[1,2,4]triazolo[1,5-α]pyridine (Intermediate 16) were used in place of 2-(2,5-dimethyl-1H-pyrrol-1-yl)-7-(6-(4,4,5,5-tetramethyl-1,3,2-dioxaborolan-2-yl)pyrazin-2-yl)-8-methyl-[1,2,4]triazolo[1,5-α]pyridine and (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-55% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-methoxyphenyl)propyl)-2H-1,2,3-triazol-4-yl)pyridin-2-yl)-[1,2,4]triazolo[1,5-α]pyridin-2-amine (4.9 mg, 17.6%). LCMS (ESI, m/z): 478.2 [M+H]⁺.

### Example 13: (±)-7-(5-(2-(2,2-difluoro-1-(4-methoxyphenyl)propyl)-2H-1,2,3-triazol-4-yl)pyridin-3-yl)-[1,2,4]triazolo[1,5-α]pyridin-2-amine

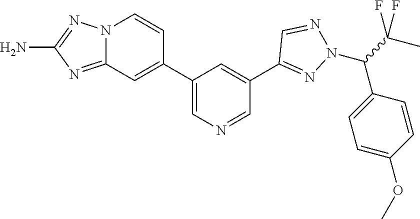 [SMILES: COc1ccc(C(n2ncc(-c3cncc(-c4ccn5nc(N)nc5c4)c3)n2)C(C)(F)F)cc1]

The title compound (±)-7-(5-(2-(2,2-difluoro-1-(4-methoxyphenyl)propyl)-2H-1,2,3-triazol-4-yl)pyridin-3-yl)-[1,2,4]triazolo[1,5-α]pyridin-2-amine was obtained in a similar fashion as described for the preparation of Example 1, where (±)-3-bromo-5-(2-(2,2-difluoro-1-(4-methoxyphenyl)propyl)-2H-1,2,3-triazol-4-yl)pyridine (Intermediate 5) and tert-butyl (tert-butoxycarbonyl)(7-(4,4,5,5-tetramethyl-1,3,2-dioxaborolan-2-yl)-[1,2,4]triazolo[1,5-α]pyridin-2-yl)carbamate (Intermediate 15) were used in place of (±)-4-bromo-2-(1-(3,5-difluorophenyl)-2,2-difluoropropyl)-2H-1,2,3-triazole and tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 18-48% B over 23 minutes; Flow Rate: 20 mL/min; Column Temperature: 25° C. Fraction collection was triggered by MS and UV signals. Fractions containing the desired product were combined and dried via centrifugal evaporation to give (±)-7-(5-(2-(2,2-difluoro-1-(4-methoxyphenyl)propyl)-2H-1,2,3-triazol-4-yl)pyridin-3-yl)-[1,2,4]triazolo[1,5-α]pyridin-2-amine (19.1 mg, 32%). ¹H NMR (500 MHz, DMSO-d₆) δ 9.16-9.11 (m, 1H), 9.09-9.02 (m, 1H), 8.71-8.60 (m, 3H), 7.93-7.87 (m, 1H), 7.78-7.72 (m, 2H), 7.42-7.33 (m, 1H), 7.05-6.95 (m, 2H), 6.65-6.50 (m, 1H), 3.78 (s, 3H), 1.80-1.60 (m, 3H). LCMS (ESI, m/z): 463.4 [M+H]⁺.

### Example 14: (±)-7-(6-(2-(2,2-difluoro-1-(4-methoxyphenyl)propyl)-2H-1,2,3-triazol-4-yl)-5-methylpyrazin-2-yl)-[1,2,4]triazolo[1,5-α]pyridin-2-amine

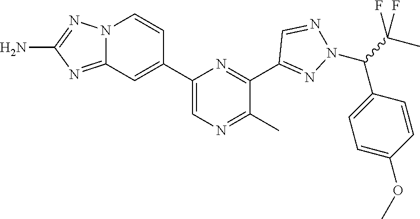 [SMILES: COc1ccc(C(n2ncc(-c3nc(-c4ccn5nc(N)nc5c4)cnc3C)n2)C(C)(F)F)cc1]

The title compound (±)-7-(6-(2-(2,2-difluoro-1-(4-methoxyphenyl)propyl)-2H-1,2,3-triazol-4-yl)-5-methylpyrazin-2-yl)-[1,2,4]triazolo[1,5-α]pyridin-2-amine was obtained in a similar fashion as described for the preparation of Example 1, where tert-butyl (tert-butoxycarbonyl)(7-(6-chloro-5-methylpyrazin-2-yl)-[1,2,4]triazolo[1,5-α]pyridin-2-yl)carbamate (Intermediate 12) and 2-(2,2-difluoro-1-(4-methoxyphenyl)propyl)-4-(4,4,5,5-tetramethyl-1,3,2-dioxaborolan-2-yl)-2H-1,2,3-triazole (Intermediate 3) were used in place of (±)-4-bromo-2-(1-(3,5-difluorophenyl)-2,2-difluoropropyl)-2H-1,2,3-triazole and tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 18-48% B over 22 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-methoxyphenyl)propyl)-2H-1,2,3-triazol-4-yl)-5-methylpyrazin-2-yl)-[1,2,4]triazolo[1,5-α]pyridin-2-amine (2.9 mg, 5.6%). ¹H NMR (500 MHz, DMSO-d₆) δ 9.39-9.28 (m, 1H), 8.76-8.65 (m, 2H), 8.33-8.23 (m, 1H), 7.85-7.70 (m, 3H), 7.08-6.98 (m, 2H), 6.67-6.50 (m, 1H), 3.78 (s, 3H), 2.92 (s, 3H), 1.79-1.62 (m, 3H). LCMS (ESI, m/z): 478.2 [M+H]⁺.

### Example 15: (Ent 1 and Ent 2)-7-(6-(2-(2,2-difluoro-1-(4-methoxyphenyl)propyl)-2H-1,2,3-triazol-4-yl)-3-methylpyrazin-2-yl)-[1,2,4]triazolo[1,5-α]pyridin-2-amine

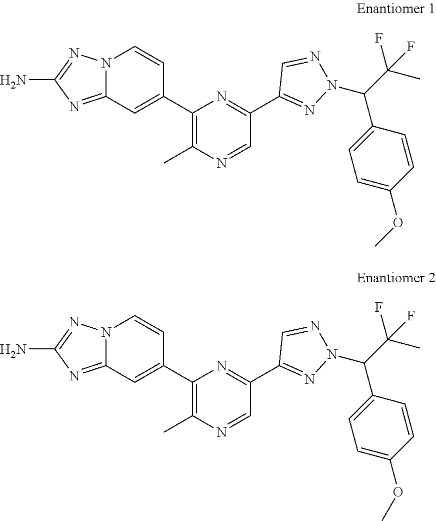 [SMILES: COc1ccc(C(n2ncc(-c3cnc(C)c(-c4ccn5nc(N)nc5c4)n3)n2)C(C)(F)F)cc1 ; COc1ccc(C(n2ncc(-c3cnc(C)c(-c4ccn5nc(N)nc5c4)n3)n2)C(C)(F)F)cc1]

(The two unassigned enantiomers are

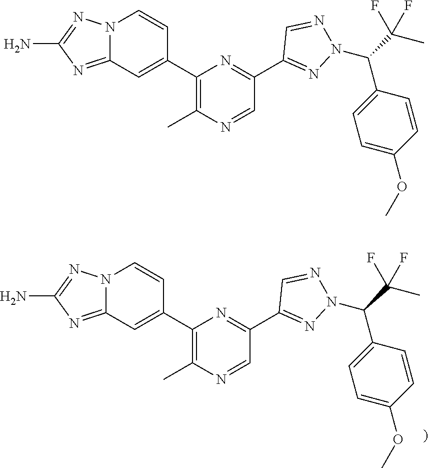

The title compounds (Ent 1 and Ent 2)-7-(6-(2-(2,2-difluoro-1-(4-methoxyphenyl)propyl)-2H-1,2,3-triazol-4-yl)-3-methylpyrazin-2-yl)-[1,2,4]triazolo[1,5-α]pyridin-2-amine were obtained in a similar fashion as described for the preparation of Example 1, where tert-butyl (tert-butoxycarbonyl)(7-(6-chloro-3-methylpyrazin-2-yl)-[1,2,4]triazolo[1,5-α]pyridin-2-yl)carbamate (Intermediate 11) and 2-(2,2-difluoro-1-(4-methoxyphenyl)propyl)-4-(4,4,5,5-tetramethyl-1,3,2-dioxaborolan-2-yl)-2H-1,2,3-triazole (Intermediate 3) was used in place of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate and (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-methoxyphenyl)propyl)-2H-1,2,3-triazol-4-yl)-3-methylpyrazin-2-yl)-[1,2,4]triazolo[1,5-α]pyridin-2-amine (17.3 mg, 27.5%). This racemic material was then further purified via preparative SFC with the following conditions: Column: Whelk-01 (R,R), 21 mm×250 mm, 5 m particles; Flow Rate:75.00 mL/min; Column Temperature: 40° C. Fraction collection was triggered by UV (230 nm). Mobile Phase A: supercritical CO₂; Mobile Phase B: i-PrOH with 0.1% NH₄O H; Elution gradient: isocratic 20% B over 22 minutes. Fractions containing the first isomer eluting from the chiral column were combined and dried via centrifugal evaporation to provide (Ent 1)-7-(6-(2-(2,2-difluoro-1-(4-methoxyphenyl)propyl)-2H-1,2,3-triazol-4-yl)-3-methylpyrazin-2-yl)-[1,2,4]triazolo[1,5-α]pyridin-2-amine (6.9 mg, 10.9%) and the second enantiomer was likewise obtained. ¹H NMR (500 MHz, DMSO-d₆) δ 8.98-8.84 (m, 1H), 8.57-8.46 (m, 1H), 8.41-8.30 (m, 1H), 7.64-7.45 (m, 3H), 7.12-6.99 (m, 1H), 6.89-6.79 (m, 2H), 6.52-6.35 (m, 1H), 5.96 (s, 2H), 3.60 (s, 3H), 2.51 (s, 3H), 1.61-1.42 (m, 3H). LCMS (ESI, m/z): 478.2 [M+H]⁺.

### Example 16: (Ent 2)-7-(6-(2-(2,2-difluoro-1-(4-methoxyphenyl)propyl)-2H-1,2,3-triazol-4-yl)-3-methylpyridin-2-yl)-[1,2,4]triazolo[1,5-α]pyridin-2-amine

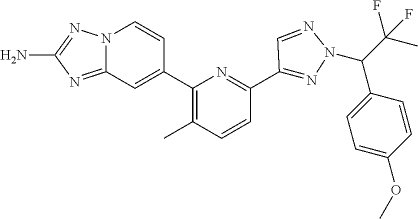 [SMILES: COc1ccc(C(n2ncc(-c3ccc(C)c(-c4ccn5nc(N)nc5c4)n3)n2)C(C)(F)F)cc1]

The title compound (Ent 2)-7-(6-(2-(2,2-difluoro-1-(4-methoxyphenyl)propyl)-2H-1,2,3-triazol-4-yl)-3-methylpyridin-2-yl)-[1,2,4]triazolo[1,5-α]pyridin-2-amine was obtained as the second eluting enantiomer from the preparative SFC column in a similar fashion as described for the preparation of Example 1, where tert-butyl (tert-butoxycarbonyl)(7-(3-methyl-6-(4,4,5,5-tetramethyl-1,3,2-dioxaborolan-2-yl)pyridin-2-yl)-[1,2,4]triazolo[1,5-α]pyridin-2-yl)carbamate (Intermediate 10) and (±)-4-bromo-2-(2,2-difluoro-1-(4-methoxyphenyl)propyl)-2H-1,2,3-triazole (Intermediate 3D) were used in place of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate and (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 19-54% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-methoxyphenyl)propyl)-2H-1,2,3-triazol-4-yl)-3-methylpyridin-2-yl)-[1,2,4]triazolo[1,5-α]pyridin-2-amine (8.5 mg, 11.8%). This racemic material was then further purified via preparative SFC with the following conditions: Column: Chiralpak 30 mm×250 mm, 5 m particles; Flow Rate:100.00 mL/min; Column Temperature: 40° C. Fraction collection was triggered by UV (230 nm). Mobile Phase A: supercritical CO₂; Mobile Phase B: i-PrOH with 0.1% NH₄O H; Elution gradient: isocratic 30% B over 18 minutes. Fractions containing the second isomer to elute from the chiral column were combined and dried via centrifugal evaporation to provide (Ent 2)-7-(6-(2-(2,2-difluoro-1-(4-methoxyphenyl)propyl)-2H-1,2,3-triazol-4-yl)-3-methylpyridin-2-yl)-[1,2,4]triazolo[1,5-α]pyridin-2-amine (2.7 mg, 3.8%). ¹H NMR (500 MHz, DMSO-d₆) δ 8.69-8.59 (m, 1H), 8.43-8.34 (m, 1H), 7.95-7.88 (m, 2H), 7.78-7.68 (m, 2H), 7.63-7.53 (m, 1H), 7.20-7.10 (m, 1H), 7.07-6.97 (m, 2H), 6.62-6.47 (m, 1H), 6.08 (s, 2H), 3.78 (s, 3H), 2.44 (s, 3H), 1.77-1.59 (m, 3H). LCMS (ESI, m/z): 477.2 [M+H]⁺.

### Example 17: (Ent 2)-7-(6-(2-(2,2-difluoro-1-(4-fluorophenyl)propyl)-2H-1,2,3-triazol-4-yl)-3-methylpyrazin-2-yl)-[1,2,4]triazolo[1,5-α]pyridin-2-amine

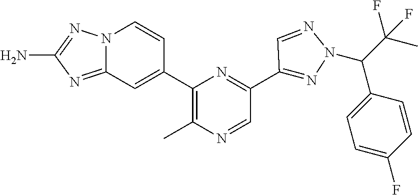 [SMILES: Cc1ncc(-c2cnn(C(c3ccc(F)cc3)C(C)(F)F)n2)nc1-c1ccn2nc(N)nc2c1]

The title compound (Ent 2)-7-(6-(2-(2,2-difluoro-1-(4-fluorophenyl)propyl)-2H-1,2,3-triazol-4-yl)-3-methylpyrazin-2-yl)-[1,2,4]triazolo[1,5-α]pyridin-2-amine was obtained as the second eluting enantiomer from the preparative SFC column in a similar fashion as described for the preparation of Example 1, where (±)-2-(2,2-difluoro-1-(4-fluorophenyl)propyl)-4-(4,4,5,5-tetramethyl-1,3,2-dioxaborolan-2-yl)-2H-1,2,3-triazole (Racemic Intermediate 25) and tert-butyl (tert-butoxycarbonyl)(7-(6-chloro-3-methylpyrazin-2-yl)-[1,2,4]triazolo[1,5-α]pyridin-2-yl)carbamate (Intermediate 9) were used in place of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate and (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 21-53% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-fluorophenyl)propyl)-2H-1,2,3-triazol-4-yl)-3-methylpyrazin-2-yl)-[1,2,4]triazolo[1,5-α]pyridin-2-amine (13.6 mg, 58.9%). This racemic material was then further purified via preparative SFC with the following conditions: Column: Chiralpak, 30 mm×250 mm, 5 m particles; Flow Rate:100.00 mL/min; Column Temperature: 40° C. Fraction collection was triggered by UV (230 nm). Mobile Phase A: supercritical CO₂; Mobile Phase B: i-PrOH with 0.1% NH₄OH; Elution gradient: isocratic 15% B over 40 minutes. Fraction collection was triggered by UV (275 nm). Fractions containing the second isomer eluting from the chiral column were combined and dried via centrifugal evaporation to provide (Ent 2)-7-(6-(2-(2,2-difluoro-1-(4-fluorophenyl)propyl)-2H-1,2,3-triazol-4-yl)-3-methylpyrazin-2-yl)-[1,2,4]triazolo[1,5-α]pyridin-2-amine (7.9 mg, 34.2%). ¹H NMR (500 MHz, DMSO-d₆) δ 9.14-9.03 (m, 1H), 8.74-8.63 (m, 1H), 8.59-8.53 (m, 1H), 7.93-7.83 (m, 2H), 7.75-7.66 (m, 1H), 7.38-7.26 (m, 2H), 7.26-7.18 (m, 1H), 6.82-6.70 (m, 1H), 6.13 (s, 2H), 2.52 (s, 3H), 1.78-1.62 (m, 3H). LCMS (ESI, m/z): 466.2 [M+H]⁺.

### Example 18: (Ent 2)-7-(6-(2-(2,2-difluoro-1-(4-fluorophenyl)propyl)-2H-1,2,3-triazol-4-yl)-3-methylpyrazin-2-yl)-[1,2,4]triazolo[1,5-α]pyridin-2-amine

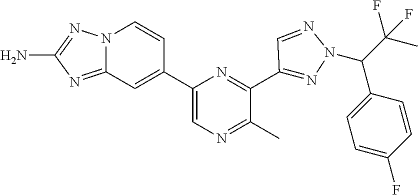 [SMILES: Cc1ncc(-c2ccn3nc(N)nc3c2)nc1-c1cnn(C(c2ccc(F)cc2)C(C)(F)F)n1]

The title compound (Ent 2)-7-(6-(2-(2,2-difluoro-1-(4-fluorophenyl)propyl)-2H-1,2,3-triazol-4-yl)-3-methylpyrazin-2-yl)-[1,2,4]triazolo[1,5-α]pyridin-2-amine was obtained as the second eluting enantiomer from the preparative SFC column in a similar fashion as described for the preparation of Example 1, where tert-butyl (tert-butoxycarbonyl)(7-(6-chloro-5-methylpyrazin-2-yl)-[1,2,4]triazolo[1,5-α]pyridin-2-yl)carbamate (Intermediate 12) and 2-(2,2-difluoro-1-(4-fluorophenyl)propyl)-4-(4,4,5,5-tetramethyl-1,3,2-dioxaborolan-2-yl)-2H-1,2,3-triazole (Racemic Intermediate 25) were used in place of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate and (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 26-66% B over 25 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-fluorophenyl)propyl)-2H-1,2,3-triazol-4-yl)-3-methylpyrazin-2-yl)-[1,2,4]triazolo[1,5-α]pyridin-2-amine (24.9 mg, 25.3%). This racemic material was then further purified via preparative SFC with the following conditions: Column: Chiralpak IA, 21 mm×250 mm, 5 m particles; Flow Rate:75.00 mL/min; Column Temperature: 40° C. Fraction collection was triggered by UV (235 nm). Mobile Phase A: supercritical CO₂; Mobile Phase B: i-PrOH with 0.1% NH₄O H; Elution gradient: isocratic 40% B over 20 minutes. Fractions containing the second isomer eluting from the chiral column were combined and dried via centrifugal evaporation to provide (Ent 2)-7-(6-(2-(2,2-difluoro-1-(4-fluorophenyl)propyl)-2H-1,2,3-triazol-4-yl)-3-methylpyrazin-2-yl)-[1,2,4]triazolo[1,5-α]pyridin-2-amine (9.6 mg, 10%). ¹H NMR (500 MHz, DMSO-d₆) δ 9.39-9.28 (m, 1H), 8.77-8.62 (m, 2H), 8.35-8.20 (m, 1H), 7.98-7.86 (m, 2H), 7.84-7.71 (m, 1H), 7.41-7.27 (m, 2H), 6.87-6.68 (m, 1H), 6.15 (sm, 2H), 2.92 (s, 3H), 1.82-1.59 (m, 3H). LCMS (ESI, m/z): 466.2 [M+H]⁺.

### Example 19: (Ent 1)-7-(6-(2-(2,2-difluoro-1-(4-fluorophenyl)propyl)-2H-1,2,3-triazol-4-yl)-5-methylpyrazin-2-yl)-[1,2,4]triazolo[1,5-α]pyridin-2-amine

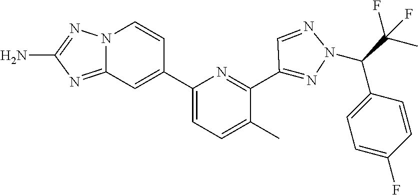 [SMILES: Cc1ccc(-c2ccn3nc(N)nc3c2)nc1-c1cnn([C@H](c2ccc(F)cc2)C(C)(F)F)n1]

The title compound (Ent 1)-7-(6-(2-(2,2-difluoro-1-(4-fluorophenyl)propyl)-2H-1,2,3-triazol-4-yl)-5-methylpyrazin-2-yl)-[1,2,4]triazolo[1,5-α]pyridin-2-amine was obtained as the first eluting enantiomer from the preparative SFC column in a similar fashion as described for the preparation of Example 1, where 6-chloro-2-(2-(2,2-difluoro-1-(4-fluorophenyl)propyl)-2H-1,2,3-triazol-4-yl)-3-methylpyridine (Intermediate 29) and tert-butyl (tert-butoxycarbonyl)(7-(4,4,5,5-tetramethyl-1,3,2-dioxaborolan-2-yl)-[1,2,4]triazolo[1,5-α]pyridin-2-yl)carbamate (Intermediate 15) were used in place of (±)-4-bromo-2-(1-(3,5-difluorophenyl)-2,2-difluoropropyl)-2H-1,2,3-triazole and tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-65% B over 25 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-fluorophenyl)propyl)-2H-1,2,3-triazol-4-yl)-5-methylpyrazin-2-yl)-[1,2,4]triazolo[1,5-α]pyridin-2-amine (11.9 mg, 43.7%). This racemic material was then further purified via preparative SFC with the following conditions: Column: Whelk-01 (R,R), 21 mm×250 mm, 5 m particles; Flow Rate:75.00 mL/min; Column Temperature: 40° C. Fraction collection was triggered by UV (235 nm). Mobile Phase A: supercritical CO₂; Mobile Phase B: i-PrOH with 0.1% NH₄O H; Elution gradient: isocratic 10% B over 45 minutes. Fractions containing the first isomer eluting from the chiral column were combined and dried via centrifugal evaporation to provide (Ent 1)-7-(6-(2-(2,2-difluoro-1-(4-fluorophenyl)propyl)-2H-1,2,3-triazol-4-yl)-5-methylpyrazin-2-yl)-[1,2,4]triazolo[1,5-α]pyridin-2-amine (3.5 mg, 12.8%). ¹H NMR (500 MHz, DMSO-d₆) δ 8.67-8.54 (m, 2H), 8.20-8.07 (m, 2H), 7.98-7.83 (m, 3H), 7.81-7.69 (m, 1H), 7.38-7.23 (m, 1H), 6.76-6.59 (m, 1H), 6.08 (s, 2H0, 2.75-2.62 (m, 3H), 1.79-1.62 (m, 3H). LCMS (ESI, m/z): 465.2 [M+H]⁺.

### Example 20: (±)-7-(6-(2-(2,2-difluoro-1-(4-fluorophenyl)propyl)-2H-1,2,3-triazol-4-yl)-3-methylpyridin-2-yl)-[1,2,4]triazolo[1,5-α]pyridin-2-amine

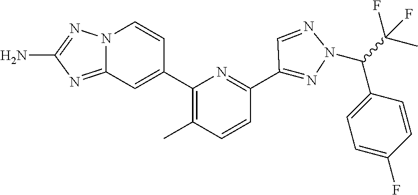 [SMILES: Cc1ccc(-c2cnn(C(c3ccc(F)cc3)C(C)(F)F)n2)nc1-c1ccn2nc(N)nc2c1]

The title compound (±)-7-(6-(2-(2,2-difluoro-1-(4-fluorophenyl)propyl)-2H-1,2,3-triazol-4-yl)-3-methylpyridin-2-yl)-[1,2,4]triazolo[1,5-α]pyridin-2-amine was obtained in a similar fashion as described for the preparation of Example 1, where tert-butyl (tert-butoxycarbonyl)(7-(3-methyl-6-(4,4,5,5-tetramethyl-1,3,2-dioxaborolan-2-yl)pyridin-2-yl)-[1,2,4]triazolo[1,5-α]pyridin-2-yl)carbamate (Intermediate 10) and (±)-4-bromo-2-(2,2-difluoro-1-(4-fluorophenyl)propyl)-2H-1,2,3-triazole (Intermediate 19) were used in place of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate and (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 26-100% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-fluorophenyl)propyl)-2H-1,2,3-triazol-4-yl)-3-methylpyridin-2-yl)-[1,2,4]triazolo[1,5-α]pyridin-2-amine (6.5 mg, 29.3%). ¹H NMR (500 MHz, DMSO-d₆) δ 8.68-8.54 (m, 1H), 8.45-8.35 (m, 1H), 7.97-7.89 (m, 2H), 7.89-7.80 (m, 2H), 7.64-7.52 (m, 1H), 7.37-7.26 (m, 2H), 7.21-7.07 (m, 1H), 6.82-6.61 (m, 1H), 6.08 (s, 2H), 2.44 (s, 3H), 1.78-1.62 (m, 3H). LCMS (ESI, m/z): 465.1 [M+H]⁺.

### Example 21: (Ent 2)-7-(4-(2-(2,2-difluoro-1-(4-fluorophenyl)propyl)-2H-1,2,3-triazol-4-yl)-5-methylpyrimidin-2-yl)-[1,2,4]triazolo[1,5-α]pyridin-2-amine

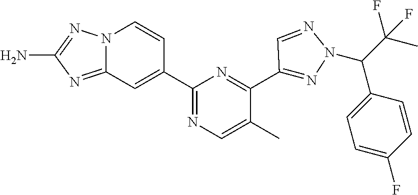 [SMILES: Cc1cnc(-c2ccn3nc(N)nc3c2)nc1-c1cnn(C(c2ccc(F)cc2)C(C)(F)F)n1]

The title compound (Ent 2)-7-(4-(2-(2,2-difluoro-1-(4-fluorophenyl)propyl)-2H-1,2,3-triazol-4-yl)-5-methylpyrimidin-2-yl)-[1,2,4]triazolo[1,5-α]pyridin-2-amine was obtained as the second eluting enantiomer from the preparative SFC column in a similar fashion as described for the preparation of Example 1, where 2-chloro-4-(2-(2,2-difluoro-1-(4-fluorophenyl)propyl)-2H-1,2,3-triazol-4-yl)-5-methylpyrimidine (Intermediate 8) and tert-butyl (tert-butoxycarbonyl)(7-(4,4,5,5-tetramethyl-1,3,2-dioxaborolan-2-yl)-[1,2,4]triazolo[1,5-α]pyridin-2-yl)carbamate (Intermediate 15) were used in place of (±)-4-bromo-2-(1-(3,5-difluorophenyl)-2,2-difluoropropyl)-2H-1,2,3-triazole and tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 28-58% B over 22 minutes; Flow Rate: 20 mL/min; Column Temperature: 25° C. Fraction collection was triggered by MS and UV signals. Fractions containing the desired product were combined and dried via centrifugal evaporation to give (±)-7-(4-(2-(2,2-difluoro-1-(4-fluorophenyl)propyl)-2H-1,2,3-triazol-4-yl)-5-methylpyrimidin-2-yl)-[1,2,4]triazolo[1,5-α]pyridin-2-amine (23.4 mg, 42.9%). This racemic material was then further purified via preparative SFC with the following conditions: Column: Chiralpak, 21 mm×150 mm, 5 m particles; Flow Rate:75.00 mL/min; Column Temperature: 40° C. Fraction collection was triggered by UV (240 nm). Mobile Phase A: supercritical CO₂; Mobile Phase B: i-PrOH with 0.1% NH₄OH; Elution gradient: isocratic 15% B over 24 minutes. Fractions containing the second isomer eluting from the chiral column were combined and dried via centrifugal evaporation to provide (Ent 2)-7-(4-(2-(2,2-difluoro-1-(4-fluorophenyl)propyl)-2H-1,2,3-triazol-4-yl)-5-methylpyrimidin-2-yl)-[1,2,4]triazolo[1,5-α]pyridin-2-amine (9.2 mg, 17.2%). ¹H NMR (500 MHz, DMSO-d₆) δ 9.00-8.92 (m, 1H), 8.92-8.81 (m, 1H), 8.73-8.62 (m, 1H), 8.44-8.33 (m, 1H), 7.99-7.83 (m, 3H), 7.42-7.29 (m, 2H), 6.87-6.69 (m, 1H), 6.17 (s, 2H), 2.69 (s, 3H), 1.71 (br t, J=19.0 Hz, 3H). LCMS (ESI, m/z): 466.2 [M+H]⁺.

### Example 22: (Ent 2)-7-(4-(2-(2,2-difluoro-1-(4-fluorophenyl)propyl)-2H-1,2,3-triazol-4-yl)-5-methylpyrimidin-2-yl)-8-methyl-[1,2,4]triazolo[1,5-α]pyridin-2-amine

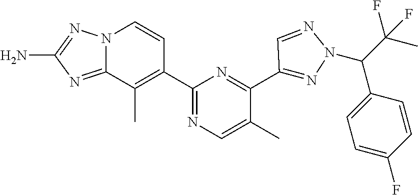 [SMILES: Cc1cnc(-c2ccn3nc(N)nc3c2C)nc1-c1cnn(C(c2ccc(F)cc2)C(C)(F)F)n1]

The title compound (Ent 2)-7-(4-(2-(2,2-difluoro-1-(4-fluorophenyl)propyl)-2H-1,2,3-triazol-4-yl)-5-methylpyrimidin-2-yl)-8-methyl-[1,2,4]triazolo[1,5-α]pyridin-2-amine was obtained as the second eluting enantiomer from the preparative SFC column in a similar fashion as described for the preparation of Example 2, where 2-chloro-4-(2-(2,2-difluoro-1-(4-fluorophenyl)propyl)-2H-1,2,3-triazol-4-yl)-5-methylpyrimidine (Intermediate 8) and 2-(2,5-dimethyl-1H-pyrrol-1-yl)-8-methyl-7-(4,4,5,5-tetramethyl-1,3,2-dioxaborolan-2-yl)-[1,2,4]triazolo[1,5-α]pyridine (Intermediate 16) were used in place of (±)-4-bromo-2-(1-(3,5-difluorophenyl)-2,2-difluoropropyl)-2H-1,2,3-triazole and 2-(2,5-dimethyl-1H-pyrrol-1-yl)-8-methyl-7-(6-(4,4,5,5-tetramethyl-1,3,2-dioxaborolan-2-yl)pyrazin-2-yl)-[1,2,4]triazolo[1,5-α]pyridine in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-55% B over 25 minutes; Flow Rate: 20 mL/min; Column Temperature: 25° C. Fraction collection was triggered by MS and UV signals. Fractions containing the desired product were combined and dried via centrifugal evaporation to give (±)-7-(4-(2-(2,2-difluoro-1-(4-fluorophenyl)propyl)-2H-1,2,3-triazol-4-yl)-5-methylpyrimidin-2-yl)-8-methyl-[1,2,4]triazolo[1,5-α]pyridin-2-amine (37.3 mg, 54.2%). This racemic material was then further purified via preparative SFC with the following conditions: Column: Chiralpak IA, 21 mm×250 mm, 5 m particles; Flow Rate:75.00 mL/min; Column Temperature: 45° C. Fraction collection was triggered by UV (235 nm). Mobile Phase A: supercritical CO₂; Mobile Phase B: i-PrOH with 0.1% NH₄O H; Elution gradient: isocratic 40% B over 25 minutes. Fractions containing the second isomer eluting from the chiral column were combined and dried via centrifugal evaporation to provide (Ent 2)-7-(4-(2-(2,2-difluoro-1-(4-fluorophenyl)propyl)-2H-1,2,3-triazol-4-yl)-5-methylpyrimidin-2-yl)-8-methyl-[1,2,4]triazolo[1,5-α]pyridin-2-amine (10.9 mg, 16.7%). ¹H NMR (500 MHz, DMSO-d₆) δ 9.04-8.94 (m, 1H), 8.68-8.61 (m, 1H), 8.53-8.47 (m, 1H), 7.97-7.84 (m, 2H), 7.55-7.45 (m, 1H), 7.37-7.27 (m, 2H), 6.85-6.69 (m, 1H), 6.09 (s, 2H), 2.76 (s, 3H), 2.69 (s, 3H), 1.79-1.62 (m, 3H). LCMS (ESI, m/z): 480.2 [M+H]⁺.

### Example 23: (±)-7-(6-(2-(2,2-difluoro-1-(4-fluorophenyl)propyl)-2H-1,2,3-triazol-4-yl)-5-methylpyrazin-2-yl)-8-methyl-[1,2,4]triazolo[1,5-α]pyridin-2-amine

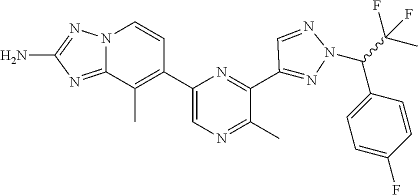 [SMILES: Cc1ncc(-c2ccn3nc(N)nc3c2C)nc1-c1cnn(C(c2ccc(F)cc2)C(C)(F)F)n1]

The title compound (±)-7-(6-(2-(2,2-difluoro-1-(4-fluorophenyl)propyl)-2H-1,2,3-triazol-4-yl)-5-methylpyrazin-2-yl)-8-methyl-[1,2,4]triazolo[1,5-α]pyridin-2-amine was obtained in a similar fashion as described for the preparation of Example 2, where 7-(6-chloro-5-methylpyrazin-2-yl)-2-(2,5-dimethyl-1H-pyrrol-1-yl)-8-methyl-[1,2,4]triazolo[1,5-α]pyridine (Intermediate 14) and 2-(2,2-difluoro-1-(4-fluorophenyl)propyl)-4-(4,4,5,5-tetramethyl-1,3,2-dioxaborolan-2-yl)-2H-1,2,3-triazole (Racemic Intermediate 25) was used in place of 2-(2,5-dimethyl-1H-pyrrol-1-yl)-8-methyl-7-(6-(4,4,5,5-tetramethyl-1,3,2-dioxaborolan-2-yl)pyrazin-2-yl)-[1,2,4]triazolo[1,5-α]pyridine and (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 25-55% B over 25 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-fluorophenyl)propyl)-2H-1,2,3-triazol-4-yl)-5-methylpyrazin-2-yl)-8-methyl-[1,2,4]triazolo[1,5-α]pyridin-2-amine (5.7 mg, 7.6%). ¹H NMR (500 MHz, DMSO-d₆) δ 8.92-8.84 (m, 1H), 8.59-8.49 (m, 2H), 7.97-7.82 (m, 2H), 7.40-7.25 (m, 2H), 7.21-7.11 (m, 1H), 6.85-6.71 (m, 1H), 6.11 (s, 2H), 2.94 (s, 3H), 2.56 (s, 3H), 1.77-1.64 (m, 3H). LCMS (ESI, m/z): 480.2 [M+H]⁺.

### Example 24: (Ent 2)-7-(6-(2-(2,2-difluoro-1-(4-fluorophenyl)propyl)-2H-1,2,3-triazol-4-yl)-3-methylpyrazin-2-yl)-8-methyl-[1,2,4]triazolo[1,5-α]pyridin-2-amine

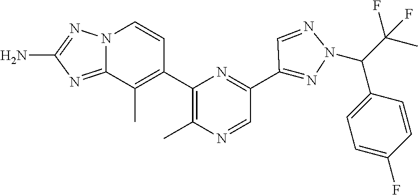 [SMILES: Cc1ncc(-c2cnn(C(c3ccc(F)cc3)C(C)(F)F)n2)nc1-c1ccn2nc(N)nc2c1C]

The title compound (Ent 2)-7-(6-(2-(2,2-difluoro-1-(4-fluorophenyl)propyl)-2H-1,2,3-triazol-4-yl)-3-methylpyrazin-2-yl)-8-methyl-[1,2,4]triazolo[1,5-α]pyridin-2-amine was obtained as the second eluting enantiomer from the preparative SFC column in a similar fashion as described for the preparation of Example 2, where 7-(6-chloro-3-methylpyrazin-2-yl)-2-(2,5-dimethyl-1H-pyrrol-1-yl)-8-methyl-[1,2,4]triazolo[1,5-α]pyridine (Intermediate 13) and 2-(2,2-difluoro-1-(4-fluorophenyl)propyl)-4-(4,4,5,5-tetramethyl-1,3,2-dioxaborolan-2-yl)-2H-1,2,3-triazole (Racemic Intermediate 25) were used in place of 2-(2,5-dimethyl-1H-pyrrol-1-yl)-8-methyl-7-(6-(4,4,5,5-tetramethyl-1,3,2-dioxaborolan-2-yl)pyrazin-2-yl)-[1,2,4]triazolo[1,5-α]pyridine and (±)-4-bromo-2-(1-(3,5-difluorophenyl)-2,2-difluoropropyl)-2H-1,2,3-triazole in the first step. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 22-52% B over 25 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(2,2-difluoro-1-(4-fluorophenyl)propyl)-2H-1,2,3-triazol-4-yl)-3-methylpyrazin-2-yl)-8-methyl-[1,2,4]triazolo[1,5-α]pyridin-2-amine (4.7 mg, 6.9%). This racemic material was then further purified via preparative SFC with the following conditions: Column: Chiralpak IC, 30 mm×250 mm, 5 m particles; Flow Rate: 75.00 mL/min; Column Temperature: 40° C. Fraction collection was triggered by UV (228 nm). Mobile Phase A: supercritical CO₂; Mobile Phase B: i-PrOH with 0.1% NH₄OH; Elution gradient: isocratic 20% B over 19 minutes. Fractions containing the second isomer eluting from the chiral column were combined and dried via centrifugal evaporation to provide (Ent 2)-7-(6-(2-(2,2-difluoro-1-(4-fluorophenyl)propyl)-2H-1,2,3-triazol-4-yl)-3-methylpyrazin-2-yl)-8-methyl-[1,2,4]triazolo[1,5-α]pyridin-2-amine (2.2 mg, 3.2%). ¹H NMR (500 MHz, DMSO-d₆) δ 9.20-9.10 (m, 1H), 8.58-8.45 (m, 2H), 7.93-7.81 (m, 2H), 7.37-7.25 (m, 2H), 6.95-6.85 (m, 1H), 6.82-6.68 (m, 1H), 6.10 (s, 2H), 2.43 (s, 3H), 2.21 (s, 3H), 1.76-1.62 (m, 3H). LCMS (ESI, m/z): 480.2 [M+H]⁺.

### Example 25: (Ent 1 and Ent 2)-7-(5-(2-(2,2-difluoro-1-(4-fluorophenyl)propyl)-2H-1,2,3-triazol-4-yl)-6-methylpyridin-3-yl)-[1,2,4]triazolo[1,5-α]pyridin-2-amine

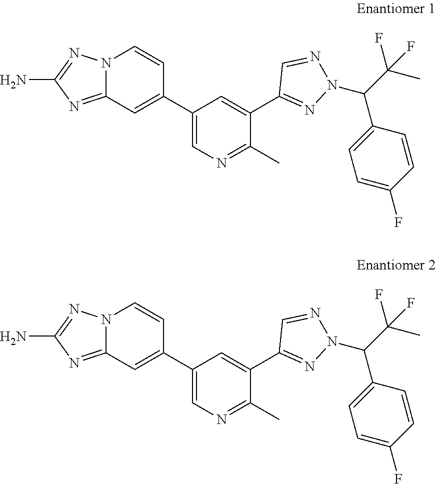 [SMILES: Cc1ncc(-c2ccn3nc(N)nc3c2)cc1-c1cnn(C(c2ccc(F)cc2)C(C)(F)F)n1 ; Cc1ncc(-c2ccn3nc(N)nc3c2)cc1-c1cnn(C(c2ccc(F)cc2)C(C)(F)F)n1]

(The two unassigned enantiomers are

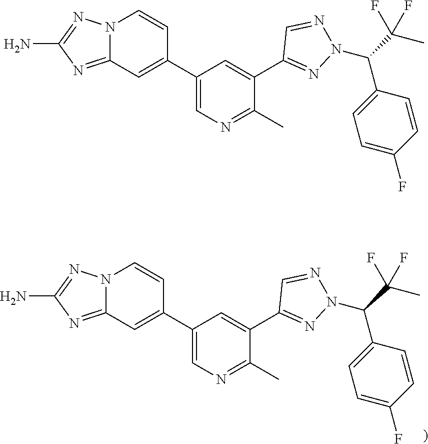 [SMILES: Cc1ncc(-c2ccn3nc(N)nc3c2)cc1-c1cnn([C@@H](c2ccc(F)cc2)C(C)(F)F)n1 ; Cc1ncc(-c2ccn3nc(N)nc3c2)cc1-c1cnn([C@H](c2ccc(F)cc2)C(C)(F)F)n1]

Step 1: To a solution of (±)-4-bromo-2-(2,2-difluoro-1-(4-fluorophenyl)propyl)-2H-1,2,3-triazole (67.78 mg, 0.212 mmol) (Intermediate 19) and bis(pinacolato)diboron (67.2 mg, 0.265 mmol) in dioxane (1694 μl) was added potassium acetate (52.0 mg, 0.529 mmol) followed by [1,1′-bis(diphenylphosphino)ferrocene]dichloropalladium(ii), complex with dichloromethane (17.29 mg, 0.021 mmol). The vessel was flushed with nitrogen, and the contents heated overnight at 85° C. LC/MS showed a large peak with m/z corresponding to the related boronic acid. Solvents were partially removed, and the residue was taken up in DCM. The organic layer was split with water, and the remaining aqueous phase was extracted with DCM. The combined organic layers were washed with brine, dried over MgSO4, filtered, and concentrated in vacuo. The residue was split into 2~equal portions and used as-is.

Step 2: To a solution of tert-butyl (tert-butoxycarbonyl)(7-(5-chloro-6-methylpyridin-3-yl)-[1,2,4]triazolo[1,5-α]pyridin-2-yl)carbamate (0.049 g, 0.106 mmol) (Intermediate 22) and (±)-2-(2,2-difluoro-1-(4-fluorophenyl)propyl)-4-(4,4,5,5-tetramethyl-1,3,2-dioxaborolan-2-yl)-2H-1,2,3-triazole (0.039 g, 0.106 mmol) in dioxane (1.325 ml) was added a 2 M solution of K3PO4, (0.159 ml, 0.318 mmol) followed by [1,1′-Bis(diphenylphosphino)ferrocene]dichloropalladium(ii), complex with dichloromethane (8.66 mg, 10.60 μmol). The vial was flushed with N₂, then sealed and heated at 70° C. for ~45 min. LC/MS showed the desired product had formed, plus some residual pyridine chloride. The reaction mixture was diluted with copious DCM, dried over MgSO₄, filtered and concentrated. The residue was azeotroped with MeOH and concentrated. The residue was used directly in the subsequent reaction.

Step 3: To a solution of the crude residue from the previous reaction in DCM (0.8 mL) was added TFA (0.408 mL, 5.30 mmol), and the resulting solution was stirred at rt. LC/MS showed the deprotection had occurred. Solvents were removed and the residue was taken up in MeOH. The crude material was purified via preparative Reverse Phase chromatography with the following conditions: Column: XBridge C18, 19 mm×200 mm, 5 μm particles; Flow Rate: 20 mL/min; Column Temperature: 25° C., 5% to 40% B in A over 25 min (solvent A=95/5 water/ACN, 0.1% TFA; solvent B=5/95 water/ACN, 0.1% TFA) Fraction collection was triggered by MS (ESI+). Fractions containing the desired product were combined and dried via centrifugal evaporation. The material was further purified via preparative Reverse Phase with the following conditions: Column: XBridge C18, 19 mm×200 mm, 5 μm particles; Flow Rate: 20 mL/min; Column Temperature: 25° C., 5% to 35% B in A over 30 min. Fraction collection was triggered by MS (ESI+). Fractions containing the desired product were combined and dried via centrifugal evaporation. The material was further purified via preparative SFC with the following conditions: Column: Chiralpak IA, 21 mm×250 mm, 5 μm particles; Flow Rate: 3.500 mL/min 70% CO₂/30% MeOH with 0.1% NH₄O H modifier for 40 min; Column Temperature: 40° C. Fraction collection was triggered by UV (220 nm). Fractions containing the first eluting product were combined and dried via centrifugal evaporation. Obtained 4.1 mg of the title compound. The second enantiomer was similarly obtained. ¹H NMR (500 MHz, DMSO-d₆) δ 8.97 (d, J=2.1 Hz, 1H), 8.66 (d, J=7.0 Hz, 1H), 8.54 (s, 1H), 8.44 (d, J=2.0 Hz, 1H), 7.92-7.83 (m, 3H), 7.39-7.26 (m, 3H), 6.72 (t, J=12.6 Hz, 1H), 6.09 (s, 2H), 2.77 (s, 3H), 1.76-1.59 (t, J=19.1 Hz, 3H). MS ESI m/z (M+H)⁺465.2.

### Example 26: (R)-7-(5-(2-(2,2-difluoro-1-(4-fluorophenyl)propyl)-2H-1,2,3-triazol-4-yl)-2-methylpyridin-3-yl)-8-methyl-[1,2,4]triazolo[1,5-α]pyridin-2-amine

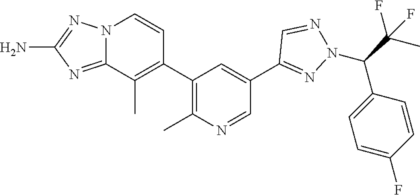 [SMILES: Cc1ncc(-c2cnn([C@H](c3ccc(F)cc3)C(C)(F)F)n2)cc1-c1ccn2nc(N)nc2c1C]

Step 1: To a solution of tert-butyl (tert-butoxycarbonyl)(8-methyl-7-(4,4,5,5-tetramethyl-1,3,2-dioxaborolan-2-yl)-[1,2,4]triazolo[1,5-α]pyridin-2-yl)carbamate (27.4 mg, 0.058 mmol) (Intermediate 24) and (R)-3-chloro-5-(2-(2,2-difluoro-1-(4-fluorophenyl)propyl)-2H-1,2,3-triazol-4-yl)-2-methylpyridine (21.20 mg, 0.058 mmol) (Intermediate 26) in dioxane (723 μl) was added a 2M solution of 97%, K₃04P, (87 μl, 0.173 mmol) followed by [1,1′-bis(diphenylphosphino)ferrocene]dichloropalladium(II), complex with dichloromethane (4.72 mg, 5.78 μmol). The vial was flushed with N₂, then sealed and heated at 75° C. overnight. Only SM2 was observed by LC/MS. Catalytic Pd(dtBuf)Cl2 and additional K3PO4 were added, and heating was resumed. LC/MS then showed robust conversion to the product. Water and EtOAc were added and the phases were split. The organic layer was extracted twice more with EtOAc. The combined organic extracts were dried over MgSO4, filtered, and concentrated. The resulting residue of tert-butyl (R)-(tert-butoxycarbonyl)(7-(5-(2-(2,2-difluoro-1-(4-fluorophenyl)propyl)-2H-1,2,3-triazol-4-yl)-2-methylpyridin-3-yl)-8-methyl-[1,2,4]triazolo[1,5-α]pyridin-2-yl)carbamate was taken forward into subsequent chemistry as-is. MS ESI m/z (M+H)⁺679.4.

Step 2: To a solution of tert-butyl (R)-(tert-butoxycarbonyl)(7-(5-(2-(2,2-difluoro-1-(4-fluorophenyl)propyl)-2H-1,2,3-triazol-4-yl)-2-methylpyridin-3-yl)-8-methyl-[1,2,4]triazolo[1,5-α]pyridin-2-yl)carbamate (39.4 mg, 0.058 mmol) in DCM (900 μL) was added TFA (313 μL, 4.06 mmol), and the solution was stirred at rt. Solvents were removed, and the residue was taken up in DMF and purified via preparative Reverse Phase chromatography with the following conditions: Gradient from 9% to 41% Solvent B in solvent A over 22 min, (solvent A=5/95 ACN/water with 0.1% TFA and solvent B=95/5 ACN/water with 0.1% TFA) Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation to yield 18 mg of the desired material. MS ESI m/z (M+H)⁺479.1. ¹H NMR (500 MHz, DMSO-d₆) δ 9.10-9.07 (m, 1H), 8.59-8.53 (m, 2H), 8.14 (d, J=2.0 Hz, 1H), 7.89-7.82 (m, 2H), 7.30 (br t, J=8.6 Hz, 2H), 6.89 (d, J=6.8 Hz, 1H), 6.68 (t, J=12.5 Hz, 1H), 2.35 (s, 3H), 2.18 (s, 3H), 1.67 (br t, J=19.0 Hz, 3H).

### Example 27: (R)-7-(5-(2-(2,2-difluoro-1-(4-fluorophenyl)propyl)-2H-1,2,3-triazol-4-yl)-2-(trifluoromethyl)pyridin-3-yl)-[1,2,4]triazolo[1,5-α]pyridin-2-amine

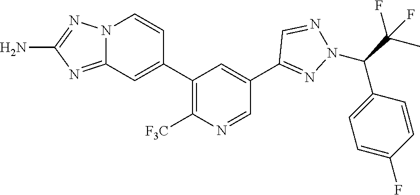 [SMILES: CC(F)(F)[C@@H](c1ccc(F)cc1)n1ncc(-c2cnc(C(F)(F)F)c(-c3ccn4nc(N)nc4c3)c2)n1]

Step 1: To a solution of (R)-2-(2,2-difluoro-1-(4-fluorophenyl)propyl)-4-(4,4,5,5-tetramethyl-1,3,2-dioxaborolan-2-yl)-2H-1,2,3-triazole (0.047 g, 0.127 mmol) (Intermediate 25) and tert-butyl (tert-butoxycarbonyl)(7-(5-chloro-2-(trifluoromethyl)pyridin-3-yl)-[1,2,4]triazolo[1,5-α]pyridin-2-yl)carbamate (0.05485 g, 0.107 mmol) (Intermediate 27) in dioxane (1.155 ml) was added sparged 2M 97%, K₃04P, (0.191 ml, 0.381 mmol), then 1,1′-bis(di-tert-butylphosphino)ferrocene palladium dichloride (6.62 mg, 10.16 μmol) was added. The reaction vessel was flushed with nitrogen, sealed, and heated to 75° C. overnight. LC/MS showed the desired product had cleanly formed. Solvents were removed and the residue was taken up in EtOAc. The residual aqueous material was extracted/rinsed with EtOAc twice, and the combined organic layers were dried over MgSO₄, filtered, and concentrated. The resulting residue was taken forward into subsequent chemistry as-is. Obtained tert-butyl (R)-(tert-butoxycarbonyl)(7-(5-(2-(2,2-difluoro-1-(4-fluorophenyl)propyl)-2H-1,2,3-triazol-4-yl)-2-(trifluoromethyl)pyridin-3-yl)-[1,2,4]triazolo[1,5-α]pyridin-2-yl)carbamate. MS ESI m/z (M+H)⁺719.5.

Step 2: To a solution of tert-butyl (R)-(tert-butoxycarbonyl)(7-(5-(2-(2,2-difluoro-1-(4-fluorophenyl)propyl)-2H-1,2,3-triazol-4-yl)-2-(trifluoromethyl)pyridin-3-yl)-[1,2,4]triazolo[1,5-α]pyridin-2-yl)carbamate (91 mg, 0.127 mmol) in DCM (2.3 mL) was added TFA (0.734 mL, 9.53 mmol), and the resulting solution was stirred at rt. Solvents were removed, and the residue was taken up in DMF, filtered, and and purified via preparative Reverse Phase chromatography with the following conditions: Gradient from 27% to 57% Solvent B in solvent A over 21 min, (solvent A=5/95 ACN/water with 10 mM ammonium acetate and solvent B=95/5 ACN/water with 10 mM ammonium acetate) Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation to yield 38.6 mg of the desired material. MS ESI m/z (M+H)⁺519.1. ¹H NMR (500 MHz, DMSO-d₆) δ 9.31 (s, 1H), 8.73 (s, 1H), 8.68 (d, J=6.9 Hz, 1H), 8.47 (s, 1H), 7.87 (app t, J=6.5 Hz, 2H), 7.48 (s, 1H), 7.31 (t, J=8.8 Hz, 2H), 6.96 (d, J=6.9 Hz, 1H), 6.75 (br t, J=12.1 Hz, 1H), 6.16 (s, 2H), 1.68 (br t, J=19.1 Hz, 3H).

### Example 28: (R)-7-(5-(2-(2,2-difluoro-1-(4-fluorophenyl)propyl)-2H-1,2,3-triazol-4-yl)-2-(trifluoromethyl)pyridin-3-yl)-8-methyl-[1,2,4]triazolo[1,5-α]pyridin-2-amine

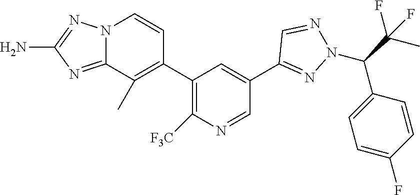 [SMILES: Cc1c(-c2cc(-c3cnn([C@H](c4ccc(F)cc4)C(C)(F)F)n3)cnc2C(F)(F)F)ccn2nc(N)nc12]

Step 1: To a solution of (R)-2-(2,2-difluoro-1-(4-fluorophenyl)propyl)-4-(4,4,5,5-tetramethyl-1,3,2-dioxaborolan-2-yl)-2H-1,2,3-triazole (0.029 g, 0.078 mmol) (Intermediate 25) and tert-butyl (tert-butoxycarbonyl)(7-(5-chloro-2-(trifluoromethyl)pyridin-3-yl)-8-methyl-[1,2,4]triazolo[1,5-α]pyridin-2-yl)carbamate (0.037 g, 0.070 mmol) (Intermediate 28) in dioxane (0.709 ml) was added sparged 2M 97%, K₃04P, (0.117 ml, 0.234 mmol), then 1,1′-bis(di-tert-butylphosphino)ferrocene palladium dichloride (4.07 mg, 6.24 μmol) was added and the reaction vessel was flushed with nitrogen. The vessel was sealed and heated to 75° C. overnight. LC/MS showed some product and a few additional peaks. Water and EtOAc were added, and the mixture was extracted 3 times into EtOAc. The combined organic extracts were dried over MgSO4, filtered, and concentrated. The residue was taken forward for subsequent chemistry as-is. MS ESI m/z (M+H)⁺733.4.

Step 2: To a solution of crude tert-butyl (R)-(tert-butoxycarbonyl)(7-(5-(2-(2,2-difluoro-1-(4-fluorophenyl)propyl)-2H-1,2,3-triazol-4-yl)-2-(trifluoromethyl)pyridin-3-yl)-8-methyl-[1,2,4]triazolo[1,5-α]pyridin-2-yl)carbamate (57.1 mg, 0.078 mmol) was added TFA (0.451 mL, 5.85 mmol), and the resulting solution was stirred at rt for 3 h. Solvents were removed, and the residue was taken up in DMF, filtered, and and purified via preparative Reverse Phase chromatography with the following conditions: Gradient from 25% to 60% Solvent B in solvent A over 25 min, (solvent A=5/95 ACN/water with 10 mM ammonium acetate and solvent B=95/5 ACN/water with 10 mM ammonium acetate) Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation to yield 11.5 mg of the desired material. MS ESI m/z (M+H)⁺533.1. ¹H NMR (500 MHz, DMSO-d₆) δ 9.33 (s, 1H), 8.71 (s, 1H), 8.50 (d, J=6.9 Hz, 1H), 8.42 (s, 1H), 7.87 (t, J=6.4 Hz, 2H), 7.30 (t, J=8.7 Hz, 2H), 6.81 (d, J=6.9 Hz, 1H), 6.74 (br t, J=12.7 Hz, 1H), 6.12 (s, 2H), 2.14 (s, 3H), 1.68 (br t, J=19.0 Hz, 3H).

### Example 29: (R)-7-(5-(2-(2,2-difluoro-1-(4-fluorophenyl)propyl)-2H-1,2,3-triazol-4-yl)-2-(methyl)pyridin-3-yl)-[1,2,4]triazolo[1,5-α]pyridin-2-amine

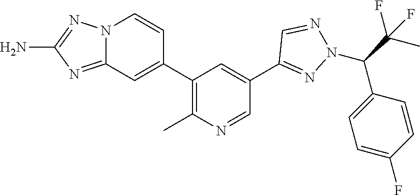 [SMILES: Cc1ncc(-c2cnn([C@H](c3ccc(F)cc3)C(C)(F)F)n2)cc1-c1ccn2nc(N)nc2c1]

Step 1: To a solution of tert-butyl (tert-butoxycarbonyl)(7-(4,4,5,5-tetramethyl-1,3,2-dioxaborolan-2-yl)-[1,2,4]triazolo[1,5-α]pyridin-2-yl)carbamate (42.4 mg, 0.092 mmol) (Intermediate 15) and (R)-3-chloro-5-(2-(2,2-difluoro-1-(4-fluorophenyl)propyl)-2H-1,2,3-triazol-4-yl)-2-methylpyridine (22.52 mg, 0.061 mmol) (Intermediate 26) in dioxane (558 μl) was added a 2M solution of 97%, K₃04P, (92 μl, 0.184 mmol) followed by 1,1′-bis(di-tert-butylphosphino)ferrocene palladium dichloride (4.00 mg, 6.14 μmol). The vial was flushed with N₂, then sealed and heated at 75° C. overnight. LC/MS shows the desired material and a fairly clean reaction. Solvents were removed and the residue was partitioned between water and EtOAc. The aqueous layer was extracted twice more with EtOAc and the combined organic fractions were dried over MgSO₄, filtered, and concentrated. The crude residue was taken forward to subsequent chemistry as-is. MS ESI m/z (M+H)⁺533.1.

Step 2: To a solution of crude tert-butyl (R)-(tert-butoxycarbonyl)(7-(5-(2-(2,2-difluoro-1-(4-fluorophenyl)propyl)-2H-1,2,3-triazol-4-yl)-2-methylpyridin-3-yl)-[1,2,4]triazolo[1,5-α]pyridin-2-yl)carbamate (40.5 mg, 0.061 mmol) was added TFA (587 μl, 7.63 mmol), and the resulting solution was stirred at rt for 60 min. Solvents were removed, and the residue was taken up in DMF, filtered, and and purified via preparative Reverse Phase chromatography with the following conditions: Gradient from 20% to 60% Solvent B in solvent A over 20 min, (solvent A=5/95 ACN/water with 10 mM ammonium acetate and solvent B=95/5 ACN/water with 10 mM ammonium acetate) Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation to yield 21.1 mg of the desired material. MS ESI m/z (M+H)⁺465.2. ¹H NMR (500 MHz, DMSO-d₆) δ 9.03 (d, J=2.0 Hz, 1H), 8.65 (d, J=6.9 Hz, 1H), 8.59 (s, 1H), 8.16 (d, J=1.9 Hz, 1H), 7.86 (br dd, J=8.4, 5.8 Hz, 2H), 7.49 (s, 1H), 7.34-7.27 (m, 2H), 7.01 (dd, J=6.9, 1.8 Hz, 1H), 6.69 (br t, J=12.7 Hz, 1H), 2.54 (s, 3H), 1.67 (br t, J=19.0 Hz, 3H).

### Example 30: (Ent 1)-7-(2-fluoro-3-(2-(1-(4-fluorophenyl)propyl)-2H-1,2,3-triazol-4-yl)phenyl)-[1,2,4]triazolo[1,5-α]pyridin-2-amine

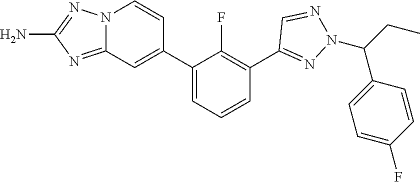 [SMILES: CCC(c1ccc(F)cc1)n1ncc(-c2cccc(-c3ccn4nc(N)nc4c3)c2F)n1]

To a solution of 4-(3-chloro-2-fluorophenyl)-2-(1-(4-fluorophenyl)propyl)-2H-1,2,3-triazole (106 mg, 0.318 mmol) (Intermediate 34) in dioxane (1.0 mL) was added a crude (2-amino-[1,2,4]triazolo[1,5-α]pyridin-7-yl)boronic acid (113 mg, 0.635 mmol) (Intermediate 30), Xphos Pd G4 (27.4 mg, 0.032 mmol) and tripotassium phosphate (0.476 mL, 0.953 mmol). The mixture was stirred at 110° C. for 1.5 h. Water was added and extracted with 3× EtOAc. The organic layer was dried over Na₂SO₄ and concentrated. The residue was purified via preparative Reverse Phase chromatography with the following conditions: Column: XBridge C18, 19 mm×200 mm, 5 m particles; Flow Rate: 20 mL/min; Column Temperature: 25° C. Fraction collection was triggered by MS (ESI+). Fractions containing the desired product were combined and dried via centrifugal evaporation. The material was further purified via preparative SFC with the following conditions: Column: Chiralcel AD-H, 30 mm×250 mm, 5 m particles; Flow Rate: 100.00 mL/min; Column Temperature: 50° C. Fraction collection was triggered by PDA (220 nm). Fractions containing the desired product were combined and dried via centrifugal evaporation. The first eluting isomer (Ent-1)-7-(2-fluoro-3-(2-(1-(4-fluorophenyl)propyl)-2H-1,2,3-triazol-4-yl)phenyl)-[1,2,4]triazolo[1,5-α]pyridin-2-amine (35.9 mg, 0.083 mmol, 26.2% yield), and the second eluting isomer (Ent-2)-7-(2-fluoro-3-(2-(1-(4-fluorophenyl)propyl)-2H-1,2,3-triazol-4-yl)phenyl)-[1,2,4]triazolo[1,5-α]pyridin-2-amine (41 mg, 0.095 mmol, 29.9% yield) were obtained.

(Ent-1): ¹H NMR (500 MHz, DMSO-d₆) δ 8.62 (d, J=6.9 Hz, 1H), 8.19 (d, J=3.7 Hz, 1H), 8.00 (br t, J=7.2 Hz, 1H), 7.68-7.62 (m, 1H), 7.56 (s, 1H), 7.51-7.46 (m, 2H), 7.44 (t, J=7.7 Hz, 1H), 7.20 (t, J=8.9 Hz, 2H), 7.11 (br d, J=7.0 Hz, 1H), 6.07 (s, 2H), 5.79 (dd, J=9.3, 6.2 Hz, 1H), 2.48-2.42 (m, 1H), 2.26-2.17 (m, 1H), 0.85 (t, J=7.3 Hz, 3H). MS ESI m/z 431.9 (M+H)⁺.

(Ent-2): ¹H NMR (500 MHz, DMSO-d₆) δ 8.64-8.59 (m, 1H), 8.19 (d, J=3.7 Hz, 1H), 8.02-7.97 (m, 1H), 7.68-7.62 (m, 1H), 7.56 (s, 1H), 7.51-7.46 (m, 2H), 7.44 (t, J=7.7 Hz, 1H), 7.19 (t, J=8.8 Hz, 2H), 7.11 (br d, J=6.9 Hz, 1H), 6.06 (s, 2H), 5.79 (dd, J=9.3, 6.2 Hz, 1H), 2.49-2.42 (m, 1H), 2.26-2.16 (m, 1H), 0.84 (t, J=7.2 Hz, 3H). MS ESI m/z 431.9 (M+H)⁺.

### Example 31: (Ent 1)-7-(6-(2-(1-(4-fluorophenyl)propyl)-2H-1,2,3-triazol-4-yl)pyridin-2-yl)-[1,2,4]triazolo[1,5-α]pyridin-2-amine

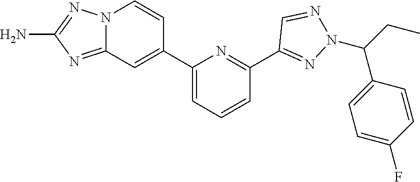 [SMILES: CCC(c1ccc(F)cc1)n1ncc(-c2cccc(-c3ccn4nc(N)nc4c3)n2)n1]

The title compound was prepared in a similar fashion to Example 30 using Intermediate and Intermediate 30 as starting materials. Chiral purification by preparative SFC was performed with the following conditions: Column: Chiralpak WHELK01, 30 mm×250 mm, 5 m particles; Flow Rate: 100.00 mL/min; Column Temperature: 50° C. 70% CO₂, 30% IPA/0.1% DEA, 28 min. Fraction collection was triggered by PDA (220 nm). Fractions containing the desired product were combined and dried via centrifugal evaporation.

(Ent-1): ¹H NMR (500 MHz, DMSO-d₆) δ 8.63 (br d, J=7.0 Hz, 1H), 8.51 (s, 1H), 8.19-8.12 (m, 2H), 8.06-8.00 (m, 1H), 7.94 (br d, J=7.7 Hz, 1H), 7.75 (dd, J=7.1, 1.9 Hz, 1H), 7.53-7.46 (m, 2H), 7.20 (br t, J=8.9 Hz, 2H), 5.82 (br dd, J=9.0, 6.3 Hz, 1H), 2.50 (br d, J=1.3 Hz, 1H), 2.22 (dt, J=13.7, 6.7 Hz, 1H), 0.86 (br t, J=7.2 Hz, 3H). ESI m/z 415.3 (M+H)⁺.

(Ent-2): ¹H NMR (500 MHz, DMSO-d₆) δ 8.63 (d, J=7.0 Hz, 1H), 8.51 (s, 1H), 8.19-8.12 (m, 2H), 8.03 (t, J=7.9 Hz, 1H), 7.94 (d, J=7.6 Hz, 1H), 7.75 (br d, J=6.9 Hz, 1H), 7.50 (dd, J=8.0, 5.7 Hz, 2H), 7.20 (t, J=8.8 Hz, 2H), 6.08 (s, 2H), 5.82 (dd, J=9.2, 6.1 Hz, 1H), 2.56-2.45 (m, 1H), 2.27-2.17 (m, 1H), 0.86 (t, J=7.2 Hz, 3H). ESI m/z 415.3 (M+H)⁺.

### Example 32: (Ent 1 and Ent 2)-7-(6-(2-(1-(4-fluorophenyl)propyl)-2H-1,2,3-triazol-4-yl)pyrazin-2-yl)-[1,2,4]triazolo[1,5-α]pyridin-2-amine

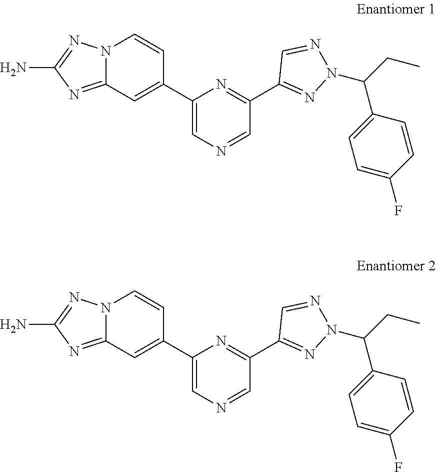 [SMILES: CCC(c1ccc(F)cc1)n1ncc(-c2cncc(-c3ccn4nc(N)nc4c3)n2)n1 ; CCC(c1ccc(F)cc1)n1ncc(-c2cncc(-c3ccn4nc(N)nc4c3)n2)n1]

(The two unassigned enantiomers are

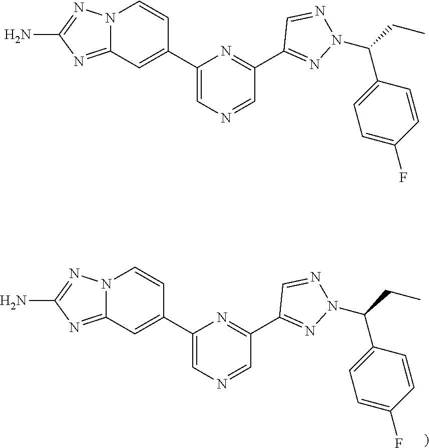 [SMILES: CC[C@H](c1ccc(F)cc1)n1ncc(-c2cncc(-c3ccn4nc(N)nc4c3)n2)n1 ; CC[C@@H](c1ccc(F)cc1)n1ncc(-c2cncc(-c3ccn4nc(N)nc4c3)n2)n1]

The title compounds were prepared in a similar fashion to Example 30 using Intermediate 36 and Intermediate 30 as starting materials. Chiral purification by preparative SFC was performed with the following conditions: Column: Chiralpak IA, 30 mm×250 mm, 5 m particles; Flow Rate:

100.00 mL/min; Column Temperature: 50° C. 70% CO₂, 30% IPA/0.1% DEA, 40 min. Fraction collection was triggered by PDA (220 nm). Fractions containing the desired product were combined and dried via centrifugal evaporation.

(Ent-1): ¹H NMR (500 MHz, DMSO-d₆) δ 9.42 (s, 1H), 9.16 (s, 1H), 8.71 (br d, J=6.8 Hz, 1H), 8.64 (s, 1H), 8.29 (s, 1H), 7.78 (br d, J=7.1 Hz, 1H), 7.56-7.51 (m, 2H), 7.22 (br t, J=8.7 Hz, 2H), 6.16 (s, 2H), 5.91-5.86 (m, 1H), 2.50-2.47 (m, 1H), 2.30-2.21 (m, 1H), 0.88 (br t, J=7.0 Hz, 3H). ESI m/z 416.1 (M+H)⁺.

(Ent-2): ¹H NMR (500 MHz, DMSO-d₆) δ 9.42 (s, 1H), 9.16 (s, 1H), 8.71 (d, J=7.0 Hz, 1H), 8.64 (s, 1H), 8.29 (s, 1H), 7.78 (dd, J=7.0, 1.7 Hz, 1H), 7.53 (dd, J=8.4, 5.6 Hz, 2H), 7.22 (t, J=8.9 Hz, 2H), 6.16 (s, 2H), 5.88 (dd, J=9.2, 6.1 Hz, 1H), 2.49-2.45 (m, 1H), 2.29-2.21 (m, 1H), 0.88 (t, J=7.2 Hz, 3H). ESI m/z 416.1 (M+H)⁺.

### Example 33: (Ent 1 and Ent 2)-7-(4-(2-(1-(4-fluorophenyl)propyl)-2H-1,2,3-triazol-4-yl)pyrimidin-2-yl)-[1,2,4]triazolo[1,5-α]pyridin-2-amine

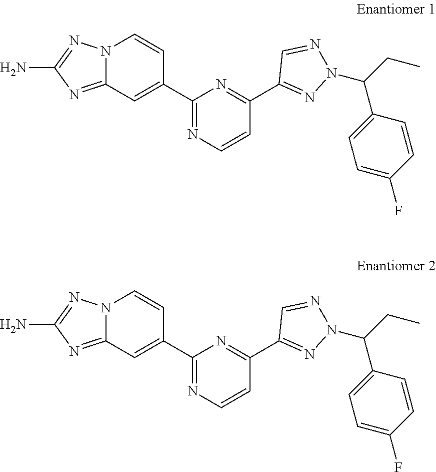 [SMILES: CCC(c1ccc(F)cc1)n1ncc(-c2ccnc(-c3ccn4nc(N)nc4c3)n2)n1 ; CCC(c1ccc(F)cc1)n1ncc(-c2ccnc(-c3ccn4nc(N)nc4c3)n2)n1]

(The two unassigned enantiomers are

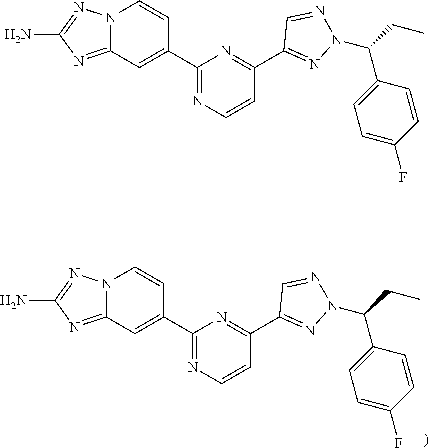 [SMILES: CC[C@H](c1ccc(F)cc1)n1ncc(-c2ccnc(-c3ccn4nc(N)nc4c3)n2)n1 ; CC[C@@H](c1ccc(F)cc1)n1ncc(-c2ccnc(-c3ccn4nc(N)nc4c3)n2)n1]

The title compounds were prepared in a similar fashion to Example 30 using Intermediate 37 and Intermediate 30 as starting materials. Chiral purification by preparative SFC was performed with the following conditions: Column: Chiralpak WHELKO1, 30 mm×250 mm, 5 m particles; Flow Rate: 100.00 mL/min; Column Temperature: 50° C. 70% CO₂, 30% IPA/0.1% DEA, 30 min. Fraction collection was triggered by PDA (220 nm). Fractions containing the desired product were combined and dried via centrifugal evaporation.

(Ent-1): ¹H NMR (500 MHz, DMSO-d₆) δ 9.03 (br d, J=5.2 Hz, 1H), 8.76 (s, 1H), 8.71-8.67 (m, 1H), 8.41 (d, J=0.9 Hz, 1H), 7.93 (br d, J=5.2 Hz, 2H), 7.55-7.49 (m, 2H), 7.22 (br t, J=8.9 Hz, 2H), 6.17 (s, 2H), 5.92-5.86 (m, 1H), 2.49-2.43 (m, 1H), 2.31-2.21 (m, 1H), 0.87 (br t, J=7.2 Hz, 3H). ESI m/z 416.0 (M+H)⁺.

(Ent-2): ¹H NMR (500 MHz, DMSO-d₆) δ 9.05-9.01 (m, 1H), 8.76 (s, 1H), 8.69 (br d, J=6.6 Hz, 1H), 8.41 (br d, J=0.9 Hz, 1H), 7.93 (br d, J=5.1 Hz, 2H), 7.56-7.49 (m, 2H), 7.25-7.19 (m, 2H), 6.20-6.14 (m, 2H), 5.89 (br dd, J=9.2, 6.6 Hz, 1H), 2.48-2.43 (m, 1H), 2.29-2.22 (m, 1H), 0.87 (br t, J=7.2 Hz, 3H). ESI m/z 416.0 (M+H)⁺.

### Example 34: (Ent 1 and Ent 2)-7-(6-(2-(2,2-difluoro-1-(4-fluorophenyl)propyl)-2H-1,2,3-triazol-4-yl)pyridin-2-yl)-[1,2,4]triazolo[1,5-α]pyridin-2-amine

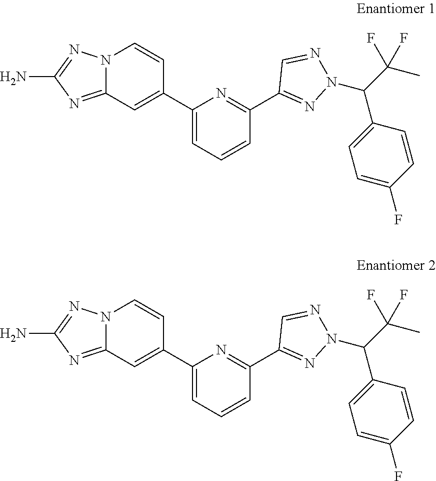 [SMILES: CC(F)(F)C(c1ccc(F)cc1)n1ncc(-c2cccc(-c3ccn4nc(N)nc4c3)n2)n1 ; CC(F)(F)C(c1ccc(F)cc1)n1ncc(-c2cccc(-c3ccn4nc(N)nc4c3)n2)n1]

(The two unassigned enantiomers are

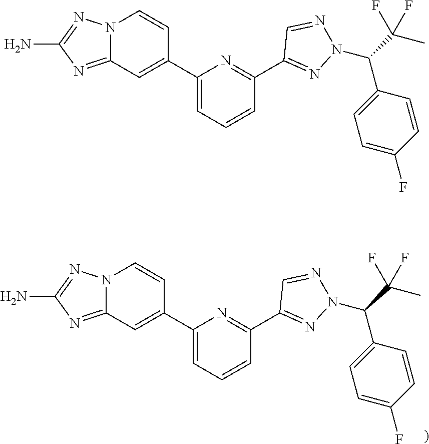 [SMILES: CC(F)(F)[C@H](c1ccc(F)cc1)n1ncc(-c2cccc(-c3ccn4nc(N)nc4c3)n2)n1 ; CC(F)(F)[C@@H](c1ccc(F)cc1)n1ncc(-c2cccc(-c3ccn4nc(N)nc4c3)n2)n1]

The title compounds were prepared in a similar fashion to Example 30 using Intermediate 38 and Intermediate 30 as starting materials. Chiral purification by preparative SFC was performed with the following conditions: Column: Chiralpak WHELKO1, 30 mm×250 mm, 5 m particles; Flow Rate: 100.00 mL/min; Column Temperature: 50° C. 75% CO₂, 25% IPA/0.1% DEA, 40 min. Fraction collection was triggered by PDA (220 nm). Fractions containing the desired product were combined and dried via centrifugal evaporation.

(Ent-1): ¹H NMR (500 MHz, DMSO-d₆) δ 8.68-8.62 (m, 2H), 8.22-8.17 (m, 2H), 8.07 (t, J=7.9 Hz, 1H), 7.99 (d, J=7.8 Hz, 1H), 7.88 (br dd, J=8.2, 5.3 Hz, 2H), 7.79-7.75 (m, 1H), 7.31 (br t, J=8.8 Hz, 2H), 6.72 (br t, J=12.5 Hz, 1H), 6.09 (s, 2H), 1.69 (br t, J=19.0 Hz, 3H). ESI m/z 451.1 (M+H)⁺.

(Ent-2): ¹H NMR (500 MHz, DMSO-d₆) δ 8.67-8.62 (m, 2H), 8.22-8.16 (m, 2H), 8.10-8.05 (m, 1H), 7.99 (d, J=7.7 Hz, 1H), 7.88 (br dd, J=8.3, 5.6 Hz, 2H), 7.77 (dd, J=7.0, 1.4 Hz, 1H), 7.31 (br t, J=8.8 Hz, 2H), 6.72 (br t, J=12.7 Hz, 1H), 6.09 (s, 2H), 1.75-1.64 (m, 3H). ESI m/z 451.1 (M+H)⁺.

### Example 35: (Ent 1 and Ent 2)-7-(6-(2-(2,2-difluoro-1-(4-fluorophenyl)propyl)-2H-1,2,3-triazol-4-yl)pyrazin-2-yl)-[1,2,4]triazolo[1,5-α]pyridin-2-amine

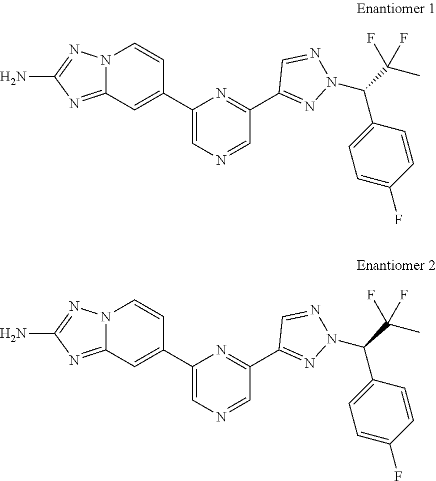 [SMILES: CC(F)(F)[C@H](c1ccc(F)cc1)n1ncc(-c2cncc(-c3ccn4nc(N)nc4c3)n2)n1 ; CC(F)(F)[C@@H](c1ccc(F)cc1)n1ncc(-c2cncc(-c3ccn4nc(N)nc4c3)n2)n1]

The title compounds were prepared in a similar fashion to Example 30 using Intermediate 39 and Intermediate 30 as starting materials. Enantiomer 2 was shown to be the (R)-enantiomer by X-ray diffraction. Chiral purification by preparative SFC was performed with the following conditions: Column: Chiralpak AD, 30 mm×250 mm, 5 m particles; Flow Rate:

100.00 mL/min; Column Temperature: 50° C. 60% CO₂, 40% IPA/0.1% DEA, 40 min. Fraction collection was triggered by PDA (220 nm). Fractions containing the desired product were combined and dried via centrifugal evaporation.

(Ent-2): ¹H NMR (500 MHz, DMSO-d₆) δ 9.46 (s, 1H), 9.20 (s, 1H), 8.77 (s, 1H), 8.72 (d, J=7.0 Hz, 1H), 8.31 (s, 1H), 7.90 (br dd, J=8.3, 5.6 Hz, 2H), 7.79 (dd, J=7.0, 1.7 Hz, 1H), 7.32 (br t, J=8.8 Hz, 2H), 6.80 (br t, J=12.6 Hz, 1H), 6.17 (s, 2H), 1.71 (br t, J=19.0 Hz, 3H). ESI m/z 452.1 (M+H)⁺.

### Example 36: (Ent 1 and Ent 2)-7-(6-(2-(3,3-difluoro-1-(4-fluorophenyl)propyl)-2H-1,2,3-triazol-4-yl)pyrazin-2-yl)-[1,2,4]triazolo[1,5-α]pyridin-2-amine

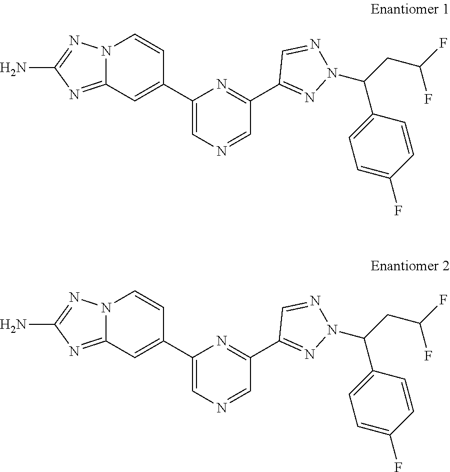 [SMILES: Nc1nc2cc(-c3cncc(-c4cnn(C(CC(F)F)c5ccc(F)cc5)n4)n3)ccn2n1 ; Nc1nc2cc(-c3cncc(-c4cnn(C(CC(F)F)c5ccc(F)cc5)n4)n3)ccn2n1]

(The two unassigned enantiomers are

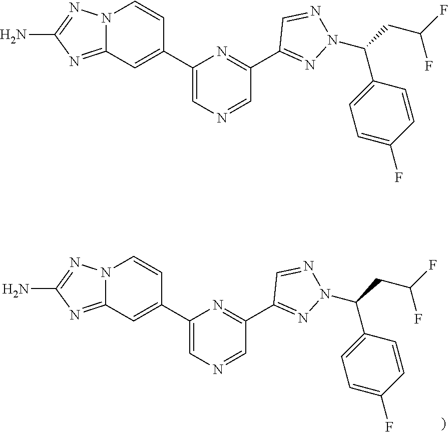 [SMILES: Nc1nc2cc(-c3cncc(-c4cnn([C@H](CC(F)F)c5ccc(F)cc5)n4)n3)ccn2n1 ; Nc1nc2cc(-c3cncc(-c4cnn([C@@H](CC(F)F)c5ccc(F)cc5)n4)n3)ccn2n1]

Step 1: To the crude reaction mixture of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate (161 mg, 0.3 mmol) (Intermediate 42) was added 4-bromo-2-(3,3-difluoro-1-(4-fluorophenyl)propyl)-2H-1,2,3-triazole (64.0 mg, 0.2 mmol) (Intermediate 40), PdCl₂(dppf) (10.98 mg, 0.015 mmol) and tripotassium phosphate (0.333 mL, 1.000 mmol). The mixture was stirred at 90° C. for 1.5 h. Water was added and extracted three times with EtOAc. The organic layer was concentrated and purified via silica gel chromatography (12 g, hexanes-100% EtOAc) to give tert-butyl (tert-butoxycarbonyl)(7-(6-(2-(3,3-difluoro-1-(4-fluorophenyl)propyl)-2H-1,2,3-triazol-4-yl)pyrazin-2-yl)-[1,2,4]triazolo[1,5-α]pyridin-2-yl)carbamate (60 mg, 0.092 mmol, 46.0% yield).

¹H NMR (400 MHz, CHLOROFORM-d) δ 9.28 (s, 1H), 9.12 (s, 1H), 8.70 (dd, J=7.2, 0.9 Hz, 1H), 8.47 (dd, J=1.9, 0.9 Hz, 1H), 8.38 (s, 1H), 7.88 (dd, J=7.2, 1.9 Hz, 1H), 7.50-7.44 (m, 2H), 7.14-7.06 (m, 2H), 6.02 (dd, J=9.5, 5.8 Hz, 1H), 5.94-5.62 (m, 1H), 3.41-3.24 (m, 1H), 2.90-2.75 (m, 1H), 1.51 (s, 18H). MS ESI m/z 652.4 (M+H)⁺.

Step 2: To a solution of tert-butyl (tert-butoxycarbonyl)(7-(6-(2-(3,3-difluoro-1-(4-fluorophenyl)propyl)-2H-1,2,3-triazol-4-yl)pyrazin-2-yl)-[1,2,4]triazolo[1,5-α]pyridin-2-yl)carbamate (60 mg, 0.092 mmol) in DCM (1 mL) was added TFA (0.5 mL, 6.49 mmol). The mixture was stirred at rt for 3 h. The mixture was concentrated. The residue was purified via preparative Reverse Phase chromatography with the following conditions: Column: XBridge C18, 19 mm×200 mm, 5 m particles; Flow Rate: 20 mL/min; Column Temperature: 25° C. Fraction collection was triggered by UV (220 nm) and MS (ESI+). Fractions containing the desired product were combined and dried via centrifugal evaporation. The material was further purified via preparative SFC with the following conditions: Column: Chiralpak WHELKO1, 30 mm×250 mm, 5 Å m particles; Flow Rate: 100.00 mL/min; Column Temperature: 50° C. Fraction collection was triggered by PDA (220 nm). Fractions containing the desired product were combined and dried via centrifugal evaporation. The first eluting isomer (Ent 1)-7-(6-(2-(3,3-difluoro-1-(4-fluorophenyl)propyl)-2H-1,2,3-triazol-4-yl)pyrazin-2-yl)-[1,2,4]triazolo[1,5-α]pyridin-2-amine (8 mg, 0.018 mmol, 19.25% yield) and the second eluting isomer (Ent 2)-7-(6-(2-(3,3-difluoro-1-(4-fluorophenyl)propyl)-2H-1,2,3-triazol-4-yl)pyrazin-2-yl)-[1,2,4]triazolo[1,5-α]pyridin-2-amine (10 mg, 0.022 mmol, 23.65% yield) were obtained.

(Ent-1): ¹H NMR (500 MHz, DMSO-d₆) δ 9.43 (s, 1H), 9.18 (s, 1H), 8.71 (d, J=6.8 Hz, 1H), 8.67 (s, 1H), 8.29 (d, J=1.0 Hz, 1H), 7.78 (dd, J=7.1, 1.8 Hz, 1H), 7.59 (dd, J=8.7, 5.4 Hz, 2H), 7.24 (t, J=8.9 Hz, 2H), 6.25-6.01 (m, 4H). MS ESI m/z 452.1 (M+H)⁺.

(Ent-2): ¹H NMR (500 MHz, DMSO-d₆) δ 9.42 (s, 1H), 9.18 (s, 1H), 8.71 (d, J=6.9 Hz, 1H), 8.67 (s, 1H), 8.29 (s, 1H), 7.78 (dd, J=7.1, 1.7 Hz, 1H), 7.59 (dd, J=8.6, 5.4 Hz, 2H), 7.24 (t, J=8.8 Hz, 2H), 6.26-6.01 (m, 4H). MS ESI m/z 452.1 (M+H)⁺.

### Example 37: (Ent 1)-7-(6-(2-(3,3-difluoro-1-(4-fluorophenyl)propyl)-2H-1,2,3-triazol-4-yl)pyridin-2-yl)-[1,2,4]triazolo[1,5-α]pyridin-2-amine

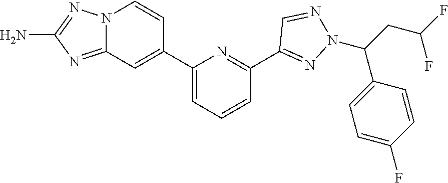 [SMILES: Nc1nc2cc(-c3cccc(-c4cnn(C(CC(F)F)c5ccc(F)cc5)n4)n3)ccn2n1]

The titled compound was prepared in a similar fashion to Example 30 using Intermediate 30 and Intermediate 43 as starting materials. Chiral purification by preparative SFC was performed with the following conditions: Column: Chiralpak WHELKO1, 30 mm×250 mm, 5 m particles; Flow Rate: 100.00 mL/min; Column Temperature: 50° C. 75% CO₂, 25% IPA/0.1% DEA, 25 min. Fraction collection was triggered by PDA (220 nm). Fractions containing the desired product were combined and dried via centrifugal evaporation.

(Ent 1): ¹H NMR (500 MHz, DMSO-d₆) δ 8.64 (br d, J=6.9 Hz, 1H), 8.55 (d, J=2.2 Hz, 1H), 8.20-8.15 (m, 2H), 8.07-8.02 (m, 1H), 7.96 (br d, J=7.6 Hz, 1H), 7.76 (br d, J=7.1 Hz, 1H), 7.56 (br t, J=5.8 Hz, 2H), 7.26-7.21 (m, 2H), 6.26-6.00 (m, 4H), 3.34-3.22 (m, 1H), 2.92-2.79 (m, 1H). MS ESI m/z 451.2 (M+H)⁺.

(Ent 2): ¹H NMR (500 MHz, DMSO-d₆) δ 8.64 (br d, J=6.4 Hz, 1H), 8.54 (s, 1H), 8.20-8.14 (m, 2H), 8.04 (br t, J=7.9 Hz, 1H), 7.98-7.94 (m, 1H), 7.79-7.74 (m, 1H), 7.56 (br dd, J=8.5, 5.3 Hz, 2H), 7.27-7.20 (m, 2H), 6.26-6.00 (m, 4H), 3.34-3.19 (m, 1H), 2.91-2.78 (m, 1H). MS ESI m/z 451.2 (M+H)⁺.

### Example 38: (Ent 1)-8-fluoro-7-(6-(2-(1-(4-fluorophenyl)propyl)-2H-1,2,3-triazol-4-yl)pyridin-2-yl)-[1,2,4]triazolo[1,5-α]pyridin-2-amine

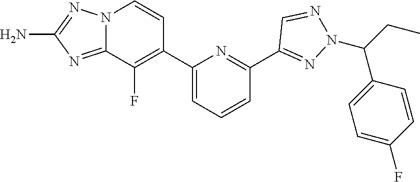 [SMILES: CCC(c1ccc(F)cc1)n1ncc(-c2cccc(-c3ccn4nc(N)nc4c3F)n2)n1]

To a solution of the crude 7-(6-chloropyridin-2-yl)-8-fluoro-[1,2,4]triazolo[1,5-α]pyridin-2-amine (80 mg, 0.303 mmol) (Intermediate 44), (2-(1-(4-fluorophenyl)propyl)-2H-1,2,3-triazol-4-yl)boronic acid (91 mg, 0.364 mmol) (Intermediate 32), and XPhos Pd G4 (13.07 mg, 0.015 mmol) in dioxane (1.5 mL) was added tripotassium phosphate (0.455 mL, 0.910 mmol). The mixture was stirred at 85° C. for 1 h. Water was added and extracted twice with EtOAc. The organic layer was dried over Na₂SO₄, then concentrated. The residue was purified via preparative Reverse Phase chromatography with the following conditions: Column: XBridge C18, 19 mm×200 mm, 5 m particles; Flow Rate: 20 mL/min; Column Temperature: 25° C. Fraction collection was triggered by MS (ESI+). Fractions containing the desired product were combined and dried via centrifugal evaporation. The material was further purified via preparative SFC with the following conditions: Column: Chromegachiral 4, 30 mm×250 mm, 5 m particles; Flow Rate: 90.5 mL/min; Column Temperature: 35° C. Fraction collection was triggered by UV (220-300) and MS (ESI+/−). Fractions containing the desired product were combined and dried via centrifugal evaporation. The first eluting isomer (Ent 1)-8-fluoro-7-(6-(2-(1-(4-fluorophenyl)propyl)-2H-1,2,3-triazol-4-yl)pyridin-2-yl)-[1,2,4]triazolo[1,5-α]pyridin-2-amine (18 mg, 0.042 mmol, 13.72% yield) and the second eluting isomer (Ent 2)-8-fluoro-7-(6-(2-(1-(4-fluorophenyl)propyl)-2H-1,2,3-triazol-4-yl)pyridin-2-yl)-[1,2,4]triazolo[1,5-α]pyridin-2-amine (26 mg, 0.060 mmol, 19.66% yield) were obtained.

(Ent 1): ¹H NMR (500 MHz, DMSO-d₆) δ 8.54 (d, J=7.0 Hz, 1H), 8.44 (s, 1H), 8.10-8.06 (m, 1H), 8.00-7.95 (m, 2H), 7.59 (t, J=6.9 Hz, 1H), 7.50 (dd, J=8.7, 5.5 Hz, 2H), 7.21 (t, J=8.9 Hz, 2H), 6.30 (s, 2H), 5.83 (dd, J=9.3, 6.1 Hz, 1H), 2.50-2.46 (m, 1H), 2.26-2.20 (m, 1H), 0.87 (t, J=7.2 Hz, 3H). MS ESI m/z 433.6 (M+H)⁺.

(Ent 2): ¹H NMR (500 MHz, DMSO-d₆) δ 8.53 (br d, J=7.0 Hz, 1H), 8.43 (s, 1H), 8.10-8.05 (m, 1H), 7.97 (br dd, J=14.5, 7.4 Hz, 2H), 7.62-7.57 (m, 1H), 7.52-7.47 (m, 2H), 7.23-7.18 (m, 2H), 6.28 (s, 2H), 5.84-5.79 (m, 1H), 2.48-2.44 (m, 1H), 2.26-2.19 (m, 1H), 0.86 (br t, J=7.0 Hz, 3H). MS ESI m/z 433.1 (M+H)⁺.

### Example 39: (Ent 1)-8-methyl-7-(6-(2-(1-(4-fluorophenyl)propyl)-2H-1,2,3-triazol-4-yl)pyridin-2-yl)-[1,2,4]triazolo[1,5-α]pyridin-2-amine

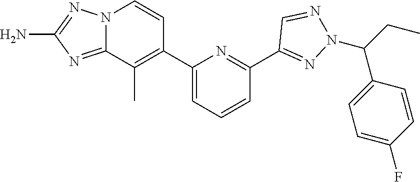 [SMILES: CCC(c1ccc(F)cc1)n1ncc(-c2cccc(-c3ccn4nc(N)nc4c3C)n2)n1]

The titled compound was prepared in a similar fashion to Example 38 using Intermediate 32 and Intermediate 45 as starting materials. Chiral purification by preparative SFC was performed with the following conditions: Column: Whelk 01 (R,R), 30 mm×250 mm, 5 m particles; Flow Rate: 90.5 mL/min; Column Temperature: 35° C. 65% CO₂/TFA, 35% EtOH/0.1% NH₄OH, 9.96 min. Fraction collection was triggered by UV (220-300 nm) and MS (ESI+/−). Fractions containing the desired product were combined and dried via centrifugal evaporation.

(Ent 1): ¹H NMR (500 MHz, DMSO-d₆) δ 8.48 (br d, J=6.7 Hz, 1H), 8.35-8.32 (m, 1H), 8.06-8.01 (m, 1H), 7.95 (br d, J=7.2 Hz, 1H), 7.64 (br d, J=7.3 Hz, 1H), 7.53-7.47 (m, 2H), 7.24-7.17 (m, 2H), 7.07-7.03 (m, 1H), 6.04 (br s, 2H), 5.84-5.78 (m, 1H), 2.51 (br s, 3H), 2.49-2.42 (m, 1H), 2.26-2.19 (m, 1H), 0.89-0.83 (m, 3H). MS ESI m/z 429.1 (M+H)⁺.

(Ent 2): ¹H NMR (500 MHz, DMSO-d₆) δ 8.28 (br d, J=6.9 Hz, 1H), 8.16-8.13 (m, 1H), 7.86-7.81 (m, 1H), 7.78-7.74 (m, 1H), 7.45 (br d, J=7.6 Hz, 1H), 7.30 (td, J=5.9, 2.4 Hz, 2H), 7.04-6.98 (m, 2H), 6.87-6.84 (m, 1H), 5.84 (br s, 2H), 5.65-5.59 (m, 1H), 2.31 (br s, 3H), 2.29-2.24 (m, 1H), 2.07-1.99 (m, 1H), 0.70-0.64 (m, 3H). MS ESI m/z 429.1 (M+H)⁺.

### Example 40: (Ent 1)-6-fluoro-7-(6-(2-(1-(4-fluorophenyl)propyl)-2H-1,2,3-triazol-4-yl)pyridin-2-yl)-[1,2,4]triazolo[1,5-α]pyridin-2-amine

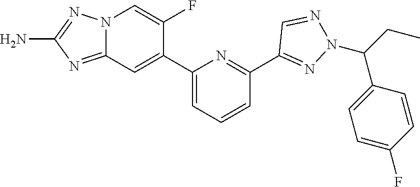 [SMILES: CCC(c1ccc(F)cc1)n1ncc(-c2cccc(-c3cc4nc(N)nn4cc3F)n2)n1]

The titled compound was prepared in a similar fashion to Example 38 using Intermediate 32 and Intermediate 46 as starting materials. Chiral purification by preparative SFC was performed with the following conditions: Column: Chiralpak IC, 30 mm×250 mm, 5 m particles; Flow Rate: 100.00 mL/min; Column Temperature: 50° C. 65% CO₂, 35% IPA/0.1% DEA, 10 min. Fraction collection was triggered by UV (220 nm). Fractions containing the desired product were combined and dried via centrifugal evaporation.

(Ent 1): ¹H NMR (500 MHz, DMSO-d₆) δ 9.05 (br d, J=5.6 Hz, 1H), 8.46 (s, 1H), 8.09-8.04 (m, 1H), 7.98 (br dd, J=13.7, 7.6 Hz, 2H), 7.86 (br d, J=7.2 Hz, 1H), 7.53-7.47 (m, 2H), 7.24-7.18 (m, 2H), 6.17 (br s, 2H), 5.86-5.80 (m, 1H), 2.50-2.44 (m, 1H), 2.28-2.18 (m, 1H), 0.90-0.84 (m, 3H). MS ESI m/z 433.1 (M+H)⁺.

(Ent 2): ¹H NMR (500 MHz, DMSO-d₆) δ 9.05 (br d, J=6.0 Hz, 1H), 8.46 (s, 1H), 8.09-8.04 (m, 1H), 8.02-7.95 (m, 2H), 7.88-7.84 (m, 1H), 7.53-7.48 (m, 2H), 7.23-7.18 (m, 2H), 6.17 (br s, 2H), 5.86-5.80 (m, 1H), 2.50-2.44 (m, 1H), 2.28-2.18 (m, 1H), 0.89-0.85 (m, 3H). MS ESI m/z 433.1 (M+H)⁺.

### Example 41: (Ent 2)-7-(4-(2-(2,2-difluoro-1-(4-fluorophenyl)propyl)-2H-1,2,3-triazol-4-yl)pyrimidin-2-yl)-[1,2,4]triazolo[1,5-α]pyridin-2-amine

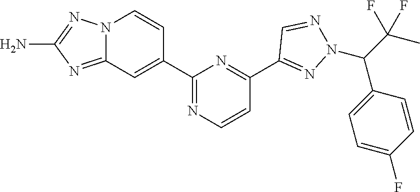 [SMILES: CC(F)(F)C(c1ccc(F)cc1)n1ncc(-c2ccnc(-c3ccn4nc(N)nc4c3)n2)n1]

The titled compound was prepared in a similar fashion to Example 30 using Intermediate 30 and Intermediate 47 as starting materials. Chiral purification by preparative SFC was performed with the following conditions: Column: Chiralpak AS-H, 30 mm×250 mm, 5 m particles; Flow Rate: 100.00 mL/min; Column Temperature: 50° C. 85% CO₂, 15% MeOH/0.1% DEA, 30 min. Fraction collection was triggered by PDA (220 nm). Fractions containing the desired product were combined and dried via centrifugal evaporation.

(Ent 1): ¹H NMR (500 MHz, DMSO-d₆) δ 9.05 (d, J=5.1 Hz, 1H), 8.84 (s, 1H), 8.68 (d, J=6.9 Hz, 1H), 8.42 (s, 1H), 7.98-7.93 (m, 2H), 7.90-7.84 (m, 2H), 7.31 (t, J=8.8 Hz, 2H), 6.77 (br t, J=12.4 Hz, 1H), 6.16 (s, 2H), 1.69 (br t, J=19.1 Hz, 3H). MS ESI m/z 452.2 (M+H)⁺.

(Ent 2): ¹H NMR (500 MHz, DMSO-d₆) δ 9.05 (d, J=5.1 Hz, 1H), 8.84 (s, 1H), 8.68 (d, J=7.0 Hz, 1H), 8.42 (s, 1H), 7.98-7.93 (m, 2H), 7.89-7.84 (m, 2H), 7.31 (t, J=8.8 Hz, 2H), 6.77 (br t, J=12.5 Hz, 1H), 6.16 (s, 2H), 1.69 (br t, J=19.1 Hz, 3H). MS ESI m/z 452.2 (M+H)⁺.

### Example 42: (Ent 2)-7-(6-(2-(2,2-difluoro-1-(4-fluorophenyl)propyl)-2H-1,2,3-triazol-4-yl)pyridin-2-yl)-8-methyl-[1,2,4]triazolo[1,5-α]pyridin-2-amine

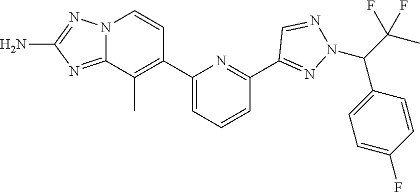 [SMILES: Cc1c(-c2cccc(-c3cnn(C(c4ccc(F)cc4)C(C)(F)F)n3)n2)ccn2nc(N)nc12]

The titled compound was prepared in a similar fashion to Example 38 using Intermediate 33 and Intermediate 45 as starting materials. Chiral purification by preparative SFC was performed with the following conditions: Column: Chiralpak AD-H, 30 mm×250 mm, 5 m particles; Flow Rate: 100.00 mL/min; Column Temperature: 50° C. 55% CO₂, 45% MeOH/0.1% DEA, 30 min. Fraction collection was triggered by PDA (220 nm). Fractions containing the desired product were combined and dried via centrifugal evaporation.

(Ent 1): ¹H NMR (500 MHz, DMSO-d₆) δ 8.47 (br d, J=6.9 Hz, 1H), 8.44 (s, 1H), 8.09-8.04 (m, 1H), 7.99 (br d, J=7.9 Hz, 1H), 7.88-7.82 (m, 2H), 7.67 (d, J=7.7 Hz, 1H), 7.30 (br t, J=8.7 Hz, 2H), 7.06 (d, J=6.9 Hz, 1H), 6.67 (br t, J=12.6 Hz, 1H), 6.01 (s, 2H), 2.50 (br s, 3H), 1.68 (br t, J=19.0 Hz, 3H). MS ESI m/z 465.3 (M+H)⁺.

(Ent 2): ¹H NMR (500 MHz, DMSO-d₆) δ 8.48 (d, J=6.8 Hz, 1H), 8.45 (s, 1H), 8.09-8.05 (m, 1H), 8.00 (d, J=7.8 Hz, 1H), 7.86 (br dd, J=8.2, 5.6 Hz, 2H), 7.68 (d, J=7.6 Hz, 1H), 7.30 (br t, J=8.8 Hz, 2H), 7.06 (d, J=6.9 Hz, 1H), 6.69 (br t, J=12.4 Hz, 1H), 6.03 (s, 2H), 2.51 (s, 3H), 1.69 (br t, J=19.0 Hz, 3H). MS ESI m/z 465.2 (M+H)⁺.

### Example 43: 7-(6-(2-(2,2-difluoro-1-(4-fluorophenyl)propyl)-2H-1,2,3-triazol-4-yl)pyridin-2-yl)-8-fluoro-[1,2,4]triazolo[1,5-α]pyridin-2-amine (Enantiomers 1 and 2)

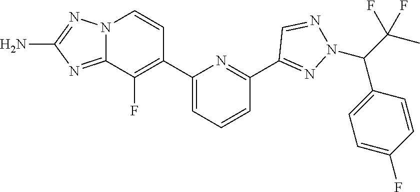 [SMILES: CC(F)(F)C(c1ccc(F)cc1)n1ncc(-c2cccc(-c3ccn4nc(N)nc4c3F)n2)n1]

The titled compound was prepared in a similar fashion to Example 38 using Intermediate 33 and Intermediate 44 as starting materials. Chiral purification by preparative SFC was performed with the following conditions: Column: Chiralpak AD-H, 30 mm×250 mm, 5 m particles; Flow Rate: 100.00 mL/min; Column Temperature: 50° C. 55% CO₂, 45% MeOH/0.1% DEA, 30 min. Fraction collection was triggered by PDA (220 nm). Fractions containing the desired product were combined and dried via centrifugal evaporation.

(Ent 1): ¹H NMR (500 MHz, DMSO-d₆) δ 8.57-8.50 (m, 2H), 8.13-8.08 (m, 1H), 8.02 (d, J=7.7 Hz, 1H), 7.98 (br d, J=7.7 Hz, 1H), 7.88-7.82 (m, 2H), 7.60 (t, J=6.9 Hz, 1H), 7.30 (br t, J=8.7 Hz, 2H), 6.68 (br t, J=12.5 Hz, 1H), 6.27 (s, 2H), 1.68 (br t, J=19.0 Hz, 3H). MS ESI m/z 469.1 (M+H)⁺.

(Ent 2): ¹H NMR (500 MHz, DMSO-d₆) δ 8.56-8.51 (m, 2H), 8.13-8.08 (m, 1H), 8.02 (d, J=7.9 Hz, 1H), 7.98 (br d, J=7.6 Hz, 1H), 7.89-7.82 (m, 2H), 7.60 (t, J=6.9 Hz, 1H), 7.30 (br t, J=8.8 Hz, 2H), 6.68 (br t, J=12.4 Hz, 1H), 6.27 (s, 2H), 1.68 (br t, J=19.0 Hz, 3H). MS ESI m/z 469.1 (M+H)⁺.

### Example 44: (Ent 1)-7-(6-(2-(2,2-difluoro-1-(4-fluorophenyl)propyl)-2H-1,2,3-triazol-4-yl)pyridin-2-yl)-6-fluoro-[1,2,4]triazolo[1,5-α]pyridin-2-amine

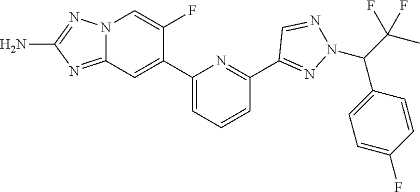 [SMILES: CC(F)(F)C(c1ccc(F)cc1)n1ncc(-c2cccc(-c3cc4nc(N)nn4cc3F)n2)n1]

The titled compound was prepared in a similar fashion to Example 38 using Intermediate 33 and Intermediate 46 as starting materials. Chiral purification by preparative SFC was performed with the following conditions: Column: Whelk 01 (R,R), 30 mm×250 mm, 5 m particles; Flow Rate: 100 mL/min; Column Temperature: 50° C. 80% CO₂, 20% IPA/0.1% DEA, 30 min. Fraction collection was triggered by UV (220-300 nm) and MS (ESI+/−). Fractions containing the desired product were combined and dried via centrifugal evaporation.

(Ent 1): ¹H NMR (500 MHz, DMSO-d₆) δ 9.04 (d, J=5.9 Hz, 1H), 8.58 (s, 1H), 8.13-8.06 (m, 1H), 8.04 (br d, J=7.7 Hz, 1H), 7.99 (d, J=7.3 Hz, 1H), 7.91-7.84 (m, 3H), 7.31 (br t, J=8.8 Hz, 2H), 6.71 (br t, J=12.6 Hz, 1H), 6.17 (s, 2H), 1.69 (br t, J=19.0 Hz, 3H). MS ESI m/z 469.1 (M+H)⁺.

(Ent 2): ¹H NMR (500 MHz, DMSO-d₆) δ 9.05 (d, J=6.0 Hz, 1H), 8.58 (s, 1H), 8.13-8.07 (m, 1H), 8.04 (d, J=7.8 Hz, 1H), 7.99 (d, J=7.2 Hz, 1H), 7.91-7.84 (m, 3H), 7.31 (br t, J=8.7 Hz, 2H), 6.72 (br t, J=12.6 Hz, 1H), 6.18 (s, 2H), 1.69 (br t, J=19.1 Hz, 3H). MS ESI m/z 469.1 (M+H)⁺.

### Example 45: (Ent 1 and Ent 2)-7-(6-(2-(2,2-difluoro-1-(4-fluorophenyl)propyl)-2H-1,2,3-triazol-4-yl)pyrazin-2-yl)-8-methyl-[1,2,4]triazolo[1,5-α]pyridin-2-amine (Enantiomers 1 and 2)

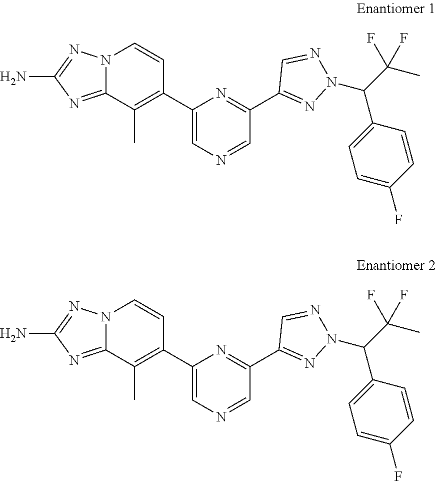 [SMILES: Cc1c(-c2cncc(-c3cnn(C(c4ccc(F)cc4)C(C)(F)F)n3)n2)ccn2nc(N)nc12 ; Cc1c(-c2cncc(-c3cnn(C(c4ccc(F)cc4)C(C)(F)F)n3)n2)ccn2nc(N)nc12]

(The two unassigned enantiomers are

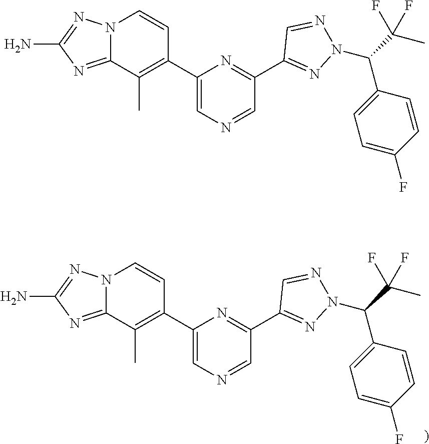 [SMILES: Cc1c(-c2cncc(-c3cnn([C@@H](c4ccc(F)cc4)C(C)(F)F)n3)n2)ccn2nc(N)nc12 ; Cc1c(-c2cncc(-c3cnn([C@H](c4ccc(F)cc4)C(C)(F)F)n3)n2)ccn2nc(N)nc12]

The titled compound was prepared in a similar fashion to Example 36 using Intermediate 24 and Intermediate 48 as starting materials. Chiral purification by preparative SFC was performed with the following conditions: Column: Chiralpak AD-H, 30 mm×250 mm, 5 m particles; Flow Rate: 100.00 mL/min; Column Temperature: 50° C. 55% CO₂, 45% IPA/0.1% DEA, 35 min. Fraction collection was triggered by PDA (220 nm). Fractions containing the desired product were combined and dried via centrifugal evaporation.

(Ent 1): ¹H NMR (500 MHz, DMSO-d₆) δ 9.22 (s, 1H), 8.96 (s, 1H), 8.61 (s, 1H), 8.55 (br d, J=6.6 Hz, 1H), 7.92-7.86 (m, 2H), 7.32 (br t, J=8.7 Hz, 2H), 7.14 (d, J=6.9 Hz, 1H), 6.78 (br t, J=12.5 Hz, 1H), 6.11 (s, 2H), 2.51 (br s, 3H), 1.70 (br t, J=19.1 Hz, 3H). MS ESI m/z 466.3 (M+H)⁺.

(Ent 2): ¹H NMR (500 MHz, DMSO-d₆) δ 9.22 (s, 1H), 8.96 (s, 1H), 8.61 (s, 1H), 8.55 (br d, J=7.0 Hz, 1H), 7.93-7.85 (m, 2H), 7.32 (br t, J=8.3 Hz, 2H), 7.14 (br d, J=6.7 Hz, 1H), 6.81-6.73 (m, 1H), 6.11 (br s, 2H), 2.51 (br s, 3H), 1.70 (br t, J=19.0 Hz, 3H) MS ESI m/z 466.3 (M+H)⁺.

### Example 46: (Ent 1)-7-(2-(2-(2,2-difluoro-1-(4-fluorophenyl)propyl)-2H-1,2,3-triazol-4-yl)pyrimidin-4-yl)-[1,2,4]triazolo[1,5-α]pyridin-2-amine

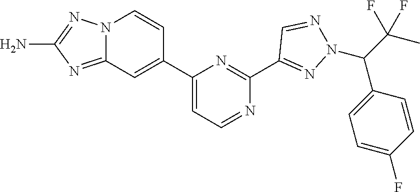 [SMILES: CC(F)(F)C(c1ccc(F)cc1)n1ncc(-c2nccc(-c3ccn4nc(N)nc4c3)n2)n1]

The titled compound was prepared in a similar fashion to Example 36 using Intermediate 33 and Intermediate 49 as starting materials. Chiral purification by preparative SFC was performed with the following conditions: Column: Whelk 01 (R,R), 30 mm×250 mm, 5 m particles; Flow Rate: 100 mL/min; Column Temperature: 50° C. 70% CO₂, 30% IPA/0.1% DEA, 30 min. Fraction collection was triggered by PDA (220 nm). Fractions containing the desired product were combined and dried via centrifugal evaporation.

(Ent 1): ¹H NMR (500 MHz, DMSO-d₆) δ 9.03 (d, J=5.3 Hz, 1H), 8.73 (d, J=6.9 Hz, 1H), 8.69 (s, 1H), 8.34 (s, 1H), 8.24 (d, J=5.4 Hz, 1H), 7.90-7.85 (m, 2H), 7.81 (dd, J=7.0, 1.8 Hz, 1H), 7.32 (t, J=8.8 Hz, 2H), 6.80 (br t, J=12.7 Hz, 1H), 6.22 (s, 2H), 1.69 (br t, J=19.0 Hz, 3H). MS ESI m/z 451.9 (M+H)⁺.

(Ent 2): ¹H NMR (500 MHz, DMSO-d₆) δ 9.03 (d, J=5.3 Hz, 1H), 8.73 (d, J=6.9 Hz, 1H), 8.69 (s, 1H), 8.34 (s, 1H), 8.23 (d, J=5.3 Hz, 1H), 7.87 (dd, J=8.2, 5.7 Hz, 2H), 7.80 (dd, J=7.1, 1.7 Hz, 1H), 7.32 (t, J=8.8 Hz, 2H), 6.79 (br t, J=12.6 Hz, 1H), 6.21 (s, 2H), 1.69 (br t, J=19.0 Hz, 3H). MS ESI m/z 451.9 (M+H)⁺.

### Example 47: (Ent 1)-7-(6-(2-(2,2-difluoro-1-(4-fluorophenyl)propyl)-2H-1,2,3-triazol-4-yl)pyrazin-2-yl)-6-fluoro-[1,2,4]triazolo[1,5-α]pyridin-2-amine

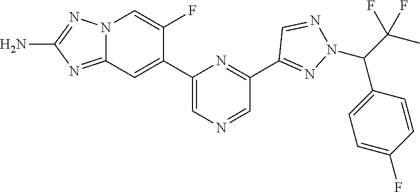 [SMILES: CC(F)(F)C(c1ccc(F)cc1)n1ncc(-c2cncc(-c3cc4nc(N)nn4cc3F)n2)n1]

The titled compound was prepared in a similar fashion to Example 38 using Intermediate 33 and Intermediate 50 as starting materials. Chiral purification by preparative SFC was performed with the following conditions: Column: Chiralpak AS-H, 30 mm×250 mm, 5 m particles; Flow Rate: 100.00 mL/min; Column Temperature: 50° C. 90% CO₂, 10% MeOH/0.1% DEA, 30 min. Fraction collection was triggered by UV (220 nm). Fractions containing the desired product were combined and dried via centrifugal evaporation.

(Ent 1): ¹H NMR (500 MHz, DMSO-d₆) δ 9.25 (s, 1H), 9.13 (d, J=5.9 Hz, 1H), 9.11 (d, J=2.1 Hz, 1H), 8.72 (s, 1H), 8.04 (d, J=7.0 Hz, 1H), 7.92-7.86 (m, 2H), 7.32 (br t, J=8.7 Hz, 2H), 6.79 (br t, J=12.5 Hz, 1H), 6.25 (s, 2H), 1.70 (br t, J=18.9 Hz, 3H). MS ESI m/z 470.1 (M+H)⁺.

(Ent 2): ¹H NMR (500 MHz, DMSO-d₆) δ 9.25 (s, 1H), 9.13 (d, J=5.9 Hz, 1H), 9.10 (d, J=2.1 Hz, 1H), 8.72 (s, 1H), 8.04 (d, J=7.1 Hz, 1H), 7.89 (br dd, J=8.4, 5.6 Hz, 2H), 7.32 (br t, J=8.8 Hz, 2H), 6.79 (br t, J=12.6 Hz, 1H), 6.24 (s, 2H), 1.70 (br t, J=19.0 Hz, 3H). MS ESI m/z 470.1 (M+H)⁺.

### Example 48: (Ent 1 and Ent 2)-7-(6-(2-(2,2-difluoro-1-(4-fluorophenyl)propyl)-2H-1,2,3-triazol-4-yl)pyrazin-2-yl)-8-fluoro-[1,2,4]triazolo[1,5-α]pyridin-2-amine

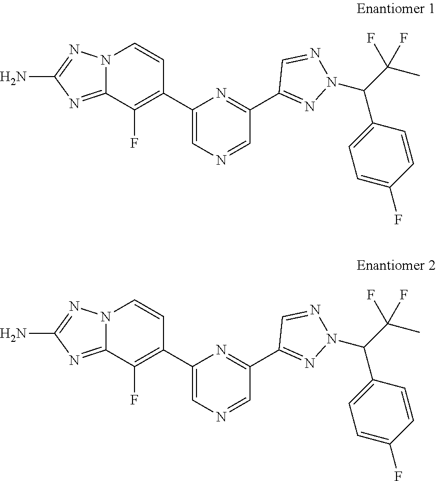 [SMILES: CC(F)(F)C(c1ccc(F)cc1)n1ncc(-c2cncc(-c3ccn4nc(N)nc4c3F)n2)n1 ; CC(F)(F)C(c1ccc(F)cc1)n1ncc(-c2cncc(-c3ccn4nc(N)nc4c3F)n2)n1]

(The two unassigned enantiomers are

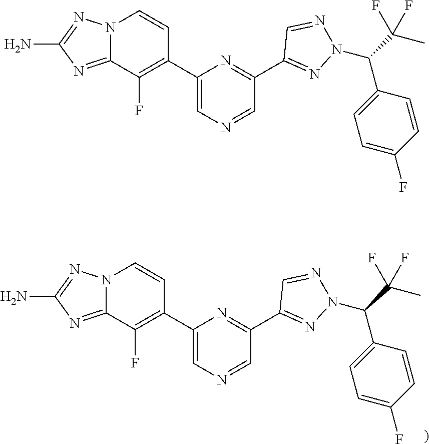 [SMILES: CC(F)(F)[C@H](c1ccc(F)cc1)n1ncc(-c2cncc(-c3ccn4nc(N)nc4c3F)n2)n1 ; CC(F)(F)[C@@H](c1ccc(F)cc1)n1ncc(-c2cncc(-c3ccn4nc(N)nc4c3F)n2)n1]

The titled compound was prepared in a similar fashion to Example 47 using Intermediate 33 and Intermediate 51 as starting materials. Chiral purification by preparative SFC was performed with the following conditions: Column: Chiralpak AD-H, 30 mm×250 mm, 5 m particles; Flow Rate: 100.00 mL/min; Column Temperature: 50° C. 65% CO₂, 35% IPA/0.1% DEA, 25 min. Fraction collection was triggered by UV (220 nm). Fractions containing the desired product were combined and dried via centrifugal evaporation.

(Ent 1): ¹H NMR (500 MHz, DMSO-d₆) δ 9.23 (s, 1H), 9.19 (s, 1H), 8.69 (s, 1H), 8.61 (d, J=6.9 Hz, 1H), 7.89 (br dd, J=8.0, 5.6 Hz, 2H), 7.57 (t, J=6.8 Hz, 1H), 7.31 (t, J=8.9 Hz, 2H), 6.78 (br t, J=12.6 Hz, 1H), 6.37 (s, 2H), 1.69 (br t, J=19.0 Hz, 3H). MS ESI m/z 470.1 (M+H)⁺.

(Ent 2): ¹H NMR (500 MHz, DMSO-d₆) δ 9.23 (s, 1H), 9.20 (s, 1H), 8.69 (s, 1H), 8.62 (d, J=6.9 Hz, 1H), 7.89 (br dd, J=8.2, 5.6 Hz, 2H), 7.57 (t, J=6.8 Hz, 1H), 7.32 (t, J=8.9 Hz, 2H), 6.79 (br t, J=12.7 Hz, 1H), 6.37 (s, 2H), 1.70 (br t, J=19.1 Hz, 3H). MS ESI m/z 470.1 (M+H)⁺.

### Example 49: (Ent 2)-7-(4-(2-(2,2-difluoro-1-(4-fluorophenyl)propyl)-2H-1,2,3-triazol-4-yl)pyrimidin-2-yl)-8-methyl-[1,2,4]triazolo[1,5-α]pyridin-2-amine

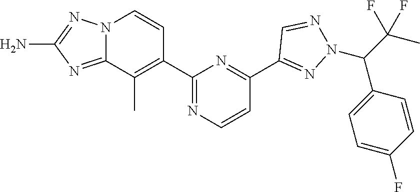 [SMILES: Cc1c(-c2nccc(-c3cnn(C(c4ccc(F)cc4)C(C)(F)F)n3)n2)ccn2nc(N)nc12]

Step 1: A mixture of 2-chloro-4-(2-(2,2-difluoro-1-(4-fluorophenyl)propyl)-2H-1,2,3-triazol-4-yl)pyrimidine (52 mg, 0.147 mmol), tert-butyl (tert-butoxycarbonyl)(8-methyl-7-(4,4,5,5-tetramethyl-1,3,2-dioxaborolan-2-yl)-[1,2,4]triazolo[1,5-α]pyridin-2-yl)carbamate (77 mg, 0.162 mmol), XPhos Pd G4 (6.33 mg, 7.35 μmol) and tripotassium phosphate (0.221 mL, 0.441 mmol) in dioxane (1.0 mL) was stirred at 60° C. for 2 h. Another 30 mg of boronic ester was added. The mixture was stirred at 70° C. for 1 h. Water was added and extracted twice with EtOAc. The organic layer was concentrated and purified via silica gel chromatography (4 g, hexanes-100% EtOAc) to give tert-butyl (tert-butoxycarbonyl)(7-(4-(2-(2,2-difluoro-1-(4-fluorophenyl)propyl)-2H-1,2,3-triazol-4-yl)pyrimidin-2-yl)-8-methyl-[1,2,4]triazolo[1,5-α]pyridin-2-yl)carbamate (90 mg, 0.135 mmol, 92% yield). MS ESI m/z 666.1 (M+H)⁺.

Step 2: The title compound was prepared in a similar fashion to Example 36 using product of step 1 as the starting material. Chiral purification by preparative SFC was performed with the following conditions: Column: Chiralpak IC, 30 mm×250 mm, 5 m particles; Flow Rate:

100.00 mL/min; Column Temperature: 50° C. 70% CO₂, 30% IPA/0.1% DEA, 25 min. Fraction collection was triggered by PDA (220 nm). Fractions containing the desired product were combined and dried via centrifugal evaporation.

(Ent 1): ¹H NMR (500 MHz, DMSO-d₆) δ 9.08 (d, J=5.1 Hz, 1H), 8.67 (s, 1H), 8.50 (br d, J=6.9 Hz, 1H), 7.97 (d, J=5.1 Hz, 1H), 7.91-7.84 (m, 2H), 7.48 (br d, J=6.9 Hz, 1H), 7.32 (br t, J=8.7 Hz, 2H), 6.78 (br t, J=12.6 Hz, 1H), 6.08 (s, 2H), 2.75 (s, 3H), 1.69 (br t, J=19.0 Hz, 3H). MS ESI m/z 466.1 (M+H)⁺.

(Ent 2): ¹H NMR (500 MHz, DMSO-d₆) δ 9.07 (d, J=5.1 Hz, 1H), 8.66 (s, 1H), 8.49 (d, J=7.1 Hz, 1H), 7.96 (d, J=5.2 Hz, 1H), 7.90-7.84 (m, 2H), 7.47 (d, J=7.1 Hz, 1H), 7.31 (br t, J=8.7 Hz, 2H), 6.76 (br t, J=12.7 Hz, 1H), 6.07 (s, 2H), 2.73 (s, 3H), 1.68 (br t, J=19.0 Hz, 3H). MS ESI m/z 466.1 (M+H)⁺.

### Example 50: (Ent 1 and Ent 2)-7-(6-(2-(1-(4-fluorophenyl)ethyl)-2H-1,2,3-triazol-4-yl)pyrazin-2-yl)-[1,2,4]triazolo[1,5-α]pyridin-2-amine

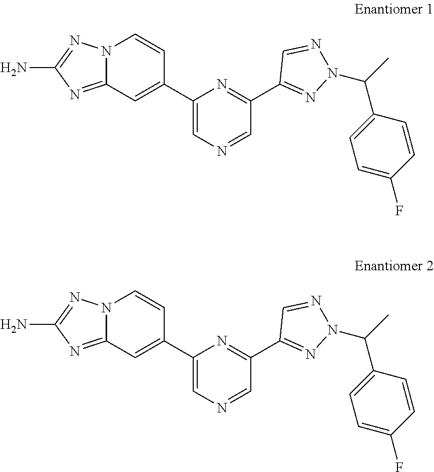 [SMILES: CC(c1ccc(F)cc1)n1ncc(-c2cncc(-c3ccn4nc(N)nc4c3)n2)n1 ; CC(c1ccc(F)cc1)n1ncc(-c2cncc(-c3ccn4nc(N)nc4c3)n2)n1]

(The two unassigned enantiomers are

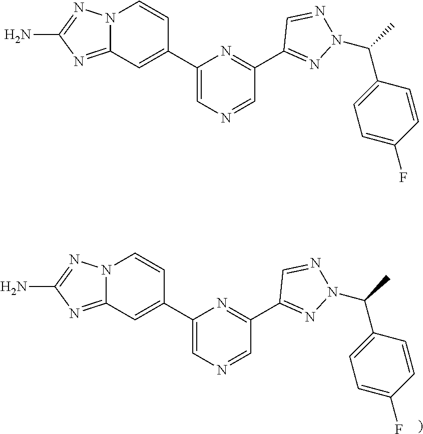 [SMILES: C[C@H](c1ccc(F)cc1)n1ncc(-c2cncc(-c3ccn4nc(N)nc4c3)n2)n1 ; C[C@@H](c1ccc(F)cc1)n1ncc(-c2cncc(-c3ccn4nc(N)nc4c3)n2)n1]

Step 1: A mixture of tert-butyl (tert-butoxycarbonyl)(7-(6-chloropyrazin-2-yl)-[1,2,4]triazolo[1,5-α]pyridin-2-yl)carbamate (105 mg, 0.235 mmol) (Intermediate 17A), (2-(1-(4-fluorophenyl)ethyl)-2H-1,2,3-triazol-4-yl)boronic acid (83 mg, 0.352 mmol) (Intermediate 54), 1,1′-Bis(di-tert-butylphosphino) ferrocene palladium chloride (4.59 mg, 7.05 μmol) and potassium phosphate (0.352 mL, 0.705 mmol) in dioxane (1.5 mL) was stirred at 65° C. for 1 h. EtOAc was added, washed with water, then concentrated. The residue was purified via silica gel chromatography (4 g, hexanes-100% EtOAc) to give tert-butyl (tert-butoxycarbonyl)(7-(6-(2-(1-(4-fluorophenyl)ethyl)-2H-1,2,3-triazol-4-yl)pyrazin-2-yl)-[1,2,4]triazolo[1,5-α]pyridin-2-yl)carbamate (140 mg, 0.233 mmol, 99% yield).

MS ESI m/z 602.1 (M+H)⁺.

Step 2: The title compound was prepared in a similar fashion to Example 36 using product of step 1 as the starting material. Chiral purification by preparative SFC was performed with the following conditions: Column: Whelk 01 (R,R), 30 mm×250 mm, 5 m particles; Flow Rate:

100 mL/min; Column Temperature: 50° C. 70% CO₂, 30% MeOH/0.1% DEA, 25 min. Fraction collection was triggered by PDA (220 nm). Fractions containing the desired product were combined and dried via centrifugal evaporation.

(Ent 1): ¹H NMR (500 MHz, DMSO-d₆) δ 9.40 (s, 1H), 9.15 (s, 1H), 8.70 (d, J=7.0 Hz, 1H), 8.62 (s, 1H), 8.28 (s, 1H), 7.77 (dd, J=7.0, 1.7 Hz, 1H), 7.44 (dd, J=8.6, 5.3 Hz, 2H), 7.21 (br t, J=8.8 Hz, 2H), 6.18-6.11 (m, 3H), 1.97 (d, J=7.0 Hz, 3H). MS ESI m/z 402.3 (M+H)⁺.

(Ent 2): ¹H NMR (500 MHz, DMSO-d₆) δ 9.41 (s, 1H), 9.15 (s, 1H), 8.70 (d, J=7.1 Hz, 1H), 8.63 (s, 1H), 8.28 (s, 1H), 7.79-7.75 (m, 1H), 7.46-7.41 (m, 2H), 7.21 (br t, J=8.9 Hz, 2H), 6.18-6.11 (m, 3H), 1.97 (br d, J=6.9 Hz, 3H). MS ESI m/z 402.1 (M+H)⁺.

### Example 51: (Ent 1)-6-(2-(2-(2,2-difluoro-1-phenylpropyl)-2H-1,2,3-triazol-4-yl)pyrimidin-2-yl)-[1,2,4]triazolo[1,5-α]pyridin-2-amine

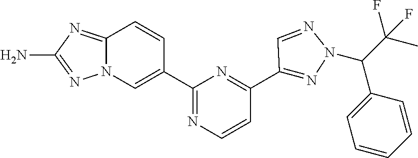 [SMILES: CC(F)(F)C(c1ccccc1)n1ncc(-c2ccnc(-c3ccc4nc(N)nn4c3)n2)n1]

Step 1: tert-butyl (tert-butoxycarbonyl)(6-(4-(2-(2,2-difluoro-1-phenylpropyl)-2H-1,2,3-triazol-4-yl)pyrimidin-2-yl)-[1,2,4]triazolo[1,5-α]pyridin-2-yl)carbamate was prepared in a similar fashion to Example 60B using Example 60A and Intermediate 58 as the starting materials. MS ESI m/z 634.0 (M+H)⁺.

Step 2: The title compound was prepared in a similar fashion to Example 36 using Example 51B. Chiral purification by preparative SFC was performed with the following conditions: Column: Chiralcel OJ-H, 30 mm×250 mm, 5 m particles; Flow Rate: 100.00 mL/min; Column Temperature: 50° C. 75% CO₂, 25% EtOH/0.1% DEA, 25 min. Fraction collection was triggered by PDA (220 nm). Fractions containing the desired product were combined and dried via centrifugal evaporation.

(Ent 1): ¹H NMR (500 MHz, DMSO-d₆) δ 9.48 (s, 1H), 8.98 (d, J=5.1 Hz, 1H), 8.88 (s, 1H), 8.47 (dd, J=9.2, 1.6 Hz, 1H), 7.88 (d, J=5.1 Hz, 1H), 7.82-7.76 (m, 2H), 7.52-7.44 (m, 4H), 6.70 (br t, J=12.6 Hz, 1H), 6.25 (s, 2H), 1.71 (br t, J=19.0 Hz, 3H). MS ESI m/z 434.2 (M+H)⁺.

(Ent 2): ¹H NMR (500 MHz, DMSO-d₆) δ 9.48 (s, 1H), 8.98 (d, J=5.1 Hz, 1H), 8.87 (s, 1H), 8.47 (dd, J=9.2, 1.6 Hz, 1H), 7.88 (d, J=5.1 Hz, 1H), 7.81-7.77 (m, 2H), 7.51-7.44 (m, 4H), 6.70 (br t, J=12.6 Hz, 1H), 6.25 (s, 2H), 1.71 (br t, J=19.0 Hz, 3H). MS ESI m/z 434.1 (M+H)⁺.

### Example 52: (Ent 2)-7-(6-(2-(2,2-difluoro-1-(4-fluorophenyl)propyl)-5-methyl-2H-1,2,3-triazol-4-yl)pyrazin-2-yl)-[1,2,4]triazolo[1,5-α]pyridin-2-amine

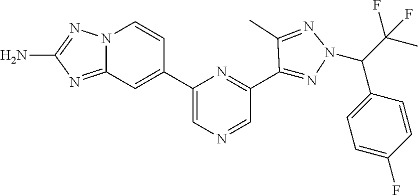 [SMILES: Cc1nn(C(c2ccc(F)cc2)C(C)(F)F)nc1-c1cncc(-c2ccn3nc(N)nc3c2)n1]

Step 1: To the reaction mixture of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate (244 mg, 0.453 mmol) (Intermediate 42) in 2 mL of dioxane was added 4-bromo-2-(2,2-difluoro-1-(4-fluorophenyl)propyl)-5-methyl-2H-1,2,3-triazole (108 mg, 0.323 mmol) (Intermediate 55), tripotassium phosphate (0.485 mL, 0.970 mmol) and 1,1′-Bis(di-tert-butylphosphino) ferrocene palladium chloride (10.53 mg, 0.016 mmol) was stirred at 85° C. for 1.5 h. Another 3×0.45 mmol of reaction mixture of tert-butyl (tert-butoxycarbonyl)(7-(6-(4,4,5,5-tetramethyl-1,3,2-dioxaborolan-2-yl)pyrazin-2-yl)-[1,2,4]triazolo[1,5-α]pyridin-2-yl)carbamate were added while the reaction was stirred 80° C. for 3 h until more of the product was formed. Water was added and extracted twice with EtOAc. The organic layer was dried over Na₂SO₄, concentrated then purified via silica gel chromatography (12 g, hexanes-100% EtOAc) to give semi-pure tert-butyl (tert-butoxycarbonyl)(7-(6-(2-(2,2-difluoro-1-(4-fluorophenyl)propyl)-5-methyl-2H-1,2,3-triazol-4-yl)pyrazin-2-yl)-[1,2,4]triazolo[1,5-α]pyridin-2-yl)carbamate (100 mg, 0.150 mmol, 46.5% yield). MS ESI m/z 666.3 (M+H)⁺.

Step 2: The title compound was prepared in a similar fashion to Example 36 using the product of step 1 as the starting material. Chiral purification by preparative SFC was performed with the following conditions: Column: Chiralcel OJ-H, 30 mm×250 mm, 5 m particles; Flow Rate:

100.00 mL/min; Column Temperature: 50° C. 85% CO₂, 15% MeOH/0.1% DEA, 25 min. Fraction collection was triggered by PDA (220 nm). Fractions containing the desired product were combined and dried via centrifugal evaporation.

(Ent 1): ¹H NMR (500 MHz, DMSO-d₆) δ 9.41 (s, 1H), 9.18 (s, 1H), 8.71 (d, J=7.2 Hz, 1H), 8.23 (s, 1H), 7.90-7.85 (m, 2H), 7.72 (br d, J=7.0 Hz, 1H), 7.31 (t, J=8.7 Hz, 2H), 6.64 (br t, J=12.8 Hz, 1H), 6.15 (s, 2H), 2.73 (s, 3H), 1.70 (br t, J=18.9 Hz, 3H). MS ESI m/z 466.2 (M+H)⁺.

(Ent 2): ¹H NMR (500 MHz, DMSO-d₆) δ 9.40 (s, 1H), 9.18 (s, 1H), 8.70 (d, J=7.0 Hz, 1H), 8.22 (s, 1H), 7.87 (dd, J=8.0, 5.5 Hz, 2H), 7.71 (d, J=7.2 Hz, 1H), 7.30 (t, J=8.9 Hz, 2H), 6.62 (t, J=12.9 Hz, 1H), 6.15 (s, 2H), 2.73 (s, 3H), 1.69 (br t, J=18.9 Hz, 3H). MS ESI m/z 466.0 (M+H)⁺.

### Example 53: (Ent 1 and Ent 2)-7-(6-(2-(2,2-difluoro-1-(4-chlorophenyl)propyl)-2H-1,2,3-triazol-4-yl)pyrazin-2-yl)-[1,2,4]triazolo[1,5-α]pyridin-2-amine

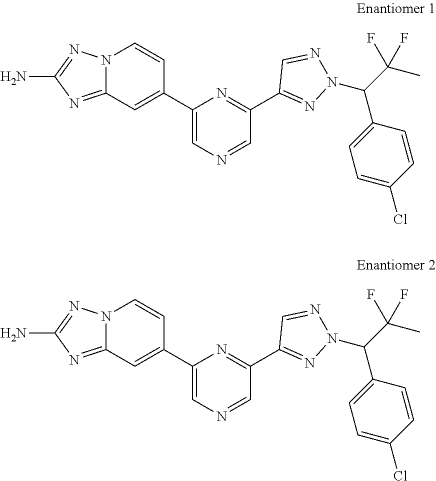 [SMILES: CC(F)(F)C(c1ccc(Cl)cc1)n1ncc(-c2cncc(-c3ccn4nc(N)nc4c3)n2)n1 ; CC(F)(F)C(c1ccc(Cl)cc1)n1ncc(-c2cncc(-c3ccn4nc(N)nc4c3)n2)n1]

(The two unassigned enantiomers are

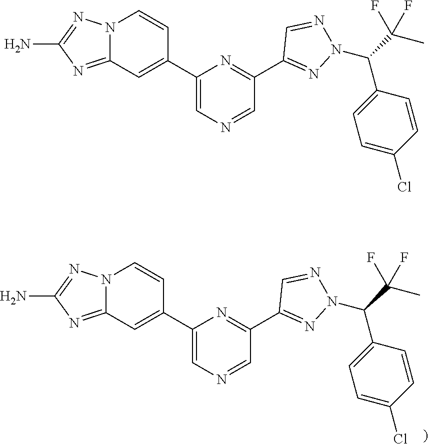 [SMILES: CC(F)(F)[C@H](c1ccc(Cl)cc1)n1ncc(-c2cncc(-c3ccn4nc(N)nc4c3)n2)n1 ; CC(F)(F)[C@@H](c1ccc(Cl)cc1)n1ncc(-c2cncc(-c3ccn4nc(N)nc4c3)n2)n1]

Step 1: To a solution of tert-butyl (tert-butoxycarbonyl)(7-(6-chloropyrazin-2-yl)-[1,2,4]triazolo[1,5-α]pyridin-2-yl)carbamate (558 mg, 1.248 mmol) (Intermediate 41), bispin (380 mg, 1.497 mmol), and PdCl₂(dppf) (43.5 mg, 0.059 mmol) in anhydrous dioxane (7 mL) was added dry potassium acetate (350 mg, 3.57 mmol). The mixture was stirred at 82° C. oil bath for 1.0 h. To the reaction mixture was added 4-bromo-2-(1-(4-chlorophenyl)-2,2-difluoropropyl)-2H-1,2,3-triazole (200 mg, 0.594 mmol) (Intermediate 56), 1,1′-Bis(di-tert-butylphosphino) ferrocene palladium chloride (19.36 mg, 0.030 mmol), and tripotassium phosphate (0.891 mL, 1.783 mmol). The mixture was stirred at 55° C. for 2 h. Water was added and extracted twice with EtOAc. The organic layers were dried over Na₂SO₄, concentrated then purified via silica gel chromatography (40 g, hexanes-100% EtOAc) to give a semi-pure tert-butyl (tert-butoxycarbonyl)(7-(6-(2-(1-(4-chlorophenyl)-2,2-difluoropropyl)-2H-1,2,3-triazol-4-yl)pyrazin-2-yl)-[1,2,4]triazolo[1,5-α]pyridin-2-yl)carbamate (360 mg, 0.539 mmol, 91% yield). MS ESI m/z 668.1 (M+H)⁺.

Step 2: The title compounds were prepared in a similar fashion to Example 36 using the product of step 1 as the starting material. Chiral purification by preparative SFC was performed with the following conditions: Column: Chiralpak AS-H, 30 mm×250 mm, 5 m particles; Flow Rate:

100.00 mL/min; Column Temperature: 50° C. 85% CO₂, 15% MeOH/0.1% DEA, 20 min. Fraction collection was triggered by PDA (220 nm). Fractions containing the desired product were combined and dried via centrifugal evaporation.

(Ent 1): ¹H NMR (500 MHz, DMSO-d₆) δ 9.45 (s, 1H), 9.20 (s, 1H), 8.77 (s, 1H), 8.71 (d, J=7.1 Hz, 1H), 8.30 (s, 1H), 7.85 (br d, J=8.3 Hz, 2H), 7.79 (dd, J=6.9, 1.7 Hz, 1H), 7.56 (br d, J=8.5 Hz, 2H), 6.80 (br t, J=12.6 Hz, 1H), 6.17 (s, 2H), 1.71 (br t, J=19.0 Hz, 3H). MS ESI m/z 468.0 (M+H)⁺.

(Ent 2): ¹H NMR (500 MHz, DMSO-d₆) δ 9.41 (s, 1H), 9.19 (s, 1H), 8.74 (s, 1H), 8.69 (d, J=6.9 Hz, 1H), 8.28 (s, 1H), 7.84 (br d, J=8.4 Hz, 2H), 7.78 (dd, J=6.9, 1.8 Hz, 1H), 7.55 (br d, J=8.5 Hz, 2H), 6.76 (br t, J=12.6 Hz, 1H), 6.15 (s, 2H), 1.70 (br t, J=19.0 Hz, 3H). MS ESI m/z 468.1 (M+H)⁺.

### Example 54: (Ent 1 and Ent 2)-7-(6-(2-(2,2-difluoro-1-phenylpropyl)-2H-1,2,3-triazol-4-yl)pyrazin-2-yl)-[1,2,4]triazolo[1,5-α]pyridin-2-amine

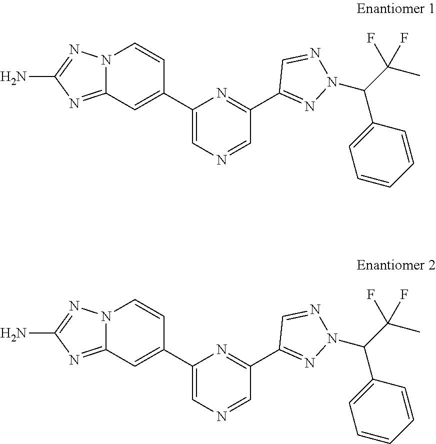 [SMILES: CC(F)(F)C(c1ccccc1)n1ncc(-c2cncc(-c3ccn4nc(N)nc4c3)n2)n1 ; CC(F)(F)C(c1ccccc1)n1ncc(-c2cncc(-c3ccn4nc(N)nc4c3)n2)n1]

(The two unassigned enantiomers are

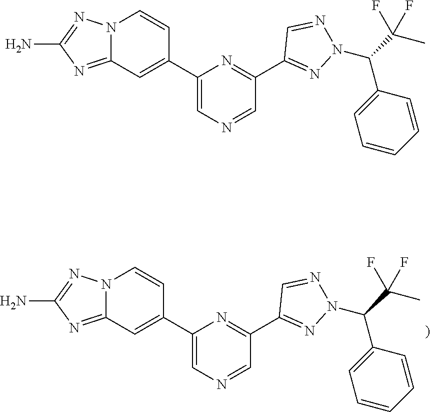 [SMILES: CC(F)(F)[C@H](c1ccccc1)n1ncc(-c2cncc(-c3ccn4nc(N)nc4c3)n2)n1 ; CC(F)(F)[C@@H](c1ccccc1)n1ncc(-c2cncc(-c3ccn4nc(N)nc4c3)n2)n1]

Step 1: To a solution of tert-butyl (tert-butoxycarbonyl)(7-(6-(2-(1-(4-chlorophenyl)-2,2-difluoropropyl)-2H-1,2,3-triazol-4-yl)pyrazin-2-yl)-[1,2,4]tri azolo[1,5-α]pyridin-2-yl)carbamate (190 mg, 0.284 mmol) (Example 53, step 1) in MeOH (3 mL) was added 1000 wt wet Pd—C(120 mg, 0.113 mmol) under N₂. A few drops of AcOH were added. The mixture was stirred under H2 balloon at rt for 5 h. Ammonium formate (359 mg, 5.69 mmol) was added. The mixture was stirred at 60° C. for 1 h, then at 70-80° C. for 2 h with additional ammonium formate (359 mg, 5.69 mmol) added. LCMS indicated most of SM was consumed. The desired de-Cl product was formed although most of bis-Boc became mono-Boc. The Pd/C was filtered off and washed with EtOAc. The filtrate was concentrated. Water was added and extracted twice with EtOAc. The organic layers were dried over Na2SO₄, then concentrated to give a crude tert-butyl (tert-butoxycarbonyl)(7-(6-(2-(2,2-difluoro-1-phenylpropyl)-2H-1,2,3-triazol-4-yl)pyrazin-2-yl)-[1,2,4]triazolo[1,5-α]pyridin-2-yl)carbamate (140 mg, 0.221 mmol, 78% yield).

Step 2: The title compounds were prepared in a similar fashion to Example 36 using the product of step 1 as the starting material. Chiral purification by preparative SFC was performed with the following conditions: Column: Chiralcel OJ-H, 30 mm×250 mm, 5 m particles; Flow Rate:

100.00 mL/min; Column Temperature: 50° C. 85% CO₂, 15% MeOH/0.1% DEA, 23 min. Fraction collection was triggered by PDA (220 nm). Fractions containing the desired product were combined and dried via centrifugal evaporation.

(Ent 1): ¹H NMR (500 MHz, DMSO-d₆) δ 9.44 (s, 1H), 9.20 (s, 1H), 8.75 (s, 1H), 8.71 (d, J=7.0 Hz, 1H), 8.30 (s, 1H), 7.83-7.77 (m, 3H), 7.50-7.43 (m, 3H), 6.71 (br t, J=12.6 Hz, 1H), 6.16 (s, 2H), 1.72 (br t, J=19.1 Hz, 3H). MS ESI m/z 434.1 (M+H)⁺.

(Ent 2): ¹H NMR (500 MHz, DMSO-d₆) δ 9.44 (s, 1H), 9.20 (s, 1H), 8.75 (s, 1H), 8.71 (d, J=7.1 Hz, 1H), 8.30 (s, 1H), 7.83-7.77 (m, 3H), 7.49-7.44 (m, 3H), 6.71 (br t, J=12.5 Hz, 1H), 6.16 (s, 2H), 1.72 (br t, J=19.0 Hz, 3H). MS ESI m/z 434.0 (M+H)⁺.

### Example 55: (Ent 1)-7-(6-(2-(2,2-difluoro-1-(4-chlorophenyl)propyl)-2H-1,2,3-triazol-4-yl)pyridin-2-yl)-[1,2,4]triazolo[1,5-α]pyridin-2-amine

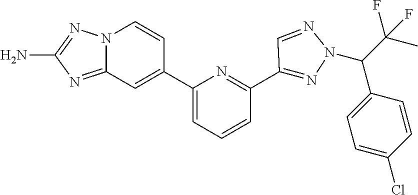 [SMILES: CC(F)(F)C(c1ccc(Cl)cc1)n1ncc(-c2cccc(-c3ccn4nc(N)nc4c3)n2)n1]

Step 1: Prepared in a similar fashion to Intermediate 38. MS ESI m/z 368.9 (M+H)⁺.

Step 2: To a solution of (2-(bis(tert-butoxycarbonyl)amino)-[1,2,4]triazolo[1,5-α]pyridin-7-yl)boronic acid (169 mg, 0.447 mmol) (Intermediate 52), semi-pure 2-chloro-6-(2-(1-(4-chlorophenyl)-2,2-difluoropropyl)-2H-1,2,3-triazol-4-yl)pyridine (150 mg, 0.406 mmol) (Example 55, step 1), and XPhos Pd G4 (17.50 mg, 0.020 mmol) in dioxane (2.0 mL) was added tripotassium phosphate (0.508 mL, 1.016 mmol). The mixture was stirred at 50° C. for 1 h. Water was added and extracted twice with EtOAc. The organic layer was concentrated, then purified via silica gel chromatography (12 g, hexanes-100% EtOAc) to give semi-pure tert-butyl (tert-butoxycarbonyl)(7-(6-(2-(1-(4-chlorophenyl)-2,2-difluoropropyl)-2H-1,2,3-triazol-4-yl)pyridin-2-yl)-[1,2,4]triazolo[1,5-α]pyridin-2-yl)carbamate. MS ESI m/z 667.2 (M+H)⁺.

Step 3: The title compounds were prepared in a similar fashion to Example 36 using the product of step 2 as the starting material. Chiral purification by preparative SFC was performed with the following conditions: Column: Chiralpak AS-H, 30 mm×250 mm, 5 m particles; Flow Rate:

100.00 mL/min; Column Temperature: 50° C. 80% CO₂, 20% MeOH/0.1% DEA, 25 min. Fraction collection was triggered by PDA (220 nm). Fractions containing the desired product were combined and dried via centrifugal evaporation.

(Ent 1): ¹H NMR (500 MHz, DMSO-d₆) δ 8.65 (t, J=3.4 Hz, 2H), 8.21-8.18 (m, 2H), 8.07 (t, J=7.7 Hz, 1H), 7.98 (d, J=7.6 Hz, 1H), 7.83 (d, J=8.5 Hz, 2H), 7.79-7.75 (m, 1H), 7.55 (d, J=8.5 Hz, 2H), 6.74 (br t, J=12.5 Hz, 1H), 6.10 (s, 2H), 1.70 (br t, J=19.2 Hz, 3H). MS ESI m/z 467.0 (M+H)⁺.

(Ent 2): ¹H NMR (500 MHz, DMSO-d₆) δ 8.64 (t, J=3.4 Hz, 2H), 8.20-8.16 (m, 2H), 8.07 (t, J=7.8 Hz, 1H), 7.98 (d, J=7.8 Hz, 1H), 7.82 (d, J=8.4 Hz, 2H), 7.77 (dd, J=7.1, 1.7 Hz, 1H), 7.55 (d, J=8.5 Hz, 2H), 6.72 (br t, J=12.7 Hz, 1H), 6.09 (s, 2H), 1.70 (br t, J=19.0 Hz, 3H).

### Example 56: (Ent 1)-7-(6-(2-(2,2-difluoro-1-phenylpropyl)-2H-1,2,3-triazol-4-yl)pyridin-2-yl)-[1,2,4]triazolo[1,5-α]pyridin-2-amine

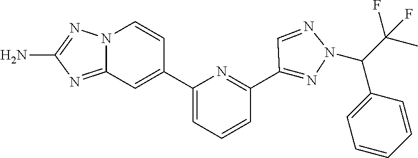 [SMILES: CC(F)(F)C(c1ccccc1)n1ncc(-c2cccc(-c3ccn4nc(N)nc4c3)n2)n1]

Step 1: Prepared in a similar fashion to Example 54, step 1 using Example 55, step 2 as starting material. MS ESI m/z 633.4 (M+H)⁺.

Step 2: The title compounds were prepared in a similar fashion to Example 36 using the product of step 1 as the starting material. Chiral purification by preparative SFC was performed with the following conditions: Column: Chiralcel OD-H, 30 mm×250 mm, 5 m particles; Flow Rate:

2 mL/min; Column Temperature: 25° C. 75% CO₂/TFA, 25% IPA/0.1% DEA, 10 min. Fraction collection was triggered by UV (220 nm) and MS (ESI+/−). Fractions containing the desired product were combined and dried via centrifugal evaporation.

(Ent 1): ¹H NMR (500 MHz, DMSO-d₆) δ 8.65-8.60 (m, 2H), 8.20-8.14 (m, 2H), 8.07 (t, J=7.8 Hz, 1H), 7.99 (d, J=7.7 Hz, 1H), 7.81-7.75 (m, 3H), 7.49-7.43 (m, 3H), 6.62 (br t, J=12.6 Hz, 1H), 6.08 (s, 2H), 1.71 (br t, J=19.1 Hz, 3H). MS ESI m/z 433.2 (M+H)⁺.

(Ent 2): ¹H NMR (500 MHz, DMSO-d₆) δ 8.64-8.60 (m, 2H), 8.19-8.13 (m, 2H), 8.07 (t, J=7.8 Hz, 1H), 7.98 (d, J=7.7 Hz, 1H), 7.77 (br dd, J=6.9, 1.9 Hz, 3H), 7.49-7.42 (m, 3H), 6.60 (br t, J=12.6 Hz, 1H), 6.07 (s, 2H), 1.70 (br t, J=19.0 Hz, 3H). MS ESI m/z 433.2 (M+H)⁺.

### Example 57: (Ent 2)-7-(4-(2-(1-(4-chlorophenyl)-2,2-difluoropropyl)-2H-1,2,3-triazol-4-yl)pyrimidin-2-yl)-[1,2,4]triazolo[1,5-α]pyridin-2-amine

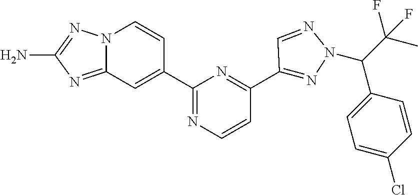 [SMILES: CC(F)(F)C(c1ccc(Cl)cc1)n1ncc(-c2ccnc(-c3ccn4nc(N)nc4c3)n2)n1]

Step 1: A mixture of 2-chloro-4-(2-(1-(4-chlorophenyl)-2,2-difluoropropyl)-2H-1,2,3-triazol-4-yl)pyrimidine (76 mg, 0.205 mmol) (Intermediate 57), (2-(bis(tert-butoxycarbonyl)amino)-[1,2,4]triazolo[1,5-α]pyridin-7-yl)boronic acid (93 mg, 0.246 mmol) (Intermediate 56), PdCl₂(dppf)-CH₂Cl₂ adduct (8.38 mg, 10.27 μmol) and tripotassium phosphate (0.257 mL, 0.513 mmol) in dioxane (1.0 mL) was stirred at 55° C. for 1 h then at 70° C. for 1 h. Another 0.05 eq. of PdCl₂(dppf)-CH₂Cl₂ adduct (8.38 mg, 10.27 μmol) was added. The mixture was stirred at 70° C. for 1.5 h. LCMS indicated the chloride was consumed. Water was added and extracted twice with EtOAc. The organic layer was concentrated and purified via silica gel chromatography (4 g, hexanes-100% EtOAc) to give semi-pure tert-butyl (tert-butoxycarbonyl)(7-(4-(2-(1-(4-chlorophenyl)-2,2-difluoropropyl)-2H-1,2,3-triazol-4-yl)pyrimidin-2-yl)-[1,2,4]triazolo[1,5-α]pyridin-2-yl)carbamate (90 mg, 0.135 mmol, 65.6% yield). MS ESI m/z 668.3 (M+H)⁺.

Step 2: The title compounds were prepared in a similar fashion to Example 36 using the product of step 1 as the starting material. Chiral purification by preparative SFC was performed with the following conditions: Column: Chiralpak IC, 30 mm×250 mm, 5 m particles; Flow Rate:

94.75 mL/min; Column Temperature: 35° C. 55% CO₂, 45% MeOH/0.1% DEA, 30.97 min. Fraction collection was triggered by UV (220-300 nm) and MS (ESI+/−). Fractions containing the desired product were combined and dried via centrifugal evaporation.

(Ent 1): ¹H NMR (500 MHz, DMSO-d₆) δ 9.07-9.04 (m, 1H), 8.86-8.84 (m, 1H), 8.68 (d, J=6.9 Hz, 1H), 8.42 (s, 1H), 7.97-7.93 (m, 2H), 7.82 (br d, J=8.5 Hz, 2H), 7.57-7.53 (m, 2H), 6.78 (br t, J=12.6 Hz, 1H), 6.16 (s, 2H), 1.69 (br t, J=19.1 Hz, 3H). MS ESI m/z 468.0 (M+H)⁺.

(Ent 2): ¹H NMR (500 MHz, DMSO-d₆) δ 9.05 (d, J=5.1 Hz, 1H), 8.84 (s, 1H), 8.67 (d, J=6.9 Hz, 1H), 8.41 (s, 1H), 7.98-7.93 (m, 2H), 7.82 (d, J=8.5 Hz, 2H), 7.54 (d, J=8.5 Hz, 2H), 6.77 (br t, J=12.4 Hz, 1H), 6.16 (s, 2H), 1.69 (br t, J=19.0 Hz, 3H). MS ESI m/z 468.0 (M+H)⁺.

### Example 58: (Ent 2)-7-(2-(2-(1-(4-chlorophenyl)-2,2-difluoropropyl)-2H-1,2,3-triazol-4-yl)pyrimidin-4-yl)-[1,2,4]triazolo[1,5-α]pyridin-2-amine

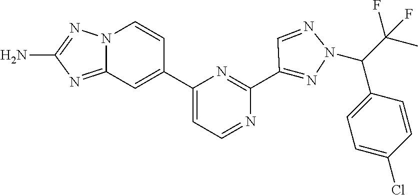 [SMILES: CC(F)(F)C(c1ccc(Cl)cc1)n1ncc(-c2nccc(-c3ccn4nc(N)nc4c3)n2)n1]

Step 1: To a solution of semi-pure 4-bromo-2-(1-(4-chlorophenyl)-2,2-difluoropropyl)-2H-1,2,3-triazole (430 mg, 1.278 mmol) (Intermediate 56) in dioxane (5 mL) was added bispin (649 mg, 2.56 mmol), potassium acetate (313 mg, 3.19 mmol) and PdCl₂(dppf)-CH₂Cl₂ adduct (104 mg, 0.128 mmol). The mixture was stirred at 95° C. for 18 h. LCMS indicated the desired B(OH)₂ peak was formed. Tert-butyl (tert-butoxycarbonyl)(7-(2-chloropyrimidin-4-yl)-[1,2,4]triazolo[1,5-α]pyridin-2-yl)carbamate (285 mg, 0.639 mmol) (Intermediate 49) was added, followed by tripotassium phosphate (1.916 mL, 3.83 mmol) and 50 mg of PdCl₂(dppf) at rt. The mixture was stirred at 70° C. for 1.5 h. Water was added, and extracted three times with EtOAc. The organic layers were concentrated and purified via silica gel chromatography (24 g, hexanes-100% EtOAc) to give tert-butyl (tert-butoxycarbonyl)(7-(2-(2-(1-(4-chlorophenyl)-2,2-difluoropropyl)-2H-1,2,3-triazol-4-yl)pyrimidin-4-yl)-[1,2,4]triazolo[1,5-α]pyridin-2-yl)carbamate (320 mg, 0.479 mmol, 37.5% yield). MS ESI m/z 668.1 (M+H)⁺.

Step 2: The title compounds were prepared in a similar fashion to Example 36 using the product of step 1 as the starting material. Chiral purification by preparative SFC was performed with the following conditions: Column: Chiralcel OD-H, 30 mm×250 mm, 5 m particles; Flow Rate:

100 mL/min; Column Temperature: 50° C. 65% CO₂, 35% IPA/0.1% DEA, 25 min. Fraction collection was triggered by UV (220 nm). Fractions containing the desired product were combined and dried via centrifugal evaporation.

(Ent 1): ¹H NMR (500 MHz, DMSO-d₆) δ 9.04 (d, J=5.3 Hz, 1H), 8.73 (d, J=6.9 Hz, 1H), 8.70 (s, 1H), 8.34 (s, 1H), 8.24 (d, J=5.3 Hz, 1H), 7.85-7.79 (m, 3H), 7.56 (d, J=8.6 Hz, 2H), 6.81 (br t, J=12.6 Hz, 1H), 1.70 (br t, J=19.0 Hz, 3H). MS ESI m/z 468.1 (M+H)⁺.

(Ent 2): ¹H NMR (500 MHz, DMSO-d₆) δ 9.04 (d, J=5.3 Hz, 1H), 8.75 (d, J=7.0 Hz, 1H), 8.70 (s, 1H), 8.35 (s, 1H), 8.24 (d, J=5.3 Hz, 1H), 7.85-7.80 (m, 3H), 7.56 (br d, J=8.5 Hz, 2H), 6.81 (br t, J=12.7 Hz, 1H), 1.70 (br t, J=19.1 Hz, 3H). MS ESI m/z 468.1 (M+H)⁺.

### Example 59: (Ent 1)-7-(2-(2-(2,2-difluoro-1-phenylpropyl)-2H-1,2,3-triazol-4-yl)pyrimidin-4-yl)-[1,2,4]triazolo[1,5-α]pyridin-2-amine

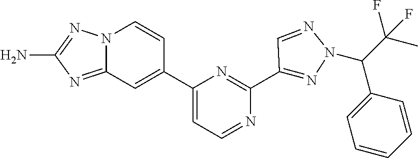 [SMILES: CC(F)(F)C(c1ccccc1)n1ncc(-c2nccc(-c3ccn4nc(N)nc4c3)n2)n1]

Step 1: To a solution of semi-pure 4-bromo-2-(1-(4-chlorophenyl)-2,2-difluoropropyl)-2H-1,2,3-triazole (570 mg, 1.694 mmol) (Intermediate 56) in dioxane (8 mL) was added bispin (1720 mg, 6.77 mmol), potassium acetate (499 mg, 5.08 mmol) and PdCl2(dppf)-CH₂Cl₂ adduct (138 mg, 0.169 mmol). The mixture was stirred at 105° C. for 6 h. Water was added and extracted four times with EtOAc. The combined organic layers were dried over Na₂SO₄ then concentrated and purified via silica gel chromatography (24 g, hexanes-100% EtOAc). The product fractions were identified via LCMS. The desired product was obtained along side a large amount of bispin which co-eluted. MS ESI m/z 301.7 (M+H)⁺.

Step 2: To a solution of contaminated 2-(1-(4-chlorophenyl)-2,2-difluoropropyl)-4-(4,4,5,5-tetramethyl-1,3,2-dioxaborolan-2-yl)-2H-1,2,3-triazole (650 mg, 1.694 mmol) (Example 59A) in MeOH (8 mL) was added 10% wt Pd—C(180 mg, 0.169 mmol) under N₂. The mixture was stirred under H2 balloon at 50° C. for 2.5 h. LCMS indicated the reaction was about halfway done. The mixture was stirred under H₂ balloon at rt for 18 h. Another 50 mg of Pd/C was added. The mixture was stirred at 65° C. under H2 balloon for 2 h. The Pd/C was filtered off. The filtrate was concentrated and azeotroped with dioxane to give a crude 2-(2,2-difluoro-1-phenylpropyl)-4-(4,4,5,5-tetramethyl-1,3,2-dioxaborolan-2-yl)-2H-1,2,3-triazole (450 mg, 1.289 mmol, 76% yield). MS ESI m/z 267.8 (M+H)⁺.

Step 3: To a solution of crude 2-(2,2-difluoro-1-phenylpropyl)-4-(4,4,5,5-tetramethyl-1,3,2-dioxaborolan-2-yl)-2H-1,2,3-triazole (103 mg, 0.295 mmol) (Example 59B) in dioxane (1.2 mL) was added tert-butyl (tert-butoxycarbonyl)(7-(2-chloropyrimidin-4-yl)-[1,2,4]triazolo[1,5-α]pyridin-2-yl)carbamate (110 mg, 0.246 mmol) (Intermediate 49), 1,1′-Bis(di-tert-butylphosphino) ferrocene palladium chloride (8.02 mg, 0.012 mmol) and tripotassium phosphate (0.369 mL, 0.738 mmol). The mixture was stirred at 75° C. for 1 h. XPhos Pd G4 (10.60 mg, 0.012 mmol) was added. The mixture was stirred 75° C. for 1 h then at 92° C. for 4 h. Water was added and extracted three times with EtOAc. The organic layer was concentrated and purified via silica gel chromatography (12 g, hexanes-100% EtOAc) to give tert-butyl (tert-butoxycarbonyl)(7-(2-(2-(2,2-difluoro-1-phenylpropyl)-2H-1,2,3-triazol-4-yl)pyrimidin-4-yl)-[1,2,4]triazolo[1,5-α]pyridin-2-yl)carbamate (115 mg, 0.181 mmol, 73.7% yield). MS ESI m/z 634.1 (M+H)⁺.

Step 4: The title compounds were prepared in a similar fashion to Example 36 using the product of step 3 as the starting material. Chiral purification by preparative SFC was performed with the following conditions: Column: Whelk 01 (R,R), 30 mm×250 mm, 5 m particles; Flow Rate:

100 mL/min; Column Temperature: 50° C. 60% CO₂, 40% IPA/0.1% DEA, 20 min. Fraction collection was triggered by PDA (220 nm). Fractions containing the desired product were combined and dried via centrifugal evaporation.

(Ent 1): ¹H NMR (500 MHz, DMSO-d₆) δ 9.02 (d, J=5.3 Hz, 1H), 8.72 (d, J=7.0 Hz, 1H), 8.68 (s, 1H), 8.32 (s, 1H), 8.21 (d, J=5.3 Hz, 1H), 7.82-7.76 (m, 3H), 7.49-7.43 (m, 3H), 6.69 (br t, J=12.8 Hz, 1H), 6.20 (s, 2H), 1.71 (br t, J=19.0 Hz, 3H). MS ESI m/z 434.0 (M+H)⁺.

(Ent 2): ¹H NMR (500 MHz, DMSO-d₆) δ 9.03 (d, J=5.3 Hz, 1H), 8.74 (d, J=7.0 Hz, 1H), 8.69 (s, 1H), 8.34 (s, 1H), 8.24 (d, J=5.5 Hz, 1H), 7.83-7.78 (m, 3H), 7.49-7.43 (m, 3H), 6.73 (br t, J=12.7 Hz, 1H), 1.71 (br t, J=19.0 Hz, 3H). MS ESI m/z 434.0 (M+H)⁺.

### Example 60: (Ent 1)-7-(2-(2-(2,2-difluoro-1-phenylpropyl)-2H-1,2,3-triazol-4-yl)pyrimidin-2-yl)-[1,2,4]triazolo[1,5-α]pyridin-2-amine

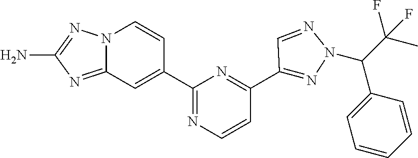 [SMILES: CC(F)(F)C(c1ccccc1)n1ncc(-c2ccnc(-c3ccn4nc(N)nc4c3)n2)n1]

Step 1: To a solution of semi-pure 2-(2,2-difluoro-1-phenylpropyl)-4-(4,4,5,5-tetramethyl-1,3,2-dioxaborolan-2-yl)-2H-1,2,3-triazole (122 mg, 0.35 mmol) (Example 59B) in dioxane (2 mL) was added tripotassium phosphate (0.525 mL, 1.050 mmol), 4-bromo-2-chloropyrimidine (122 mg, 0.630 mmol) and PdCl₂(dppf)-CH₂Cl₂ adduct (8.57 mg, 10.50 μmol) at rt. The mixture was stirred at 85° C. for 1 h. Water was added, then extracted three times with EtOAc. The organic layer was concentrated then purified via silica gel chromatography (4 g, hexanes-100% EtOAc) to give a single major peak containing both desired product and pyrimidine SM. After the material was aezotroped with dioxane, the pyrimidine was removed to give 2-chloro-4-(2-(2,2-difluoro-1-phenylpropyl)-2H-1,2,3-triazol-4-yl)pyrimidine (80 mg, 0.238 mmol, 68.1% yield). MS ESI m/z 335.8 (M+H)⁺.

Step 2: A mixture of 2-chloro-4-(2-(2,2-difluoro-1-phenylpropyl)-2H-1,2,3-triazol-4-yl)pyrimidine (80 mg, 0.238 mmol) (Example 60A), (2-(bis(tert-butoxycarbonyl)amino)-[1,2,4]triazolo[1,5-α]pyridin-7-yl)boronic acid (135 mg, 0.357 mmol) (Intermediate 52), XPhos Pd G4 (10.26 mg, 0.012 mmol) and tripotassium phosphate (0.357 mL, 0.715 mmol) in dioxane (1.5 mL) was stirred at 75° C. for 2 h. Water was added, and extracted twice with EtOAc. The organic layers were concentrated and purified via silica gel chromatography (4 g, hexanes-100% EtOAc) to give tert-butyl (tert-butoxycarbonyl)(7-(4-(2-(2,2-difluoro-1-phenylpropyl)-2H-1,2,3-triazol-4-yl)pyrimidin-2-yl)-[1,2,4]triazolo[1,5-α]pyridin-2-yl)carbamate (120 mg, 0.189 mmol, 79% yield). MS ESI m/z 634.2 (M+H)⁺.

Step 3: The title compounds were prepared in a similar fashion to Example 36 using the product of step 2 as the starting material. Chiral purification by preparative SFC was performed with the following conditions: Column: Chiralcel OD-H, 30 mm×250 mm, 5 m particles; Flow Rate:

100 mL/min; Column Temperature: 50° C. 60% CO₂, 40% IPA/0.1% DEA, 12 min. Fraction collection was triggered by PDA (220 nm). Fractions containing the desired product were combined and dried via centrifugal evaporation.

(Ent 1): ¹H NMR (500 MHz, DMSO-d₆) δ 9.05 (d, J=5.1 Hz, 1H), 8.83 (s, 1H), 8.67 (d, J=6.9 Hz, 1H), 8.42 (s, 1H), 7.98-7.93 (m, 2H), 7.79 (br d, J=6.1 Hz, 2H), 7.49-7.43 (m, 3H), 6.69 (br t, J=12.6 Hz, 1H), 6.16 (s, 2H), 1.71 (br t, J=19.1 Hz, 3H). MS ESI m/z 434.1 (M+H)⁺.

(Ent 2): ¹H NMR (500 MHz, DMSO-d₆) δ 9.05 (d, J=5.1 Hz, 1H), 8.84 (s, 1H), 8.68 (d, J=7.0 Hz, 1H), 8.42 (d, J=0.9 Hz, 1H), 7.98-7.93 (m, 2H), 7.81-7.76 (m, 2H), 7.49-7.44 (m, 3H), 6.69 (br t, J=12.6 Hz, 1H), 6.16 (s, 2H), 1.71 (br t, J=19.1 Hz, 3H). MS ESI m/z 434.2 (M+H)⁺.

### Example 61: (Ent 1)-7-(6-(2-(1-(4-fluorophenyl)ethyl)-2H-1,2,3-triazol-4-yl)pyridin-2-yl)-[1,2,4]triazolo[1,5-α]pyridin-2-amine

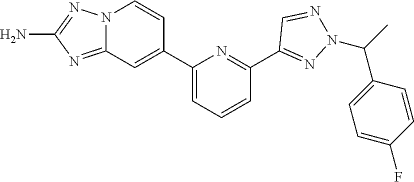 [SMILES: CC(c1ccc(F)cc1)n1ncc(-c2cccc(-c3ccn4nc(N)nc4c3)n2)n1]

Step 1: To a solution of 2-chloro-6-iodopyridine (2.117 g, 8.84 mmol) in dioxane (30 mL) was added Intermediate 15 (3.7 g, 8.04 mmol), dichloro[1,1′-bis(di-t-butylphosphino)ferrocene]palladium(II) (0.419 g, 0.643 mmol) and tripotassium phosphate (12.06 mL, 24.11 mmol). The mixture was stirred at 70° C. for 1 h. Water was added and extracted twice with EtOAc. The combined organic layers were concentrated. The residue was purified via silica gel chromatography (120 g, hexanes-50% EtOAc) to give tert-butyl (tert-butoxycarbonyl)(7-(6-chloropyridin-2-yl)-[1,2,4]triazolo[1,5-α]pyridin-2-yl)carbamate (2.2 g, 4.93 mmol, 61.4% yield). ¹H NMR (500 MHz, CHLOROFORM-d) δ 8.63 (dd, J=7.2, 0.8 Hz, 1H), 8.28 (dd, J=1.9, 0.8 Hz, 1H), 7.87 (dd, J=7.2, 1.9 Hz, 1H), 7.86-7.79 (m, 2H), 7.42 (dd, J=7.6, 1.0 Hz, 1H), 1.50 (s, 18H). MS ESI m/z 446.0 (M+H)⁺.

Step 2: To a solution of (2-(1-(4-fluorophenyl)ethyl)-2H-1,2,3-triazol-4-yl)boronic acid (78 mg, 0.332 mmol) (Intermediate 54) in dioxane (1.2 mL) was added tert-butyl (tert-butoxycarbonyl)(7-(6-chloropyridin-2-yl)-[1,2,4]triazolo[1,5-α]pyridin-2-yl)carbamate (178 mg, 0.398 mmol) (Example 61A), XPhos Pd G4 (14.30 mg, 0.017 mmol) and tripotassium phosphate (0.498 mL, 0.996 mmol). The mixture was stirred at rt for 4 h. Water was added and extracted three times with EtOAc. The combined organic layers were concentrated and purified via silica gel chromatography (4 g, hexanes-100% EtOAc) to give tert-butyl (tert-butoxycarbonyl)(7-(6-(2-(1-(4-fluorophenyl)ethyl)-2H-1,2,3-triazol-4-yl)pyridin-2-yl)-[1,2,4]triazolo[1,5-α]pyridin-2-yl)carbamate (150 mg, 0.250 mmol, 75% yield). MS ESI m/z 601.4 (M+H)⁺.

Step 3: The title compounds were prepared in a similar fashion to Example 36 using the product of step 2 as the starting material. Chiral purification by preparative SFC was performed with the following conditions: Column: Whelk 01 (R,R), 30 mm×250 mm, 5 m particles; Flow Rate:

100 mL/min; Column Temperature: 50° C. 75% CO₂, 25% MeOH/0.1% DEA, 20 min. Fraction collection was triggered by PDA (220 nm). Fractions containing the desired product were combined and dried via centrifugal evaporation.

(Ent 1): ¹H NMR (500 MHz, DMSO-d₆) δ 8.63 (d, J=7.0 Hz, 1H), 8.50 (s, 1H), 8.17 (s, 1H), 8.14 (d, J=7.9 Hz, 1H), 8.03 (t, J=7.8 Hz, 1H), 7.93 (d, J=7.7 Hz, 1H), 7.75 (dd, J=7.0, 1.4 Hz, 1H), 7.40 (dd, J=8.4, 5.6 Hz, 2H), 7.19 (t, J=8.8 Hz, 2H), 6.11-6.05 (m, 3H), 1.94 (d, J=7.0 Hz, 3H). MS ESI m/z 401.2 (M+H)⁺.

(Ent 2): ¹H NMR (500 MHz, DMSO-d₆) δ 8.63 (d, J=7.0 Hz, 1H), 8.50 (s, 1H), 8.17 (s, 1H), 8.14 (br d, J=7.9 Hz, 1H), 8.03 (t, J=7.8 Hz, 1H), 7.93 (d, J=7.7 Hz, 1H), 7.75 (br d, J=7.0 Hz, 1H), 7.44-7.37 (m, 2H), 7.19 (t, J=8.8 Hz, 2H), 6.13-6.04 (m, 3H), 1.95 (d, J=6.9 Hz, 3H). MS ESI m/z 401.4 (M+H)⁺.

### Example 62: (Ent 1)-7-(4-(2-(1-(4-fluorophenyl)ethyl)-2H-1,2,3-triazol-4-yl)pyrimidin-2-yl)-[1,2,4]triazolo[1,5-α]pyridin-2-amine

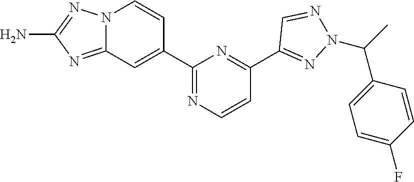 [SMILES: CC(c1ccc(F)cc1)n1ncc(-c2ccnc(-c3ccn4nc(N)nc4c3)n2)n1]

Step 1: To a solution of (2-(1-(4-fluorophenyl)ethyl)-2H-1,2,3-triazol-4-yl)boronic acid (129 mg, 0.55 mmol) (Intermediate 54) in dioxane (2 mL) was added tripotassium phosphate (0.825 mL, 1.650 mmol), 4-bromo-2-chloropyrimidine (160 mg, 0.825 mmol) and PdCl₂(dppf)-CH₂Cl2 adduct (13.47 mg, 0.017 mmol) at rt. The mixture was stirred at 75° C. for 3 h. Water was added, and was washed three times with EtOAc. The combined organic layers were concentrated then purified via silica gel chromatography (4 g, hexanes-100% EtOAc) to give semi-pure 2-chloro-4-(2-(1-(4-fluorophenyl)ethyl)-2H-1,2,3-triazol-4-yl)pyrimidine (100 mg, 0.329 mmol, 59.9% yield). MS ESI m/z 303.8 (M+H)⁺.

Step 2: The title compound was prepared in a similar fashion to Example 60B using Example 62A as the starting material. MS ESI m/z 602.2 (M+H)⁺.

Step 3: The title compounds were prepared in a similar fashion to Example 36 using the product of step 2 as the starting material. Chiral purification by preparative SFC was performed with the following conditions: Column: Whelk 01 (R,R), 30 mm×250 mm, 5 m particles; Flow Rate:

100 mL/min; Column Temperature: 50° C. 75% CO₂, 25% MeOH/0.1% DEA, 17 min. Fraction collection was triggered by PDA (220 nm). Fractions containing the desired product were combined and dried via centrifugal evaporation.

(Ent 1): ¹H NMR (500 MHz, DMSO-d₆) δ 9.01 (d, J=5.2 Hz, 1H), 8.72 (s, 1H), 8.67 (d, J=6.9 Hz, 1H), 8.40 (s, 1H), 7.93 (br d, J=7.0 Hz, 1H), 7.91 (d, J=5.2 Hz, 1H), 7.42 (dd, J=8.3, 5.6 Hz, 2H), 7.20 (t, J=8.8 Hz, 2H), 6.18-6.10 (m, 3H), 1.96 (d, J=7.0 Hz, 3H). MS ESI m/z 402.2 (M+H)⁺.

(Ent 2): ¹H NMR (500 MHz, DMSO-d₆) δ 9.01 (d, J=5.2 Hz, 1H), 8.72 (s, 1H), 8.67 (d, J=7.0 Hz, 1H), 8.40 (s, 1H), 7.93 (dd, J=7.0, 1.0 Hz, 1H), 7.91 (d, J=5.2 Hz, 1H), 7.42 (dd, J=8.3, 5.6 Hz, 2H), 7.20 (t, J=8.8 Hz, 2H), 6.18-6.10 (m, 3H), 1.96 (d, J=7.0 Hz, 3H). MS ESI m/z 402.3 (M+H)⁺.

### Example 63: (Ent 2)-7-(4-(2-(1-(4-fluorophenyl)ethyl)-2H-1,2,3-triazol-4-yl)pyrimidin-4-yl)-[1,2,4]triazolo[1,5-α]pyridin-2-amine

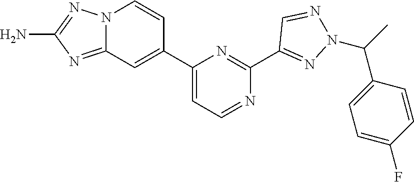 [SMILES: CC(c1ccc(F)cc1)n1ncc(-c2nccc(-c3ccn4nc(N)nc4c3)n2)n1]

Step 1: Tert-butyl (tert-butoxycarbonyl)(7-(2-(2-(1-(4-fluorophenyl)ethyl)-2H-1,2,3-triazol-4-yl)pyrimidin-4-yl)-[1,2,4]triazolo[1,5-α]pyridin-2-yl)carbamate was prepared in a similar fashion to Example 59C using Intermediate 49 and Intermediate 54 as the starting material. MS ESI m/z 602.2 (M+H)⁺.

Step 2: The title compounds were prepared in a similar fashion to Example 36 using the product of step 1 as the starting material. Chiral purification by preparative SFC was performed with the following conditions: Column: Chiralcel OJ-H, 30 mm×250 mm, 5 m particles; Flow Rate:

100.00 mL/min; Column Temperature: 50° C. 65% CO₂, 35% MeOH/0.1% DEA, 16 min. Fraction collection was triggered by PDA (220 nm). Fractions containing the desired product were combined and dried via centrifugal evaporation.

(Ent 1): ¹H NMR (500 MHz, DMSO-d₆) δ 9.00 (d, J=5.3 Hz, 1H), 8.72 (d, J=6.9 Hz, 1H), 8.57 (s, 1H), 8.32 (d, J=1.0 Hz, 1H), 8.20 (d, J=5.3 Hz, 1H), 7.80 (dd, J=7.0, 1.8 Hz, 1H), 7.43 (dd, J=8.6, 5.5 Hz, 2H), 7.21 (t, J=8.9 Hz, 2H), 6.20 (s, 2H), 6.13 (q, J=6.9 Hz, 1H), 1.96 (d, J=7.0 Hz, 3H). MS ESI m/z 402.1 (M+H)⁺.

(Ent 2): ¹H NMR (500 MHz, DMSO-d₆) δ 9.00 (d, J=5.3 Hz, 1H), 8.71 (d, J=7.0 Hz, 1H), 8.56 (s, 1H), 8.32 (d, J=1.0 Hz, 1H), 8.20 (d, J=5.3 Hz, 1H), 7.80 (dd, J=7.1, 1.9 Hz, 1H), 7.43 (dd, J=8.7, 5.5 Hz, 2H), 7.21 (t, J=8.9 Hz, 2H), 6.20 (s, 2H), 6.13 (q, J=6.6 Hz, 1H), 1.96 (d, J=7.0 Hz, 3H). MS ESI m/z 401.9 (M+H)⁺.

### Example 64: (R)-6-(6-(2-(2,2-difluoro-1-(4-fluorophenyl)propyl)-2H-1,2,3-triazol-4-yl)pyridin-2-yl)-[1,2,4]triazolo[1,5-α]pyridin-2-amine

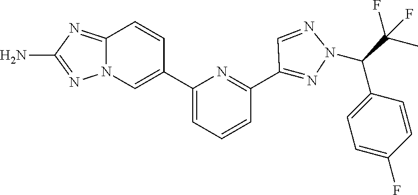 [SMILES: CC(F)(F)[C@@H](c1ccc(F)cc1)n1ncc(-c2cccc(-c3ccc4nc(N)nn4c3)n2)n1]

Step 1: (R)-2-chloro-6-(2-(2,2-difluoro-1-(4-fluorophenyl)propyl)-2H-1,2,3-triazol-4-yl)pyridine was prepared in a similar fashion to Intermediate 38. MS ESI m/z 353.0 (M+H)⁺.

Step 2: (R)-tert-butyl (tert-butoxycarbonyl)(6-(6-(2-(2,2-difluoro-1-(4-fluorophenyl)propyl)-2H-1,2,3-triazol-4-yl)pyridin-2-yl)-[1,2,4]triazolo[1,5-α]pyridin-2-yl)carbamate was prepared in a similar fashion to Example 55 step 2 using Intermediate 58 and Example 64A. MS ESI m/z 651.3 (M+H)⁺.

Step 3: To a solution of (R)-tert-butyl (tert-butoxycarbonyl)(6-(6-(2-(2,2-difluoro-1-(4-fluorophenyl)propyl)-2H-1,2,3-triazol-4-yl)pyridin-2-yl)-[1,2,4]triazolo[1,5-α]pyridin-2-yl)carbamate (Example 64B) (160 mg, 0.246 mmol) in DCM (1.5 mL) was added TFA (1 mL, 12.98 mmol). The mixture was stirred at rt for 5 h, then concentrated. The residue was purified via preparative Reverse Phase chromatography with the following conditions: Column: XBridge C18, 19 mm×200 mm, 5 μm particles; Flow Rate: 20 mL/min; Column Temperature: 25° C. Fraction collection was triggered by MS (ESI+). Fractions containing the desired product were combined and dried via centrifugal evaporation. 6-(6-(2-(2,2-difluoro-1-(4-fluorophenyl)propyl)-2H-1,2,3-triazol-4-yl)pyridin-2-yl)-[1,2,4]triazolo[1,5-α]pyridin-2-amine (32 mg, 0.071 mmol, 28.9% yield) was obtained.

¹H NMR (500 MHz, DMSO-d₆) δ 9.35 (s, 1H), 8.65 (s, 1H), 8.30 (dd, J=9.2, 1.4 Hz, 1H), 8.07-7.99 (m, 2H), 7.91 (d, J=7.6 Hz, 1H), 7.86 (br dd, J=8.0, 5.8 Hz, 2H), 7.48 (d, J=9.2 Hz, 1H), 7.30 (t, J=8.7 Hz, 2H), 6.67 (br t, J=12.5 Hz, 1H), 6.14 (s, 2H), 1.68 (br t, J=19.0 Hz, 3H). MS ESI m/z 451.1 (M+H)⁺.

### Example 65: (R)-6-(6-(2-(2,2-difluoro-1-(4-fluorophenyl)propyl)-2H-1,2,3-triazol-4-yl)pyrazin-2-yl)-[1,2,4]triazolo[1,5-α]pyridin-2-amine

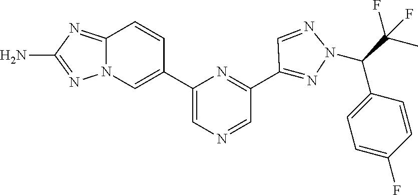 [SMILES: CC(F)(F)[C@@H](c1ccc(F)cc1)n1ncc(-c2cncc(-c3ccc4nc(N)nn4c3)n2)n1]

Step 1: Tert-butyl (tert-butoxycarbonyl)(6-(6-chloropyrazin-2-yl)-[1,2,4]triazolo[1,5-α]pyridin-2-yl)carbamate was prepared in a similar fashion to Intermediate 42 using Intermediate 58.

MS ESI m/z 447.0 (M+H)⁺.

Step 2: A mixture of tert-butyl (tert-butoxycarbonyl)(6-(6-chloropyrazin-2-yl)-[1,2,4]triazolo[1,5-α]pyridin-2-yl)carbamate (134 mg, 0.3 mmol) (Example 65A), bispin (99 mg, 0.390 mmol), tricyclohexylphosphine (5.89 mg, 0.021 mmol), and potassium acetate (88 mg, 0.900 mmol) in dioxane (1.5 mL) was stirred in a 96° C. oil bath for 1 h. Another 1 eq. of bispin was added. After stirring at 100° C. for 1 h, to the mixture was added (R)-4-bromo-2-(2,2-difluoro-1-(4-fluorophenyl)propyl)-2H-1,2,3-triazole (96 mg, 0.300 mmol) (Intermediate 20), 1,1′-Bis(di-tert-butylphosphino)ferrocene palladium dichloride (9.78 mg, 0.015 mmol), and tripotassium phosphate (0.450 mL, 0.900 mmol). The mixture was stirred at 70° C. for 2 h. Water was added and extracted twice with EtOAc. The organic layer was concentrated and purified via silica gel chromatography (12 g, hexanes-100% EtOAc) to give tert-butyl (tert-butoxycarbonyl)(6-(6-(2-(2,2-difluoro-1-(4-fluorophenyl)propyl)-2H-1,2,3-triazol-4-yl)pyrazin-2-yl)-[1,2,4]triazolo[1,5-α]pyridin-2-yl)carbamate (105 mg, 0.161 mmol, 53.7% yield). MS ESI m/z 652.2 (M+H)⁺.

Step 3: The title compound was prepared in a similar fashion to Example 64 using Example 65B. MS ESI m/z 452.0 (M+H)⁺.

### Example 66: (Ent 2)-6-(2-(2-(2,2-difluoro-1-phenylpropyl)-2H-1,2,3-triazol-4-yl)pyrimidin-4-yl)-[1,2,4]triazolo[1,5-α]pyridin-2-amine

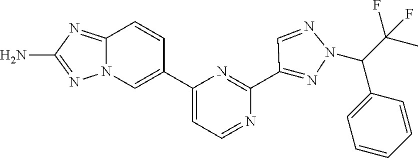 [SMILES: CC(F)(F)C(c1ccccc1)n1ncc(-c2nccc(-c3ccc4nc(N)nn4c3)n2)n1]

Step 1: tert-butyl (tert-butoxycarbonyl)(6-(2-chloropyrimidin-4-yl)-[1,2,4]triazolo[1,5-α]pyridin-2-yl)carbamate was prepared in a similar fashion to Intermediate 49 using Intermediate 58. MS ESI m/z 447.0 (M+H)⁺.

Step 2: To a solution of tert-butyl (tert-butoxycarbonyl)(6-(2-chloropyrimidin-4-yl)-[1,2,4]triazolo[1,5-α]pyridin-2-yl)carbamate (92 mg, 0.206 mmol) (Intermediate 66A), 2-(2,2-difluoro-1-phenylpropyl)-4-(4,4,5,5-tetramethyl-1,3,2-dioxaborolan-2-yl)-2H-1,2,3-triazole (60 mg, 0.172 mmol) (Example 59B) and 1,1′-Bis(di-tert-butylphosphino)ferrocene palladium dichloride (5.60 mg, 8.59 μmol) in dioxane (1.5 mL) was added tripotassium phosphate (0.215 mL, 0.430 mmol). The mixture was stirred at 40° C. for 18 h. XPhos Pd G4 (7.40 mg, 8.59 μmol) was added. The mixture was stirred at 90° C. for 1 h. Water was added and extracted three times with EtOAc. The combined organic layers were dried over Na₂SO₄ then concentrated to give a crude tert-butyl (tert-butoxycarbonyl)(6-(2-(2-(2,2-difluoro-1-phenylpropyl)-2H-1,2,3-triazol-4-yl)pyrimidin-4-yl)-[1,2,4]triazolo[1,5-α]pyridin-2-yl)carbamate (109 mg, 0.172 mmol, 100% yield). MS ESI m/z 634.3 (M+H)⁺.

Step 3: The title compound was prepared in a similar fashion to Example 36 using Example 66B. Chiral purification by preparative SFC was performed with the following conditions: Column: Chiralcel OJ-H, 30 mm×250 mm, 5 m particles; Flow Rate: 100.00 mL/min; Column Temperature: 50° C. 80% CO₂, 20% MeOH/0.1% DEA, 25 min. Fraction collection was triggered by PDA (220 nm). Fractions containing the desired product were combined and dried via centrifugal evaporation.

(Ent 1): ¹H NMR (500 MHz, DMSO-d₆) δ 9.52 (s, 1H), 8.94 (d, J=5.4 Hz, 1H), 8.70 (s, 1H), 8.38-8.34 (m, 1H), 8.12 (d, J=5.4 Hz, 1H), 7.78 (br d, J=6.3 Hz, 2H), 7.53 (d, J=9.2 Hz, 1H), 7.49-7.43 (m, 3H), 6.69 (br t, J=12.7 Hz, 1H), 6.29 (s, 2H), 1.71 (br t, J=19.0 Hz, 3H). MS ESI m/z 434.2 (M+H)⁺.

(Ent 2): ¹H NMR (500 MHz, DMSO-d₆) δ 9.51 (s, 1H), 8.94 (d, J=5.4 Hz, 1H), 8.70 (s, 1H), 8.35 (dd, J=9.3, 1.6 Hz, 1H), 8.10 (d, J=5.4 Hz, 1H), 7.78 (br d, J=6.8 Hz, 2H), 7.52 (d, J=9.3 Hz, 1H), 7.49-7.43 (m, 3H), 6.67 (br t, J=12.5 Hz, 1H), 6.28 (s, 2H), 1.71 (br t, J=19.1 Hz, 3H). MS ESI m/z 434.2 (M+H)⁺.

### Example 67: (R)-7-(5-(2-(2,2-difluoro-1-(4-fluorophenyl)propyl)-2H-1,2,3-triazol-4-yl)-6-methylpyridin-3-yl)-8-methyl-[1,2,4]triazolo[1,5-α]pyridin-2-amine

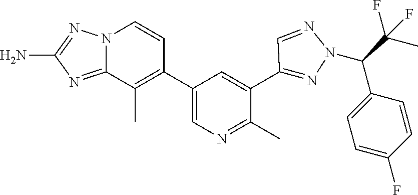 [SMILES: Cc1ncc(-c2ccn3nc(N)nc3c2C)cc1-c1cnn([C@H](c2ccc(F)cc2)C(C)(F)F)n1]

The title compound was prepared in a similar manner as Example 27 utilizing (R)-2-(2,2-difluoro-1-(4-fluorophenyl)propyl)-4-(4,4,5,5-tetramethyl-1,3,2-dioxaborolan-2-yl)-2H-1,2,3-triazole (Intermediate 25) and tert-butyl (tert-butoxycarbonyl)(7-(5-chloro-6-methylpyridin-3-yl)-8-methyl-[1,2,4]triazolo[1,5-α]pyridin-2-yl)carbamate (Intermediate 59). Purification via preparative Reverse Phase chromatography was performed with the following conditions: Gradient from 23% to 55% Solvent B in solvent A over 22 min, (solvent A=5/95 ACN/water with 10 mM ammonium acetate and solvent B=95/5 ACN/water with 10 mM ammonium acetate) Column: XBridge C18, 19 mm×200 mm, 5 μm particles; Flow Rate: 20 mL/min; Column Temperature: 25° C. Fraction collection was triggered by MS (ESI+). Fractions containing the desired product were combined and dried via centrifugal evaporation to yield the title compound. ¹H NMR (500 MHz, DMSO-d₆) δ 8.60 (d, J=2.0 Hz, 1H), 8.50 (d, J=6.8 Hz, 1H), 8.45 (s, 1H), 8.14 (d, J=2.0 Hz, 1H), 7.89 (br dd, J=8.0, 5.6 Hz, 2H), 7.32 (t, J=8.8 Hz, 2H), 6.90 (d, J=6.9 Hz, 1H), 6.70 (br t, J=12.3 Hz, 1H), 6.06 (s, 2H), 2.78 (s, 3H), 2.40 (s, 3H), 1.68 (br t, J=19.0 Hz, 3H). MS ESI m/z 479.1 (M+H)⁺.

### Example 68: (R)-7-(5-(2-(2,2-difluoro-1-(4-fluorophenyl)propyl)-2H-1,2,3-triazol-4-yl)-6-(trifluoromethyl)pyridin-3-yl)-[1,2,4]triazolo[1,5-α]pyridin-2-amine

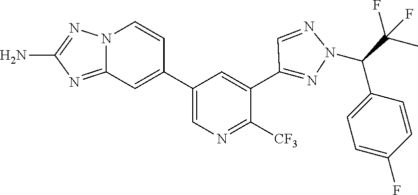 [SMILES: CC(F)(F)[C@@H](c1ccc(F)cc1)n1ncc(-c2cc(-c3ccn4nc(N)nc4c3)cnc2C(F)(F)F)n1]

The title compound was prepared in a similar manner as Example 26 utilizing tert-butyl (tert-butoxycarbonyl)(7-(4,4,5,5-tetramethyl-1,3,2-dioxaborolan-2-yl)-[1,2,4]triazolo[1,5-α]pyridin-2-yl)carbamate (Intermediate 15) and (R)-5-chloro-3-(2-(2,2-difluoro-1-(4-fluorophenyl)propyl)-2H-1,2,3-triazol-4-yl)-2-(trifluoromethyl)pyridine (Intermediate 60). Purification via preparative Reverse Phase chromatography was performed with the following conditions: Gradient from 30% to 61% Solvent B in solvent A over 22 min, (solvent A=5/95 ACN/water with 10 mM ammonium acetate and solvent B=95/5 ACN/water with 10 mM ammonium acetate) Column: XBridge C18, 19 mm×200 mm, 5 μm particles; Flow Rate: 20 mL/min; Column Temperature: 25° C. Fraction collection was triggered by MS (ESI+). Fractions containing the desired product were combined and dried via centrifugal evaporation to yield the title compound. ¹H NMR (500 MHz, DMSO-d₆) δ 9.29 (s, 1H), 8.73 (br d, J=7.1 Hz, 1H), 8.64 (s, 1H), 8.38 (s, 1H), 8.02 (s, 1H), 7.92-7.85 (m, 2H), 7.46 (dd, J=6.9, 1.8 Hz, 1H), 7.32 (t, J=8.9 Hz, 2H), 6.73 (br t, J=12.1 Hz, 1H), 6.17 (s, 2H), 1.68 (br t, J=18.9 Hz, 3H). MS ESI m/z 519.1 (M+H)⁺.

### Example 69: 7-(6-(2-(4-fluorobenzyl)-2H-1,2,3-triazol-4-yl)pyrazin-2-yl)-[1,2,4]triazolo[1,5-α]pyridin-2-amine

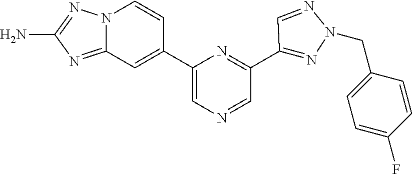 [SMILES: Nc1nc2cc(-c3cncc(-c4cnn(Cc5ccc(F)cc5)n4)n3)ccn2n1]

Step 1: To a solution of 4-bromo-2H-1,2,3-triazole (150 mg, 1.014 mmol) and 1-(bromomethyl)-4-fluorobenzene (230 mg, 1.217 mmol) in CH₂Cl₂ (4 mL) was added DIPEA (0.266 mL, 1.521 mmol). The mixture was stirred at rt for 3 days. The mixture was concentrated and purified via silica gel chromatography (12 g, hexanes-100% DCM) to give a major product 4-bromo-2-(4-fluorobenzyl)-2H-1,2,3-triazole (160 mg, 0.625 mmol, 61.6% yield). ¹H NMR (400 MHz, CHLOROFORM-d) δ 7.58 (s, 1H), 7.38-7.32 (m, 2H), 7.09-7.03 (m, 2H), 5.53 (s, 2H).

Step 2: A mixture of tert-butyl (tert-butoxycarbonyl)(7-(6-chloropyrazin-2-yl)-[1,2,4]triazolo[1,5-α]pyridin-2-yl)carbamate (115 mg, 0.257 mmol) (Intermediate 41), bispin (196 mg, 0.772 mmol), potassium acetate (76 mg, 0.772 mmol), PdOAc₂ (4.62 mg, 0.021 mmol), and tricyclohexylphosphine (10.10 mg, 0.036 mmol) in dioxane (1.5 mL) was stirred in a 100° C. oil bath for 1 h. To the reaction mixture was added 4-bromo-2-(4-fluorobenzyl)-2H-1,2,3-triazole (46.1 mg, 0.180 mmol) (Example 69A), 1,1′-Bis(di-tert-butylphosphino)ferrocene palladium dichloride (8.39 mg, 0.013 mmol), and tripotassium phosphate (0.386 mL, 0.772 mmol). The mixture was stirred at 70° C. for 1 h. Water was added and the mixture was extracted twice with EtOAc. The combined organic layers were concentrated and purified via silica gel chromatography (12 g, hexanes-100% EtOAc) to give tert-butyl (tert-butoxycarbonyl)(7-(6-(2-(4-fluorobenzyl)-2H-1,2,3-triazol-4-yl)pyrazin-2-yl)-[1,2,4]triazolo[1,5-α]pyridin-2-yl)carbamate (60 mg, 0.102 mmol, 39.7% yield). MS ESI m/z 588.1 (M+H)⁺.

Step 3: To a solution of tert-butyl (tert-butoxycarbonyl)(7-(6-(2-(4-fluorobenzyl)-2H-1,2,3-triazol-4-yl)pyrazin-2-yl)-[1,2,4]triazolo[1,5-α]pyridin-2-yl)carbamate (60 mg, 0.102 mmol) (Example 69B) in DCM (1.5 mL) was added TFA (1.0 mL, 12.98 mmol). The mixture was stirred at rt for 5 h and concentrated. The residue was suspended in DMSO. The solid was filtered and air dried under vacuum suction to give 7-(6-(2-(4-fluorobenzyl)-2H-1,2,3-triazol-4-yl)pyrazin-2-yl)-[1,2,4]triazolo[1,5-α]pyridin-2-amine (20 mg, 0.052 mmol, 50.6% yield). MS ESI m/z 387.9 (M+H)⁺.

### Example 70: (±)-2-(4-(6-(2-amino-[1,2,4]triazolo[1,5-α]pyridin-7-yl)pyrazin-2-yl)-2H-1,2,3-triazol-2-yl)-2-(4-fluorophenyl)ethan-1,1-d2-1-ol

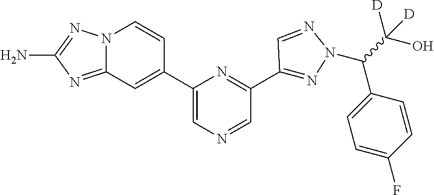 [SMILES: [2H]C([2H])(O)C(c1ccc(F)cc1)n1ncc(-c2cncc(-c3ccn4nc(N)nc4c3)n2)n1]

Step 1: [1,1′-Bis(diphenylphosphino)ferrocene]dichloropalladium(II), complex with dichloromethane (58.5 mg, 0.072 mmol), Bis(pinacolato)diboron (182 mg, 0.716 mmol), methyl 2-(4-bromo-2H-1,2,3-triazol-2-yl)-2-(4-fluorophenyl)acetate (171 mg, 0.525 mmol) (Intermediate 61), and potassium acetate (141 mg, 1.433 mmol) were added to a vial with dioxane (2 mL). The reaction was bubbled with nitrogen for 1 minute and then heated to 80° C. for 2 hours. LCMS showed the reaction was complete. The reaction vial was brought to room temperature and 7-(6-chloropyrazin-2-yl)-2-(2,5-dimethyl-1H-pyrrol-1-yl)-[1,2,4]triazolo[1,5-α]pyridine (171 mg, 0.525 mmol) (made in analogous fashion to Intermediate 18A), potassium phosphate tribasic (0.478 mL, 1.433 mmol) and [1,1′-Bis(diphenylphosphino)ferrocene]dichloropalladium(II), complex with dichloromethane (58.5 mg, 0.072 mmol) were added. The reaction was heated at 80° C. overnight. The reaction was brought to room temp, filtered, and concentrated. MS ESI m/z 524.1 (M+H)⁺.

Step 3: In a 8 mL vial was added methyl 2-(4-(6-(2-(2,5-dimethyl-1H-pyrrol-1-yl)-[1,2,4]triazolo[1,5-α]pyridin-7-yl)pyrazin-2-yl)-2H-1,2,3-triazol-2-yl)-2-(4-fluorophenyl)acetate (50 mg, 0.096 mmol) (Example 70B) in MeOH (2 mL), then sodium borodeuteride (15.99 mg, 0.382 mmol) was added. After 1 hour, LCMS revealed the desired product had been generated. The crude was directly taken to the next step. MS ESI m/z 498.0 (M+H)⁺.

Step 4: To the crude solution of 2-(4-(6-(2-(2,5-dimethyl-1H-pyrrol-1-yl)-[1,2,4]triazolo[1,5-α]pyridin-7-yl)pyrazin-2-yl)-2H-1,2,3-triazol-2-yl)-2-(4-fluorophenyl)ethan-1,1-d2-1-ol (50 mg, 0.100 mmol) (Example 70C) was added hydroxylamine hydrochloride (0.402 mL, 2.010 mmol) in ethanol (2 mL). The reaction was heated to 100° C. under microwave for 10 h. LCMS revealed the desired product. The crude racemate was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 8-37% B over 25 minutes; Flow Rate: 20 mL/min; Column Temperature: 25° C. Fraction collection was triggered by MS and UV signals. Fractions containing the desired product were combined and dried via centrifugal evaporation to give (±)-2-(4-(6-(2-amino-[1,2,4]triazolo[1,5-α]pyridin-7-yl)pyrazin-2-yl)-2H-1,2,3-triazol-2-yl)-2-(4-fluorophenyl)ethan-1,1-d2-1-ol. ¹H NMR (500 MHz, DMSO-d₆) δ 9.46-9.40 (m, 1H), 9.20 (s, 1H), 8.72 (br d, J=7.1 Hz, 1H), 8.66 (s, 1H), 8.31 (br s, 1H), 7.80 (d, J=6.9 Hz, 1H), 7.53 (t, J=6.6 Hz, 2H), 7.27-7.19 (m, 2H), 6.18 (s, 2H), 5.96 (s, 1H). MS ESI m/z 419.8 (M+H)⁺.

### Example 71: (Ent 1)-7-(6-(2-(1-(4-fluorophenyl)-2,2-dimethylpropyl)-2H-1,2,3-triazol-4-yl)pyrazin-2-yl)-[1,2,4]triazolo[1,5-α]pyridin-2-amine

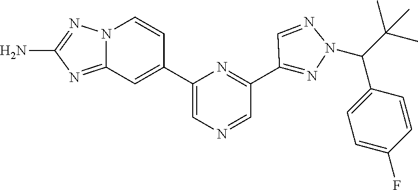 [SMILES: CC(C)(C)C(c1ccc(F)cc1)n1ncc(-c2cncc(-c3ccn4nc(N)nc4c3)n2)n1]

Step 1: [1,1′-Bis(diphenylphosphino)ferrocene]dichloropalladium(II), complex with dichloromethane (27.4 mg, 0.034 mmol), Bis(pinacolato)diboron (85 mg, 0.336 mmol), tert-butyl (tert-butoxycarbonyl)(7-(6-chloropyrazin-2-yl)-[1,2,4]triazolo[1,5-α]pyridin-2-yl)carbamate (100 mg, 0.224 mmol) (Intermediate 17A), and potassium acetate (65.9 mg, 0.671 mmol) were added to a vial with dioxane (2 mL). The reaction was bubbled with nitrogen for 1 minute and then heated to 80° C. for 2 hours. LCMS showed the reaction was complete. The reaction vial was brought to room temp and 4-bromo-2-(1-(4-fluorophenyl)-2,2-dimethylpropyl)-2H-1,2,3-triazole (105 mg, 0.336 mmol) (Intermediate 62), potassium phosphate tribasic (0.224 mL, 0.671 mmol) and [1,1′-Bis(diphenylphosphino)ferrocene]dichloropalladium(II), complex with dichloromethane (27.4 mg, 0.034 mmol) were added. The reaction mixture was heated at 80° C. overnight. The reaction was brought to room temp, filtered, and concentrated.

Step 2: To the material from the previous step was added TFA, which was heated at 50° C. for 10 mins. The reaction was concentrated. The crude racemate prepared in the final synthetic step was purified via prep HPLC. Fractions containing the desired product were combined and dried via centrifugal evaporation to give (±)-7-(6-(2-(1-(4-fluorophenyl)-2,2-dimethylpropyl)-2H-1,2,3-triazol-4-yl)pyrazin-2-yl)-[1,2,4]triazolo[1,5-α]pyridin-2-amine. This racemic material was then further purified via preparative SFC with the following conditions: Column: Chiralpak AD-H, 30 mm×250 mm, 5 m particles; Flow Rate: 75.00 mL/min; Mobile Phase A: supercritical CO₂; Mobile Phase B: MeOH with 0.1% NH₄O H; Elution gradient: isocratic 15% B over 80 minutes; Column Temperature: 40° C. Fraction collection was triggered by UV (235 nm). Fractions containing the first isomer to elute from the chiral column were combined and dried via centrifugal evaporation to provide (Ent 1)-7-(6-(2-(1-(4-fluorophenyl)-2,2-dimethylpropyl)-2H-1,2,3-triazol-4-yl)pyrazin-2-yl)-[1,2,4]triazolo[1,5-α]pyridin-2-amine

(12.7 mg, 32%) and the second enantiomer was likewise obtained. ¹H NMR (500 MHz, DMSO-d₆) δ 9.40 (s, 1H), 9.20 (s, 1H), 8.70 (d, J=7.1 Hz, 1H), 8.64 (s, 1H), 8.28 (s, 1H), 7.82-7.75 (m, 3H), 7.23 (br t, J=8.8 Hz, 2H), 6.15 (s, 2H), 5.87 (s, 1H), 1.02 (s, 9H). MS ESI m/z 444.1 (M+H)⁺.

### Example 72: (Ent 1)-7-(6-(2-(1-(4-fluorophenyl)-2,2-dimethylpropyl)-2H-1,2,3-triazol-4-yl)pyridin-2-yl)-[1,2,4]triazolo[1,5-α]pyridin-2-amine

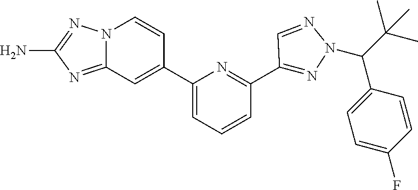 [SMILES: CC(C)(C)C(c1ccc(F)cc1)n1ncc(-c2cccc(-c3ccn4nc(N)nc4c3)n2)n1]

Step 1: In a 40 mL Chemglass pressure vial were combined tert-butyl (tert-butoxycarbonyl)(7-(4,4,5,5-tetramethyl-1,3,2-dioxaborolan-2-yl)-[1,2,4]triazolo[1,5-α]pyridin-2-yl)carbamate (500 mg, 1.086 mmol) (Intermediate 15) and 2-chloro-6-iodopyridine (260 mg, 1.086 mmol) in 1,4-dioxane (10 mL). To the mixture was added potassium phosphate tribasic, 2M aqueous solution (1.629 mL, 3.26 mmol). The mixture was sparged with nitrogen for 5 min. Then, dichloro[1,1′-bis(di-t-butylphosphino)ferrocene]palladium(II), 99% (42.5 mg, 0.065 mmol) was added, and the mixture was sparged with a stream of nitrogen with stirring for 5 additional min. The vial was then capped and the mixture was heated to 65° C. LCMS taken after heating 3.5 h showed a major product peak with correct m/z for desired product. The crude mixture was diluted with 1:1 DCM:EtOAc (20 mL) and the resulting black susension was filtered through Celite and rinsed with 1:1 EtOAc:DCM. Concentration in vacuo gave a black oil. The residue was dissolved in minimum DCM and loaded atop a DCM-preequilibrated Isco 40 g silica cartridge. Elution gradient 100% DCM to 100% EtOAc over 15 column volumes, hold 100% EtOAc for 6 CVs.

The product eluted as a fairly sharp peak followed by a small broader peak which was the mono-Boc desired material m/z by LCMS. Fractions containing both bis and mono N-Boc products were combined and concentrated in vacuo to an off-white powder. LCMS of the combined purified material showed >95% bis-N-Boc desired product, with a small amount of mono-N-Boc material also present, and a couple of very small impurity peaks. Isolated tert-butyl (tert-butoxycarbonyl)(7-(6-chloropyridin-2-yl)-[1,2,4]triazolo[1,5-α]pyridin-2-yl)carbamate (448 mg, 93% yield) as an off-white powder. MS ESI m/z 445.95/447.9 (M+H)⁺.

Step 2: The steps of Example 71 were followed in a similar way using the product of the previous step as starting material. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 37-67% B over 20 minutes; Flow Rate: 20 mL/min; Column Temperature: 25° C. Fraction collection was triggered by MS and UV signals. Fractions containing the desired product were combined and dried via centrifugal evaporation to give (±)-7-(6-(2-(1-(4-fluorophenyl)-2,2-dimethylpropyl)-2H-1,2,3-triazol-4-yl)pyridin-2-yl)-[1,2,4]triazolo[1,5-α]pyridin-2-amine (16.7 mg, 18% yield). This racemic material was then further purified via preparative SFC with the following conditions: Column: Whelk-01 (R,R) 21 mm×250 mm, 5 m particles; Flow Rate: 75.00 mL/min; Column Temperature: 40° C. Fraction collection was triggered by UV (240 nm). Mobile Phase A: supercritical CO₂; Mobile Phase B: i-PrOH with 0.1% NH₄OH; Elution gradient: isocratic 20% B over 15 minutes. Fractions containing the first isomer eluting from the chiral column were combined and dried via centrifugal evaporation to provide (Ent 1)-7-(6-(2-(1-(4-fluorophenyl)-2,2-dimethylpropyl)-2H-1,2,3-triazol-4-yl)pyridin-2-yl)-[1,2,4]triazolo[1,5-α]pyridin-2-amine (3.6 mg, 4% yield), along with the other enantiomer. ¹H NMR (500 MHz, DMSO-d₆) δ 8.64 (d, J=7.0 Hz, 1H), 8.52 (s, 1H), 8.18 (s, 1H), 8.15 (d, J=7.9 Hz, 1H), 8.05 (t, J=7.8 Hz, 1H), 7.99 (d, J=7.8 Hz, 1H), 7.80-7.73 (m, 3H), 7.22 (t, J=8.7 Hz, 2H), 6.09 (s, 2H), 5.82 (s, 1H), 1.01 (s, 9H). MS ESI m/z 443.4 (M+H)⁺.

### Example 73: (Ent 1)-4-(1-(4-(6-(2-amino-[1,2,4]triazolo[1,5-α]pyridin-7-yl)pyrazin-2-yl)-2H-1,2,3-triazol-2-yl)ethyl)benzonitrile

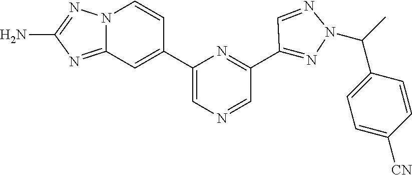 [SMILES: CC(c1ccc(C#N)cc1)n1ncc(-c2cncc(-c3ccn4nc(N)nc4c3)n2)n1]

The title compound was obtained in a similar manner as Example 2, utilizing (±)-4-(1-(4-bromo-2H-1,2,3-triazol-2-yl)ethyl)benzonitrile (Intermediate 63) and 7-(6-chloropyrazin-2-yl)-2-(2,5-dimethyl-1H-pyrrol-1-yl)-[1,2,4]triazolo[1,5-α]pyridine as a starting materials. The crude racemate prepared in the final synthetic step was purified via preparative LC/MS with the following conditions: Column: Waters XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-40% B over 30 minutes; Flow Rate: 20 mL/min; Column Temperature: 25° C. Fraction collection was triggered by MS and UV signals. Fractions containing the desired product were combined and dried via centrifugal evaporation to give (±)-4-(1-(4-(6-(2-amino-[1,2,4]triazolo[1,5-α]pyridin-7-yl)pyrazin-2-yl)-2H-1,2,3-triazol-2-yl)ethyl)benzonitrile (18.4 mg, 44% yield). This racemic material was then further purified via preparative SFC with the following conditions: Column: Whelk-01 (R,R) 21 mm×250 mm, 5 m particles; Flow Rate: 75.00 mL/min; Column Temperature: 40° C. Fraction collection was triggered by UV (240 nm). Mobile Phase A: supercritical CO₂; Mobile Phase B: MeOH with 0.1% NH₄O H; Elution gradient: isocratic 30% B over 18 minutes. Fractions containing the first isomer eluting from the chiral column were combined and dried via centrifugal evaporation to provide (Ent 1)-4-(1-(4-(6-(2-amino-[1,2,4]triazolo[1,5-α]pyridin-7-yl)pyrazin-2-yl)-2H-1,2,3-triazol-2-yl)ethyl)benzonitrile (6.7 mg, 16% yield), along with the other enantiomer. ¹H NMR (500 MHz, DMSO-d₆) δ 9.45-9.41 (m, 1H), 9.17 (s, 1H), 8.74-8.67 (m, 2H), 8.33-8.28 (m, 1H), 7.87 (br d, J=7.5 Hz, 2H), 7.79 (d, J=7.2 Hz, 1H), 7.54 (d, J=8.5 Hz, 2H), 6.30-6.23 (q, J=7.0 Hz, 1H), 6.20-6.15 (m, 2H), 2.00 (d, J=7.0 Hz, 3H). MS ESI m/z 409.2 (M+H)⁺.

Numerous modifications and variations of the present invention are possible in light of the above teachings. It is therefore to be understood that within the scope of the appended claims, the invention may be practiced otherwise than as specifically described herein.

What is claimed is:

1. A compound having formula (I), or salt thereof, 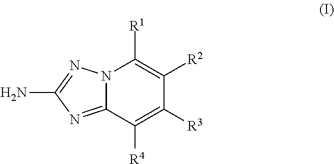
wherein, independently for each occurrence:
R¹ is —H, -T, —F, —OH, C₁₋₃ alkyl, or C₁₋₃ alkoxy;
one of R² or R³ is —H, -T, —F, —OCH₃, or C₁₋₃ alkyl; and the other is 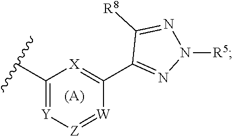
W and Y are —N═ or —C(R¹⁰)═;
Z is —N═ or —C(R¹¹)═;
X is —N═ or —C(R⁹)═;
wherein ring A contains 0-2 nitrogen atoms;
R⁴ is —H, —F, —OH, C₁₋₃ alkyl, or C₁₋₃ alkoxy;
R⁵ is, —(CR^{a}R^{b})—(CR^{c}H)ₙ—R^{5a};
n, at each occurrence, is independently 0 or 1;
R^{a} is —H, -D, F, C₁₋₄ alkyl, C₃₋₆ cycloalkyl, C₃₋₆ cycloalkyl substituted with 0-2 of F or C₁₋₃ alkyl, C₁₋₄ deuteroalkyl, C₁₋₄ haloalkyl, C₁₋₄ hydroxyalkyl, C₂₋₄ alkoxyalkyl, or oxetanyl;
R is —H, -D, or C₁₋₃ alkyl;
alternatively, R^{a} and R^{b}, along with the carbon to which they are attached, join to form a C₃₋₆ cycloalkyl substituted with 0-2 F;
R^{c} is —H, C₁₋₃ alkyl, or —OH;
R^{5a} is C₆₋₁₀ carbocycle or a 5-6 membered heterocycle, the carbocycle or heterocycle being substituted with 0-3 R⁶;
R⁶ is H, -T, —F, ¹⁸F, —Cl, —I, —CN, C₁₋₃ alkyl, C₁₋₃ alkoxy, nitro, C₁₋₃ haloalkyl, or C₁₋₃ haloalkoxy;
R⁸ is —H, -T, -halo, —CN, C₁₋₃ alkyl, C₁₋₃ alkoxy, or C₁₋₃ haloalkyl;
R⁹ is —H, -halo, C₁₋₃ alkyl, C₁₋₃ alkoxy, or C₁₋₃ haloalkyl;
R¹⁰ is —H, -T, -halo, C₁₋₃ alkyl, C₁₋₃ alkoxy, or C₁₋₃ haloalkyl; and
R¹ is —H, —CN, —C(O)NH₂, —C(O)OH, -halo, C₁₋₃ alkyl, C₁₋₃ alkoxy, or C₁₋₃ haloalkyl.

2. The compound of claim 1, or salt thereof, wherein
X and Z are N;
Y is C(R¹⁰); and
W is C(R¹⁰).

3. The compound of claim 1, or salt thereof, wherein
X is N;
Y is C(R¹⁰);
Z is C(R¹¹); and
W is C(R¹⁰).

4. The compound of claim 1, or salt thereof, wherein
X is C(R⁹);
Y is C(R¹⁰);
Z is C(R¹¹); and
W is C(R¹⁰).

5. The compound of claim 2, or salt thereof, wherein
R² is H; and
R³ is

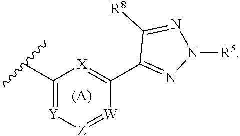

6. The compound of claim 5, or salt thereof, wherein
R^{5a} is phenyl, pyridyl, oxanyl, tetrahydronaphthalene, or dihydroindene, any of which are substituted with 0-2 R⁶; and
R⁶ is H, —F, —Cl, —CN, C₁₋₃ alkyl, C₁₋₃ alkoxy, or C₁₋₃ haloalkyl.

7. The compound of claim 6, or salt thereof, wherein
R⁵ is —(CR^{a}R^{b})—R^{5a}; and
R^{a} is —H, -D, C₁₋₄ alkyl, C₁₋₃ alkoxy, or C₁₋₂ haloalkyl.

8. The compound of claim 7, or salt thereof, wherein
R⁵ is —(CR^{a}R^{b})—R^{5a}.

9. The compound of claim 8, or salt thereof, wherein
R⁸ is H or CH₃;
R⁹ is H or F;
R¹⁰ is H, F, Cl, CH₃, OCH₃, or CF₃; and;
R¹¹ is H, F, Cl, or CF₃.

10. The compound of claim 9, or salt thereof, wherein
R⁴ is H, —F, or CH₃;
R⁸ is H or CH₃;
R⁹ is H or F;
R¹⁰ is H, CH₃, or CF₃; and
R¹¹ is H.

11. The compound of claim 10, or salt thereof, wherein
R⁵ is —(CR^{a}R^{b})—R^{5a};
R^{5a} is phenyl or pyridyl, any of which are substituted with 0-2 R⁶;
R⁶ is H, —F, —Cl, —CN, CH₃, or OCH₃.

12. The compound of claim 11, or salt thereof, wherein
R⁵ is

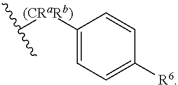

13. The compound of claim 12, or salt thereof, wherein
R⁵ is

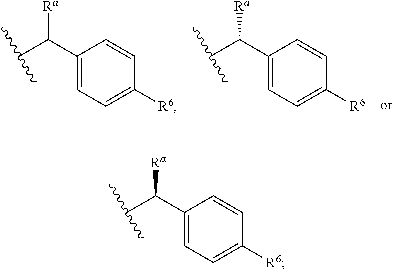

14. The compound of claim 1, or a salt thereof, wherein the compound is selected from the Examples.

15. A pharmaceutical composition comprising one or more compounds of claim 1, or pharmaceutically acceptable salt thereof, and a pharmaceutically acceptable carrier.

16. A method of inhibiting casein kinase RIPK1 activity in a patient, comprising administering to the patient in need thereof, a therapeutically effective amount of one or more compounds of claim 1.

17. A method for treating a disease comprising the administration to a subject in need thereof a therapeutically effective amount of at least one compound of claim 1, wherein the disease is selected from inflammatory bowel disease, ulcerative colitis, Crohn's disease, psoriasis, rheumatoid arthritis (RA), NASH, and heart failure.

18. A method for treating a disease comprising the administration to a subject in need thereof a therapeutically effective amount of at least one compound of claim 1, wherein the disease is selected from multiple sclerosis, amyotrophic lateral sclerosis, or Alzheimer's.

19. A method for treating a disease comprising the administration to a subject in need thereof a therapeutically effective amount of at least one compound of claim 1, wherein the disease is selected from multiple sclerosis.

20. A method for treating a disease comprising the administration to a subject in need thereof a therapeutically effective amount of at least one compound of claim 1, wherein the disease is selected from amyotrophic lateral sclerosis.

21. A method for treating a disease comprising the administration to a subject in need thereof a therapeutically effective amount of at least one compound of claim 1, wherein the disease is selected from Alzheimer's.
